(19) United States
(12) Patent Application Publication
(10) Pub. No.: US 2023/0149035 A1
(43) Pub. Date: May 18, 2023

(54) DEVICES AND METHODS FOR ASPIRATION OF THROMBUS

(71) Applicant: Elixir Medical Corporation, Milpitas (US)

(72) Inventors: Motasim Sirhan, Los Altos, CA (US); Vinayak Bhat, Cupertino, CA (US); Benjamyn Serna, Gilroy, CA (US); Brett Cryer, Pleasanton, CA (US); Kim Nguyen, Union City, CA (US); John Yan, Los Gatos, CA (US)

(73) Assignee: Elixir Medical Corporation, Milpitas (US)

(21) Appl. No.: 18/156,921

(22) Filed: Jan. 19, 2023

Related U.S. Application Data
(63) Continuation of application No. PCT/US21/42398, filed on Jul. 20, 2021.
(60) Provisional application No. 63/054,189, filed on Jul. 20, 2020.

(51) Int. Cl. A61B 17/221; A61M 1/00; A61M 25/00

(52) U.S. Cl. CPC A61B 17/221; A61M 1/77; A61M 25/005; A61B 2017/22079

(57) ABSTRACT

Clot aspiration systems intended for removing clot from a blood vessel include an aspiration assembly which will have two or more of the following components: an aspiration catheter, an inner catheter, an intermediate catheter, and an outer catheter, the latter typically being a guiding or other sheath. A transition structure is coupled to a distal end of the aspiration assembly to cover or fill an open distal end of one or more of the components of the aspiration assembly. The transition structure may be configured to facilitate introduction of the aspiration catheter into the patient's vasculature and/or advancement of the aspiration catheter through the vasculature to a target site, such as a cerebral target site which may be occluded with clot, thrombus, or other occlusive material.

### CROSS-REFERENCE TO RELATED APPLICATION

This application is a continuation of PCT Application No. PCT/US21/42398, (Attorney Docket No. 32016-727.601), filed Jul. 20, 2021, which claims the benefit of U.S. Provisional No. 63/054,189, (Attorney Docket No. 32016-727.101), filed Jul. 20, 2020, the entire content of which is incorporated herein by reference.

### BACKGROUND OF THE INVENTION

### 1. Field of the Invention

Every year millions of people worldwide suffer strokes caused by blood clots in the brain. Even when not fatal, these clots can lead to severe and permanent disability. Until recently the only means of treating patients presenting with symptoms of an occlusive stroke was pharmaceutical, in which tissue plasminogen activator (tPA) is given to the patient intravenously to dissolve the clot and restore blood flow in the brain. However, since a vascular thrombus (clot) tends to become more fibrous and/or firm up with time, the efficacy window for tPA is just a few hours after the clot first forms. Considering the time involved with recognizing an individual may be having a stroke, transporting them to the hospital, and performing the diagnosis and applying treatment, many patients' clots are too mature to respond to tPA, such that perhaps two thirds of stroke victims were not being significantly helped by pharmaceutical treatment.

Advances in medical technology led to the development of various mechanical thrombectomy techniques, in which the blood clot is physically extracted from the brain. Mechanical thrombectomy has the major advantage over pharmaceutical treatment in that it can remove clots many hours after the efficacy window for pharmaceutical treatment has passed and still provide benefit to the patient.

There are two primary approaches to mechanical thrombectomy, which may be used independently or in combination with each other depending on patient characteristics and physician preference. The first is to use a catheter to apply a vacuum to the clot, in a technique known as direct aspiration. The second is to use a stent retriever to snare and physically pull out the thrombus, optionally in combination with applying a vacuum to the clot through a separate aspiration catheter.

Both mechanical thrombectomy methods have their limitations. While stent retrievers are small and flexible enough to access most clots, their ability to snag and remove a clot varies. In some cases only a portion of the clot can be removed, and debris from the procedure can be released downstream causing secondary occlusions. Stent retrievers can also induce trauma to the vessel as they are dragged proximal pulling the clot with them. The struts of the retriever scrape the endothelium off the vessel walls, creating areas more prone to generating future occlusions. Procedure time is also an issue with stent retrievers, since in addition to delivery and extraction time they typically require a significant time to settle into and secure the clot before the first removal attempt can be made. In an environment of blood-starved brain tissue, the difference in procedure times is very clinically significant on successful outcomes.

The effectiveness of aspiration catheters depends on the ability of the catheter to vacuum the clot through the aspiration lumen of the catheter. Current aspiration catheters are limited in diameter by the size of the introducer sheath and guiding catheter used by the physician to introduce the aspiration catheter into the anatomy. Since most clots tend to be significantly larger than the aspiration catheter size, the small size of conventional aspiration catheters represents a challenge to successful aspiration, due to their inability to fully aspirate the clot on the first vacuum attempt and in the absence of breaking or fragmenting the clot. Current aspiration catheters are also bulky which limits the ability of such catheters to navigate the tortuous anatomy of the brain to reach the common target occluded segments. Such catheters are even less successful in reaching the more distal clots due to the bulky size of these catheters and the very tortuous neurovascular anatomy. Smaller aspiration catheters designed specifically to access the more distal clots often fail to extract the clot due to lack of sufficient suction force at the tip due to the small tip area, and/or because the aspiration lumen of the smaller catheters is too narrow to absorb the clot. Therefore stent retrievers are utilized more often for such distal occluded vessels, alone or in combination with aspiration catheters. Despite the combination use of a stent retriever and aspiration catheters together, the failure to remove the clot completely or partially is still occurs in a significant number of patients and the procedure length of time is extended, potentially compromising the patient brain cells in that occluded area.

What is needed is a device that is capable of reaching clots in the brain in both the proximal and distal neuro anatomy, a device able to remove clots without fragmenting or without substantially fragmenting the clot, a device able to remove clots without causing secondary occlusions, a device able to remove clots reliably without requiring the use of a stent retriever or other supplementary device, a device able to reach the occlusion and retrieve the clots quickly, a device that does not scrape or otherwise induce trauma to the vessel wall at any point during the procedure, a device that is successful in retrieving the clot during the first aspiration attempt, and a device that requires less vacuum pressure to retrieve a clot. The present invention will address at least some of these needs.

### 2. Listing of Background References

Relevant patents and publications include WO1995/31149; US 2008/0086110; U.S. Pat. Nos. 5,403,334. 10,231,751; 10,500,047; US2021153883; U.S. Ser. No. 10/792,056; US2017274180; and US2016220741 WO1995/31149; US 2008/0086110; and U.S. Pat. No. 5,403,334. The disclosure of this application relates to the disclosures of the following commonly owned applications: WO2021/016213; WO2019/033121; WO2017/200956; and U.S. Provisional Application 63/184,719, filed on May 5, 2021, the full disclosures of which are incorporated herein by reference.

### SUMMARY OF THE INVENTION

In a first aspect, the present invention provides a clot aspiration system for aspirating clot from a blood vessel. The clot aspiration system comprises an aspiration assembly which includes at least an aspiration catheter and an inner catheter. The aspiration assembly has a proximal end and a distal end, and the aspiration catheter has at least a proximal end, an open distal end, and an aspiration lumen therebetween. The inner catheter has a proximal end, a distal end, and a guide wire lumen therebetween, and the inner catheter may be slidably received in the aspiration lumen of the aspiration catheter. A transition structure may be coupled to a distal segment of the inner catheter and cover or fill the open distal end of the aspiration catheter when the distal end of the inner catheter may be positioned at or near the distal end of the aspiration catheter. The transition structure provides a number of advantages including facilitating introduction of the aspiration catheter into the patient's vasculature as well as advancement of the aspiration catheter through the vasculature to a target site, such as a cerebral target site which may be occluded with clot, thrombus, or other occlusive material. In a preferred aspect, at least a distal portion of the scaffold is configured to be radially expandable from a delivery configuration to an extraction configuration. In another aspect or examples, the distal portion of the scaffold is delivered in an extraction configuration.

The phrase “aspiration assembly” as used herein and in the claims refers to any structure, combination, construction, or other assembly of any two or more of the following components of the clot aspiration systems of the present invention: aspiration catheters; inner catheters; intermediate catheters; and outer catheters such as sheaths, guiding sheath, and the like, as well as transition structures which are located at or near the distal end of the aspiration assemblies. While the individual components of the aspiration assemblies will often (although not always) be formed separately, the aspiration assemblies will typically be introduced into a patient's vasculature and/or through a blood vessel when assembled, coupled, connected, joined, attached, introduced, and/or advanced at least partially together.

The transition structure may have a variety of forms to the inner catheter and aspiration assembly in a variety of ways. For example, the transition structure may be coupled to a distal tip of the aspiration catheter, often a location within 10 cm of a distal tip of the distal segment, preferably within 5 cm of the distal tip. A proximal portion of the transition structure may be coupled to the distal segment of the inner catheter, and a distal portion of the transition structure may be detachably coupled to the distal end of the aspiration catheter. The transition structure may be coupled to the distal segment by any one or more of attaching, adhering, soldering, overlaying, wedging, stretching over, suturing, gluing, crimped, constraining, heat bonding, fusing, molding, and extruding. Alternatively, the transition structure may be an integral component of the inner catheter.

The transition structure may be formed from one or a combination of a wide variety of materials, sometimes being formed at least partly from a material which may be the same as a material used to form the inner catheter. In other instances, the transition structure may be formed at least partly from a material which may be different than a material used to form the inner catheter.

An annular gap may be typically formed between an outer surface of the inner catheter and an inner surface of the aspiration lumen at a distal end of the clot aspiration system. In this way, the transition structure may be able to cover or fill the annular gap when the distal end of the inner catheter may be positioned at or near the distal end of the aspiration catheter by a preselected distance. In some instances, the distal end of the inner catheter protrudes beyond the distal end of the aspiration catheter by a preselected distance and the transition structure covers the open distal end of the aspiration catheter, the inner catheter may be flush with the distal end of the aspiration catheter and the transition structure fills the open distal end of the aspiration catheter. The annular gap may have a mean width in a range from 0.025 mm to 2 mm, from 0.05 mm to 1 mm, or from 0.1 mm 1.25 mm. The annular gap may extend from the distal end over a length in a range from 1 cm to 110 cm, 1 cm to 50 cm, or 1 cm to 25 cm.

The aspiration catheter and the inner catheter may be arranged coaxially about an axis of the guide wire lumen along at least a distal segment of the aspiration catheter and/or the inner catheter, where the distal segment may have a length ranging from 1 cm to 40 cm, preferably ranges from 1 cm to 25 cm.

The inner catheter may comprise a push tube or rod proximal to said distal segment which may be typically configured to be coupled to the proximal end of the distal segment of the inner catheter in order to advance and/or withdraw the inner catheter through the aspiration catheter lumen.

An outer surface of the inner catheter closely may conform to an inner surface of the aspiration lumen of the aspiration, over all or at least a portion of, typically over a majority of, the length of the aspiration lumen, leaving a minimal or no gap over at least a partial length of the clot aspiration system. Such examples with a minimal gap can enhance stiffness and improve pushability of the aspiration assembly.

In many examples, the aspiration assembly further includes a sheath or other outer catheter having a proximal end, a distal end, and a central passage therethrough and being slidably disposed over an outer surface of the aspiration catheter. Typically, the sheath or other outer catheter, the aspiration catheter, and the inner catheter are arranged coaxially about an axis of the guide wire lumen over at least a portion of their lengths. Advantageously, the transition structure may cover or fill an open end of the sheath or other outer catheter (in addition to the open and of the aspiration catheter) when the distal end of the inner catheter may be positioned at or near the distal end of the aspiration assembly.

The transition structure may have any one of a variety of shapes, forms, designs, and constructions. In many instances, the transition structure will be tapered to lower a pushing force necessary to advance the clot aspiration system into or through the blood vessel. Such a tapered structure may be also advantageous in lowering the pushing force necessary to advance the aspiration assembly into and through the patient's vasculature. In other instances, however, the transition structure may have a blunt distal tip or end.

In specific examples, the transition structure may be configured to form a distal tip at the distal end of the aspiration assembly when the inner catheter may be disposed in the aspiration lumen of the aspiration catheter. In such instances, the distal tip may be configured to be retracted proximally to leave the distal end of the aspiration catheter open when the inner catheter may be retracted. Further optionally, a proximal end of the transition structure may be shaped to conformed to the open distal end of the aspiration catheter, a proximal end of the transition structure may be larger than the open distal end of the aspiration catheter and covers the open distal end of the aspiration catheter when the inner catheter may be in the aspiration lumen, and/or the proximal end of the transition may be smaller or the same as the open distal end of the aspiration catheter and fills the open distal end to at least partially seal the aspiration lumen. In further examples, a proximal end of the transition structure may be detachably or fixedly coupled to the inner catheter.

In many examples, the inner catheter may be configured to be advanced to displace the transition structure to uncover the distal end of the aspiration catheter. For example, the transition structure may be configured to be retracted through the aspiration lumen of the aspiration catheter by pulling proximally on the inner catheter. In a particular example, the transition structure comprises a collapsible shell having a proximal end which overlaps an outer surface of the aspiration assembly to close the distal end of the aspiration assembly during advancement of the aspiration assembly through the blood vessel. The collapsible shell typically has a conical or bullet-shape profile to facilitate entry into passage through the patient's vasculature. Such conical, bullet shaped, and other transition structures may be configured to additionally cover (evert or fold over) a distal length of the aspiration catheter outer surface over a distance typically in a range from 1 mm to 10 cm.

In other examples, the transition structure may comprise an inflatable occlusion member configured to fill the open distal end of the aspiration catheter when inflated. Such inflatable occlusion members will typically fill or cover the distal end of the aspiration assembly when inflated.

Transition structures may have a variety of shapes in the configured to engage a distal end or tip of the aspiration catheter and/or outer catheter or sheath and a variety of ways. For example, the outer surface of an inflatable or other transition member may be configured with a step or shoulder forming a nest which sealingly engages (engages and seals against) the distal tip of the aspiration catheter.

The aspiration catheter may have dimensions and be formed from materials suitable for any desired vascular intervention, particularly those used for catheters accessing the cerebral vasculature for clot and thrombus aspiration. Exemplary aspiration catheters may have an inner diameter in a range from 1 mm to 3.25 mm, preferably ranging from 1.5 mm to 2.35 mm. The distal end inner diameter of the aspiration catheter may range from 1.5 mm to 30 mm, preferably from 2 mm to 5 mm while the distal end outer diameter ranges from 1.55 mm to 30.05 mm, preferably from 2.05 mm to 5.05 mm. In such instances, the inner catheter may have an outer diameter in a range from 0.25 mm to 2 mm, preferably ranging from 0.5 mm to 1.54 mm. The preselected distance between the distal end of the inner catheter and the distal end of the aspiration catheter may be in a range from 5 mm to 100 mm, preferably ranging from 30.5 mm and 100 mm. The distal end of the inner catheter may have an inner diameter ranging from 0.5 mm to 25 mm, preferably from 1 mm to 2.5 mm and an outer diameter ranging from 0.55 mm to 3 mm, preferably from 1.05 mm to 3 mm.

In still other examples, the inner catheter may have the same (constant) inner diameter and/or outer diameter along the length of the inner catheter. In other instances, the inner catheter may have a variable inner diameter and/or outer diameter along the length of the inner catheter. In particular examples, the inner catheter may comprise a structure similar to a blood vessel dilator catheter.

In many examples, the aspiration catheter may be slidably received within the central passage of the outer catheter so that the outer catheter can axially move along the length or a length segment of the aspiration catheter. The outer catheter typically comprises a sheath such as an access sheath configured to seal in a vascular access penetration, or a guiding sheath, or a catheter of the type typically configured to access the cerebral vasculature. Depending on the intended use, the sheath length may be shorter than a length of the aspiration catheter length. For example, the sheath may have a working length which ranges from 1 cm to 110 cm, preferably from 10 cm to 100 cm, more preferably from 10 cm to 90 cm. In many instances, the outer catheter or sheath may be configured to be introduced into the blood vessel with the aspiration catheter.

In some examples, the transition structure may comprise a solid spherical or ovoidal plug that fills the annular gap and provides a rounded surface projecting distally from the distal end of the aspiration catheter when the distal end of the inner catheter may be near the distal end of the aspiration catheter by the preselected distance.

In some examples, the transition structure may comprise a solid spherical or ovoidal plug that fills the annular gap and has a shoulder that covers an exposed distal surface of the aspiration catheter when the distal end of the inner member protrudes beyond the distal end of the aspiration catheter by the preselected distance, said plug having a rounded surface projecting distally from the distal end of the aspiration catheter.

In some examples, the transition structure comprises a conical cap that covers the annular gap when the distal end of the inner member protrudes beyond the distal end of the aspiration catheter by the preselected distance.

In some examples, the transition structure may comprise an inflated balloon that fills the annular gap when the distal end of the inner member protrudes beyond the distal end of the aspiration catheter by the preselected distance.

In some examples, the transition structure comprises one or more materials selected from a group consisting of an elastic material, a polymeric material, a metallic material, and a shape memory material, individually or in combination.

In some examples, the transition structure covers, partially covers, fills, or partially fills the open distal end of the aspiration catheter.

In some examples, the transition structure comprises a metallic scaffold, a polymeric membrane, a polymeric scaffold, a combination of metallic scaffold and polymeric membrane, a shape memory alloy scaffold, or other structure that cover or partially covers or fills or partially fills, the open distal end or a distal segment of the lumen of the aspiration catheter.

In some examples, the transition structure may be detachably coupled to the distal segment of the inner catheter and may be configured to be slidably retracted through the lumen of the aspiration catheter lumen after detaching. For example, the transition structure when detached may be smaller than the open distal end of the aspiration catheter. For example, the transition structure when detached may be larger than the open distal end of the aspiration catheter and may be configured to be compressible to slidably be retracted through the aspiration catheter lumen. In some instances, a proximal end of the transition structure may be configured to be decoupled from a distal tip of the distal segment of the inner catheter. In other instances, a distal end of the transition structure may be configured to be decoupled from a distal tip of the distal segment of the inner catheter. After the transition structure decouples, the inner catheter may be configured to retract the transition structure proximally into the aspiration lumen causing the transition structure to invert as it may be retracted.

In some examples, the clot aspiration system further comprises an elongated stiffening member configured to be disposed over the inner catheter and inside the aspiration catheter lumen. For example, the elongated stiffening member comprises an elongated rod having a tapered tip and a guidewire lumen.

In a second aspect, the present invention provides a method for aspirating clot from a patient's blood vessel. Such methods utilize an aspiration assembly including (1) an aspiration catheter having a proximal end, an open distal end, and an aspiration lumen therebetween and (2) an inner catheter having a proximal end, a distal end, and a guide wire lumen therebetween. A distal end of the aspiration assembly is advanced into the blood vessel over a guide wire passing through the guide wire lumen of the inner catheter, where a transition structure covers, fills, and/or abuts the open distal end of the aspiration assembly retracting the transition structure positioning the open end of the aspiration catheter near the clot; applying a negative pressure through the aspiration lumen to draw clot through the open end into said lumen.

In some examples, the transition structure comprises a cone having a base covering the open distal end of the aspiration catheter, wherein retracting the transition structure comprises advancing the cone so that the base radially collapses and pulling the inner catheter proximally out of the aspiration lumen.

In some examples, the transition structure comprises an inflatable structure inflated in a distal end of the aspiration lumen, wherein retracting the transition structure comprises dilating the inflatable structure and pulling the inner catheter proximally out of the aspiration lumen.

In many examples, the aspiration assembly further comprises an outer catheter having a distal end which may be initially covered, filled, and/or abutted by the transition structure. For example, the outer catheter may comprise a sheath such as an access sheath configured to seal in a vascular access penetration.

In many examples, the transition structure may be withdrawn from the blood vessel prior to aspiration of the clot.

In many examples, the transition structure supports the aspiration catheter during advancement into or through the blood vessel.

In some examples, the transition structure enhances tracking and/or pushability of the aspiration catheter through the blood vessel.

In different examples, the transition structure may be formed from one or more of an elastic material, polymeric material, or shape memory material, individually or in combination.

In some examples, retracting the transition structure comprises detaching the transition structure from the aspiration catheter assembly and withdrawing the detached transition structure through the aspiration lumen. For example, retracting the transition may comprise deforming and/or radially collapsing the structure to fit in the aspiration lumen.

In third aspect, the present invention provides a clot aspiration system for aspirating clot from a blood vessel comprising an aspiration assembly and a tapered transition structure. The aspiration assembly typically has a proximal end and a distal end and includes (1) an outer sheath having a proximal end, an open distal end, and a central lumen therebetween and (2) an aspiration catheter having a proximal end, an open distal end, and an aspiration lumen therebetween. The aspiration catheter may be slidably mounted in the central lumen of the outer sheath, and the tapered transition structure may be positioned over the open distal ends of the outer sheath and the aspiration catheter, wherein the tapered transition structure may be attached to at least one of the outer sheath and the aspiration catheter and may be configured to radially open to allow the open distal end of the aspiration catheter to be distally advanced beyond the open distal end of the outer sheath.

In some examples, a distal tip of the tapered transition structure may be configured to be advanced over a guidewire prior to being radially opened.

In some examples, an inner catheter having a distal portion may be configured to pass through a distal tip of the tapered transition prior to being radially opened, and the inner catheter may be configured to be advanced over a guidewire.

In some examples, the distal tip of the tapered transition structure may be detachably attached to the inner catheter by a tether, such as a suture loop.

In some examples, the transition structure may be coupled to the distal end of the outer sheath. For example, the tapered transition structure may be preformed into a conical shape which opens in response to distal advancement of the aspiration catheter through the tapered transition structure.

In some examples, the transition structure may be detachably coupled to the distal end of the aspiration assembly and configured to be slidably retracted through the lumen of the aspiration catheter lumen after detaching said transition structure from the distal end of the aspiration assembly. For example, the transition structure when detached has a smaller configuration than the open distal end of the aspiration catheter. For example, the transition structure when detached has a larger configuration than the open distal end of the aspiration catheter but may be configured to be compressible to slidably be retracted through the aspiration catheter lumen. For example, a proximal end of the transition structure may be configured to be retracted into the aspiration lumen first when retracting the transition structure into the aspiration lumen. For example, a distal end of the transition structure may be configured to be retracted into the aspiration lumen first when retracting the transition structure into the aspiration lumen.

In some examples, the transition structure may be configured to invert or evert when retracting the structure into the aspiration lumen. Typically, the transition structure will remain attached to the inner catheter or other retracting member in such instances.

In fourth aspect, the present invention provides a method for aspirating clot from a patient's blood vessel. The method typically comprises providing an aspiration assembly including (1) an aspiration catheter having a proximal end, an open distal end, and an aspiration lumen therebetween and (2) an outer catheter having a proximal end, an open distal end, and a central lumen therebetween. A distal end of the aspiration assembly is advanced into the blood vessel over a guide wire passing through the aspiration lumen of the aspiration catheter to position a transition structure to cover, fill, and/or abut against the open distal ends of the aspiration and outer catheters. The transition structure is opened or removed to allow passage of the open distal end of the aspiration catheter through the distal end of the outer catheter. The open distal end of the aspiration catheter is positioned near the clot, and a negative pressure is applied through the aspiration lumen to draw clot through the open end into said lumen.

In some examples, the transition structure comprises a cone having a base attached to the distal end of the outer catheter.

In some examples, the transition structure comprises advancing the open distal end of the aspiration catheter through the cone to open said cone.

In some examples, the transition structure is detached from a distal end of the aspiration assembly and slidably retracting the detached transition structure through the lumen of the aspiration catheter lumen.

In some examples, the aspiration assembly further comprises an inner catheter having a distal end initially passing through the transition structure.

In some examples, the outer catheter comprises a sheath, such as an access sheath configured to seal in a vascular access penetration.

In fifth aspect, the present invention provides an aspiration catheter for removing clot from a blood vessel. The aspiration catheter typically comprises a catheter body having a proximal end, a distal end, and an aspiration lumen therebetween. A distal tip structure extends distally from the distal end of the catheter body and has a central clot-receiving passage contiguous with the aspiration lumen of the catheter body, and the distal tip structure may be formed at least partly from a malleable material so that the distal tip structure non-elastically reshapes from a furled configuration to an unfurled configuration in response to being engaged against clot as the aspiration catheter may be advanced in a blood vessel.

In some examples, the distal tip structure unfurls to conical shape with an enlarged, open distal end.

In some examples, the malleable material comprises a polymer, a metal, an unset shape memory alloy, an unset nickel-titanium alloy, or the like.

In some examples, the distal tip structure in its unfurled configuration may comprise a pleated cone having a truncated end attached to the distal end of the catheter body and a radially expanded, open distal end.

In some examples, the distal tip structure may be configured to be introduced in low profile configuration and to expand to a higher profile configuration in response to being engaged against clot as the aspiration catheter may be advanced in a blood vessel.

In some examples, the distal tip structure may be configured to inspire clot into an interior volume and to collapse over the inspired clot in response to application of a negative pressure of at least 0.9 atm, at least 0.8 atm, at least 0.7 atm, at least 0.6 atm, at least 0.5 atm, at least 0.4 atm, at least 0.3 atm, at least 0.1 atm, at least 0.1 atm, and at least 0.05 atm in the central clot-receiving passage of the distal tip structure.

In some examples, the distal tip structure may be configured to collapse after the clot has move proximally of the central clot-receiving passage of the distal tip structure in response to application of a negative pressure of at least 0.9 atm, at least 0.8 atm, at least 0.7 atm, at least 0.6 atm, at least 0.5 atm, at least 0.4 atm, at least 0.3 atm, at least 0.1 atm, at least 0.1 atm, and at least 0.05 atm in the central clot-receiving passage of the distal tip structure.

In a sixth aspect, the present invention provides clot aspiration system for aspirating clot from a blood vessel. The system typically comprises an aspiration catheter having a proximal end, a distal end, and an aspiration lumen therebetween. An elongated inner stiffening member has a proximal end, a distal end, and a guide wire lumen therebetween, and the elongated inner stiffening member is typically removably received in the aspiration lumen of the aspiration catheter. At least one frictional anchor is disposed on a distal region of the elongated inner stiffening member, and the frictional anchors are configured to reversibly engage an inner wall of the aspiration lumen to enhance pushability of the aspiration catheter as it may be being advanced through a blood vessel.

In some examples, the system further comprises a plurality of frictional anchors axially spaced-apart over at least a distal region of the elongated inner stiffening member.

In some examples, the frictional anchors may comprise one or more inflatable balloons wherein said balloons are configured to be inflated to engage and deflated to reversibly disengage the inner wall of the aspiration catheter to enhance stiffness and pushability. In such instances, the elongated inner stiffening member typically comprises one or more lumens configured to inflate said one or more inflatable balloons.

In some other examples, the frictional anchors may comprise one or more of a solid, hollow, solid flared area, bumps, bulge, or other protruding structures. The structure maybe discrete or circumferentially covering an outer surface. The structures preferably are protruding radially from a distal segment of the outer surface of the inner catheter and are configured to reduce or eliminate an annular gap between the aspiration lumen inner surface of the aspiration catheter and the inner catheter outer surface and/or to enhance the track and/or to enhance the push of the aspiration catheter, along a distal segment of the aspiration catheter. The structures may be formed from polymeric, metallic, combination, or other type material. The structures are typically attached or to the outer surface of the inner catheter and can be formed during the formation of the inner catheter or attached to the outer surface separately. The structures can be formed from the same material as the inner catheter or a different material. The axial length of the structures typically ranges from 0.5 mm to 5 mm, preferably ranges from 0.5 mm to 3 mm. The diameter of the structures typically ranges from 0.5 mm to 5 mm, preferably ranges from 0.5 mm to 3 mm. The height of the structures typically ranges from 0.1 mm to 2 mm, preferably ranges from 0.1 mm to 1 mm. The base of the structure may be wider than the peak of the structure forming a cone shape, or other shape types. The structures can be positioned along one axis of the outer surface or have a pattern along a length of a distal segment of the inner catheter outer surface. Such pattern includes helical pattern, offset pattern such as 60, 90, 120, 180 degree offset pattern, or other. In other example, the pattern is circumferential pattern such as a donut shape or other shapes. The structures are typically located on a distal segment of the outer surface of the inner catheter, where the segment length ranges from 1 cm to 25 cm, preferably ranges from 3 cm to 15 cm. The segment typically is located from 1 mm to 5 cm proximal to the distal end of the inner catheter.

In some examples, the structures or balloons may have the same initial diameter and/or expanded diameter. In other examples, the structures or balloons may have different initial diameter or expanded diameter so that larger diameters or inflated balloons may engage the inner wall of the aspiration catheter while smaller diameters or inflated balloons do not engage the inner wall of the aspiration catheter.

In some examples, the aspiration catheter has a diameter in a range from 1.5 mm to 3 mm, the inner stiffening member has a diameter in a range from 0.5 mm to 2.75 mm, the anchors are distributed over a distance in a range from 0.5 cm to 50 cm, preferably over a range from 15 cm to 25 cm, or alternatively from 0.5 cm to 15 cm of the distal end of the aspiration catheter, and the anchors are axially spaced-apart by a distance in a range from 2 mm to 25 mm, preferably from 5 mm and 25 mm.

In some examples, the elongated inner stiffening member comprises an elongated rod having a tapered tip and a guidewire lumen.

In some examples, the elongated inner stiffening member comprises an inner catheter, such as any of the inner catheter examples described previously.

In some examples, the plurality of expandable frictional anchors are configured not to engage the inner wall of the aspiration catheter when fully expanded.

In some examples, the plurality of expandable frictional anchors are configured to have a variable engagement with the inner walls of the aspiration catheters ranging from no engagement, to partial engagement, to full engagement when fully expanded.

In seventh aspect, the present invention provides an aspiration catheter for removing clot from a blood vessel comprising a catheter body having a proximal end, a distal end, and an aspiration lumen therebetween. A scaffold extends distally from the distal end of the catheter body and has a central clot-receiving passage contiguous with the aspiration lumen of the catheter body. A vacuum-resistant membrane covers the scaffold to establish a clot aspiration path from a distal end of the scaffold to a proximal end of the aspiration lumen in the catheter body so that applying a vacuum to the proximal end of the aspiration lumen can draw clot into the central clot-receiving passage. The scaffold may be configured to elongate and radially collapse in response to a proximal tension.

In some examples, the aspiration catheter further comprises at least one pull strut connecting a distal end of the catheter body to a proximal end of the scaffold, wherein the at least one pull strut may be configured to apply a localized stress on the scaffold which causes the scaffold to elongate and collapse radially. In some instances, the aspiration catheter comprise a single pull strut connected to one location on a closed-loop scaffold. In other instances, the aspiration catheter may comprise at least first and second pull struts connected to first and second spaced-apart locations on a proximal periphery of a closed-loop scaffold.

In some examples, the aspiration catheter further comprises first and second pull struts connecting a distal end of the catheter body to a proximal end of the scaffold, wherein the scaffold consists of an open loop having first and second ends and the first pull strut may be connected to the first end of the open loop scaffold and the second pull strut may be connected at the second end of the open loop scaffold.

In some examples, the scaffold comprises a single member free from branching with a single pull strut connected to a proximal end of a single member having a free distal end. In some instances, the single member may be formed into a cylinder or cone with undulating regions.

In some examples, the scaffold comprises a malleable material that allows irreversible elongation and radial collapse. In other examples, the scaffold comprises an elastic material that allows reversible elongation and radial collapse.

In some examples, the scaffold comprises a plurality of circumferential rings arranged along an axis and patterned from a non-degradable material, said scaffold being configured to expand from a crimped configuration to an expanded configuration. The circumferential rings may be joined by axial links, and each of the axial links may include a circumferential separation region. The scaffold may be configured to circumferentially separate along separation interfaces and to form one continuous structure after all axial links have separated along said circumferential separation regions.

In an eighth aspect, the present invention provides an aspiration catheter for removing clot from a blood vessel comprising a catheter body having a proximal end, a distal end, and an aspiration lumen therebetween. A scaffold extends distally from the distal end of the catheter body and has a central clot-receiving passage contiguous with the aspiration lumen of the catheter body. A membrane comprises an elastic sleeve covering the scaffold to establish a clot aspiration path from a distal end of the scaffold to a proximal end of the lumen in the catheter body so that applying a vacuum to a proximal end of the aspiration lumen can draw clot into the central clot-receiving passage. At least a distal portion of the scaffold may be radially expandable from a delivery configuration to an extraction configuration or alternatively the distal portion is delivered in the extraction configuration, where the distal portion of the scaffold may be configured to controllably collapse from the extraction configuration to a partially collapsed configuration in response to a vacuum applied within the central clot-receiving passage, where said collapsed configuration may be sufficient to allow the aspiration of the clot into the aspiration lumen.

In some examples, the scaffold may be embedded in the membrane. In other examples, the membrane may be attached to the scaffold.

In some examples, the partially collapsed configuration has an average width in a range from 0.25 to 0.75 of a width of the radially expanded configuration.

In some examples, the scaffold has may be configured to partially collapse in response to a vacuum in a range from 0.2 atm to 1 atm applied to the central clot-receiving passage. In such instances, the scaffold may be configured to self-expand to the extraction configuration when a pressure in the central clot-receiving passage returns to above 0.2 atm.

In some examples, the radially expandable distal portion of the scaffold may be configured to be reversibly driven between a radially contracted configuration and a radially expanded configuration, and a partially collapsed configuration.

In some examples, the radially expanded extraction configuration comprises a substantially cylindrical distal region configured to engage an inner wall of the blood vessel and a tapered transition region between the cylindrical distal region and the distal end of the catheter body, wherein the cylindrical distal region has an open distal end configured to direct clot into the central clot-receiving passage when the vacuum may be applied to a proximal end of the aspiration lumen.

In some examples, the radially expanded extraction configuration comprises a substantially conical region with a proximally oriented apical opening attached to the distal end of the catheter body and a distally oriented open base configured to engage an inner wall of the blood vessel and direct clot into the central clot-receiving passage when the vacuum may be applied to a proximal end of the aspiration lumen.

In some examples, the scaffold comprises struts joined by crown, further comprising stops on adjacent struts to limit the collapse of the scaffold under pressure. For example, the stops may comprise circumferentially aligned tabs.

In some examples, the scaffold comprises a polymeric material. In other examples, the scaffold comprises a shape memory material. In still other examples, the scaffold comprises an elastomeric material, and in further examples, the scaffold comprises a combination of elastomeric, polymeric and/or shape memory material.

In ninth aspect, the present invention provides a method for extracting clot from a blood vessel. The method comprises positioning a radially expandable distal portion of an aspiration catheter in a blood vessel proximal to the clot or alternatively delivering said distal portion in the expanded configuration. The radially expandable distal portion of the aspiration catheter is radially expanded in the blood vessel to form an enlarged central clot-receiving passage through the radially expandable distal portion contiguous with an aspiration lumen in the aspiration catheter. A first level of vacuum is applied to a proximal portion of the aspiration lumen to draw clot from the blood vessel into the radially expandable distal portion of the aspiration catheter. After the clot has been drawn into the radially expandable distal portion of the aspiration catheter, the level of vacuum is increased to cause the radially expandable distal portion to partially collapse to disrupt the clot.

In some examples, the radially expandable distal portion of the aspiration catheter comprises a scaffold covered with a vacuum-resistant membrane and wherein struts of the scaffold struts act to break and/or shear the clot as the radially expandable distal portion may be partially collapsed by increasing the vacuum level.

In some examples, the radially expandable distal portion of the aspiration catheter may be partially collapsed to an average width in a range from 0.25 to 0.75 of an initial width of the radially expandable distal portion of the aspiration catheter.

In some examples, wherein the first level of vacuum may be in a range from 0 atm to 0.5 atm, usually being in a range from 0.2 atm to 1 atm.

In some examples, the vacuum level may be cycled up and down to enhance clot disruption after the clot has been drawn into the radially expandable distal portion of the aspiration catheter.

In tenth aspect, the present invention provides a clot disruption catheter for resecting and aspirating clot from a blood vessel. The catheter comprises a catheter body having a proximal end, a distal end, and an aspiration lumen therebetween. A radially expandable distal portion of the catheter body has an expandable central clot-receiving passage contiguous with the aspiration lumen, or alternatively said distal portion of the catheter body having said central clot-receiving passage being delivered in the expanded configuration, and a clot disruption means is coupled to the radially expandable distal portion of the catheter body configured to disrupt clot sufficiently pass into and through the aspiration lumen when a vacuum may be applied to a proximal end of the aspiration lumen.

In some examples, the clot disruption means comprises at least one cutting element disposed across the expandable central clot-receiving passage when the expandable central clot-receiving passage may be expanded to cut clot as the clot may be aspirated into the aspiration lumen when the vacuum may be applied to the proximal end of the aspiration lumen.

In some examples, the cutting element comprises a wire attached across the distal opening of the radially expandable distal portion of the catheter body, wherein the wire may be folded when said radially expandable distal portion may be closed and the wire may be tensioned across the distal opening when said radially expandable distal portion may be opened.

In some examples, the cutting element comprises folding blades attached across the distal opening of the radially expandable distal portion of the catheter body, wherein the blades are folded closed when said radially expandable distal portion may be in the collapsed configuration and the folding blades are substantially non-parallel when said radially expandable distal portion may be in an expanded configuration.

In some examples, the clot disruption means comprises a clot constriction structure disposed inside at least one of the central clot-receiving passage and the aspiration lumen, wherein the clot constriction structure may be actuable to radially constrict a segment of clot after said segment has been aspirated into said at least one of the central clot-receiving passage and the aspiration lumen.

In some examples, the clot constriction structure comprises a coil disposed coaxially in the at least one of the central clot-receiving passage and the aspiration lumen, wherein prior to actuation the coil lies adjacent an inner wall of the at least one of the central clot-receiving passage and the aspiration lumen and upon actuation the coils closes radially inwardly to constrict the clot.

In an eleventh aspect, the present invention provides a method for disrupting and extracting clot from a blood vessel. The method comprises radially expanding a radially expandable distal potion of an aspiration catheter proximal to a region of clot in the blood vessel, or alternatively delivering said distal portion of an aspiration catheter in an expanded configuration. A vacuum is applied to a proximal portion of an aspiration lumen in the aspiration catheter to draw a segment of clot into at least one of the central clot-receiving passage and the aspiration lumen. The clot is disrupted as it is drawn into the central clot-receiving passage or after it has been received in the at least one of the central clot-receiving passage and the aspiration lumen. A vacuum is applied to a proximal portion of an aspiration lumen in the aspiration catheter to draw the disrupted clot through the aspiration lumen to a proximal portion of the aspiration catheter.

In some examples, disrupting the clot comprises drawing the clot across a cutting element disposed across the central clot-receiving passage as the vacuum may be applied to a proximal portion of an aspiration lumen in the aspiration catheter. For example, a wire may be tensioned across the central clot-receiving passage as the radially expandable distal potion of an aspiration catheter may be radially expanded. In other examples, blades may be unfolded or otherwise opened across the central clot-receiving passage as the radially expandable distal portion of an aspiration catheter is radially expanded.

In some examples, disrupting the clot may comprise actuating a clot constriction structure disposed inside at least one of the central clot-receiving passage and the aspiration lumen to radially constrict a segment of clot after said segment has been aspirated into said at least one of the central clot-receiving passage and the aspiration lumen, wherein the clot constriction structure may be released to allow the clot to pass to a proximal portion of the aspiration lumen. For example, actuating the clot constriction structure may comprise closing coils over the clot.

In some examples, radially expanding the radially expandable distal potion of the aspiration catheter may comprise releasing the radially expandable distal potion from a constraining sheath.

In some examples, radially expanding the radially expandable distal potion of the aspiration catheter may comprise actuating the structure on the aspiration catheter to radially expand the radially expandable distal potion.

In a twelfth aspect, the present invention provides an aspiration catheter for removing clot from a blood vessel. The aspiration catheter comprises a catheter body having a proximal end, a distal end, and an aspiration lumen therebetween. An expandable distal tip extends distally from the distal end of the catheter body and has a central clot-receiving passage contiguous with the aspiration lumen of the catheter body, and at least a distal portion of the expandable distal tip may be radially expandable from a delivery configuration to an extraction configuration, or alternatively the distal tip or the at least distal portion of the distal tip being delivered in the expanded configuration. An expandable seal is disposed circumferentially about an exterior surface of the catheter body, and the expandable seal may be located a preselected distance proximal of the expandable distal tip and wherein one or more vacuum ports are formed in a wall of the catheter body in a region between the expandable distal tip and the expandable seal.

In some examples, the expandable seal may comprise an inflatable balloon. In other examples, wherein the expandable seal comprises a deployable cuff. In further examples, the expandable distal tip comprises a self-expanding scaffold.

In some examples, the preselected distance may be in a range from 5 mm to 50 mm.

In some examples, the self-expanding scaffold may be covered by a pressure-resistant membrane to establish a clot aspiration path from a distal end of the scaffold to a proximal end of the aspiration lumen in the catheter body so that applying a vacuum to a proximal end of the aspiration lumen can draw clot into the central clot-receiving passage.

In a thirteenth aspect, the present invention provides a method for extracting clot from a blood vessel. The method comprises positioning a radially expandable or delivered in an expanded configuration distal portion of an aspiration catheter in a blood vessel proximal to the clot. The radially expandable distal portion of the aspiration catheter is radially expanded in the blood vessel to form a clot-receiving passage through the radially expandable distal portion contiguous with an aspiration lumen in the aspiration catheter. A circumferential seal is radially expanded about the aspiration catheter at preselected distance proximal of the expanded distal tip. A vacuum is applied to a proximal portion of the aspiration lumen to draw clot from the blood vessel through the radially expandable distal portion and into the lumen of the aspiration catheter, where at least one vacuum port disposed in a wall of the catheter body in a buffer region between the expandable distal tip and the expandable seal to draw a vacuum in said region.

In another aspect of the present invention, an aspiration catheter for removing clot from a blood vessel comprises a catheter body having a proximal end, a distal end, and an aspiration lumen therebetween. A scaffold extends distally from the distal end of the catheter body and has a central clot-receiving passage contiguous with the aspiration lumen of the catheter body. A vacuum-resistant membrane covering the scaffold establishes a clot aspiration path from a distal end of the scaffold to a proximal end of the aspiration lumen in the catheter body so that applying a vacuum to the proximal end of the aspiration lumen can draw clot into the central clot-receiving passage. At least a distal portion of the scaffold is configured to be radially expandable from a delivery configuration to an extraction configuration.

The delivery configuration is typically a low profile configuration to allow advancement through a patient's vasculature, typically the neuro vasculature but optionally in the cardiac and peripheral vasculature as well. The extraction configuration is usually radially expanded or enlarged with an open port or passage at the distal end of the scaffold to engage and collect clot, thrombus, atheroma, and other obstructive material from the blood vessel when vacuum is applied to the aspiration limen. The scaffold provides mechanical support while the vacuum-resistant membrane establishes a vacuum through the scaffold.

In some instances, the scaffold will be at least partially self-expanding, typically being formed in whole or in part from an elastic material, such as a shape or heat memory metal or plastic, e.g. a nickel-titanium alloy. A sheath may be configured to radially constrain such self-expanding distal portions, where translation of the sheath relative to the catheter body releases the constraint and allows the radially expandable distal portion of the scaffold to radially expand. Other forms of constraint, such as constraining hoops, suture loops, dissolvable adhesives, and the like may also be used for deploying self-expanding scaffold.

In other instances, the radially expandable distal portion of the scaffold is configured to be reversibly driven between a radially contracted configuration and a radially expanded configuration. As described below, such mechanism may comprise a rotating coil, a pair of counter-rotating coils, or the like.

The scaffold in its radially expanded configuration may have a substantially cylindrical distal region configured to engage an inner wall of the blood vessel and a tapered transition region disposed between the cylindrical distal region and the distal end of the catheter body. The cylindrical distal region typically has an open distal end configured to direct clot into the central clot-receiving passage when the vacuum is applied to a proximal end of the aspiration lumen. The cylindrical distal region may have a diameter, when expanded, in a range from 2 mm to 6 mm, typically from 2.2 mm to 5.5 mm and length, when expanded, in a range from 1 mm and 150 mm, preferably in a range from 2 mm to 100 mm, more preferably in a range from 3 mm to 50 mm.

Alternatively the radially expanded configuration may have a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen. The distally oriented open base may have a diameter, when expanded, in a range from 2 mm to 6 mm, typically from 2.2 mm to 5.5 mm, while the length between the apical end and the open base, when expanded, in a range from 1 mm and 10 mm, preferably in a range from 2 mm to 5 mm, more preferably in a range from 3 mm to 4 mm.

In other instances, the membrane of the aspiration catheter may cover all or a portion of an inner surface of the scaffold. A distal end of the vacuum-resistant membrane may be located proximally of a distal end of the scaffold, leaving an uncovered distal portion of the scaffold. A distal or other portion of the scaffold may be uncovered (not covered by the vacuum-resistant membrane) and configured to do at least one of invaginate the clot, break the clot, and facilitate extraction of the clot.

In still other instances, an open port of the distal tip of the scaffold in its extraction configuration may has an area which is 1.5 to 10 times greater than the open port area when the scaffold is in its delivery configuration. The entire scaffold may comprise an expandable distal segment. The vacuum-resistant membrane may be coupled to at least the distal portion the scaffold. The delivery configuration of the distal portion of scaffold may smaller than the distal end of the catheter body, and an inner surface of the distal portion of scaffold may be coated with a lubricious material.

In further instances, the scaffold in its extraction configuration may be expanded from a size in a range from that of the clot to that of the vessel. A catheter or a wire may be placed to extend through the aspiration lumen to provide retraction or advancement of the sheath to deploy the scaffold to the expanded configuration. The distal portion of the scaffold in said extraction configuration may be configured to engage an inner wall of the blood vessel to substantially prevent blood proximal to the clot from entering the clot aspiration path when said vacuum is applied, or a proximal portion of the scaffold in said extraction configuration may be configured to engage an inner wall of the blood vessel to substantially reduce blood proximal to the clot from entering the clot aspiration path when said vacuum is applied.

In many instances, the scaffold in the extraction configuration is configured to draw the clot into the central clot-receiving passage when distal end of said scaffold is placed proximal to the clot and vacuum is applied. Additionally, the distal portion of the scaffold may be configured to engage and break up clot when said distal portion is expanded to facilitate suction of said clot into the aspiration lumen. For example, the expandable scaffold may comprise one or more features selected from the group consisting of sharp edges, metallic protrusions, fins, hook elements, and slots to improve cutting of or gripping the clot.

In another example, a thrombectomy catheter for removing occlusive material from a blood vessel includes a catheter body and a radially expandable separator scaffold. The catheter body has a proximal end, a distal end, and an aspiration lumen therebetween. The radially expandable separator scaffold extends distally from the distal end of the catheter body and includes helically arranged cutting elements which define a central clot-receiving passage. The separator scaffold may be radially expanded in the blood vessel and rotated and advanced to resect clot. The aspiration lumen of the catheter body and the central clot-receiving passage of the radially expandable separator scaffold are arranged in-line so that clot resected by rotating the separator scaffold may be aspirated into the aspiration lumen of the catheter body by applying a vacuum to a proximal end of the aspiration lumen.

In those examples where the scaffold is configured to be reversibly driven, the radially expandable distal portion of the scaffold may comprise at least a first coil which is configured to be torqued in at least one rotational direction to radially open or close the radially expandable at least distal portion of the scaffold. In such cases, the vacuum-resistant membrane may comprise an expandable sleeve which covers the at least first coil to enclose the central clot-receiving passage to create a continuous vacuum path from the aspiration lumen to a distal end of the radially distal expandable segment. For example, the expandable sleeve may comprise at least one of an elastic section, a folded section, and a furled section. The at least first coil may be configured to be torqued in both rotational directions to radially open and close the radially expandable portion of the scaffold. The cylindrical distal region of the scaffold further may comprises a rotatable inner member, where the first coil is fixed at its proximal end to a distal end of the catheter body and at its distal end to a distal end of the inner member. In this way, rotation of a proximal end of the inner member rotates the distal end of the first coil. In some instances, the cylindrical distal region of the scaffold may further comprise a rotatable outer member, where the first coil is fixed at its proximal end to a distal end of the catheter body and at its distal end to a distal end of the outer member, wherein rotation of a proximal end of the outer member rotates the distal end of the first coil. The scaffold may still further comprises a second coil rotatably and coaxially mounted within the at least first coil, where the at least one coil is fixed at its proximal end to a distal end of the catheter body and at its distal end to a distal end of the second coil and where the first and second coils are wound in opposite helical directions so that rotation of a proximal end of the second coil in a first direction causes both the first and second coils to radially expand.

In such coiled examples, at least one coil may comprise a helically wound elongated member formed from struts joined by crowns in a serpentine pattern, wherein rotation of a proximal end of the at least one coil releases said struts from a crimped configuration to allow the helically wound elongated member to radially expand.

Optionally, even when the scaffold is coiled and configured to be reversibly driven, the aspiration catheter may still comprise a sheath or cap constraining the at least one coil in its crimped configuration. In those examples where the conical region of the scaffold comprises a plurality of struts having proximal ends disposed about the proximally oriented apical opening and distal ends disposed about the distally oriented open base, such struts may be arranged individually with free proximal ends coupled only by the vacuum-resistant membrane. Alternatively, such struts may be interconnected. In other examples, the struts may be arranged in a serpentine pattern with crown regions disposed about both the proximally oriented apical opening and the distally oriented open base. In still other instances, the struts the scaffold may be configured to be reversibly driven ay diverge radially outwardly in the distal direction to define the conical region when unconstrained.

In those examples where the distal portion of the scaffold in its extraction configuration may have a substantially conical region with a distally oriented apical opening attached to the distal end of the catheter body and a proximally oriented open base configured to engage an inner wall of the blood vessel, a scaffold constraint and release mechanism comprising a sheath may be configured to be advanced distally to cover and constrain the struts and retracted proximally to uncover and release the struts to expand radially. Alternatively or additionally, a scaffold constraint and release mechanism comprising a cap covers and constrains the distal ends of the struts in a first position and uncovers and releases the distal ends of the struts in a second position. An alternative scaffold constraint and release mechanism may comprise a length of material attached to an inner member and wrapping around the struts, wherein the inner member is configured to pull the length of material off the struts to allow them to self-expand. A further alternative may comprise a scaffold constraint and release mechanism comprising an inner member, wherein the struts are initially bonded to the inner member with a frangible material that can be mechanically broken to release the struts to self-expand. A still further alternative scaffold constraint and release mechanism may comprise a filament held under tension around the struts, wherein the tension can be released to allow the struts to self-expand. In yet other examples, the struts may be folded entirely inside the aspiration lumen of the catheter body and are configured to be pushed distally to deploy and open.

In another aspect of the present invention, an aspiration catheter for removing clot from a blood vessel, said aspiration catheter comprises a catheter body having a proximal end, a distal end, and an aspiration lumen therebetween. A scaffold extends distally from the distal end of the catheter body and has a central clot-receiving passage contiguous with the aspiration lumen of the catheter body. A membrane covering the scaffold to establish a clot aspiration path from a distal end of the scaffold to a proximal end of the lumen in the catheter body so that applying a vacuum to a proximal end of the aspiration lumen can draw clot into the central clot-receiving passage while substantially preventing blood proximal to the clot from entering the aspiration lumen. At least a proximal portion of the scaffold in a preferred example is radially expandable from a delivery configuration to an extraction configuration, or alternatively the scaffold is delivered in the expanded configuration wherein the expanded configuration is the extraction configuration. The radially expanded configuration has a substantially conical region with a distally oriented apical opening attached to the distal end of the catheter body and a proximally oriented open base configured to engage an inner wall of the blood vessel and direct clot into the central clot-receiving passage when the vacuum is applied to a proximal end of the aspiration lumen.

In a still further aspect of the present invention, a method for extracting clot from a blood vessel, said method comprises positioning a radially expandable distal portion of an aspiration catheter in a blood vessel proximal to the clot. A distal portion of the aspiration catheter is radially expanded in the blood vessel to form an enlarged central clot-receiving passage through the radially expandable distal portion contiguous with an aspiration lumen in the aspiration catheter. A vacuum is applied to a proximal portion of the aspiration lumen to draw clot from the blood vessel into the radially expandable distal portion of the aspiration catheter, where the radially expandable distal portion of the aspiration catheter comprises a scaffold covered with a vacuum-resistant membrane with sufficient strength to maintain patency of the central clot-receiving passage while applying the vacuum.

In such methods, a distal end of the radially expandable distal portion may engage the clot when the vacuum is applied. Alternatively, a distal end of the radially expandable distal portion may be spaced proximally of the clot when the vacuum is applied. Alternatively or additionally, a distal end of the radially expandable distal portion may engaged against the clot and manipulated to at least partly breakup the clot prior to or while the vacuum is applied. Optionally, a distal end of the radially expandable distal portion may be positioned to inhibit blood located proximally of the distal portion of an aspiration catheter from entering the aspiration lumen.

With further respect to such method, the radially expandable distal portion of the aspiration catheter may be self-expanding and radially expanding the radially expandable distal portion comprises releasing the radially distal expandable segment from a constraining sheath. Often, radially expanding the radially expandable distal portion of the aspiration catheter comprises actuating a structure on the aspiration catheter to open the central clot-receiving passage. For example, the structure may be actuated to radially constrict the radially distal segment of the aspiration catheter in the blood vessel to close central clot-receiving passage. Actuating the structure on the aspiration catheter to expand or constrict the central clot-receiving passage may comprise torquing at least a first coil in a first rotational direction to radially open or close the radially distal expandable segment. The first coil may be torqued in a first direction to radially expand the radially distal segment of the aspiration catheter and torqued in a second rotational direction to radially constrain the radially distal segment of the aspiration catheter. Torquing the first coil may comprise rotating an inner member or an outer member attached to a distal end of the first coil, optionally further comprising rotating a second coil attached to a distal end of the first coil.

The methods may result in the clot being extracted substantially intact or in other instances may result in a proximal portion of the clot being extracted substantially intact. Often substantially all clot may be extracted in a first extraction attempt. Often, the extracted clot comprises hard clot.

In still further instances of the methods herein, the scaffold may comprise an element which follows a single path to form a cylindrical or conical envelope. The single path may have anyone or a combination of a closed loop, an open path, and the like.

In particular instances, radially expanding the distal portion of the aspiration catheter comprises rotating an inner member attached to the scaffold, wherein the scaffold comprises a cylindrical distal region having a first coil is fixed at its proximal end to a distal end of the catheter body and at its distal end to a distal end of the inner member, wherein rotation of a proximal end of the inner member rotates the distal end of the first coil. The cylindrical distal region of the scaffold may further comprise a rotatable outer member, where the first coil is fixed at its proximal end to a distal end of the catheter body and at its distal end to a distal end of the outer member such that rotation of a proximal end of the outer member rotates the distal end of the first coil.

In yet another aspect of the present invention, an endoluminal prosthesis comprises a scaffold having a plurality of circumferential rings arranged along an axis. The rings comprise struts joined by crowns, typically being patterned from a non-degradable material. The scaffold may be configured to expand from a crimped configuration to an expanded configuration, and at least some of the circumferential rings may be circumferentially separable, typically being joined by circumferentially separable axial links. Thus the scaffold may be configured to circumferentially separate along separation interfaces, where circumferentially separable regions of the circumferential rings and the axial links often comprise a biodegradable polymer and/or adhesive configured to hold said separations regions together during expansion and to thereafter form at least one discontinuity in the circumferential ring and the axial link after expansion of the scaffold in a physiologic environment; As a particular feature, the scaffold is formed one (a single) continuous structure so that it will remain intact along a length of the element after all discontinuities are formed.

In a still further aspect of the present invention, an endoluminal prosthesis comprises a scaffold having a plurality of circumferential rings arranged along an axis. The rings comprise struts joined by crowns and are typically patterned from a non-degradable material. The scaffold being is typically configured to expand from a crimped configuration to an expanded configuration, where at least some of the circumferential rings may be circumferentially separated, often be joined by circumferentially separable axial links, such that the scaffold may be expandable from the crimped configuration to an expanded configuration in a physiologic environment. As a particular feature, the scaffold is formed from one (a single) continuous patterned structure enhancing strength in the expanded configuration to support a body lumen.

In yet an additional aspect of the present invention, an aspiration catheter for removing clot from a blood vessel comprises a catheter body having a proximal end, a distal end, and an aspiration lumen therebetween. A scaffold extends distally from the distal end of the catheter body and typically includes a central clot-receiving passage contiguous with the aspiration lumen of the catheter body. An elastic membrane covering the scaffold establishes a clot aspiration path from a distal end of the scaffold to a proximal end of the lumen in the catheter body so that applying a vacuum to a proximal end of the aspiration lumen can draw clot into the central clot-receiving passage, where at least a distal portion of the scaffold is radially expandable from a delivery configuration to an extraction configuration. As a particular feature, the scaffold comprises two or more circumferential bisected rings with at least one bisected axial connection connecting said bisected rings.

In one example of the present invention, the device comprises an elongated tubular body comprising a distal segment and a proximal segment, wherein the distal segment is expandable from an initial small configuration to a larger configuration and then back down to a final small configuration, wherein the final small configuration is smaller than the larger configuration and may be equal to or larger than the initial small configuration. In the device of the present example the distal end of said distal segment is configured to engage a clot and/or to substantially engage the vessel wall adjacent to a clot, and the elongated tubular body comprises an aspiration lumen, and the device is able to retrieve the clot by applying a vacuum force through the aspiration lumen to the distal end of said distal segment. In an exemplary example, the vacuum force applied is between 10 mmHg and 760 mmHg, more preferably between 10 mmHg and 380 mmHg, and more preferably between 10 mm and 200 mmHg. In a further example of the present example, the elongated tubular body comprises a distal segment, and intermediate or middle segment, and a proximal segment. In another example, the distal segment extends substantially the entire length of the elongated tubular body, and has a length ranging from 1 cm to 50 cm, preferable having a length ranging from 2 cm to 20 cm, more preferably having a length ranging from 3 cm to 15 cm.

In another example, the aspiration lumen diameter of the proximal segment is larger than the aspiration lumen diameter of the distal segment in the contracted configuration but smaller than the aspiration lumen diameter of the distal segment in the expanded configuration.

In an exemplary example, the distal segment small configurations comprise one or more of the following: crimped configuration, collapsed configuration, contracted configuration, unexpanded configuration, unopened configuration, delivery configuration, or other. In another exemplary example, the distal segment larger configuration comprises one or more of the following: deployed configuration, expanded configuration, aspiration configuration, or other.

In an exemplary example, the distal segment is controllably expandable from a smaller configuration to the larger configuration and then controllably contractible to a smaller configuration. In another example, the distal segment is controllably contractible or crimpable to small configuration prior to insertion in a body lumen, then controllably expandable to a larger configuration in a body lumen, and then controllably contractible to a smaller configuration prior to withdrawal of said distal segment from a body lumen.

In an exemplary example, the distal segment is expandable and/or contractible by means of twisting or rotating torque elements attached to each end of a single coil structure in the distal segment, and the torque applied to at least one of the torque elements attached to the single coil structure causes the single coil structure to unwind to expand in diameter, or to wind to contract in diameter.

In another exemplary example, the distal segment is expandable and/or contractible by means of twisting or rotating torque elements attached to two or more coil structures in the distal segment, wherein said two or more coil structures are connected to each other in at least in one location at the distal end of said distal segment, and the proximal ends of the coil structures are connected to said torque elements, and opposing torques applied to at least one of the two or more coil structures cause them to unwind to expand in diameter or to wind to contract in diameter.

In another exemplary example, the distal segment is expandable and/or contractible by means of twisting or rotating torque elements and/or axially compressing or tensioning linear force elements connected to a braided wire structure in the distal segment, wherein the wires of the braid are then forced against each other in order to achieve the expansion or contraction.

In another exemplary example, the distal segment is expandable and/or contractible by means of axially compressing or tensioning linear force elements connected to a removable and replaceable sleeve over a braided wire structure in the distal segment, wherein the braid is designed to be self-expanding when not constrained by the sheath.

In another exemplary example, the distal segment is expandable and/or contractible by means of axially compressing or tensioning linear force elements connected to a removable and replaceable sleeve over a structure in the distal segment comprising a slotted tube or sinusoidal ring structure, wherein the slotted tube or sinusoidal wire structure is designed to be self-expanding when not constrained by the sheath.

In another exemplary example, the distal segment is expandable and/or contractible by means of axially compressing or tensioning linear force elements connected to a structure in the distal segment comprising of three or more longitudinally aligned ribs, which when put in compression causes them to bow outwards thereby expanding their profile, and when put in tension causes them to stretch flatter thereby contracting their profile. In a preferred variant of the present example, one or more V links or other means are used to attach the ribs to each other in order to maintain their circumferential alignment.

In an exemplary example, the expansion and contraction of the distal segment is controllable by torque elements and/or linear force elements comprising of one or more of the following: wires, rods, tubes, or other, and the torque elements and/or linear force elements extend substantially along the length of the elongated tubular body. In an exemplary example, the torque elements and/or linear force elements are formed from a metallic, polymeric, or composite material. In the preferred example, at least one torque element and/or linear force element comprises the catheter shafts.

In an exemplary example, the coil, braided wire, sinusoidal ring, or longitudinal rib structure comprises one or more of round wire, tubular wire, flat ribbon, contoured ribbon, or the like. In an exemplary example, the coil, braided wire, sinusoidal ring, or longitudinal rib structure are formed from a metallic material such as stainless steel, cobalt chrome, or other. In an exemplary example, the coil, braided wire, sinusoidal ring, or longitudinal rib structure are formed from a shape memory material such as a nickel-titanium alloy (“NiTi”) or the like.

In an exemplary example, a covering sleeve extends over the distal segment of the elongated tubular body, preferably covering substantially the entire length of the distal segment, wherein said covering sleeve accommodates the expansion and contraction of the distal segment while functionally maintaining vacuum pressure integrity in the aspiration lumen of the elongated tubular body. In an exemplary example, the covering sleeve comprises one or more of the following: spray coated sleeve, dip coated sleeve, elastic sleeve, radially expandable elastic sleeve, polymeric sleeve, foldable sleeve, silicone-based material sleeve, polyurethane based sleeve, and other. The sleeve is preferably attached to the distal segment in one or more locations but can alternatively be press fit onto the distal segment without attachment.

In an exemplary example, the covering sleeve only partially covers the distal segment of the elongated tubular body, such that a distal portion of the distal segment is uncovered and the expansion/contraction structure is able to directly engage the clot. An associated method of use is to advance the device until the portion of the distal segment without the covering sleeve is within the clot, such that expansion of said portion of the distal segment causes the uncovered structure to directly engage the clot, thereby aiding with breaking up the clot for improved aspiration or snaring it for withdrawal from the anatomy. In this method the distal segment may also be manipulated linearly or rotationally as part of the procedure to improve such engagement and effects, and the distal uncovered portion of the expansion structure may furthermore incorporate features to improve cutting of or gripping the clot such as sharper edges, metallic protrusions, fins, hook elements, slots in the coil ribbon, and other.

In an exemplary example, the proximal and/or intermediate segments of the elongated tubular body are comprised of a polymeric material, which may or may not contain a polymeric or metallic coil or braid within or adjacent to the polymeric material.

In an exemplary example, the distal segment is expandable from a contracted configuration to an expanded configuration, wherein the outer diameter or the aspiration lumen diameter of the distal segment in said expanded configuration is substantially the same as a non-occluded lumen diameter of the vessel adjacent to said expanded distal segment. In another example, the distal segment is controllably expandable from a contracted configuration to an expanded configuration wherein the outer diameter or the aspiration lumen diameter of the distal segment in the expanded configuration ranges from 0.5 times the non-occluded vessel lumen diameter to 1.2 times the non-occluded vessel lumen diameter, preferably the expanded configuration ranges from 0.75 times the non-occluded vessel lumen diameter to 1.2 times the non-occluded vessel lumen diameter, more preferably is substantially the same diameter of the non-occluded vessel lumen.

In another example, the present invention comprises an aspiration catheter having a distal segment configured to expand to a range of diameters ranging from 0.5 mm outer diameter in the fully collapsed state to 5.0 mm outer diameter in the fully expanded state. The device is advanced in a patient body with the distal segment in a small collapsed state to navigate the tortuous vasculature until reaching the occluded vessel and/or clot. Once the distal end of the distal segment or tip is positioned adjacent to or contacting the clot or thrombus, the distal segment of the device is expanded to a larger diameter to increase its tip area and the vacuum effectiveness. In an exemplary example, the expandable distal segment is expanded until it substantially contacts the vessel wall, in order to enhance clot separation from the vessel wall and clot removal. Advantages of expanding the distal segment or distal end of the catheter to substantially the vessel size include one or more of the following: separating the clot from the vessel wall, ease of retrieving the clot with small to modest suction force, retrieving the clot substantially intact or with less fragments, removal of the clot substantially from first attempt. The distal segment may be expanded larger than the vessel in order to further enhance clot separation from the vessel wall and clot removal. Once clot retrieval into the catheter is accomplished, the device is then reduced in size again to aid withdrawal of the aspiration system from the anatomy and minimize vascular trauma.

In another example, the device provides improved distal access in tortuous anatomy, greater revascularization success rate, shorter procedure time due to improved first pass revascularization rates and immediate clot retrieval, and reduced risk of distal emboli, all with a single device treatment approach. Furthermore, in many cases a clot may be able to be removed using a low to medium vacuum pressure, potentially further reducing vessel trauma.

Thus, in accordance with at least some of the principles of the present invention as set forth above, an aspiration catheter for removing clot from a blood vessel comprises a catheter body having a proximal end, a distal end, and an aspiration lumen therebetween, a radially distal expandable segment having a central clot-receiving passage extends distally from the distal end of the catheter body, and a means is integrated with the catheter body for expanding and/or constricting the radially distal expandable segment between a radially expanded configuration and a radially contracted configuration. The aspiration lumen of the catheter body and the central clot-receiving passage of the radially distal expandable segment are contiguous so that applying a vacuum to a proximal end of the aspiration lumen can draw clot into the central clot-receiving passage.

The radially distal expandable segment may be self-expanding, for example where the expanding means comprises a sheath configured to constrain the radially distal expandable segment in a radially constrained configuration, where retraction of the sheath allows the radially distal expandable segment to radially expand.

Alternatively or additionally, the expanding means may be integrated with the catheter body, for example comprising (1) at least a first coil which is configured to be torqued in at least one rotational direction to radially open or close the radially distal expandable segment and (2) an elastic sleeve which covers the first coil to enclose the central clot-receiving passage to create a continuous vacuum path from the aspiration lumen to a distal end of the radially distal expandable segment. The first coil may be configured to be torqued in both rotational directions to radially open and radially close the radially distal expandable segment, respectively. In a first instance, torquing may be accomplished by a rotatable inner member, wherein the first coil is fixed at its proximal end to a distal end of the catheter body and at its distal end to a distal end of the inner member, wherein rotation of a proximal end of the inner member rotates the distal end of the first coil. In a second instances, torquing may be accomplished by a second coil rotatably and coaxially mounted within the at least first coil, wherein the at least one coil is fixed at its proximal end to a distal end of the catheter body and at its distal end to a distal end of the second coil, wherein the first and second coils are wound in opposite helical directions so that rotation of a proximal end of the second coil in a first direction causes both the first and second coils to radially expand.

Further in accordance with at least some of the principles of the present invention as set forth above, a method for extracting clot from a blood vessel comprises positioning a radially expandable distal segment of an aspiration catheter in a blood vessel proximal to the clot. The radially distal segment of the aspiration catheter is radially expanded in the blood vessel to form an enlarged central clot-receiving passage contiguous with an aspiration lumen in the aspiration catheter. A vacuum is applied to a proximal portion of the aspiration lumen to draw clot from the blood vessel into the enlarged central clot-receiving passage. The radially expandable distal segment of the aspiration catheter is radially constricted in the blood vessel to close the central clot-receiving passage, and at least one of the radially expanding step and the radially constricting step comprises actuating structure on the aspiration catheter to open or close the central clot-receiving passage.

These methods may comprise any of the features of the present invention described previously with respect to the apparatus. For example, the radially expandable distal segment may be self-expanding and radially expanding the radially expandable distal segment may comprise releasing the radially distal expandable segment from a constraining sheath. Alternatively, radially expanding/contracting the radially expandable distal segment may comprise actuating a structure on the aspiration catheter to radially expand/contract the radially expandable distal segment. For example, actuating the structure on the aspiration catheter to expand or constrict the central clot-receiving passage may comprise torqueing at least a first coil in a first rotational direction to radially open or close the radially distal expandable segment. Optionally, the first coil may be torqued in a first direction to radially expand the radially distal segment of the aspiration catheter comprises and further torqued in a second rotational direction to radially constrain the radially distal segment of the aspiration catheter. Torqueing the first coil comprises may comprise rotating an inner member attached to a distal end of the first coil. Alternatively, torqueing the first coil may comprise rotating a second coil attached to a distal end of the first coil.

In some instances, a self-expanding radially expandable distal segment may be expanded by release from a constraining sheath and constricted by actuating a structure on the aspiration catheter to radially contract the radially expandable distal segment.

Still further in accordance with at least some of the principles of the present invention as set forth above, a catheter for resecting and aspirating clot from a blood vessel comprises a catheter body having a proximal end, a distal end, and an aspiration lumen therebetween. A radially expandable scaffold having a central clot-receiving passage extends distally from the distal end of the catheter body, and at least a distal portion of the radially expandable scaffold is configured be disrupt a region of clot when radially expanded therein. The central clot-receiving passage is configured to pass disrupted clot into the aspiration lumen when a vacuum is applied to a proximal end of the aspiration lumen.

The catheter for resecting and aspirating clot of the present invention may further comprise an elastic sleeve that covers at least a proximal portion of the radially expandable scaffold, typically leaving a distal resection portion uncovered. The elastic sleeve is typically configured to cover central clot-receiving passage to create a continuous vacuum path through the central clot-receiving passage and into a distal end of the aspiration lumen, thus allowing resected clot to be aspirated directly from the central clot-receiving passage, through the aspiration lumen in the aspiration catheter, and to an external vacuum collection receptacle.

The aspiration catheter body may further comprise means integrated with the catheter body for expanding and/or constricting the radially expandable scaffold between a radially expanded configuration and a radially contracted configuration. For example, the means integrated with the catheter body for expanding and/or constricting the radially distal expandable segment may comprise at least a first coil which is configured to be torqued in at least one rotational direction to radially open or close the radially distal expandable segment, wherein the first coil is typically configured to be torqued in both rotational directions to radially open and close the radially distal expandable segment. For example, the first coil may be fixed at its proximal end to a distal end of the catheter body and at its distal end to a distal end of the inner member, wherein rotation of a proximal end of the inner member rotates the distal end of the first coil. Alternatively, a second coil may be rotatably and coaxially mounted within the at least first coil, where the at least one coil is fixed at its proximal end to a distal end of the catheter body and at its distal end to a distal end of the second coil. The first and second coils may be wound in opposite helical directions so that rotation of a proximal end of the second coil in a first direction causes both the first and second coils to radially expand.

In other instances, the radially expandable scaffold may self-expanding, and the catheter may further comprise a sheath configured to constrain the radially expandable scaffold in a radially constrained configuration, wherein retraction of the sheath allows the radially distal expandable segment to radially expand. The radially expandable scaffold may comprise closed cells, serpentine rings, axial struts, or may have any of a variety of other scaffold constructions commonly used in constructing vascular prostheses.

In yet further accordance with at least some of the principles of the present invention as set forth above, a method for disrupting and extracting clot from a blood vessel comprises positioning a radially expandable scaffold or expandable coil at a distal end of an aspiration catheter within a region of clot in a blood vessel. The radially expandable scaffold or expandable coil is radially expanded within the region of clot and disrupted clot is collected within a central clot-receiving passage of the scaffold which is contiguous with an aspiration lumen in the aspiration catheter. By applying a vacuum to a proximal portion of the aspiration lumen, disrupted clot may be extracted from the central clot-receiving passage into the lumen.

The radially distal expandable segment may be self-expanding and expanding the radially expandable distal segment of the aspiration catheter may comprise releasing the radially distal expandable segment from a constraining sheath. Alternatively, radially expanding the radially expandable distal segment of the aspiration catheter may comprise actuating structure on the aspiration catheter to radially expand the radially expandable distal segment. For example, actuating structure on the aspiration catheter to expand or constrict the central clot-receiving passage may comprise torquing at least a first coil in a first rotational direction to radially open or close the radially distal expandable segment. The first coil may be torqued in a first direction to radially expand the radially distal segment of the aspiration catheter and torqued in a second rotational direction to radially constrain the radially distal segment of the aspiration catheter. Torquing the first coil may comprise rotating an inner member attached to a distal end of the first coil. Alternatively, torquing the first coil may comprise rotating a second coil attached to a distal end of the first coil.

In an exemplary example, the expandable distal segment comprises a self-expanding structure which is constrained in the smaller configuration and then released and/or allowed to self-expand to the larger configuration.

In an exemplary example, the expandable distal segment comprises a self-expanding structure which is constrained by an outer sheath partially or fully covering the expandable distal segment, and the outer sheath is moved proximally relative to the expandable distal segment and/or the expandable distal segment is moved distally relative to the outer sheath, thereby releasing the constraint and allowing the self-expanding structure to self-expand.

In an exemplary example, the expandable distal segment comprises a self-expanding structure which is constrained by a sheath (or cap) at its distal end partially or fully covering the expandable distal segment, and the sheath or cap is moved distally relative to the expandable distal segment and/or the sheath or cap is moved proximally relative to the expandable distal segment and/or the expandable distal segment is moved proximally relative to the sheath or cap, thereby releasing the constraint and allowing the self-expanding structure to self-expand. In one example, the sheath (or cap) are controlled by a wire or tube slidably movable inner to the expandable structure.

In an exemplary example, the expandable distal segment comprises a self-expanding structure comprising struts, tines, hooks, or other means by which the expandable distal segment is constrained from the inside by a wire or inner elongated tubular body inside the outer elongated tubular body, and the wire or inner elongated tubular body is moved proximally within the outer elongated tubular body to release the constraint and allowing the self-expanding structure to self-expand.

In an exemplary example, the expandable distal segment comprises a self-expanding structure comprising holes or loops within its structure and the expandable distal segment is constrained from the inside by wires or inner elongated tubular body inside the outer elongated tubular body shaped to engage such holes or loops, and the wire or inner elongated tubular body is moved proximally within the outer elongated tubular body to release the constraint and allowing the self-expanding structure to self-expand.

In an exemplary example, the expandable distal segment comprises a self-expanding structure, which is covered by a constraining ring over the distal portion of the expandable distal segment, and the ring is moved proximally to partially or fully uncover the distal portion of the expandable distal segment, thereby allowing the self-expanding structure to self-expand.

In an exemplary example, the expandable distal segment comprises a self-expanding structure which naturally remains in the smaller configuration until exposed to heat such as about 37 degrees Celsius and/or moisture such as body moisture, which allows it to self-expand to the larger configuration.

In an exemplary example, the expandable distal segment comprises a self-expanding structure which naturally remains in the smaller configuration until charged with an electric current, which allows it to self-expand to the larger configuration.

In an exemplary example, the expandable distal segment comprises a self-expanding structure comprising linear elements or axial tines which are in a neutral state when in the larger configuration, and yield elastically when bent or compressed into the smaller configuration, from which they seek to elastically expand back to the larger configuration.

In an exemplary example, the expandable distal segment comprises a self-expanding structure comprising one or more sinusoidal rings which are in a neutral state when in the larger configuration, and yield elastically when compressed into the smaller configuration, from which they seek to elastically expand back to the larger configuration.

In an exemplary example, the expandable distal segment comprises a self-expanding structure comprising linear elements or axial tines and one or more sinusoidal rings which are in a neutral state when in the larger configuration, and yield elastically when compressed into the smaller configuration, from which they seek to elastically expand back to the larger configuration.

In an exemplary example, the expandable distal segment comprises an expandable structure which can be mechanically manipulated from the smaller configuration to the larger configuration by means of one or more of pushing, pulling, or torqueing wires, rods or tubes incorporated into the device, by means of pneumatic or hydraulic pressure, or by other means.

In an exemplary example, the expandable distal segment comprises a sleeve covering part or all of the expandable distal segment separate from the constraining means (or constraining sheath). This sleeve allows one or more of the following: hold a vacuum during aspiration, hinder back flow of blood into the aspiration device, and maximize the pressure gradient to aspirate the clot.

In an exemplary example, the expandable distal segment is expanded to a larger configuration substantially apposing the vessel wall and sufficiently holding a vacuum to aspirate a clot or hinder back flow of blood into the aspiration catheter. In this example, the vessel wall behaves like a sleeve to offer the holding of vacuum, prevent blood from substantially getting into the aspiration catheter, and/or maximize the pressure gradient to aspirate a clot. In one example, substantially all of the expandable distal segment apposes the vessel wall.

In an exemplary example, the expandable distal segment of any of the examples, wherein it expands from a crimped configuration to an expanded configuration, said expandable configuration being larger than the constraining means configuration and smaller than 1.1 times the configuration of the vessel adjacent to the expandable distal end. In a preferred example, the expandable configuration of the distal end is expanded to about the inner vessel configuration adjacent to the expandable distal end. In another example, the configuration is the diameter of the expandable segment, the vessel, and/or the sheath.

In an exemplary example, the expandable distal segment of any of these examples, comprises one or more circumferential rings, wherein said one or more circumferential rings being expandable from a crimped configuration to an expanded configuration. In one example, the circumferential rings comprise struts joined by crowns. In another example, the circumferential rings comprise two or more rings wherein adjacent rings are joined by one or more links. In another example, the circumferential rings comprise two or more rings wherein adjacent rings are joined by one or more axial links. In another example, the expandable distal segment comprises an expandable funnel-like structure, typically comprising three or more axial elements shiftable between a cylindrical configuration where the elements are axially aligned and an expanded configuration where they diverge outwardly in a distal direction. In another example, the expandable distal segment is an umbrella-like structure comprising two or more axial struts expandable from a crimped configuration to an expanded configuration and wherein one or more expandable rings joins said two or more axial struts. In yet another example, the expandable distal segment comprises one or more circumferential rings wherein the rings expand circumferentially from a crimped configuration to an expanded configuration.

In yet another example, the expandable distal segment extends proximally a length ranging from 1 mm to 150 cm, preferably ranging from 2 mm to 20 cm, more preferably ranging from 3 mm to 10 cm, and most preferably ranging from 3 to 10 mm.

In yet another example, the expandable distal segment is deployed to an expanded configuration from a crimped configuration to aspirate a clot distal to the expanded segment, and then said expanded distal segment is optionally collapsed to a smaller configuration by prior to repositioning the device within the anatomy for further aspiration procedures or withdrawing the aspiration catheter system. Means for collapsing the expandable distal segment include pulling or pushing said expanded segment into a sheath, pulling a drawstring-type thread or wire, rotating torque members to wind a coil to a tighter diameter, and other means described elsewhere herein.

In another example, the distal expandable segment has a flexibility and bendability sufficient to allow said segment to reach one or more of a blocked blood vessel in the brain, a location adjacent to a blocked blood vessel in the brain, and a location proximal to a blocked blood vessel in the brain, often a middle cerebral artery.

In another example, the distal expandable segment is configured to have flexibility sufficient to allow the distal expandable segment to reach one or more of a blocked artery in the brain, adjacent to a blocked artery in the brain, proximal to a blocked artery in the brain, middle cerebral artery.

In another example, the distal expandable segment is configured to have flexibility in all axis wherein said flexibility in said two or more axis is sufficient to allow said segment to reach one or more of a blocked artery in the brain, adjacent to a blocked artery in the brain, proximal to a blocked artery in the brain, middle cerebral artery.

In yet another example, the expandable distal segment is substantially tubular in the crimped configuration.

In yet another example, the expandable distal segment is substantially tubular in the crimped configuration and is expandable into a funnel shaped structure comprising one or more expandable elements and a sleeve covering said expandable elements.

In yet another example, the expandable distal segment is substantially tubular in the crimped configuration and is expandable into a funnel shaped structure comprising an expandable elements and a sleeve covering said expandable elements, wherein said funnel has an angle ranging from 100 degrees to 150 degrees from the delivery system, or from 10 degrees to 80 degrees from the delivery system, wherein said funnel angle is configured to inhibit collapse of said funnel when a vacuum force ranging from 50 mmHg to 760 mmHg is applied proximally to said funnel. In one example, the funnel expands distally towards the clot. In another example, the funnel expands proximally away from the clot.

In yet another example, the expandable distal segment is substantially tubular in the crimped configuration and is expandable into a funnel shaped structure comprising one or more expandable elements and a sleeve covering said expandable elements and wherein the funnel comprises an end segment configured to be substantially parallel to the vessel wall.

In a preferred example, the expandable distal segment is expandable to a configuration ranging from 0.7 times the adjacent vessel inner configuration to 1.1 times the adjacent inner vessel inner configuration to allow sufficient vacuum to remove a clot, preferably expandable to about the adjacent inner vessel configuration to allow sufficient vacuum to remove a clot.

In another example, the distal expandable segment is formed from metal, metal alloy, or polymeric material, wherein said expandable material comprises shape memory alloy or shape memory polymeric material.

Note: the terms flexibility and stiffness are commonly used when describing performance of medical devices, especially those like the present invention which need to track through blood vessels to reach the site of treatment. Stiffness is most commonly quantitatively characterized by a Three Point Bend Test, in which a portion of the scaffold, shaft, or other device component is supported on its edges by a rigid fixture while an anvil is pressed against the center of the component between the supports to force it into a curve. A load cell or other force measuring tool attached to the anvil measures the force required to bend the test unit. The stiffness of the test unit can therefore be characterized in terms of Force per Distance, such as Newtons per millimeter (N/mm). The stiffness of the device is sometimes referred to simply in terms of force, i.e. 0.6 N, when the test setup is the same for all samples in the test group such that the “per distance” aspect is common to all. As an example of a particular Three Point Bend Test setup, a product designed to be tracked in a tortuosity with a mean radius of curvature of 6.5 mm uses a three point bend test fixture with the side supports 13 mm apart, and an anvil compression distance of 2 mm in order to fully load the test samples while keeping the bending substantially in the elastic range. In such an example, a peak load of 0.6 N would correspond to an average stiffness of 0.3 N/mm. Flexibility is the qualitative inverse of stiffness, that is, a device which is more stiff than whatever it is being compared to is less flexible, and vice versa.

Other methods commonly used to assess the acute delivery performance of medical devices include Track and Push tests. A Track Test involves clamping the test device to a fixture connected to a load cell, which advances the catheter through a tortuosity while measuring the force to do so with the load cell. In this case the Force per Distance output data tends to form a series of sinusoids with peaks of increasing elevation, where each rise in data corresponds to the force required to advance the device through a particular curve in the anatomy. Typically, the data in analyzed in terms of peak force—the greatest amount of force required to advance the device through any point in the fixture. Data can also be analyzed in terms of average force over the whole distance, average force for a section (such as around a particular curve), or even distance advanced until a certain force ceiling is hit. A Push test uses a generally similar test setup, except a second load cell is anchored somewhere in the tortuosity, and the test device advanced until its tip is in contact with the second load cell. The test device is then further advanced thereby putting the device in compression between the load cells, and the efficiency of force transmission from the proximal to the distal load cell determined. For example, if the proximal load cell reads 1.0 N applied force and the distal load cell reads 0.3 N at the catheter tip, push transmission is 30%.

The inventions claimed herein are further set forth and described in the following numbered clauses:

Clause 1. A clot aspiration system for aspirating clot from a blood vessel, said clot aspiration system comprising:
an aspiration assembly having a proximal end and a distal end and including at least an aspiration catheter having a proximal end, an open distal end, and an aspiration lumen therebetween;
an inner catheter having a proximal end, a distal end, and a guide wire lumen therebetween, wherein the inner catheter is slidably received in the aspiration lumen of the aspiration catheter; and
a transition structure coupled to a distal segment of the inner catheter wherein the transition structure covers or fills the open distal end of the aspiration catheter when the distal end of the inner catheter is positioned at or near the distal end of the aspiration catheter.

Clause 2. The clot aspiration system of clause 1, wherein the transition structure is coupled to a distal end of the aspiration catheter.

Clause 3. The clot aspiration system of clause 1, wherein the transition structure is coupled to a location within 10 cm of a distal tip of the distal segment, preferably within 5 cm of the distal tip.

Clause 4. The clot aspiration system of clause 1, wherein a proximal portion of the transition structure is coupled to the distal segment of the inner catheter and a distal portion of the transition structure is detachably coupled to the distal end of the aspiration catheter.

Clause 5. The clot aspiration system of clause 1 to 4, wherein the transition structure is coupled to the distal segment by any one or more of attaching, adhering, soldering, overlaying, wedging, stretching over, suturing, gluing, crimped, constraining, heat bonding, fusing, molding, and extruding.

Clause 6. The clot aspiration system of clause 1 to 4, wherein the transition structure is an integral component of the inner catheter.

Clause 7. The clot aspiration system of clause 1 to 6, wherein the transition structure is formed at least partly from a material which is the same as a material used to form the inner catheter.

Clause 8. The clot aspiration system of clause 1 to 6, wherein the transition structure is formed at least partly from a material which is different than a material used to form the inner catheter.

Clause 9. The clot aspiration system of clause 1 to 8, further comprising an annular gap between an outer surface of the inner catheter and an inner surface of the aspiration lumen at a distal end of the clot aspiration system, wherein the transition structure covers or fills the annular gap when the distal end of the inner catheter is positioned at or near the distal end of the aspiration catheter by a preselected distance.

Clause 10. The clot aspiration system of clause 1 to 9, wherein the distal end of the inner catheter protrudes beyond the distal end of the aspiration catheter by a preselected distance and the transition structure covers the open distal end of the aspiration catheter.

Clause 11. The clot aspiration system of clause 1 to 9, wherein the inner catheter is flush with the distal end of the aspiration catheter and the transition structure fills the open distal end of the aspiration catheter.

Clause 12. The clot aspiration system of clause 9 to 11, wherein the annular gap has a mean width in a ranges from 0.025 mm to 2 mm, from 0.05 mm to 1 mm, or from 1 mm to 1.25 mm.

Clause 13. The clot aspiration system of clause 9 to 12, wherein the annular gap extends from the distal end over a length in a range from 1 cm to 110 cm, 1 cm to 50 cm, or 1 cm to 25 cm.

Clause 14. The clot aspiration system of clause 9 to 13, wherein the aspiration catheter and the inner catheter are arranged coaxially about an axis of the guide wire lumen along at least a distal segment of the aspiration catheter and/or the inner catheter.

Clause 15. The clot aspiration system of clause 14, wherein the distal segment has a length ranging from 1 cm to 40 cm, preferably ranges from 1 cm to 25 cm.

Clause 16. The clot aspiration system of clause 1 to 15, wherein the inner catheter further comprises a push tube or rod, proximal to said distal segment and configured to be coupled to the proximal end of the distal segment of the inner catheter, to advance and/or withdraw the inner catheter through the aspiration catheter lumen.

Clause 17. The clot aspiration system of clause 1 to 15, wherein an outer surface of the inner catheter closely conforms to an inner surface of the aspiration lumen of the aspiration, leaving a minimal gap over at least a partial length of the clot aspiration system.

Clause 18. The clot aspiration system of clause 1 to 119, wherein the aspiration assembly further includes a sheath having a proximal end and a distal end and being slidably disposed over an outer surface of the aspiration catheter.

Clause 19. The clot aspiration system of clause 18, wherein the sheath, the aspiration catheter, and the inner catheter are arranged coaxially about an axis of the guide wire lumen over at least a portion of their lengths.

Clause 20. The clot aspiration system of clause 18 or 19, wherein the transition structure covers or fills an open end of the sheath when the distal end of the inner catheter is positioned at or near the distal end of the aspiration catheter.

Clause 21. The clot aspiration system of clause 1 to 20, wherein the transition structure is tapered to lower a pushing force to advance the clot aspiration system into or through the blood vessel.

Clause 22. The clot aspiration system of clause 1 to 20, wherein the transition structure is blunt.

Clause 23. The clot aspiration system of clause 1 to 22, wherein the transition structure is configured to form a distal tip at the distal end of the aspiration assembly when the inner catheter is disposed in the aspiration lumen of the aspiration catheter, wherein the distal tip is configured to be retracted proximally to leave the distal end of the aspiration catheter open when the inner catheter is retracted.

Clause 24. The clot aspiration catheter of clause 1 to 23, wherein a proximal end of the transition structure is shaped to conform to the open distal end of the aspiration catheter.

Clause 25. The clot aspiration catheter of clause 1 to 23, wherein a proximal end of the transition structure is larger than the open distal end of the aspiration catheter and covers the open distal end of the aspiration catheter when the inner catheter is in the aspiration lumen.

Clause 26. The clot aspiration catheter of clause 1 to 23, wherein the proximal end of the transition is smaller or the same as the open distal end of the aspiration catheter and fills or partially fills the open distal end of the aspiration lumen.

Clause 27. The clot aspiration catheter of clause 1 to 26, wherein a proximal end of the transition structure is detachably coupled to the aspiration catheter.

Clause 28. The clot aspiration catheter of clause 1 to 26, wherein a distal end of the transition structure is fixedly coupled to the inner catheter.

Clause 29. The clot aspiration system of clause 1 to 28 wherein the inner catheter is configured to be advanced to displace the transition structure to uncover the distal end of the aspiration catheter.

Clause 30. The clot aspiration system of clause 1 to 29, wherein the transition structure is configured to be retracted through the aspiration lumen of the aspiration catheter by pulling proximally on the inner catheter.

Clause 31. The clot aspiration system of clause 1 to 23, wherein transition structure comprises a collapsible shell having a proximal end which overlaps an outer surface of the aspiration assembly to close or partially close the distal end of the aspiration assembly during advancement of the aspiration assembly through the blood vessel.

Clause 32. The clot aspiration system of clause 31, wherein the shell has a conical, prong, scaffold, or bullet-shape profile.

Clause 33. The clot aspiration system of clause 1 to 32 wherein the transition structure is configured to additionally cover a distal length of the aspiration catheter outer surface in a range from 1 mm to 10 cm.

Clause 34. The clot aspiration system of clause 1 to 33 wherein transition structure comprises an inflatable occlusion member which fills the open distal end of the aspiration catheter when inflated.

Clause 35. The clot aspiration system of clause 34, wherein the inflatable occlusion member covers the distal end of the aspiration assembly when inflated.

Clause 36. The clot aspiration system of clause 34, wherein the distal tip of the aspiration catheter nests in a step formed in an outer surface of the inflatable occlusion member.

Clause 37. The clot aspiration system of clause 1 to 36, wherein the aspiration catheter has an inner diameter in a range from 1 mm to 3.25 mm, preferably ranging from 1.5 mm to 2.35 mm, wherein the inner catheter has an outer diameter in a range from 0.25 mm to 2.35 mm, preferably ranging from 0.5 mm to 1.54 mm, and wherein the preselected distance is in a range from 5 mm to 100 mm, preferably ranging from 30.5 mm and 100 mm.

Clause 38. The clot aspiration catheter system of clause 1 to 37, wherein the inner catheter distal end inner diameter ranges from 0.5 mm to 25 mm, preferably from 1 mm to 2.5 mm, and wherein the distal end outer diameter ranges from 0.55 mm to 3 mm, preferably from 1.05 mm to 3 mm.

Clause 39. The clot aspiration catheter system of clause 1 to 38, wherein the aspiration catheter distal end inner diameter ranges from 1.5 mm to 30 mm, preferably from 2 mm to 5 mm, and wherein the distal end outer diameter ranges from 1.55 mm to 30.05 mm, preferably from 2.05 mm to 5.05 mm.

Clause 40. The clot aspiration catheter system of clause 1 to 39, wherein the inner catheter has the same inner diameter and/or outer diameter along the length of the inner catheter.

Clause 41. The clot aspiration catheter system of clause 1 to 40, wherein the inner catheter has variable inner diameter and/or outer diameter along the length of the inner catheter.

Clause 42. The clot aspiration system of clause 1 to 37, wherein the inner catheter comprises an elongated rod having a tapered tip and a guidewire lumen.

Clause 43. The aspiration system of clause 1 to 42, further comprising an outer catheter having a proximal end, a distal end, and a central passage therethrough.

Clause 44. The aspiration system of clause 43, wherein the aspiration catheter is slidably received within the central passage of the outer catheter and wherein the outer catheter can axially move along the entire length or a length segment of the aspiration catheter.

Clause 45. The aspiration system of clause 44, wherein the outer catheter comprises a sheath.

Clause 46. The aspiration system of clause 45, wherein the sheath comprises an access sheath configured to seal in a vascular access penetration.

Clause 47. The clot aspiration system of clause 45 or 46, wherein the sheath length is shorter than a length of the aspiration catheter length.

Clause 48. The clot aspiration system of clause 43 to 47, wherein the sheath has a working length which ranges from 1 cm to 110 cm, preferably from 10 cm to 100 cm, more preferably ranges from 10 cm to 90 cm.

Clause 49. The aspiration system of clause 43 to 48, wherein the outer catheter is configured to be introduced into the vessel with the aspiration catheter.

Clause 50. The aspiration system of clause 49, wherein there is a gap between the outer surface of the aspiration catheter and the inner surface of the central passage of the outer catheter along a majority of their lengths.

Clause 51. The aspiration system of clause 49, wherein the outer surface of the aspiration catheter closely conforms to the inner surface of the central passage of the outer catheter along a majority of their lengths.

Clause 52. The aspiration system of clause 1 to 51, wherein the transition structure is configured to cover a distal segment of the outer catheter.

Clause 53. The clot aspiration system of clause 1, wherein the transition structure comprises a solid spherical or ovoidal plug that fills the annular gap and provides a rounded surface projecting distally from the distal end of the aspiration catheter when the distal end of the inner catheter is near the distal end of the aspiration catheter by the preselected distance.

Clause 54. The clot aspiration system of clause 1, wherein the transition structure comprises a solid spherical or ovoidal plug that fills the annular gap and has a shoulder that covers an exposed distal surface of the aspiration catheter when the distal end of the inner member protrudes beyond the distal end of the aspiration catheter by the preselected distance, said plug having a rounded surface projecting distally from the distal end of the aspiration catheter.

Clause 55. The clot aspiration system of clause 1, wherein the transition structure comprises a conical cap that covers the annular gap when the distal end of the inner member protrudes beyond the distal end of the aspiration catheter by the preselected distance.

Clause 56. The clot aspiration system of clause 55, wherein the transition structure comprises an inflated balloon that fills the annular gap when the distal end of the inner member protrudes beyond the distal end of the aspiration catheter by the preselected distance.

Clause 57. The clot aspiration system of clause 1 to 56, wherein the transition structure comprises one or more materials selected from a group consisting of an elastic material, a polymeric material, a metallic material, and a shape memory material, individually or in combination.

Clause 58. The clot aspiration system of clause 1 to 57, wherein the transition structure covers, partially covers, fills, or partially fills the open distal end of the aspiration catheter.

Clause 59. The clot aspiration system of clause 1 to 58, wherein the transition structure comprises a metallic scaffold, a polymeric membrane, a polymeric scaffold, a combination of metallic scaffold and polymeric membrane, a malleable material, a shape memory alloy scaffold, a multi-prong metallic or polymeric structure, or other structure that cover or partially covers or fills or partially fills, the open distal end or a distal segment of the aspiration catheter.

Clause 60. The clot aspiration system of clause 1 to 59, wherein the transition structure is detachably coupled to the distal segment of the aspiration catheter and is configured to be slidably retracted through the lumen of the aspiration catheter lumen after detaching.

Clause 61. The clot aspiration system of clause 60, wherein the transition structure when detached is smaller than the open distal end of the aspiration catheter.

Clause 62. The clot aspiration system of clause 60, wherein the transition structure when detached is larger than the open distal end of the aspiration catheter and is configured to be compressible to slidably be retracted through the aspiration catheter lumen.

Clause 63. The clot aspiration system of clause 60 to 62, wherein a proximal end of the transition structure is configured to be decoupled from a distal tip of the distal segment of the aspiration catheter.

Clause 64. The clot aspiration system of clause 60 to 62, wherein a proximal end of the transition structure is configured to be decoupled from a distal tip of the distal segment of the aspiration catheter.

Clause 65. The clot aspiration system of clause 60 to 62, wherein the inner catheter is configured to retract the transition structure proximally into the aspiration lumen causing the transition structure to invert as it is retracted.

Clause 66. The clot aspiration system of clause 1 to 65, further comprising an elongated stiffening member configured to be disposed over the inner catheter and inside the aspiration catheter lumen.

Clause 67. The clot aspiration system of clause 66, wherein the elongated stiffening member comprises an elongated rod having a tapered tip and a guidewire lumen.

Clause 68. A method for aspirating clot from a patient's blood vessel, said method comprising:
providing an aspiration assembly including (1) an aspiration catheter having a proximal end, an open distal end, and an aspiration lumen therebetween and (2) an inner catheter having a proximal end, a distal end, and a guide wire lumen therebetween; and
advancing a distal end of the aspiration assembly into the blood vessel over a guide wire passing through the guide wire lumen of the inner catheter, wherein a transition structure covers, fills, partially covers, partially fills, and/or abuts the open distal end of the aspiration assembly;
retracting the transition structure;
positioning the open end of the aspiration catheter near the clot;
applying a negative pressure through the aspiration lumen to draw clot through the open end into said lumen.

Clause 69. The method of clause 68, wherein the transition structure comprises a cone having a base covering the open distal end of the aspiration catheter, wherein retracting the transition structure comprises advancing the cone so that the base radially collapses and pulling the inner catheter proximally out of the aspiration lumen.

Clause 70. The method of clause 68, wherein the transition structure comprises an inflatable structure inflated in a distal end of the aspiration lumen, wherein retracting the transition structure comprises delating the inflatable structure and pulling the inner catheter proximally out of the aspiration lumen.

Clause 71. The method of clause 68 to 70, wherein the aspiration assembly further comprises an outer catheter having a distal end which is initially covered, partially covered, filled, partially filled, and/or abutted by the transition structure.

Clause 72. The method of clause 68 to 71, wherein the outer catheter comprises a sheath.

Clause 73. The method of clause 72, wherein the outer catheter comprises an access sheath configured to seal in a vascular access penetration.

Clause 74. The method of clause 68 to 73, wherein the transition structure is withdrawn from the blood vessel prior to aspiration of the clot.

Clause 75. The method of clause 68 to 74, wherein the transition structure supports the aspiration catheter during advancement into or through the blood vessel.

Clause 76. The method of clause 68 to 75, wherein the transition structure enhances tracking of the aspiration catheter through the blood vessel to position the open end of the aspiration catheter near the clot prior to retracting the transition structure.

Clause 77. The method of clause 68 to 76, wherein the transition structure enhances pushability of the aspiration catheter through the blood vessel.

Clause 78. The method of clause 68 to 77, wherein the transition structure is formed from one or more of an elastic material, polymeric material, metallic material, or shape memory material, individually or in combination.

Clause 79. The method of clause 78, wherein retracting the transition structure comprises detaching the transition structure from the aspiration catheter assembly and withdrawing the detached transition structure through the aspiration lumen.

Clause 80. The method of clause 79, wherein retracting the transition comprises deforming the structure to fit in the aspiration lumen.

Clause 81. The method of clause 79, wherein retracting the transition comprises radially collapsing the structure to fit in the aspiration lumen.

Clause 82. A clot aspiration system for aspirating clot from a blood vessel, said catheter comprising:
an aspiration assembly having a proximal end and a distal end and including (1) an outer sheath having a proximal end, an open distal end, and a central lumen therebetween and (2) an aspiration catheter having a proximal end, an open distal end, and an aspiration lumen therebetween, wherein the aspiration catheter is slidably received in the central lumen of the outer sheath; and
a tapered transition structure positioned over the open distal ends of the outer sheath and the aspiration catheter, wherein the tapered transition structure is attached to at least one of the outer sheath and the aspiration catheter and is configured to radially open to allow the open distal end of the aspiration catheter to be distally advanced beyond the open distal end of the outer sheath.

Clause 83. The clot aspiration system of clause 82, wherein a distal tip of the tapered transition structure is configured to be advanced over a guidewire prior to being radially opened.

Clause 84. The clot aspiration system of clause 82, further comprising an inner catheter having a distal portion configured to pass through a distal tip of the tapered transition prior to being radially opened.

Clause 85. The clot aspiration system of clause 84, wherein the inner catheter is configured to be advanced over a guidewire.

Clause 86. The clot aspiration system of clause 84 or 85, wherein the distal tip of the tapered transition structure is detachably attached to the inner catheter by a tether, such as a suture loop.

Clause 87. The clot aspiration system of clause 82 to 86, wherein the transition structure is coupled to the distal end of the outer sheath.

Clause 88. The clot aspiration system of clause 87, wherein the tapered transition structure is preformed into a conical shape which opens in response to distal advancement of the aspiration catheter through the tapered transition structure.

Clause 89. The clot aspiration system of clause 82 to 88, wherein the transition structure is detachably coupled to the distal end of the aspiration assembly and configured to be slidably retracted through the lumen of the aspiration catheter lumen after detaching said transition structure from the distal end of the aspiration assembly.

Clause 90. The clot aspiration system of clause 89, wherein the transition structure when detached has a smaller configuration than the open distal end of the aspiration catheter.

Clause 91. The clot aspiration system of clause 89, wherein the transition structure when detached has a larger configuration than the open distal end of the aspiration catheter but is configured to be compressible to slidably be retracted through the aspiration catheter lumen.

Clause 92. The clot aspiration system of clause 89 to 91, wherein a proximal end of the transition structure is configured to be retracted into the aspiration lumen first when retracting the transition structure into the aspiration lumen.

Clause 93. The clot aspiration system of clause 89 to 91, wherein a distal end of the transition structure is configured to be retracted into the aspiration lumen first when retracting the transition structure into the aspiration lumen.

Clause 94. The clot aspiration system of clause 82 to 93, wherein the transition structure is configured to invert when retracting the structure into the aspiration lumen.

Clause 95. A clot method for aspirating clot from a patient's blood vessel, said method comprising:
providing an aspiration assembly including (1) an aspiration catheter having a proximal end, an open distal end, and an aspiration lumen therebetween and (2) an outer catheter having a proximal end, an open distal end, and a central lumen therebetween; and
advancing a distal end of the aspiration assembly into the blood vessel over a guide wire passing through the aspiration lumen of the aspiration catheter, wherein a transition structure covers, partially covers, fills, or partially fills, the open distal ends of the aspiration and outer catheters;
opening or removing the transition structure;
passing the open distal end of the aspiration catheter through the distal end of the outer catheter;
positioning the open distal end of the aspiration catheter near the clot;
applying a negative pressure through the aspiration lumen to draw clot through the open end into said lumen.

Clause 96. The method of clause 95, wherein the transition structure comprises a cone having a base attached to the distal end of the outer catheter.

Clause 97. The method of clause 95, wherein opening the transition structure comprises advancing the open distal end of the aspiration catheter through the cone to open said cone.

Clause 98. The method of clause 95 to 97, further comprising detaching the transition structure from a distal end of the aspiration assembly and slidably retracting the detached transition structure through the lumen of the aspiration catheter lumen or over the aspiration catheter.

Clause 99. The method of clause 95 to 98, wherein the aspiration assembly further comprises an inner catheter having a distal end initially passing through the transition structure.

Clause 100. The method of clause 95 to 99, wherein the outer catheter comprises a sheath.

Clause 101. The method of clause 100, wherein the outer catheter comprises an access sheath configured to seal in a vascular access penetration.

Clause 102. An aspiration catheter for removing clot from a blood vessel, said aspiration catheter comprising:
a catheter body having a proximal end, a distal end, and an aspiration lumen therebetween; and
a distal tip structure extending distally from the distal end of the catheter body and having a central clot-receiving passage contiguous with the aspiration lumen of the catheter body;
wherein the distal tip structure is formed at least partly from a malleable material so that the distal tip structure non-elastically reshapes from a furled configuration to an unfurled configuration in response to being engaged against clot as the aspiration catheter is advanced in a blood vessel.

Clause 103. The aspiration catheter of clause 102, wherein the distal tip structure unfurls to conical shape with an enlarged, open distal end.

Clause 104. The aspiration catheter of clause 102 or 103, wherein the malleable material comprises a polymer.

Clause 105. The aspiration catheter of clause 102 to 104, wherein malleable material comprises a metal.

Clause 106. The aspiration catheter of clause 105, wherein the metal comprises an unset shape memory alloy.

Clause 107. The aspiration catheter of clause 106, wherein unset shape memory alloy comprises an unset nickel-titanium alloy.

Clause 108. The aspiration catheter of clause 102 to 107, wherein the distal tip structure in its unfurled configuration comprises a pleated cone having a truncated end attached to the distal end of the catheter body and a radially expanded, open distal end.

Clause 109. The aspiration catheter of clause 102 to 108, wherein the distal tip structure is configured to be introduced in low profile configuration and to expand to a higher profile configuration in response to being engaged against clot as the aspiration catheter is advanced in a blood vessel.

Clause 110. The aspiration catheter of clause 102 to 109, wherein the distal tip structure is configured to inspire clot into an interior volume and to collapse over the inspired clot in response to application of a negative pressure of at least 0.9 atm, at least 0.8 atm, at least 0.7 atm, at least 0.6 atm, at least 0.5 atm, at least 0.4 atm, at least 0.3 atm, at least 0.1 atm, at least 0.1 atm, and at least 0.05 atm in the central clot-receiving passage of the distal tip structure.

Clause 111. The aspiration catheter of clause 102 to 109, wherein the distal tip structure is configured to collapse after the clot has move proximally of the central clot-receiving passage of the distal tip structure in response to application of a negative pressure of at least 0.9 atm, at least 0.8 atm, at least 0.7 atm, at least 0.6 atm, at least 0.5 atm, at least 0.4 atm, at least 0.3 atm, at least 0.1 atm, at least 0.1 atm, and at least 0.05 atm in the central clot-receiving passage of the distal tip structure.

Clause 112. A clot aspiration catheter for aspirating clot from a blood vessel, said catheter comprising:
an aspiration catheter having a proximal end, a distal end, and an aspiration lumen therebetween;
an elongated inner stiffening member having a proximal end, a distal end, and a guide wire lumen therebetween, said elongated inner stiffening member being removably received in the aspiration lumen of the aspiration catheter; and
one or more frictional anchors disposed on a distal region of the elongated inner stiffening member, wherein said one or more frictional anchors are configured to reversibly engage an inner wall of the aspiration lumen to enhance pushability and/or trackability of the aspiration catheter as it is being advanced through a blood vessel.

Clause 113. The clot aspiration catheter of clause 112, comprising a plurality of frictional anchors axially spaced-apart over at least a distal region of the elongated inner stiffening member.

Clause 114. The clot aspiration catheter of clause 112, wherein the frictional anchors comprise one or more inflatable balloons wherein said balloons are configured to be inflated to engage and deflated to reversibly disengage the inner wall of the aspiration catheter to enhance stiffness and pushability.

Clause 115. The clot aspiration catheter of clause 112, wherein the elongated inner stiffening member comprises one or more lumens configured to inflate said one or more inflatable balloons.

Clause 116. The clot aspiration catheter of clause 114 or 115, wherein said balloons have the same expanded diameters.

Clause 117. The clot aspiration catheter of clause 114 or 115, wherein said balloons have different expanded diameters so that larger inflated balloons may engage the inner wall of the aspiration catheter while smaller inflated balloons do not engage the inner wall of the aspiration catheter.

Clause 118. The clot aspiration catheter of clause 112 to 117, wherein the aspiration catheter has a diameter in a range from 1.5 mm to 3.25 mm, the inner stiffening member has a diameter in a range from 0.5 mm to 2.35 mm, the anchors are distributed over a distance in a range from 0.5 cm to 50 cm, preferably over a range from 15 cm to 25 cm, or alternatively from 0.5 cm to 15 cm of the distal end of the aspiration catheter, and the anchors are axially spaced-apart by a distance in a range between 5 mm and 25 mm.

Clause 119. The clot aspiration catheter of clause 1 to 3, wherein the elongated inner stiffening member comprises an elongated rod having a tapered tip and a guidewire lumen.

Clause 120. The clot aspiration catheter of clause 112 to 119, wherein the elongated inner stiffening member comprises an inner catheter.

Clause 121. The clot aspiration catheter of clause 120, wherein the plurality of expandable frictional anchors are configured not to engage the inner wall of the aspiration catheter when fully expanded.

Clause 122. The clot aspiration catheter of clause 120, wherein the plurality of expandable frictional anchors are configured to have a variable engagement with the inner walls of the aspiration catheters ranging from no engagement, to partial engagement, to full engagement when fully expanded.

Clause 123. An aspiration catheter for removing clot from a blood vessel, said aspiration catheter comprising:
a catheter body having a proximal end, a distal end, and an aspiration lumen therebetween;
a scaffold extending distally from the distal end of the catheter body and having a central clot-receiving passage contiguous with the aspiration lumen of the catheter body; and
a vacuum-resistant membrane covering the scaffold to establish a clot aspiration path from a distal end of the scaffold to a proximal end of the aspiration lumen in the catheter body so that applying a vacuum to the proximal end of the aspiration lumen can draw clot into the central clot-receiving passage;
wherein the scaffold is configured elongate and radially collapse in response to a proximal tension.

Clause 124. The aspiration catheter of clause 123, wherein further comprising at least one pull strut connecting a distal end of the catheter body to a proximal end of the scaffold, wherein the at least one pull strut is configured to apply a localized stress on the scaffold which causes the scaffold to elongate and collapse radially.

Clause 125. The aspiration catheter of clause 124, comprising a single pull strut connected to one location on a closed-loop scaffold.

Clause 126. The aspiration catheter of clause 124, comprising at least first and second pull struts connected to first and second spaced-apart locations on a proximal periphery of a closed-loop scaffold.

Clause 127. The aspiration catheter of clause 123, wherein further comprising first and second pull struts connecting a distal end of the catheter body to a proximal end of the scaffold, wherein the scaffold consists of an open loop having first and second ends and the first pull strut is connected to the first end of the open loop scaffold and the second pull strut is connected at the second end of the open loop scaffold.

Clause 128. The aspiration catheter of clauses 123 to 127, wherein the scaffold comprises a single member free from branching.

Clause 129. The aspiration catheter of clause 124, wherein comprising a single pull strut connected to a proximal end of a single member having a free distal end.

Clause 130. The aspiration catheter of clause 128 or 129, wherein the single member is formed into a cylinder or cone with undulating regions.

Clause 131. The aspiration catheter of clause 123 to 130, wherein the scaffold comprises a malleable material that allows irreversible elongation and radial collapse.

Clause 132. The aspiration catheter of clause 123 to 130, wherein the scaffold comprises an elastic material that allows reversible elongation and radial collapse.

Clause 133. The aspiration catheter of clause 123 to 132, wherein the scaffold comprises:
a plurality of circumferential rings arranged along an axis and patterned from a non-degradable material, said scaffold being configured to expand from a crimped configuration to an expanded configuration;
wherein the circumferential rings are joined by axial links and each of the axial links includes a circumferential separation region; and
wherein said scaffold is configured to circumferentially separate along separation interfaces and to form one continuous structure after all axial links have separated along said circumferential separation regions.

Clause 134. An aspiration catheter for removing clot from a blood vessel, said aspiration catheter comprising:

a catheter body having a proximal end, a distal end, and an aspiration lumen therebetween;
a scaffold extending distally from the distal end of the catheter body and having a central clot-receiving passage contiguous with the aspiration lumen of the catheter body; and
a membrane comprising an elastic sleeve covering the scaffold to establish a clot aspiration path from a distal end of the scaffold to a proximal end of the lumen in the catheter body so that applying a vacuum to a proximal end of the aspiration lumen can draw clot into the central clot-receiving passage;
wherein at least a distal portion of the scaffold is radially expandable from a delivery configuration to an extraction configuration and wherein the distal portion of the scaffold is configured to controllably collapse from the extraction configuration to a partially collapsed configuration in response to a vacuum applied within the central clot-receiving passage, wherein said collapsed configuration is sufficient to allow the aspiration of the clot into the aspiration lumen.

Clause 135. The aspiration catheter as in clause 134, wherein the scaffold is embedded in the membrane.

Clause 136. The aspiration catheter as in clause 134, wherein membrane is attached to the scaffold.

Clause 137. The aspiration catheter as in clause 134, wherein the partially collapsed configuration has an average width in a range from 0.25 to 0.75 of a width of the radially expanded configuration.

Clause 138. The aspiration catheter as in clause 134 to 137, wherein the scaffold has is configured to partially collapse when a vacuum in a range from 0.2 atm to 1 atm is applied to the central clot-receiving passage.

Clause 139. The aspiration catheter as in clause 134 to 138, wherein the scaffold self-expands to the extraction configuration when a pressure in the central clot-receiving passage is above 0.2 atm.

Clause 140. The aspiration catheter of clause 134 to 139, wherein the radially expandable distal portion of the scaffold is configured to be reversibly driven between a radially contracted configuration and a radially expanded configuration, and a partially collapsed configuration.

Clause 141. The aspiration catheter of clause 134 to 140, wherein the radially expanded extraction configuration comprises a substantially cylindrical distal region configured to engage an inner wall of the blood vessel and a tapered transition region between the cylindrical distal region and the distal end of the catheter body, wherein the cylindrical distal region has an open distal end configured to direct clot into the central clot-receiving passage when the vacuum is applied to a proximal end of the aspiration lumen.

Clause 142. The aspiration catheter of clause 134 to 141, wherein the radially expanded extraction configuration comprises a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen.

Clause 143. The aspiration catheter of clause 134 to 142, wherein the scaffold comprises struts joined by crown, further comprising stops on adjacent struts to limit the collapse of the scaffold under pressure.

Clause 144. The aspiration catheter of clause 143, wherein the stops comprises circumferentially aligned tabs.

Clause 145. The aspiration catheter of clause 134 to 144, wherein the scaffold comprises a polymeric material.

Clause 146. The aspiration catheter of clause 134 to 144, wherein the scaffold comprises a shape memory material.

Clause 147. The aspiration catheter of clause 134 to 144, wherein the scaffold comprises an elastomeric material.

Clause 148. The aspiration catheter of clause 134 to 144, wherein the scaffold comprises a combination of elastomeric, polymeric and/or shape memory material.

Clause 149. A method for extracting clot from a blood vessel, said method comprising:
positioning a radially expandable distal portion of an aspiration catheter in a blood vessel proximal to the clot;
radially expanding the radially expandable distal portion of the aspiration catheter in the blood vessel to form an enlarged central clot-receiving passage through the radially expandable distal portion contiguous with an aspiration lumen in the aspiration catheter; and
applying a first level of vacuum to a proximal portion of the aspiration lumen to draw clot from the blood vessel into the radially expandable distal portion of the aspiration catheter;
increasing the vacuum level after the clot has been drawn into the radially expandable distal portion of the aspiration catheter, wherein the increased level of vacuum causes the radially expandable distal portion to partially collapse to disrupt the clot.

Clause 150. The method of clause 149, wherein the radially expandable distal portion of the aspiration catheter comprises a scaffold covered with a vacuum-resistant membrane and wherein struts of the scaffold struts act to break and/or shear the clot as the radially expandable distal portion is partially collapsed by increasing the vacuum level.

Clause 151. The method of clause 149, wherein the radially expandable distal portion of the aspiration catheter is partially collapsed to an average width in a range from 0.25 to 0.75 of an initial width of the radially expandable distal portion of the aspiration catheter.

Clause 152. The method of clause 149 or 151, wherein the first level of vacuum is in a range from 0 to 0.5 atmospheres.

Clause 153. The method of clause 152, wherein the increased vacuum level is in a range from 0.2 atm to 1 atm.

Clause 154. The method of clause 149 to 153, wherein the vacuum level is cycled up and down to enhance clot disruption after the clot has been drawn into the radially expandable distal portion of the aspiration catheter.

Clause 155. A clot disruption catheter for resecting and aspirating clot from a blood vessel, said catheter comprising:
a catheter body having a proximal end, a distal end, and an aspiration lumen therebetween;
a radially expandable distal portion of the catheter body having an expandable central clot-receiving passage contiguous with the aspiration lumen; and
clot disruption means coupled to the radially expandable distal portion of the catheter body configured to disrupt clot sufficiently pass into and through the aspiration lumen when a vacuum is applied to a proximal end of the aspiration lumen.

Clause 156. The clot disruption catheter of clause 155, wherein the clot disruption means comprises at least one cutting element disposed across the expandable central clot-receiving passage when the expandable central clot-receiving passage is expanded to cut clot as the clot is aspirated into the aspiration lumen when the vacuum is applied to the proximal end of the aspiration lumen.

Clause 157. The clot disruption catheter of clause 156, wherein the cutting element comprises a wire attached across the distal opening of the radially expandable distal portion of the catheter body, wherein the wire is folded when said radially expandable distal portion is closed and the wire is tensioned across the distal opening when said radially expandable distal portion is opened.

Clause 158. The clot disruption catheter of clause 155, wherein the cutting element comprises folding blades attached across the distal opening of the radially expandable distal portion of the catheter body, wherein the blades are folded closed when said radially expandable distal portion is in the collapsed configuration and the folding blades are substantially non-parallel when said radially expandable distal portion is in an expanded configuration.

Clause 159. The clot disruption catheter of clause 155, wherein the clot disruption means comprises a clot constriction structure disposed inside at least one of the central clot-receiving passage and the aspiration lumen, wherein the clot constriction structure is actuable to radially constrict a segment of clot after said segment has been aspirated into said at least one of the central clot-receiving passage and the aspiration lumen.

Clause 160. The clot disruption catheter of clause 155, wherein the clot constriction structure comprises a coil disposed coaxially in the at least one of the central clot-receiving passage and the aspiration lumen, wherein prior to actuation the coil lies adjacent an inner wall of the at least one of the central clot-receiving passage and the aspiration lumen and upon actuation the coils closes radially inward to constrict the clot.

Clause 161. A method for disrupting and extracting clot from a blood vessel, said method comprising:
radially expanding a radially expandable distal potion of an aspiration catheter proximal to a region of clot in the blood vessel;
applying a vacuum to a proximal portion of an aspiration lumen in the aspiration catheter to draw a segment of clot into at least one of the central clot-receiving passage and the aspiration lumen;
disrupting the clot as it is drawn into the central clot-receiving passage or after it has been received in the at least one of the central clot-receiving passage and the aspiration lumen; and
applying a vacuum to a proximal portion of an aspiration lumen in the aspiration catheter to draw a the disrupted clot through the aspiration lumen to a proximal portion of the aspiration catheter.

Clause 162. The method of clause 161, wherein disrupting the clot comprises drawing the clot across a cutting element disposed across the central clot-receiving passage as the vacuum is applied to a proximal portion of an aspiration lumen in the aspiration catheter.

Clause 163. The method of clause 162, further comprising tensioning a wire across the central clot-receiving passage as the radially expandable distal potion of an aspiration catheter is radially expanded.

Clause 164. The method of clause 162, further comprising blades being folded open across the central clot-receiving passage as the radially expandable distal portion of an aspiration catheter is radially expanded.

Clause 165. The method of clause 161, wherein disrupting the clot comprises actuating a clot constriction structure disposed inside at least one of the central clot-receiving passage and the aspiration lumen to radially constrict a segment of clot after said segment has been aspirated into said at least one of the central clot-receiving passage and the aspiration lumen, wherein the clot constriction structure is released to allow the clot to pass to a proximal portion of the aspiration lumen.

Clause 166. The method of clause 165, wherein actuating the clot constriction structure comprises closing coils over the clot.

Clause 167. The method of clause 161, wherein radially expanding the radially expandable distal potion of the aspiration catheter comprises releasing the radially expandable distal potion from a constraining sheath.

Clause 168. The method of clause 167, wherein radially expanding the radially expandable distal potion of the aspiration catheter comprises actuating the structure on the aspiration catheter to radially expand the radially expandable distal potion.

Clause 169. An aspiration catheter for removing clot from a blood vessel, said aspiration catheter comprising:
a catheter body having a proximal end, a distal end, and an aspiration lumen therebetween;
an expandable distal tip extending distally from the distal end of the catheter body and having a central clot-receiving passage contiguous with the aspiration lumen of the catheter body, wherein at least a distal portion of the expandable distal tip is radially expandable from a delivery configuration to an extraction configuration; and
an expandable seal disposed circumferentially about an exterior surface of the catheter body;
wherein the expandable seal is located a preselected distance proximal of the expandable distal tip and wherein one or more vacuum ports are formed in a wall of the catheter body in a region between the expandable distal tip and the expandable seal.

Clause 170. The aspiration catheter of clause 169, wherein the expandable seal comprises an inflatable balloon.

Clause 171. The aspiration catheter of clause 169, wherein the expandable seal comprises a deployable cuff.

Clause 172. The aspiration catheter of clause 169, wherein the expandable distal tip comprises a self-expanding scaffold.

Clause 173. The aspiration catheter of clause 169, wherein the preselected distance is in a range from 5 mm to 50 mm.

Clause 174. The aspiration catheter of clause 169, wherein the self-expanding scaffold is covered by a pressure-resistant membrane to establish a clot aspiration path from a distal end of the scaffold to a proximal end of the aspiration lumen in the catheter body so that applying a vacuum to a proximal end of the aspiration lumen can draw clot into the central clot-receiving passage.

Clause 175. A method for extracting clot from a blood vessel, said method comprising:
positioning a radially expandable distal portion of an aspiration catheter in a blood vessel proximal to the clot;
radially expanding the radially expandable distal portion of the aspiration catheter in the blood vessel to form a clot-receiving passage through the radially expandable distal portion contiguous with an aspiration lumen in the aspiration catheter;
radially expanding a circumferential seal bout the aspiration catheter at preselected distance proximal of the expanded distal tip;
applying a vacuum to a proximal portion of the aspiration lumen to draw clot from the blood vessel through the radially expandable distal portion and into the lumen of the aspiration catheter; and
wherein at least one vacuum port is disposed in a wall of the catheter body in a buffer region between the expandable distal tip and the expandable seal to draws a vacuum in said region.

Clause 176. A clot aspiration system for aspirating clot from a blood vessel, said clot aspiration system comprising:
an aspiration catheter having a proximal end, an open distal end, and an aspiration lumen therebetween;
an inner catheter having a proximal end, a distal end, and a guide wire lumen therebetween, wherein the inner catheter is slidably received in the aspiration lumen of the aspiration catheter; and
a transition structure coupled to a distal segment of the inner catheter wherein the transition structure covers a distal segment of the aspiration catheter when the distal end of the inner catheter is positioned at or near the distal end of the aspiration catheter.

Clause 177. The clot aspiration system of clause 176, wherein the transition structure is coupled to the distal segment of the aspiration catheter.

Clause 178. The aspiration system of clause 1 to 43, wherein the inner catheter is introduced into the vessel with the aspiration catheter.

Clause 179. The aspiration system of clause 1 to 67, wherein the inner catheter, the outer catheter, and the aspiration catheter are introduced into the vessel together.

Clause 180. The clot aspiration system of clause 1 to 56, wherein the transition structure comprises one or more materials selected from a group consisting of an elastic material, a polymeric material, a metallic material, and a shape memory material, individually or in combination, and wherein said material configured to radially shrink, contract, and/or have a smaller configuration compared to the open distal end of the aspiration catheter.

Clause 181. The clot aspiration system of clause 82, wherein the transition structure is attached to the outer sheath and coupled to the aspiration catheter, and wherein the transition structure is configured to radially open by advancing the aspiration catheter and/or retracting the outer sheath to allow the open distal end of the aspiration catheter to be distally advanced beyond the open distal end of the outer sheath.

Clause 182. The clot aspiration system of clause 82, wherein the transition structure is attached to the outer sheath, and wherein the transition structure distal end is configured to radially open by advancing the aspiration catheter and/or retracting the outer sheath to allow the open distal end of the aspiration catheter to be distally advanced beyond the open distal end of the outer sheath.

Clause 183. The clot aspiration system of clause 82, wherein the transition structure distal end is configured to radially open by advancing the aspiration catheter and/or retracting the outer sheath to allow the open distal end of the aspiration catheter to be distally advanced beyond the open distal end of the outer sheath.

Clause 184. The clot aspiration system of clause 82 to 86, wherein the transition structure comprises one or more materials selected from a group consisting of an elastic material, a polymeric material, a metallic material, malleable material, and a shape memory material, a multi-pronged structure, individually or in combination, or other structure that cover or partially covers or fills or partially fills, the open distal end or a distal segment of the aspiration catheter and/or the distal end of the outer sheath and/or the distal end of the inner catheter, and wherein said transition structure when opened radially expands to smaller, same, or larger than the open distal end of the aspiration catheter, and wherein the transition structure is retracted over the aspiration catheter.

Clause 185. The clot aspiration system of clause 82, wherein the transition structure is retracted over the aspiration catheter by retracting the outer sheath to allow the aspiration catheter to advance distally to aspirate the clot.

Clause 186. The clot aspiration system of clause 82 to 89, wherein the transition structure when detached has a smaller configuration than the open distal end of the aspiration catheter but is configured to be to be slidably retractable over the aspiration catheter.

Clause 187. The clot aspiration system of clause 82 to 89, wherein the transition structure when detached has a larger configuration than the open distal end of the aspiration catheter.

Clause 188. The clot aspiration system of clause 82 to 89, wherein the transition structure when detached has a larger configuration than the open distal end of the outer sheath.

Clause 189. The clot aspiration system of clause 82, wherein the transition structure is retracted over the aspiration catheter by retracting the outer sheath to allow the aspiration catheter to advance distally to aspirate the clot, and wherein the transition structure after being retracted remains in the blood vessel during aspiration of the clot.

Clause 190. The clot aspiration system of clause 82, wherein the transition structure is retracted over the aspiration, and wherein the transition structure and outer sheath are removed from the blood vessel prior to aspiration of the clot.

Clause 191. The clot aspiration catheter of clause 112, wherein the inner stiffening member and aspiration catheter are advanced together into the blood vessel and/or through the blood vessel and wherein the stiffening member has a distal segment extending beyond the distal end of the aspiration catheter and is free from the one or more frictional anchors, and wherein said segment length ranges from 5 mm to 15 cm, preferably ranges from 1 cm to 10 cm, and more preferably ranges from 1 cm to 3 cm from the distal end of the stiffening member.

Clause 192. The aspiration catheter of clause 112, further comprising an annular gap between an outer surface of the inner stiffening member and an inner surface of the aspiration lumen at a distal segment of the clot aspiration catheter, wherein the annular gap has a mean width in a ranges from 0.025 mm to 5 mm, from 0.05 mm to 2 mm, or from 0.1 mm 1.25 mm.

Clause 193. An aspiration catheter for removing clot from a blood vessel, said aspiration catheter comprising:
a catheter body having a proximal end, a distal end, and an aspiration lumen therebetween;
a scaffold extending distally from the distal end of the catheter body and having a central clot-receiving passage contiguous with the aspiration lumen of the catheter body; and
a membrane comprising an elastic sleeve covering the scaffold to establish a clot aspiration path from a distal end of the scaffold to a proximal end of the lumen in the catheter body so that applying a vacuum to a proximal end of the aspiration lumen can draw clot into the central clot-receiving passage;
wherein at least a distal portion of the scaffold is delivered in an extraction configuration and wherein the distal portion of the scaffold is configured to controllably collapse from the extraction configuration to a partially collapsed configuration in response to a vacuum applied within the central clot-receiving passage, wherein said collapsed configuration is sufficient to allow the aspiration of the clot into the aspiration lumen.

Clause 194. The aspiration catheter as in clause 193, wherein the scaffold is embedded in the membrane.

Clause 195. The aspiration catheter as in clause 193, wherein membrane is attached to the scaffold.

Clause 196. The aspiration catheter as in clause 193, wherein the partially collapsed configuration has an average width in a range from 0.25 to 0.75 of a width of the radially expanded configuration.

Clause 197. The aspiration catheter as in clause 193 to 196, wherein the scaffold has is configured to partially collapse when a vacuum in a range from 0.2 atm to 1 atm is applied to the central clot-receiving passage.

Clause 198. The aspiration catheter as in clause 193 to 197, wherein the scaffold self-expands to the extraction configuration when a pressure in the central clot-receiving passage is above 0.2 atm.

Clause 199. The aspiration catheter of clause 193 to 198, wherein the distal portion of the scaffold is configured to be reversibly driven between a radially contracted configuration and a radially expanded configuration, and a partially collapsed configuration.

Clause 200. The aspiration catheter of clause 193 to 199, wherein the extraction configuration comprises a substantially cylindrical distal region configured to engage an inner wall of the blood vessel and a tapered transition region between the cylindrical distal region and the distal end of the catheter body, wherein the cylindrical distal region has an open distal end configured to direct clot into the central clot-receiving passage when the vacuum is applied to a proximal end of the aspiration lumen.

Clause 201. The aspiration catheter of clause 193 to 200, wherein the extraction configuration comprises a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen.

Clause 202. The aspiration catheter of clause 193 to 201, wherein the scaffold comprises struts joined by crown, further comprising stops on adjacent struts to limit the collapse of the scaffold under pressure.

Clause 203. The aspiration catheter of clause 202, wherein the stops comprises circumferentially aligned tabs.

Clause 204. The aspiration catheter of clause 193 to 203, wherein the scaffold comprises a polymeric material.

Clause 205. The aspiration catheter of clause 193 to 204, wherein the scaffold comprises a shape memory material.

Clause 206. The aspiration catheter of clause 193 to 205, wherein the scaffold comprises an elastomeric material.

Clause 207. The aspiration catheter of clause 193 to 206, wherein the scaffold comprises a combination of elastomeric, polymeric and/or shape memory material.

Clause 208. A method for extracting clot from a blood vessel, said method comprising:
positioning a distal portion of an aspiration catheter in a blood vessel proximal to the clot;
said distal portion of the aspiration catheter comprise a central clot-receiving passage through the distal portion and is contiguous with an aspiration lumen in the aspiration catheter; and
applying a first level of vacuum to a proximal portion of the aspiration lumen to draw clot from the blood vessel into the distal portion of the aspiration catheter;
increasing the vacuum level after the clot has been drawn into the distal portion of the aspiration catheter, wherein the increased level of vacuum causes the distal portion to partially collapse to disrupt and/or extract the clot.

Clause 209. The method of clause 208, wherein the distal portion of the aspiration catheter comprises a scaffold covered with a vacuum-resistant membrane and wherein struts of the scaffold struts act to break and/or shear the clot as the distal portion is partially collapsed by increasing the vacuum level.

Clause 210. The method of clause 209, wherein the distal portion of the aspiration catheter is partially collapsed to an average width in a range from 0.25 to 0.75 of an initial width of the distal portion of the aspiration catheter.

Clause 211. The method of clause 208 to 210, wherein the first level of vacuum is in a range from 0 to 0.5 atmospheres.

Clause 212. The method of clause 208 to 210, wherein the increased vacuum level is in a range from 0.2 atm to 1 atm.

Clause 213. A clot disruption catheter for resecting and aspirating clot from a blood vessel, said catheter comprising:
a catheter body having a proximal end, a distal end, and an aspiration lumen therebetween;
a distal portion of the aspiration catheter having a central clot-receiving passage contiguous with the aspiration lumen; and
clot disruption means coupled to the distal portion of the catheter body configured to disrupt clot sufficiently pass into and through the aspiration lumen when a vacuum is applied to a proximal end of the aspiration lumen.

Clause 214. The clot disruption catheter of clause 213, wherein the clot disruption means comprises at least one cutting element disposed across the distal central clot-receiving passage when the central clot-receiving passage cuts the clot as the clot is aspirated into the aspiration lumen when the vacuum is applied to the proximal end of the aspiration lumen.

Clause 215. The clot disruption catheter of clause 214, wherein the cutting element comprises a wire attached across the distal opening of the distal portion of the catheter body, wherein the wire is folded when said distal portion is closed and the wire is tensioned across the distal opening when said distal portion is opened.

Clause 216. The clot disruption catheter of clause 213, wherein the cutting element comprises folding blades attached across the distal opening of the distal portion of the catheter body, wherein the blades are folded closed when said radially expandable distal portion is in the collapsed configuration and the folding blades are substantially non-parallel when said distal portion is in an expanded configuration.

Clause 217. The clot disruption catheter of clause 214, wherein the clot disruption means comprises a clot constriction structure disposed inside at least one of the central clot-receiving passage and the aspiration lumen, wherein the clot constriction structure is actuable to radially constrict a segment of clot after said segment has been aspirated into said at least one of the central clot-receiving passage and the aspiration lumen.

Clause 218. The clot disruption catheter of clause 213, wherein the clot constriction structure comprises a coil disposed coaxially in the at least one of the central clot-receiving passage and the aspiration lumen, wherein prior to actuation the coil lies adjacent an inner wall of the at least one of the central clot-receiving passage and the aspiration lumen and upon actuation the coils closes radially inward to constrict the clot.

Clause 219. An aspiration catheter for removing clot from a blood vessel, said aspiration catheter comprising:
a catheter body having a proximal end, a distal end, and an aspiration lumen therebetween;
a distal tip extending distally from the distal end of the catheter body and having a central clot-receiving passage contiguous with the aspiration lumen of the catheter body, wherein at least a distal portion of the distal tip is delivered in an extraction configuration; and
an expandable seal disposed circumferentially about an exterior surface of the catheter body;
wherein the expandable seal is located a preselected distance proximal to the distal tip and wherein one or more vacuum ports are formed in a wall of the catheter body in a region between the distal tip and the expandable seal.

Clause 220. The aspiration catheter of clause 219, wherein the expandable seal comprises an inflatable balloon.

Clause 221. The aspiration catheter of clause 219, wherein the expandable seal comprises a deployable cuff.

Clause 222. The aspiration catheter of clause 219, wherein the distal tip comprises a self-expanding scaffold.

Clause 223. The aspiration catheter of clause 219, wherein the preselected distance is in a range from 5 mm to 50 mm.

Clause 224. The aspiration catheter of clause 219, wherein the scaffold is covered by a pressure-resistant membrane to establish a clot aspiration path from a distal end of the scaffold to a proximal end of the aspiration lumen in the catheter body so that applying a vacuum to a proximal end of the aspiration lumen can draw clot into the central clot-receiving passage.

Clause 225. A method for extracting clot from a blood vessel, said method comprising:
positioning a distal portion of an aspiration catheter in a blood vessel proximal to the clot;
wherein the distal portion of the aspiration catheter forms a clot-receiving passage through the distal portion contiguous with an aspiration lumen in the aspiration catheter;
radially expanding a circumferential seal about the aspiration catheter at preselected distance proximal to the distal tip;
applying a vacuum to a proximal portion of the aspiration lumen to draw clot from the blood vessel through the distal portion and into the lumen of the aspiration catheter; and
wherein at least one vacuum port is disposed in a wall of the catheter body in a buffer region between the distal tip and the expandable seal to draws a vacuum in said region.

Clause 226. The clot aspiration system of clause 23 to 28, wherein the aspiration catheter is configured to be retracted while keeping the inner catheter stationary to displace the transition structure to uncover the distal end of the aspiration catheter.

Clause 227. The clot aspiration system of clause 1 to 29, wherein the transition structure is held firmly to the aspiration catheter by pulling vacuum on the aspiration catheter during delivery.

Clause 228. The clot aspiration system of clause 84 or 85, wherein the distal tip of the tapered transition structure is detachably attached to the inner catheter by a vacuum within the aspiration catheter lumen.

Clause 229. A clot aspiration system for aspirating clot from a blood vessel, said clot aspiration system comprising:
an aspiration catheter having a proximal end, an open distal end, and an aspiration lumen therebetween;
an intermediate catheter having a proximal end, a distal end, and a central passage therebetween, wherein the intermediate catheter is slidably received in the aspiration lumen of the aspiration catheter;
an inner catheter having a proximal end, a distal end, and a guide wire lumen therebetween,
wherein the inner catheter is slidably received in the central passage of the aspiration catheter;
a transition structure coupled to a distal segment of the inner catheter wherein the transition structure covers or fills the open distal end of the aspiration catheter when the distal end of the inner catheter is positioned at or near the distal end of the aspiration catheter.

Clause 230. The clot aspiration system of clause 229, wherein the transition structure has a tapered shape.

Clause 231. The clot aspiration system of clause 230, wherein the inner catheter has a tapered distal end configured to nest in a tapered cavity on a proximal side of the transition structure.

Clause 232. A method for aspirating clot from a blood vessel, said method comprising:
providing a clot aspiration system as in any one of clauses 229 to 231; and
advancing an assembly of the aspiration catheter, the intermediate catheter, and the inner catheter into a patient's vasculature, over a guidewire, wherein the intermediate catheter is fully advanced within the aspiration lumen of the aspiration catheter to enhance pushability and the transition structure covers or fills the open distal end of the aspiration catheter during at least a portion of the advancement step.

Clause 233. The method of clause 232, wherein the intermediate catheter remains fully advanced within the aspiration lumen of the aspiration catheter until a distal end of the assembly has reached a target location within the patient's vasculature.

Clause 234. The method of clauses 232 or 233, wherein the intermediate catheter is at least partially retracted to enhance flexibility when a distal end of the assembly has reached a tortious region within the patient's vasculature.

Clause 235. The method of clauses 232 to 234, wherein the intermediate catheter wherein the transition structure is collapsed and removed by retracting the inner catheter after the distal end of the assembly has reached a target location within the patient's vasculature.

Clause 236. The method of clause 235, wherein the target location comprises clot or thrombus and a negative pressure is applied to the aspiration lumen to at least partly aspirate the clot or thrombus after the inner catheter has been retracted.

Clause 237. A clot aspiration catheter for aspirating clot from a blood vessel, said catheter comprising:
an aspiration catheter having a proximal end, a distal end, and an aspiration lumen therebetween;
an elongated inner stiffening member having a proximal end, a distal end, and a guide wire lumen therebetween, said elongated inner stiffening member being removably received in the aspiration lumen of the aspiration catheter; and
one or more frictional anchors disposed on a distal segment of the elongated inner stiffening member, wherein said one or more frictional anchors are configured to reduce an annular space or engage an inner wall of the aspiration lumen to enhance support, pushability, and/or trackability of the aspiration catheter as it is being advanced through a blood vessel.

Clause 238. The clot aspiration catheter of clause 237, wherein the frictional anchors comprise one or more protruding structures.

Clause 239. The clot aspiration catheter of clause 237, wherein the stiffening member comprises an inner catheter.

All aspects, examples, embodiments, and paragraphs can be combined in whole or in part without departing from the intent of this invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative examples, in which the principles of the invention are utilized, and the accompanying drawings of which:

FIGS. 1A and 1B show an aspiration catheter constructed in accordance with the principles of the present invention having a distal scaffold portion in radially collapsed and expanded configurations, respectively.

FIGS. 2A and 2B show detailed views of a helical scaffold as used in the aspiration catheters of FIGS. 1A and 1B.

FIG. 3 shows a close up of one example of the catheter outer shaft.

FIG. 4 shows a close up of one example of the device handle comprising a fixed handle body 40 and rotating handle knob 41

FIGS. 5A-5G depict an example of the device of the present invention in use.

FIG. 6A shows a helical scaffold comprising a ribbon with a counter-clockwise wind as viewed from proximal to distal, such that the inner torque member would be rotated counter-clockwise to collapse the coil and clockwise to expand it.

FIG. 6B shows a ribbon similar to that of FIG. 6A with a clockwise wind as viewed from proximal to distal, such that the inner torque member would be rotated clockwise to collapse the coil and counter-clockwise to expand it.

FIG. 7 shows an example of a coil with a variable ribbon width along its length.

FIG. 8 shows an example of a double helix coil.

FIG. 9 shows an example of a triple helix coil with the first ribbon and second ribbon having slots cut in the core of the ribbon.

FIGS. 10A and 10B show a collapsed coil and expanded coil, respectively, featuring laser cut notches and bumps.

FIG. 11A shows an example of a coil with a sinusoidal ring ribbon comprising struts joined by crowns.

FIG. 11B shows a side cross section of the distal end of the device of FIG. 11A in an expanded configuration showing the sinusoidal ring ribbon along with an inner torque member and an intermediate outer segment.

FIG. 11C shows a side cross section of the distal end of the device FIG. 11A in a collapsed configuration including the presence of a constraining sleeve.

FIGS. 12 and 13 illustrate a preferred example of the present invention, in which the distal part of the inner torque member has been replaced with a second inner coil wound in direction opposite to that of the outer coil in expanded and collapsed configurations, respectively.

FIG. 14 shows another example of the present invention in which tubular attachments with slots are employed to maintain a constant spacing of the coil ribbon through diametric changes.

FIG. 15 shows an example of a self-expanding conical scaffold with a distal end and a proximal end, comprising a plurality of struts 152 radiating in a distal direction from a common circular base 153.

FIG. 16 shows a self-expanding scaffold having conical and cylindrical segments in which the struts have bends allowing the expanded scaffold to better conform to the vessel in the expanded state for superior vacuum sealing.

FIG. 17 shows a variant of the scaffold of FIG. 16 in which the rounded tips of the struts have flat portions on the leading edge to further reduce vessel trauma and/or better distribute loads against the vacuum-resistant membrane.

FIG. 18 shows another self-expanding scaffold having conical and cylindrical segments in which two or more struts are connected by arcs to an adjacent strut thereby forming loops.

FIG. 19 shows a self-expanding conical scaffold in which the proximal ends of the struts are connected by arc forming a sinusoidal ring or serpentine structure.

FIG. 20 shows a variant of the self-expanding conical scaffold of FIG. 19 in which the struts have bends near the crown tips allowing the expanded scaffold to better conform to the vessel in the expanded state for superior vacuum sealing.

FIG. 21 shows a self-expanding scaffold comprising a plurality of struts connected by arcs at both ends to form a sinusoidal ring structure where a proximal end is attached to a scaffold base by struts.

FIG. 22 shows a self-expanding conical scaffold similar to FIG. 21 in which the links include spring elements to increase flexibility of the self-expanding scaffold as a whole.

FIG. 23 shows an example of conical scaffold formed with a tapered serpentine body attached to a base ring which expands radially outwardly at an angle in a distal direction.

FIG. 24 shows a conical scaffold having a proximal region oriented at a first relative to an axis and a distal region oriented at a second angle relative to the axis, where the first angle is greater than the second angle.

FIG. 25 shows a conical scaffold with radially inward-pointing tips.

FIG. 26 shows a variant of the conical scaffold of FIG. 25 in which the scaffold has more gradually curved inward-pointing tips.

FIG. 27 shows a self-expanding scaffold mounted with the radially converging apical end of the scaffold oriented in a distal direction and the radially diverging end of the scaffold oriented in a proximal direction.

FIGS. 28A-28C show a preferred aspiration catheter of the present invention comprising a self-expanding scaffold attached to an inner elongated tubular body translatably received in an outer elongated tubular body.

FIG. 29 shows an aspiration catheter in which the self-expanding scaffold is radially restrained by a distal cap attached to a removable inner elongated tubular body.

FIG. 30 shows an aspiration catheter having a self-expanding scaffold attached to a distal end of an outer elongated tubular body and constrained by a wire, filament, or ribbon wrapped around the at least distal end of the self-expanding.

FIG. 31 shows an aspiration catheter having a self-expanding scaffold attached to the distal end of the outer elongated tubular body and held in a constrained state by a frangible material.

FIGS. 32A and 32B show an aspiration catheter in which a self-expanding scaffold is attached to a distal end of an elongated tubular body and is constrained state by a drawstring filament.

FIGS. 33A and 33B show an aspiration catheter in which a self-expanding scaffold includes struts of different lengths.

FIG. 34 shows an aspiration catheter in which a self-expanding scaffold is attached to the distal end of an elongated tubular body and constrained state by a ring.

FIGS. 35A and 35B shows an aspiration catheter in which a self-expanding scaffold is compressed and folded into a fixed-diameter aspiration lumen (FIG. 35A) and expanded upon distal advance (FIG. 35B).

FIGS. 36A and 36B are side and end views of an aspiration catheter in which the scaffold may constrained in an aspiration lumen.

FIG. 37 shows an aspiration catheter having a scaffold comprising sinusoidal rings made from a swellable polymer.

FIG. 38 shows a flexible junction design in which a distal expanding segment is coupled to a base which may be attached to a catheter shaft by a coil or pigtail structure.

FIG. 39 shows a distal expanding structure connected to a distal end of a catheter shaft by flexible links.

FIG. 40 shows an expanding structure connected to an adjacent catheter by ball-and-socket type joints.

FIG. 41 shows a distal expanding structure which is disconnected from an adjacent catheter shaft.

FIG. 42 shows a distal expandable segment comprising an ovoidal braided structure which flares outwards from the catheter shaft.

FIG. 43 shows a distal expandable segment comprising a flared braided structure extending from an outer catheter shaft.

FIGS. 44A and 44B are side and end views, respectively, showing a distal expandable segment comprising a longitudinal ribbed structure.

FIG. 45 shows a distal expandable segment comprising multiple rings connected on opposite sides to spines.

FIG. 46 illustrates a distal expandable segment having rings and a single spine covered by a tubular structure with cuts.

FIG. 47 shows an aspiration catheter in which a distal expandable segment comprises a plastically deformable scaffold mounted on an end of an outer elongated tubular body where a balloon catheter may be inflated to expand the scaffold.

FIG. 48 shows another example of the present invention in which the distal expandable segment is constructed from a coiled polymer tubing 480 wherein the coil loops are bonded together.

FIG. 49 shows a distal expanding segment comprising a coil attached to a catheter shaft in which a vacuum resistant membrane extends from an end of the catheter shaft to a point substantially proximal to the distal end of the coil.

FIG. 50 shows a distal expanding segment comprising a self-expanding scaffold attached to a catheter shaft in which a vacuum resistant membrane 502 extends from an end of the catheter shaft to a point substantially proximal to the distal end of a self-expanding scaffold.

FIGS. 51A-51C show a distal expandable scaffold consisting of a single undulating element. The pattern in a flattened (rolled out) state is shown in FIG. 51A and in a rolled configuration in FIGS. 51B (collapsed) and 51C (expanded).

FIGS. 52A and 52B show another example of a distal expandable scaffold consisting of a single undulating element where the scaffold comprises multiple continuous undulating elements which are maintained in position by tab-and-slot joints.

FIG. 53 shows another example of a distal expandable scaffold consisting of a single undulating element attached to a segment of an aspiration catheter and covered with the vacuum resistant membrane.

FIG. 54 shows a scaffold having a single continuous undulating element.

FIG. 55 shows a variant of a scaffold with a single continuous undulating element with two pull struts.

FIGS. 56A-56C show a perspective view of the scaffold of FIG. 54.

FIG. 57 shows an example of an unraveling scaffold.

FIG. 58 shows an alternative unraveling scaffold structure.

FIGS. 59A and 59B show a scaffold having an expandable unraveling distal scaffold.

FIGS. 60A and 60B show a self-expanding scaffold configured to controllably collapse under vacuum.

FIGS. 61A-61C show an alternative example of a self-expanding scaffold configured to controllably collapse under vacuum.

FIGS. 62A-62B show a cone-shaped self-expanding scaffold configured to controllably collapse under vacuum.

FIGS. 63A-63B show a self-expanding scaffold featuring bent tips intended to limit collapse of the scaffold and/or cut a clot during such collapse, in the expanded state.

FIG. 64 shows an aspiration catheter having multiple expandable scaffolds.

FIG. 65 shows the device of FIG. 64 with vacuum lumens for multiple vacuum regions.

FIG. 66 shows an aspiration catheter having multiple scaffolds deployed in an artery adjacent clot.

FIG. 67 shows a malleable conformable scaffold having a conical shape.

FIG. 68 shows a cross-sectional view of a malleable conformable scaffold as it unfurls.

FIG. 69 shows an end view of the device of FIG. 67, in which the malleable structure has been furled with a spiral wrap.

FIGS. 70A and 70B show a malleable conforming scaffold as it is advanced into clot.

FIGS. 71A and 71B show passive cutting elements attached to a distal end of an aspiration catheter.

FIGS. 72A-72D show and the use of an aspiration lumen containing various clot-disrupting elements.

FIG. 73A-73B show an apparatus according to the present invention having a collapsible clot disrupting element.

FIGS. 74A-74B show a further example of a collapsible clot disrupting element.

FIG. 75A-75C show a clot compression coil within an aspiration lumen of a catheter of the present invention.

FIG. 76 shows a catheter assembly having a removable inner catheter.

FIG. 77 shows a removable inner catheter having a “rapid exchange” guidewire lumen.

FIG. 78 shows a removable inner catheter positioned within an aspiration lumen of an aspiration catheter.

FIG. 79 shows a removable inner catheter having a stepped configuration at its distal end.

FIG. 80 shows a removable inner catheter having a distal interface section.

FIG. 81 shows a removable inner catheter having an inflatable balloon interface.

FIG. 82 shows a removable inner catheter having an inflatable interface with a stepped region for engaging a distal end of an aspiration catheter.

FIG. 83 shows a removable inner catheter having multiple friction anchors for contacting an inner surface of an aspiration lumen of an aspiration catheter.

FIGS. 84A-84I show an aspiration assembly according to the present invention being used to remove clot from a blood vessel occluded by the clot.

FIGS. 85A-85D show an alternative aspiration assembly being used to remove clot from a blood vessel.

FIG. 86 shows aspiration catheter assembly supported by an outer guiding sheath and a removable inner catheter.

FIG. 87 shows an aspiration catheter assembly having an aspiration catheter supported by an outer guiding sheath and a removable inner catheter having a collapsing sleeve transition structure.

FIGS. 88A-88D provide an additional example of use of an aspiration catheter assembly according to the present invention for moving clot from a patient's vasculature.

FIGS. 89A-89C provide a further example of use of an aspiration catheter assembly according to the present invention for moving clot from a patient's vasculature.

FIGS. 90A-90C provide yet another example of use of an aspiration catheter assembly according to the present invention for moving clot from a patient's vasculature.

FIGS. 91A-91C provide an example of use of an aspiration catheter assembly according to the present invention for moving clot from a patient's vasculature.

FIGS. 92A-92C provide an example of use of an aspiration catheter assembly having a transition structure on the aspiration catheter for moving clot from a patient's vasculature.

FIGS. 93A-93C illustrate the operation of a clot aspiration system incorporating an intermediate catheter for moving clot from a patient's vasculature.

### DETAILED DESCRIPTION OF THE INVENTION

### Example 1: Reversibly Expanding Coil

The novel features of the invention are set forth with particularity in the appended claims. A better understanding of the features and advantages of the present invention will be obtained by reference to the following detailed description that sets forth illustrative examples, in which the principles of the invention are utilized, and the accompanying drawings of which:

FIG. 1A depicts an aspiration catheter constructed in accordance with the principles of the present invention. The device comprises a distal expandable segment 1, the intermediate segment or intermediate shaft 2, the proximal segment or proximal shaft 3, and the handle 4. The shafts are joined to each other at bonds 5 and to the handle at a strain relief bond 6. The handle comprises a distal handle section 7, a rotating middle handle section 8, and a proximal handle section 9. The distal handle section or the proximal handle section has an aspiration port 10. The proximal end of the device has a lumen 11, which may be configured to receive a guidewire and/or used for aspiration. The middle handle section can rotate relative to the other parts of the handle due to the presence of Swivels 12. FIG. 1A illustrates the distal expandable segment in the collapsed state as it would be introduced into the body and delivered to the target vascular, while FIG. 1B illustrates the distal expandable segment 1 in the expanded state as would be used during aspiration of the clot. FIG. 1B also shows the inner torque member 13 by which the distal expandable segment is expanded and collapsed, and the vacuum resistant membrane 14 which covers the distal expandable segment and connects to the non-expandable sections of the device in order to provide a continuous vacuum path and prevent leakage under vacuum, which could compromise device effectiveness.

The distal expandable segment 1 comprises an expandable and contractible structure which in the contracted state provides a low distal segment profile for superior deliverability, and in the expanded state increases the distal section diameter for improved aspiration. In the preferred example, the distal expandable segment has an outer diameter in the delivery configuration of 2 mm of less, preferably 1.5 mm or less, and most preferably 1 mm or less, and is preferably also less than the outer diameter of the intermediate segment 2 to which it is attached. The distal expandable segment is capable of expanding to a diameter equal to or larger than the clot and/or the vessel occluded by the clot. In the preferred example the scaffold engages the inner wall of the blood vessel to prevent blood leakage past the end of the scaffold when vacuum is applied. The scaffold may be designed to expand such that only the desired portions of the expanded scaffold engage the vessel wall, as desired to balance aspiration performance and risk of vessel trauma. For example only the distal portion of the scaffold may engage the vessel wall, or only the proximal portion, or only a middle section. The scaffold may be intended to be expanded immediately adjacent to the clot or some distance proximal to that. Upon application of vacuum pressure the clot is then drawn into the aspiration lumen of the device.

The distal expandable segment may be configured to expand to a diameter between 2 and 6 mm, more preferably from 3 to 5 mm, and most preferably from 4 to 4.5 mm. Therefore the device of the present invention provides an aspiration lumen in the expandable segment with a cross-sectional area between 1.5× and 10× higher than a conventional aspiration catheter with a fixed diameter aspiration lumen in the 1.4-2.0 mm range. Since the vacuum force applied equals the vacuum pressure times the cross-sectional area, the vacuum force capable of being applied by the device of the present invention is 1.5× to 10× higher than that provided by conventional aspiration catheters, with concurrently superior clot extraction capabilities.

In the example shown in FIGS. 1A and 1B the distal expandable segment 1 is constructed from a single coil structure, the proximal end of which is attached to the catheter intermediate segment 2 and the distal end of which is attached to an inner torque member 13 running through inside of the single coil and attached to the handle 4. Rotation of the inner torque member via the handle causes the coil to be either wrapped tighter thereby decreasing its diameter for delivery (as depicted in FIG. 1A), or to unwrapped to increase its diameter for aspiration (as depicted in FIG. 1B).

The inner torque member 13 may be a solid wire, a tube, or a composite structure such as a polymer shaft with an embedded coil or braid, or a combination thereof. It will typically be as small as possible in order to maximize the area of the aspiration lumen in which it is contained, since a larger inner member occupies more space in the lumen and may negatively affect aspiration efficiency. Solid wires or mandrels of stainless steel, nickel-titanium, or cobalt chrome alloys are most suitable for this application due to having the greatest torque to profile ratio. Ideally such solid members would decrease in diameter towards their distal end in order to minimize impact to system flexibility. However the inner torque member may be tubular and sized to accommodate a guidewire, rather than requiring the guidewire to run adjacent to the inner member and through the vacuum lumen. To minimize wall thickness for flexibility and minimize occlusion of aspiration lumen area while maintaining excellent torque transmission, a tubular inner torque member may be a spiral cut hypotube, with a thin polymer jacket to prevent unspooling when torqued. An inner torque member may comprise more than one of the examples described above, such as a tapered wire in the distal segment which connects to a tubular member more proximally.

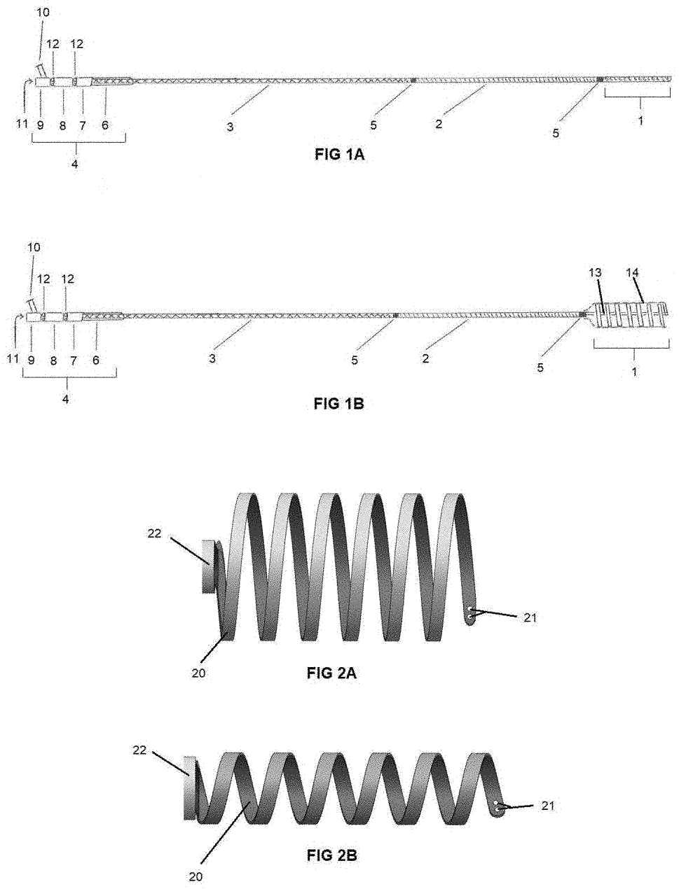

FIGS. 2A and 2B show a close-up of a distal expandable segment consisting of a single coil structure, as shown in the expanded state (FIG. 2A) and in a substantially collapsed state (FIG. 2B). The single coil comprises a coil ribbon 20 and may optionally feature distal holes 21 and/or a proximal ring 22 for improving ease of attachment to the inner torque member and catheter intermediate shaft respectively.

The coil structure may be designed in a variety of ways in order to achieve the functional requirements of the device to (i.) deliver to the site of treatment, (ii.) smoothly expand from the collapsed state to the expanded state, (iii.) maintain the lumen shape and resist collapse forces during application of vacuum for aspiration, (iv.) smoothly collapse from the expanded state, and (v.) withdraw the device from the site of treatment.

The coil ribbon 20 may be manufactured from round wire or flat ribbon. Round wire coils would typically be made by wrapping wire around a mandrel and then removing the mandrel, while flat ribbon coils would typically by made by laser cutting a hypotube. Flat ribbon coils may also be wound from flat ribbon wire. Coils may be manufactured from any materials of sufficient strength, flexibility, and biocompatibility for the application. In the exemplary example the coils are made from stainless steel, cobalt chrome, nickel-titanium (NiTi), or titanium alloys. For the same dimensions, stainless steel and cobalt chrome coils provide better torque response than nickel-titanium, but NiTi coils have superior flexibility and are less likely to be damaged during manufacture or use. Coils may also be manufactured from high strength polymers including PEEK, polyimide, and select nylons, polyurethanes, and PETs.

Nickel-titanium (NiTi) alloys in particular are desirable since the super-elastic material is very resistant to kinking and fracture, and also because the NiTi coils and others made from shape memory materials can be heat set into a desired shape. Coils may be heat set into a cone, flared cone, stepped shape, exponential taper and other shapes in order to improve clot engagement and/or coil expansion dynamics. In a preferred example, the distal end of the coil is substantially cylindrical in shape, and the proximal end of distal expandable segment tapers smoothly down to the catheter shafts. The coil may also be heat set to be smallest at the distal end and get progressively larger to the proximal end, or largest at the distal end and get progressively smaller to the proximal end, or even heat set such that in the expanded state it is largest in the middle with smaller ends, or the reverse in which the middle is smallest and the ends are largest. Such heat set geometries play an important role in coil expansion, and can be used to ensure consistent expansion performance in tortuosity and to prevent twisting of the vacuum resistant membrane covering the coil during expansion. The heat set process can also be used to alter the neutral state of the coil (similar to using a hypotube of a different diameter) and to control spacing between loops of the coil. The coil may also be heat set into an oblong or oval cross-section (when viewed from the end-on) rather than maintaining a circular lumen. This results in a coil of variable profile with a tendency to intermittently lift the vacuum resistant membrane during expansion, reducing potential clinging and twisting of the membrane.

In the exemplary example the coil is constructed from a laser cut hypotube, such that a variety of design attributes come into play. First, the starting tube diameter determines the neutral properties of the coil—larger tubes result in a coil with more strength and uniformity in the expanded state but may be more difficult to collapse to a low profile. The tube and therefore coil ribbon wall thickness also significantly impacts the strength, flexibility, collapsibility, and radiopacity of the coil. Tubes suitable for this application are typically in the range of 1.0 to 3.5 mm outer diameter, with a wall thickness of 0.0015″ to 0.004″. Thicker tubes up to 0.008″ wall thickness may also be suitable, especially if significant material may be removed during processing such as electropolishing. Depending on the designed geometry, laser angle, and electropolishing process (if any), the coil cross-section geometry can be circular, square, rectangular, trapezoidal, etc.

The length of the coil structure and hence of the distal expandable segment may be as desired. In the exemplary example the length may be as short as 1 mm or as long as 150 mm. Shorter elements require fewer rotations to open yet still provide the full increased tip vacuum force of the present invention, while longer distal expandable segments create a larger chamber to take in and hold larger/longer and more fibrous clots which need to be pulled out intact. The length of the distal expandable segment may also impact deliverability.

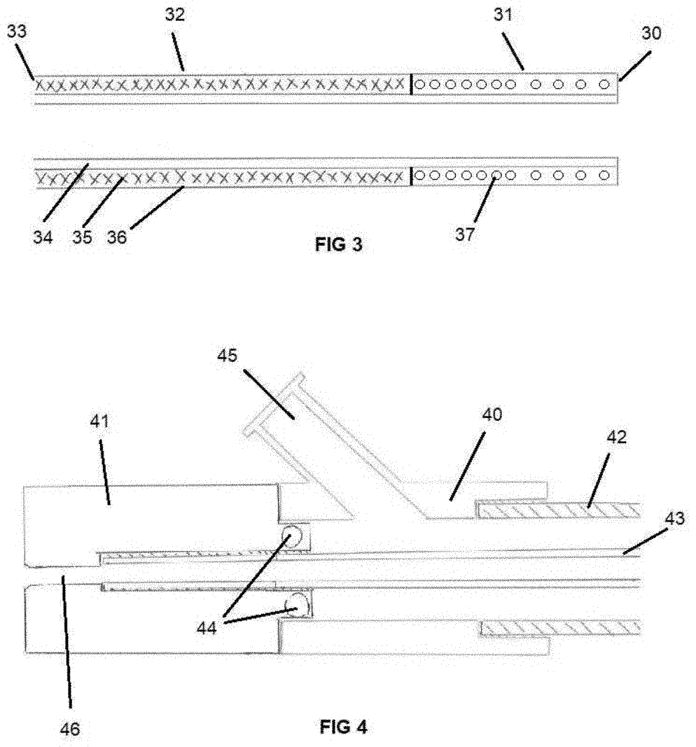

FIG. 3 shows a close up of one example of the catheter outer shaft, comprising an elongated tubular body with a distal end 30, an intermediate outer shaft 31, a proximal outer shaft 32, and a proximal end 33. The purpose of the catheter shaft is to allow the distal expandable segment to be advanced to and withdrawn from the target area, to permit torque to be transmitted from the handle mechanism to the distal expandable segment so it can be expanded and collapsed, and to provide a fluid-impermeable lumen through which vacuum pressure can be applied to the distal end of the device. In the exemplary example, the catheter comprises two segments with distinct properties, the intermediate shaft (segment) and the proximal shaft (segment), although variants with more than two shaft sections are envisioned and may be superior for some applications.

In general, the proximal outer shaft 32 of the device runs from the user-operated handle on the proximal end of the device (and outside the patient's body) through the femoral artery access point, up the aorta, and into the base of the carotid or vertebral arteries. The proximal outer shaft will be firmer than the intermediate segment and optimized for torque and/or linear force transmission. The intermediate segment of the device will be optimized for flexibility such that the distal segment and the intermediate segment can be tracked through the tortuous intracranial neurovascular anatomy to the site of the clot. The intermediate segment must retain sufficient torque and/or linear force transmission capability to allow the distal expandable segment to be expanded and collapsed.

A variety of metal and polymer technology well known within the industry may be used to manufacture the catheter shafts. In the exemplary example proximal outer shaft 32 comprises lubricious polymer inner liner 34, a metallic or polymeric braid 35 in the core, and a firmer polymer outer jacket 36. The inner liner is typically made from PTFE, FEP, HDPE, or another lubricious polymer to allow the underlying inner member or guidewire to rotate smoothly, the braid is made from stainless steel or nickel-titanium alloy to provide strength, kink resistance, and efficient torque transmission, and the outer jacket is made from Polyether block amide (Pebax®), nylon, polyether ether ketone (PEEK,), or polyamide. In the exemplary example the intermediate outer shaft will be of similar construction to the proximal outer shaft, except the core layer will contain an embedded support coil 37 rather than a braid in order to maximize the flexibility of this portion of the shaft while maintaining lumen integrity and prevent kinking around tight corners. The outer jacket of the intermediate outer shaft will also be manufactured from softer and more flexible materials like low durometer (25D-55D) Pebax or similar. The embedded support coil may be a spring guide in which the adjacent loops of the coil are in direct contact with each other in order to provide maximum axial stiffness, shaft pushability, collapse resistance, and radiopacity.

For single coil and some other device designs, it may be advantageous in an example to use a multilumen shaft design in the intermediate and/or proximal segments where the largest lumen is used for aspiration, and the smaller lumen(s) used for guidewire passage, contrast injection, or to sequester the inner torque member. This provides a continuous and unobstructed aspiration lumen which may aspirate clot more effectively than a lumen partially occluded by one or more objects inside of it.

Any elongated tubular member can be shaped into an accordion or convoluted form to increase flexibility. The accordion or convoluted form can also reduce surface contact to minimize surface friction between different moving components within the system or between the elongated tubular member and the wall of the blood vessel.

FIG. 4 shows a detail of one example of the device handle comprising a fixed handle body 40 and rotating handle knob 41. The catheter shaft proximal segment 42 attaches to the fixed handle body, while the inner torque member 43 attaches to the rotating handle knob. Smooth rotation of the handle knob is facilitated by ball bearings 44. The handle body contains an aspiration port 45, while the entire assembly preferably has an inner lumen 46 for guidewire passage.

The handle mechanism connects to both the inner and the outer members of the proximal shaft and allows the physician to rotate one with respect to the other, thereby transmitting torque to the intermediate segment and expandable distal segment. In the exemplary example, the outer member is fixed and only the inner member rotates such that the outer member is stationary versus the vessel wall for minimal vessel trauma, although the reverse is envisioned, as is a variant in which both shafts are rotated simultaneously.

The handle may be designed for manual operation, with the inner and outer members connecting to different elements of the handle with a swivel between them to maintain integrity and alignment. The handle may contain a gearbox mechanism to reduce the number of turns needed by the physician to expand the expandable distal segment. The handle may also incorporate a motor which eliminates the need for manual manipulation. In some design examples, the proximal ends or toward the proximal ends of the elongated tubular member(s) and/or torque elements may terminate in simple proximal hubs, allowing the physician more freedom of operation. Such hubs may incorporate side-arms for aspiration, luer locks to keep all parts in position during device advance and/or withdrawal, and/or Tuohy-type hemostatic valves to anchor guidewires or microcatheters and to minimize blood loss during the procedure.

In another example, the handle is designed such that the inner torque element attached to the distal end of the coil is held fixed and the outer shaft is torqued to rotate the proximal end of the coil, thereby tending to unwrap and expand the coil from a substantially proximal to distal direction. This design may provide superior expansion performance in tortuous anatomy.

In another example, the handle also causes the inner torque element attached to the distal end of the coil to move distally and proximally instead of or in addition to rotating the coil to cause it to collapse or expand. Distal movement of the inner member causes the coil to lengthen and collapse in profile, while proximal movement of the inner member causes the coil to shorten and expand in profile. This approach may provide superior expansion performance in tortuous anatomy and allow for an overall lower profile of the fully collapsed device.

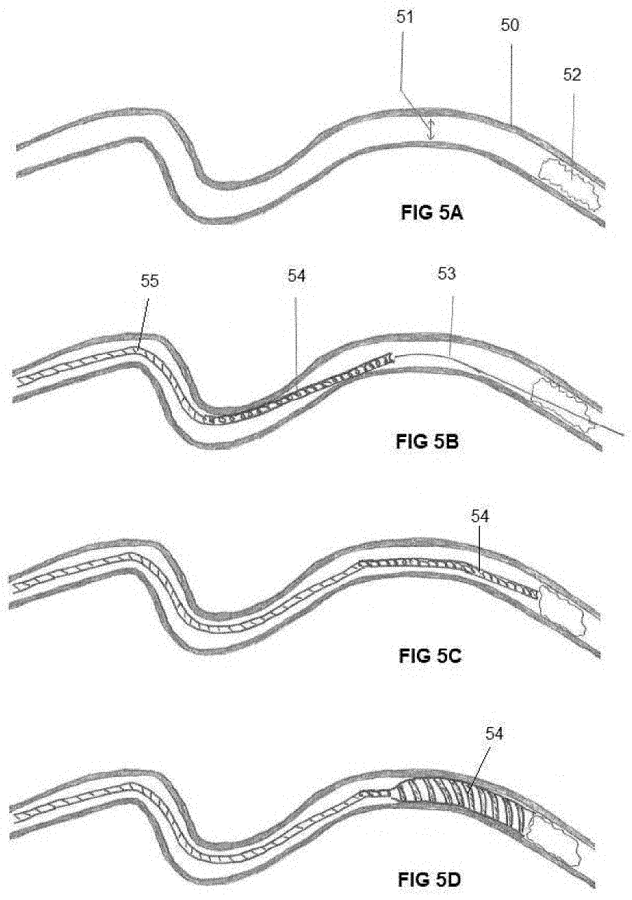

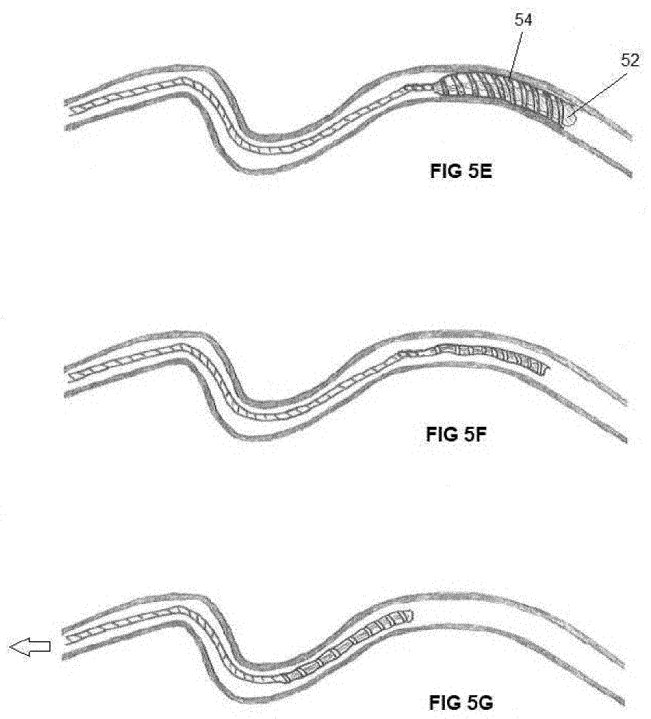

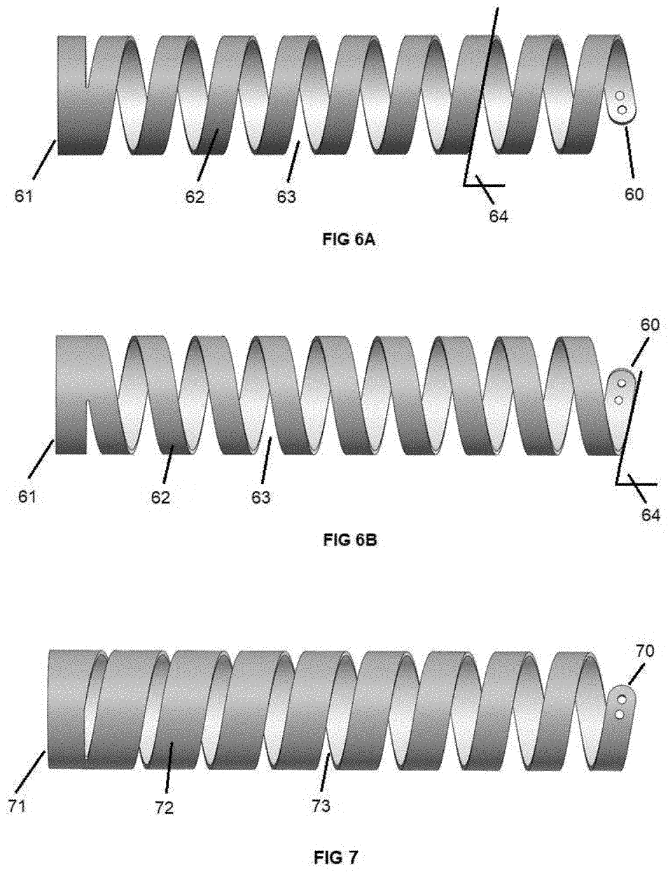

FIGS. 5A-5G depict an example of the device of the present invention in use.

FIG. 5A shows the anatomy as the patient presents to the physician, consisting of a blood vessel 50 with an inner lumen 51 which is occluded by a clot 52.

FIG. 5B shows the next steps in the procedure, in which a guidewire 53 has been advanced through the blood vessel inner lumen in through the clot, thereby providing a rail upon which the device of the present invention can be advanced. In the figure, the device including the distal expanding segment 54 in its collapsed state and the intermediate segment 55 have been threaded over the guidewire and advanced into the vascular anatomy.

FIG. 5C shows the next steps in the procedure, in which the device has been advanced until the collapsed distal expanding segment 54 is adjacent to the clot, and the guidewire has been withdrawn.

FIG. 5D shows the distal expanding segment 54 after expansion and prior to application of vacuum pressure to aspiration the clot.

FIG. 5E shows the distal expanding segment 54 after aspiration, wherein the clot 52 has been pulled within the distal expanding segment. Ideally the clot would be broken up during the aspiration process and fully removed from the body, but aged and/or fibrous clots may be exceptionally cohesive and may need to be physically pulled from the anatomy by the device, as shown.

FIG. 5F shows the distal expanding segment 54 after it has been recollapsed to trap any clot which was not fully aspirated through the device.

FIG. 5G shows the device as it is being withdrawn from the anatomy with any remaining clot trapped within it.

### Coil Variants

There are numerous aspects of the coil design which can be used to optimize its performance in particular anatomies and/or in conjunction with other parts of the system such as the inner torque member and the distal sleeve. In particular the performance of the coil will depend on the direction of coil wind, ribbon width, pitch angle, gap between ribbon loops, and number of ribbons in the wind. These design attributes may be constant along the length of the coil, or vary to provide improved collapsed or expanded properties.

FIGS. 6A and 6B show an example of a standard coil with a distal end 60, a proximal end 61, ribbon 62, ribbon gap 63, and pitch angle 64. In this example the ribbon width, pitch angle, and ribbon gap distance are constant through the length of the coil such that in absence of other factors the coil will tend to expand simultaneously and uniformly along its length. Ribbon width will typically range between 0.008″ and 0.065″. Wider ribbons result in stronger coils which resist collapse from vacuum pressure better, but they are stiffer and less deliverable than narrower ribbons. The thickness of the coil ribbon also impacts these properties. In one example the ribbon width is about 0.030″ and thickness is about 0.002″, resulting in a 15:1 ratio of width to thickness.

The coil structure helix will typically have a pitch angle 64 in a range from 50° to 85° from the longitudinal axis. Higher pitch angles result in more loops per linear length and generally less gap when the coil is expanded but require more rotations to open. The pitch angle can be determined at laser cutting, or, for NiTi coils, at heat set. In one variant of the design, the distal loops of the coil are heat set into a 90° angle such that they provide an aspiration lumen mouth that is perpendicular to the vessel axis. Such loops can be stacked for greater radial strength and may or may not overlap when in the collapsed state. The coil loops can be cut or heat set into a reverse angle in parts or all of the coil, such that the contact between the coil and the sleeve varies as the coil opens.

In the fully collapsed state there is typically little or no ribbon gap 63 between ribbon loops. Depending on ribbon width, expansion diameter, and length change allowed, the gap between the ribbon loops in the expanded state may be less than the ribbon width or up to several times greater than the ribbon width. Tighter gaps in the expanded state typically correspond to designs which allow the expandable element to shorten during expansion. Gaps between ribbon loops in the collapsed state can also increase flexibility for improved device deliverability, and/or be used to influence expansion, particular with respect to promoting distal sleeve stretching or unfolding when around a bend in vascular tortuosity.

FIG. 6A shows the ribbon with a counter-clockwise wind as viewed from proximal to distal, such that the inner torque member would be rotated counter-clockwise to collapse the coil and clockwise to expand it. FIG. 6B shows the ribbon with a clockwise wind as viewed from proximal to distal, such that the inner torque member would be rotated clockwise to collapse the coil and counter-clockwise to expand it. The direction of rotation matters primarily from an intuitiveness and ergonomic aspect for manually-operated handles. For a physician operating the handle with his right hand, it is most intuitive to rotate the handle knob clockwise to expand the distal expandable segment, such that a counter-clockwise ribbon would be used if there is a direct connection from the handle knob to the inner torque member. If the handle contains gearing which causes the inner torque member to rotate in a direction opposite to the direction in which the handle knob is turned, then a clockwise ribbon wind would be used to maintain the clockwise knob rotation for device expansion.

FIG. 7 shows an example of a coil with variable ribbon width featuring a distal end 70, a proximal end 71, ribbon 72, and ribbon gap 73. In the example illustrated, the ribbon width decreases from 0.040″ at the proximal end (20:1 width to thickness ratio on a 0.002″ tube) to 0.020″ at the distal end (10:1 ratio). Because the pitch angle of the ribbon width is constant, the ribbon gap increases from proximal to distal as the ribbon width narrows. Alternatively, the increase in width can progress in the opposite direction from proximal to distal end. Variance in coil ribbon width may be linear with length or non-linear, such that the increase or decrease in width occurs more or less rapidly down the length. Such variances in ribbon width can significantly impact coil flexibility and expansion, particularly in combination with the coil pitch angle and any taper to the heat set coil. Used separately or together, these features may promote coil opening and/or reclosing from distal to proximal, proximal to distal, or evenly, and be used to balance the impact of the presence of the distal sleeve on coil expansion. In example shown with the distal end of the coil having a narrower ribbon than the proximal end, the device will be slightly more trackable through the anatomy due the progressively increasing flexibility towards the distal end, and it will also seek to expand first at the distal end and propagate proximally. Depending on other factors, the final expanded coil will tend to have a slightly tapered shape in the expanded state, being larger at the distal end and smaller at the proximal end.

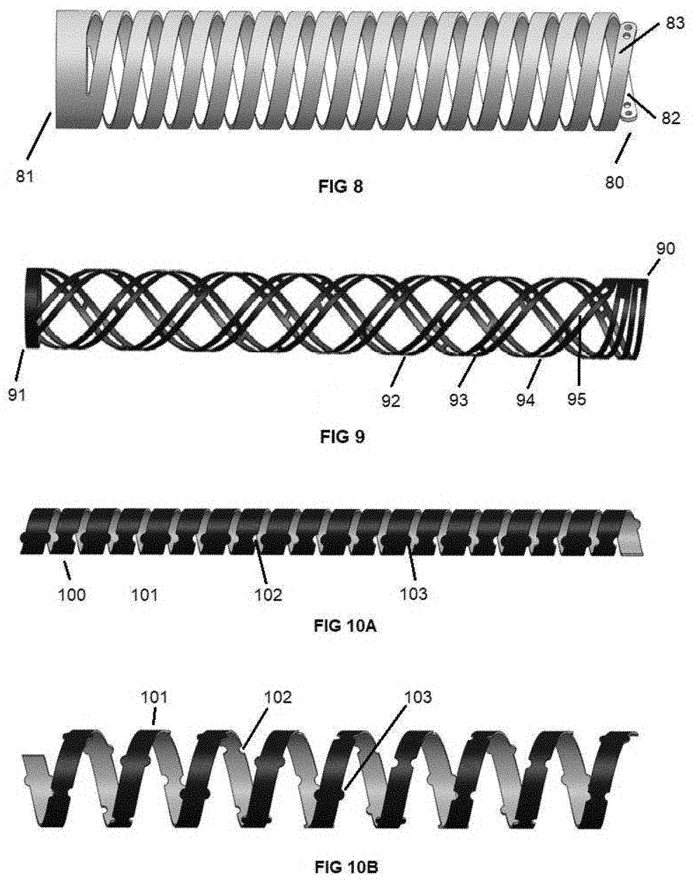

FIG. 8 shows an example of a double helix coil with a distal end 80, a proximal end 81, a first ribbon 82, and a second ribbon 83. Whereas a coil with a conventional spring construction consists of a single ribbon spiraling helically around the center axis, this Figure illustrates a double helix construction (i.e. like DNA) with two the parallel ribbons 82 and 83 spiraling about the center axis. Other examples have three or more helixes. In general more helixes provide greater coverage such that pitch helix angle (versus the axis) is decreased, resulting in fewer winds necessary for a given length and fewer rotations at the distal end needed to expand/collapse the coil, at the cost of potentially decreased flexibility.

FIG. 9 shows an example of a triple helix coil with a distal end 90, a proximal end 91, a first ribbon 92, a second ribbon 93, and a third ribbon 94 in which all ribbons feature slots 95 cut into the core of the ribbon, thereby creating a coiled ladder structure. Adding slots to the ribbon of any coil may provide varying contact area against the distal sleeve for improved expansion. A ladder structure to the ribbon would also allow for a coil with wider ribbons which would remain flexible in the crimped state but would be more resistant to axial elongation and rotational distortion, potentially enabling designs with shallower pitch angles and more spirals to reduce the number of rotations needed to expand the coil.

FIGS. 10A and 10B shows an example of a collapsed coil 100 and expanded coil 101 featuring laser cut notches 102 and bumps 103. The edges of the coil ribbon may be laser cut into contours such as waves, bumps, notches, or other geometric features. As the coil expands and the loops rotate past each other, these features provide varying contact area against the distal sleeve to reduce propensity of the sleeve to twist and to enhance expansion uniformity.

In another example, the coil is a radially expandable separator scaffold extending distally from the distal end of the catheter body and includes helically arranged cutting elements which define a central clot-receiving passage. The separator scaffold may feature a smooth ribbon profile, or contoured edges of the type shown in FIGS. 10A and 10B. The separator scaffold may be radially expanded in the blood vessel and rotated and advanced to resect clot. The aspiration lumen of the catheter body and the central clot-receiving passage of the radially expandable separator scaffold are arranged coaxially so that clot resected by rotating the separator scaffold may be aspirated into the aspiration lumen of the catheter body by applying a vacuum to a proximal end of the aspiration lumen. The separator scaffold may also be used to press clot against the vessel wall and/or squeeze it within the coils as may be desirable to disrupt the clot prior to or during aspiration.

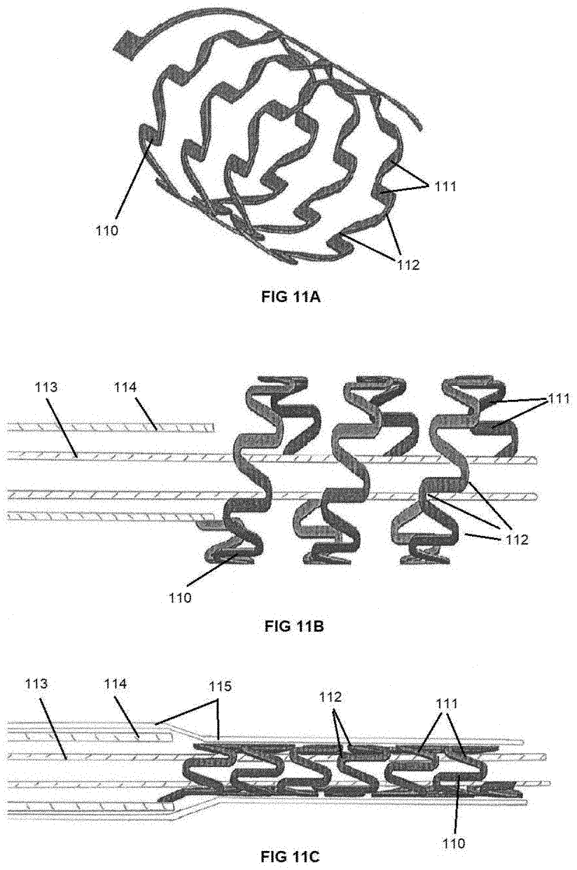

FIG. 11A shows an example of a coil with a sinusoidal ring ribbon 110 comprising struts 111 joined by crowns 112. FIG. 11A shows an oblique view of the stand alone coil. FIG. 11B shows a side cross section of the distal end of the device in the expanded configuration showing the sinusoidal ring ribbon 110 along with the inner torque member 113 and intermediate outer segment 114. FIG. 11C shows a side cross section of the distal end of the device in the collapsed configuration including the presence of a constraining sleeve 115. (In FIGS. 11B and 11C the vacuum resistant membrane which would normally cover the coil and distal end of the intermediate outer shaft has been omitted for clarity).

The main advantages of this example are that, in addition to conventional winding/unwinding to expand/collapse the coil, the sinusoidal ring of the present example can itself can expand in length, thereby assisting in expansion of the structure. The effectively wider width of the ribbon of the sinusoidal coil may also provide benefits with regards to supporting the distal sleeve during vacuum application.

In one example the sinusoidal ring ribbon 110 is made from nickel-titanium or other shape memory material cut into a sinusoidal pattern and heat set with the sinusoids open and the coil ribbon in the expanded position, such that the sinusoids are pressed into a closed position when the device is compressed into the collapsed state. The coil is then sheathed, capped, or otherwise captured in the constrained state. After delivery to the site of treatment, the sheath or cap is removed allowing the sinusoids to open to increase the diameter of the expandable segment, after which the coil can then be torqued normally to provide additional diameter control. In another example, the sinusoidal ring coil is made from a polymer which seeks to expand when exposed to moisture and/or heat. Such materials typically take a few minutes to fully expand, such that no constraint method is needed other than through torque control at the ends of the coil. The device of this example is advanced to the site of treatment, then the coil is torqued to expand it to contact the vessel, and then as the material further warms and hydrates it will seek to expand further, improving the seal against the vessel to prevent blood leakage during aspiration. After aspiration, the sinusoidal ribbon coils are fully or partially collapsed by applying a torque to them as has been described previously with non-sinusoidal coil designs.

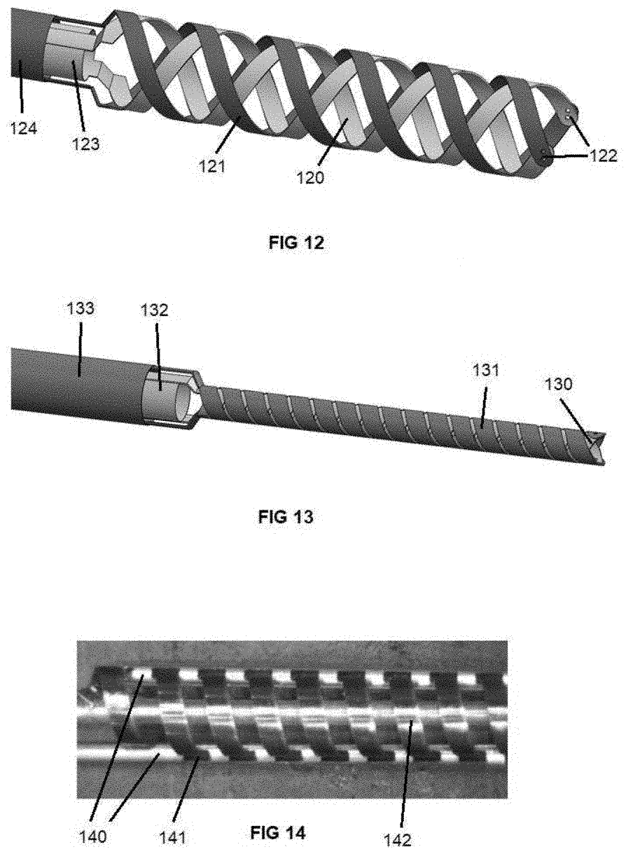

FIGS. 12 and 13 illustrate a preferred example of the present invention, in which the distal part of the inner torque member has been replaced with a second inner coil 120, 130 wound in direction opposite to that of the outer coil 121, 131. FIG. 12 shows the dual coil system in the expanded state, and FIG. 13 shows the dual coil system in the collapsed state. The two coils are joined at their distal ends 122 and hence act in unison. The two coils in the dual coil design may be attached to each other using a variety of techniques including welding, crimping, wrapping/tying with a strap or wire, rivets, or with a tab-and-slot interface. In this configuration, one coil torques against the other (usually the inner against the outer) causing both coils to open. In the neutral state the outer coil is of larger diameter than the inner coil and an optimal clearance between the two is maintained to achieve smooth rotation and the desired diameter changes.

The remainder of the catheter is substantially the same as previously described, except that the inner torque member 123, 133 terminates at approximately the end of the intermediate segment where it is then bonded to the proximal end of the inner coil. The inner torque member (of both the intermediate and proximal shafts) will typically be as large as possible in order to maximize the vacuum lumen area which lies within, and where any guidewires or supplementary devices will be tracked. Size of the proximal and intermediate inner member will be limited by the inside diameter of the outer member 124, 134 and the clearance needed between the two to allow smooth rotation and expansion and collapse of the distal expandable segment.

While the single coil example has the advantage of simplicity of manufacture, potentially lowest profile, and increased distal robustness in the collapsed state which may aid in delivery (especially if the torque element is tubular and sized to accommodate a guidewire which the device may be tracked along), the torque element takes up space in the aspiration lumen which reduces the effective tip surface are and vacuum force that can be applied. Depending on the stiffness of the inner torque element, the single coil example may also be less deliverable. In comparison, the main advantages of the dual coil example are greatest flexibility in the distal expandable segment due to the absence of any solid wire or tubular element therein, and maximum tip area in the expanded state.

The coils in the dual coil system are preferably made from NiTi due to its superior robustness, and also because NiTi is heat treatable which provides an easy-to-manufacture means of obtaining tapered coils. Tapered coils may be of benefit in achieving ideal spacing between the inner and outer coils and ensuring smooth expansion/contraction of the distal segment. In the exemplary example of the dual coil design, both the inner and outer coils are heat set to impart a tapered or conical shape, with the distal end of the coils being larger in diameter than the proximal end by about a 1.5:1 ratio. Typically, the outer coil and inner coil of such a dual coil design are heat set into different tapers intended to control spacing and friction between the two during expansion.

The coils in a two coil system may differ with respect to coil ribbon thickness, ribbon width, pitch angle, ribbon gap, etc., and either or both coils may utilize any of the other features and variants previously described, such as variable ribbon widths, multiple helixes, edge contours, sinusoidal rings.

FIG. 13 also illustrates a major advantage of the design of the present invention, which is that when the distal expandable segment is in its collapsed and constrained state it may be of significantly smaller profile than the intermediate segment with the fixed diameter aspiration lumen, thereby allowing for improved deliverability with less vessel trauma.

FIG. 14 shows another example of the present invention in which tubular attachments 140 with slots 141 are employed to maintain a constant spacing of the coil ribbon 142 through diametric changes. The slots have widths sufficient to fit the width of the coil ribbon. The slots are positioned 180 degrees apart in each tube and designed to have the coil spiral enter and exit freely in a radial direction. When fully closed, the coil tightly wraps to the tube diameter. When fully open, the coil spiral diameter increases yet spacing between loops is maintained by the tube attachment. Alternately a single slotted tube can be added to further control loop spacing of the expanded coil.

In alternate example of the coil design of the present invention, the distal end of the coil is attached to a wire, tube, other member located outside of the coil and the proximal end of the coil is attached to the catheter shaft. The outer member runs the length of the device such that torque applied to the proximal end of the outer member is transmitted to the distal end of the coil, thereby causing it to rotate to expand or collapse. If the outer member is tubular, it can serve as a secondary lumen for contrast injection, guidewire passage, or other purposes.

In an alternate example of the coil design of the present invention, a wire, tube, other member is located outside of the coil, with the distal end of this member attached to the distal end of the coil and the proximal end of this member is attached to the distal end of the intermediate segment. The proximal end of the coil is then attached to a rotating tubular torque element inside the intermediate segment outer member, such that the coil is rotated from its proximal end while the distal end is held fixed. This arrangement promotes coil expansion in the tight tortuosity, and furthermore the wire, tube, or other member running outside the coil provides an anchor for the vacuum resistant membrane. If the design features a tubular member running outside the coil, the tubular member can extend to the proximal end of the device and serve as a secondary lumen for contrast injection, guidewire passage, or other purposes.

In another example of the single coil design of the present invention, the device shafts comprise 3 elongated tubular members running the length of the device. The innermost elongated tubular member attaches to the distal end of the coil, the outermost elongated tubular member attaches to the proximal end of the coil, and the elongated tubular member between the other two tubular members attaches to the single coil somewhere in the middle of the coil. This additional shaft and attachment point allows the distal and proximal sections of the coil to be expanded and collapsed separately, to provide for variable expansion diameters best suited for vessel and clot properties, and/or to assist with distal sleeve expansion without twisting. In an alternate example utilizing the same shaft setup, the distal and proximal sections of the coil have opposite winds, such that the coil can be entirely expanded and collapsed by rotating the middle member attached to the center of the coil while the innermost and outermost elongated tubular members attached to the distal and proximal ends of the coil respectively are held fixed.

### Example 2: Distal Expandable Segment Comprising a Self-Expanding Scaffold

In a preferred example of the present invention, the distal expandable segment comprises a self-expanding scaffold. In one variant of the design, the self-expanding scaffold is in the neutral state when full expanded and is elastically compressed into the collapsed state and then constrained, and re-opens to the expanded state upon removal of the constraint. In another variant of the design, the scaffold naturally remains in the collapsed state without a constraint and only expands upon application of external stimuli such as heat, moisture, electricity, etc.

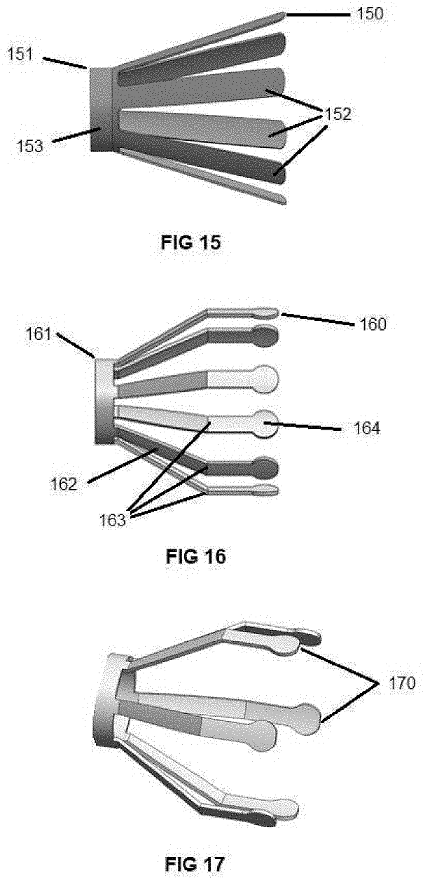

FIG. 15 shows an example of a self-expanding scaffold with a distal end 150 and a proximal end 151, comprising a plurality of struts 152 radiating in a distal direction from a common circular base 153. The base 153 is attached to the elongated tubular body of the catheter shaft. The scaffold is substantially conical in profile with a proximally oriented apex providing a taper to smoothly channel clot into the smaller lumen of the intermediate segment.

The scaffold may contain between 3 and 20 of the linear struts 152, more preferably between 5 and 12 struts, and most preferably between 6 and 8 struts. The widths of the struts may be the same for all struts in the scaffold or may vary between struts or within struts as designed to affect the profile properties of the scaffold. In one version of this example, the width of the struts can be designed to encircle the circumference of the tube. For example, for a scaffold cut from a tube with an outer diameter of 1.8 mm, thereby having an outer perimeter of 5.65 mm, the scaffold may have 6 struts each of 0.94 mm width. In another version of this example, the struts can have a width less than the maximum allowed by the tube's circumference in order to allow the struts in the self-expanding scaffold to collapse to a crimped configuration smaller than the diameter of the tube from which the scaffold is cut. In a preferred example of the present invention in which the self-expanding scaffold comprises linear struts or struts, the targeted crimped profile is 1 mm in diameter. In a self-expanding scaffold with six linear struts of equivalent width, the width of each strut would be approximately 0.5 mm.

The self-expanding scaffold may be of a length from 1 to 10 mm, more preferably from 1 to 5 mm, and most preferably from 2 to 3 mm. Shorter length scaffolds are more trackable through tortuous vessels, while longer length scaffolds will have a lower angle of opening and will funnel clot easier into the aspiration lumen.

The self-expanding scaffold is manufactured such that it will expand to a diameter equal to or larger than the vessel diameter it is intended to treat. The scaffold may be configured to expand to a diameter between 2 and 6 mm, more preferably from 3 to 5 mm, and most preferably from 4 to 4.5 mm. In one preferred example, the expandable scaffold expands to a diameter larger than the adjacent non-expandable segment of the delivery system ranging from 1.1 times to 3 times the non-expandable segment, and more preferably expands from 1.2 times to 2 times the diameter of non-expandable segment. Therefore, the device of the present invention provides an aspiration lumen in the expandable segment with a cross-sectional area between 1.5× and 10× higher than a conventional aspiration catheter with a fixed diameter aspiration lumen in the 1.4-2.0 mm range. Since the vacuum force applied equals the vacuum pressure times the cross-sectional area, the vacuum force applied by the device of the present invention is 1.5× to 10× higher than that provided by conventional aspiration catheters, with concurrently superior clot extraction capabilities.

In another example, the self-expanding scaffold is contoured for maximum performance in the desired anatomy. The self-expanding scaffold may be conical, hemispherical, or substantially cylindrical in shape, or may be a combination of the described shafts. Furthermore, the distal edge of the self-expanding scaffold may be further contoured with a flare to increase expansion diameter and aid vessel sealing, or with a taper to minimize vessel trauma during advance or withdrawal of the device.

FIG. 16 shows an example of a self-expanding scaffold with a distal end 160 and a proximal end 161, and in which the struts 162 have bends 163 allowing the expanded scaffold to better conform to the vessel in the expanded state for superior vacuum sealing. The struts also have rounded tips 164 in order to minimize vessel trauma and/or provide a larger surface area for membrane attachment. The radii of curvature of the rounded tips may be one half the width of the strut such that the strut terminates in a semi-circle, or the radii of curvature may be larger such that the struts terminate in oversize rounded tips. In another example, the self-expanding scaffold comprises struts having ovalized ends. In a preferred example, the struts terminate in oversize rounded tips of diameter approximately 1.5 times to twice the width of the strut.

FIG. 17 shows a variant of the above example in which the rounded tips of the struts can have flats 170 on the leading edge to further reduce vessel trauma and/or better distribute loads against the vacuum-resistant membrane. An alternative example the flats are on one or both sides of the tips in order to allow for tighter crimping. In a preferred example, the flat edge length is approximately ¼ to ¾ the diameter of the rounded tip.

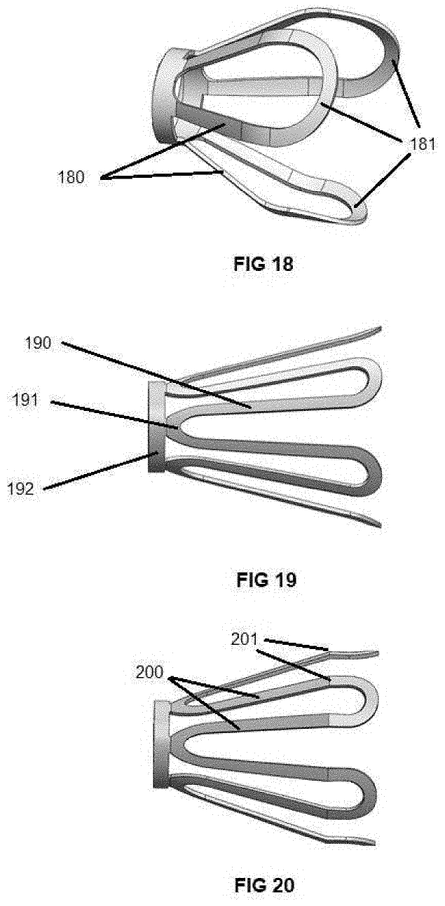

FIG. 18 shows an example of a self-expanding scaffold comprising a plurality of struts 180 and in which two or more struts are connected by arcs 181 to an adjacent strut thereby forming loops. For example, a self-expanding scaffold containing 12 struts could be formed into 6 independent loops, or four wings of 3 connected struts each, two wings of 6 connected struts each, etc. The connected arc angle can be a tangential half circle equivalent to 180 degrees such that the struts remain parallel to the axis. Alternatively, the connecting arc of the loop is designed with an arc angle different than 180 degrees, such that the linear struts used to form each loop are no longer parallel to one another or the axis. A smaller arc would draw the tips of the linear struts together such that the scaffold can crimp to a lower profile at the distal end, or a larger arc (shown) would provide for a larger starting distal profile and possibly improved expansion and clot engagement. The width of each loop and total number of loops in the self-expanding scaffold system can be used to determine a final crimped profile when pressed to contact (in the absence of strut/loop overlap). For example, a self-expanding scaffold with six equal loops of 0.6 mm outer loop width would allow for a 1.2 mm crimped profile to be attained.

FIG. 19 shows a variant of the above example in which the proximal ends of the struts 190 are also connected by arcs 191, thereby forming a sinusoidal ring or serpentine structure. In this example the curved ends, or “crowns”, of the sinusoidal ring structure are connected directly to the base 192. The sinusoidal ring structure may contain from 3 and 12 crowns, more preferably from 4 and 8 crowns, and most preferably from 4 to 6 crowns. The width of the struts in the sinusoidal ring may be between 0.005″ and 0.020″, more preferably 0.006″ and 0.014″, and most preferably 0.008″ and 0.012″. As such the ratio of ring strut width to linear strut width (when the latter are present) may vary from approximately 0.5:1 to 2:1. A flat strut may added to each crown apex of the sinusoidal ring feature in order to convert bending stress into compressive stress to enhance fracture resistance of the sinusoidal ring. In another example, the crowns at the distal end of the scaffold have a larger radius of curvature than the crowns at the proximal end of the scaffold such that the struts of expanded scaffold more gradually taper to the shaft.

FIG. 20 shows a variant of the above example in which the struts 200 have bends 201 near the crown tips allowing the expanded scaffold to better conform to the vessel in the expanded state for superior vacuum sealing.

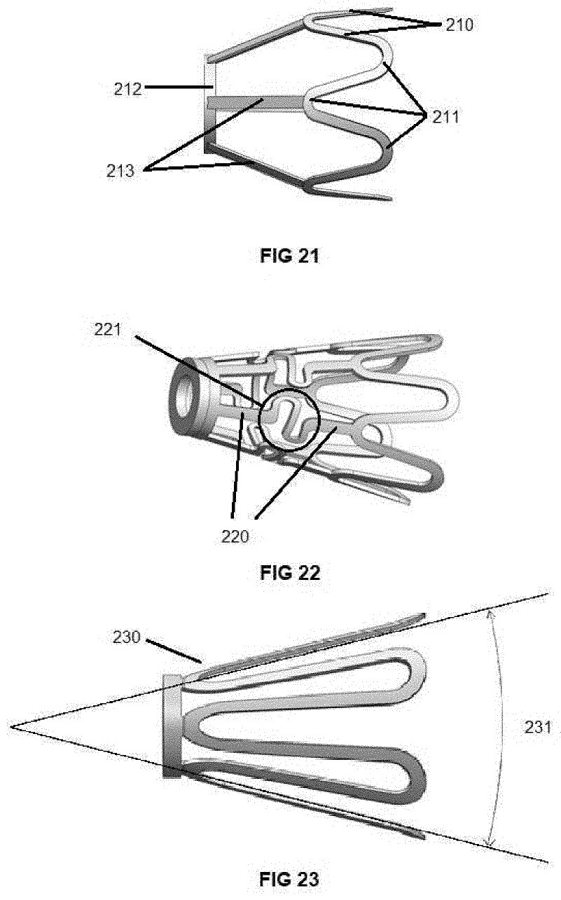

FIG. 21 shows an example of a self-expanding scaffold comprising a plurality of struts 210 connected by arcs 211 at both ends to form a sinusoidal ring structure, and in which the proximal end of the ring is attached to the scaffold base 212 by linear strut links 213. This design allows the sinusoidal ring to more evenly share compression load between the distal and proximal crowns thereby increasing the expansion force and resistance to vacuum collapse. Furthermore, the sinusoidal ring aids in maintaining a circular entrance to the aspiration lumen.

The sinusoidal ring axial length may be from 30% to 60% of the total scaffold length, more preferably from 40% to 50% of the total scaffold length. For example, if the total length of a self-expanding scaffold is 5 mm, the sinusoidal ring may be 2 mm and the linear struts connecting it to the elongated tubular body may be 3 mm.

In the preferred example, each proximal-facing crown tip in the sinusoidal ring scaffold is anchored by a linear strut link to prevent unanchored crown tips from interfering with sheath advancement or from potentially inducing vessel trauma during device pullback in the expanded state. In another example, the sinusoidal ring scaffold has more crowns than there are linear struts, allowing for greater scaffold flexibility for device delivery in the patient. In an alternate example the links connect to the middle of the struts in the sinusoidal ring or to the distal end crowns rather than to the proximal crowns.

In another example, the links are not coaxial with the centerline of the elongated tubular body and wrap in a spiral configuration to improve system flexibility or evenness of expansion in tortuous anatomy. For instance, the base of the link can be aligned to one crown of the sinusoidal ring with the ring attachment at the adjacent ring crown. Alternatively, the wrapping angle is increased by further offsetting the link attachment to the next adjacent ring crown. In another example, one or more of the links attaching the sinusoidal ring to the scaffold base are split through the axial length producing a sinusoidal ring having multiple crown members. This configuration reduces rigidity of the self-expanding scaffold to aid vessel conformability during track and expansion.

In an alternate example of the present invention, the scaffold may be composed from more than one sinusoidal ring attached to each other and/or the catheter shafts directly and/or with straight, curves, or articulated links. In a parallel design well suited for ease of manufacture, a tube is cut with alternating slots in order to create a structure of conjoined serpentine rings in the expanded state, in a pattern well known to those in the industry.

FIG. 22 shows a variant of the above example in which the links 220 include a ‘U’, “M”, ‘Z’ or ‘S’ or similar geometry 221 in order to increase flexibility of the linear strut and the self-expanding scaffold as a whole. The flexibility-increasing geometry may be in middle of the linear strut or positioned nearer to the proximal end of the linear strut (near the elongated tubular body) or nearer to the distal end of the linear strut, close to the attached sinusoidal ring, if applicable. The strut width of the flexibility-increasing geometry portion of the linear strut may be the same as that of the straight sections of the linear strut or they may be thinner. In the preferred example, the strut width of the flexibility-increasing geometry portion of the linear strut is approximately half that of the straight sections of the linear strut.

### Effect of Self-Expanding Scaffold Geometry

The combination of the length, diameter, and contour of the self-expanding scaffold is important in determining the delivery, expansion, aspiration, and re-collapse (if applicable) performance of the device. Since the expandable scaffold portion of the device is typically stiffer than any guidewire and/or adjacent device components, the length of the expandable scaffold may impact deliverability. Shorter scaffolds can articulate more easily through a tortuous vessel than longer scaffolds. Shorter lengths are also better suited to resisting collapse during aspiration, since during aspiration the applied vacuum results in a pressure differential between the ambient blood pressure on the outside of the scaffold and the lower blood pressure under vacuum on the inside of the scaffold. This pressure differential seeks to recollapse the scaffold back into the crimped state. Shorter lengths provide for both less total force applied to the scaffold (less area for the pressure to act upon) and for a shorter lever arm against which that force is applied. However shorter scaffolds have to expand wider in order to contact the vessel wall for proper sealing and aspiration, which may decrease clot aspiration efficiency. The width of expansion can be characterized by the “included angle” of the expanded scaffold.

FIG. 23 shows an example of conical scaffold 230 formed with a tapered serpentine body attached to a base ring which expands radially outwardly at an angle 231 in a distal direction. While an included angle of 180° (where the scaffold has expanded into a disk perpendicular to the axis of the catheter) would functional and feasible as it will seal the vessel and perform superior to a conventional aspiration catheter, such a configuration may not funnel clots into the aspiration lumen as well as a design with a more tapered entry would. Preferably, the self-expanding scaffold has an included angle between 20° and 120°, more preferably between 30° and 90°, and most preferably between 40° and 60°, in order to provide the best balance of deliverability and clot aspiration while maintaining sufficient vacuum resistance to avoid collapse. In a preferred example, the expandable scaffold is 2-3 mm long in the crimped state and expands to 4-5 mm diameter when unconstrained, which depending on the inner diameter of the aspiration lumen at the proximal end of the scaffold yields an included angle in the expanded state between 40° and 60°.

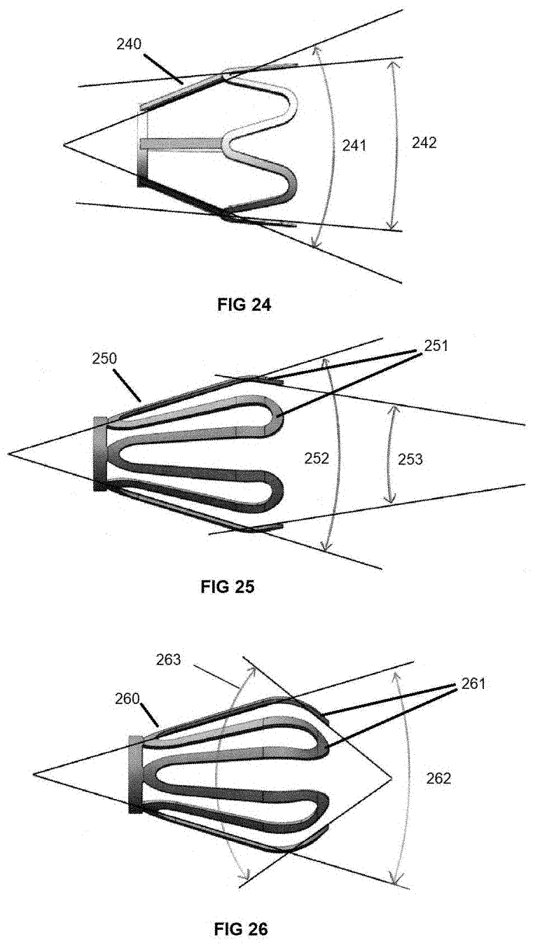

Some scaffold contours result in more than one angle within the scaffold which may result in a gentler and less potentially traumatic contact with the vessel and/or positively impact aspiration efficiency. Typically, the distal portion of the scaffold will have a shallower angle while the proximal portion of the scaffold would have a steeper angle. FIG. 24 shows an example of conical scaffold 240 with a first and steeper proximal region included angle 241 and a second and shallower distal region included angle 242.

If the scaffold has been manufactured in a hemispherical or similar curved shape, the angle will increase smoothly along the length of the scaffold. In another example of the invention, the distal end of the scaffold has a reverse angle and in the expanded state the tips point into the lumen, such that if the expanded scaffold is advanced in the lumen the tip of the scaffold will help guide it along the vessel. FIG. 25 shows an example of conical scaffold 250 with inward-pointing tips 251, resulting in proximal scaffold region with included angle 252 and a distal scaffold region with reverse included angle 253. FIG. 26 shows a variant of the above example in which the scaffold 260 has more gradually curved and significantly inward-pointing tips 261, resulting in proximal scaffold region with included angle 262 and a distal scaffold region with steeper reverse included angle 263.

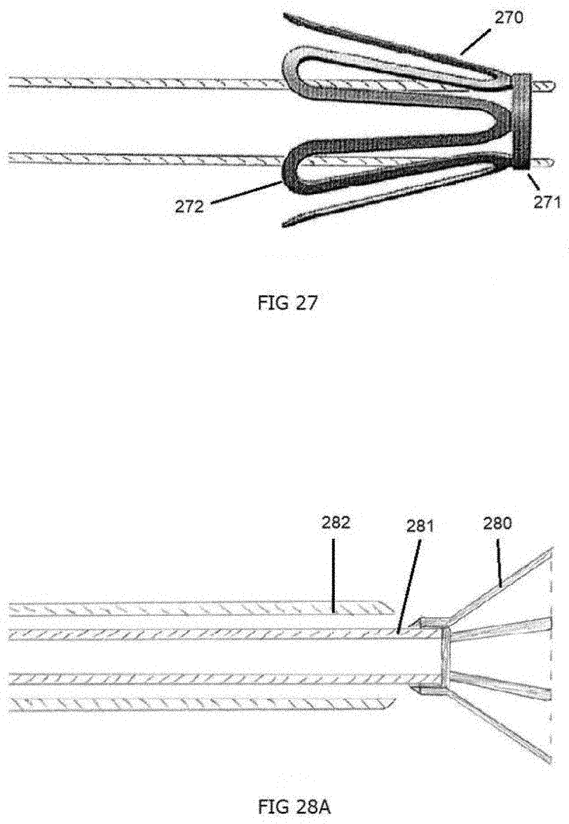

FIG. 27 shows another example of the present invention in which the self-expanding scaffold 270 is mounted with the apical end of the scaffold 271 distal and largest expanded diameter end 272 proximal. This example may utilize any of the self-expanding scaffold designs described elsewhere herein, as well as most of the constraint techniques discussed below. One advantage to using a reversed scaffold of this sort is that during aspiration the pressure gradient between the ambient blood pressure proximal to the expanded scaffold and the vacuum region distal to it seek to further open the scaffold and press it into the vessel wall, thereby providing a superior seal between the device and the vessel and maximizing vacuum levels and aspiration efficiency. Another advantage is that the device can be easily advanced deeper into the vessel, even during aspiration, in order to press into the clot or to capture more distal fragments not initially aspirated. In the preferred example, the system uses a drawstring design to facilitate collapse of the expanded umbrella after aspiration and prior to withdrawal from the anatomy.

### Means of Constraint and Release for Self-Expanding Scaffold

There are multiple means in which a self-expanding scaffold may be constrained during delivery through the vasculature to the site of treatment and thereafter expanded, and in some cases optionally may be collapsed after the aspiration treatment is complete.

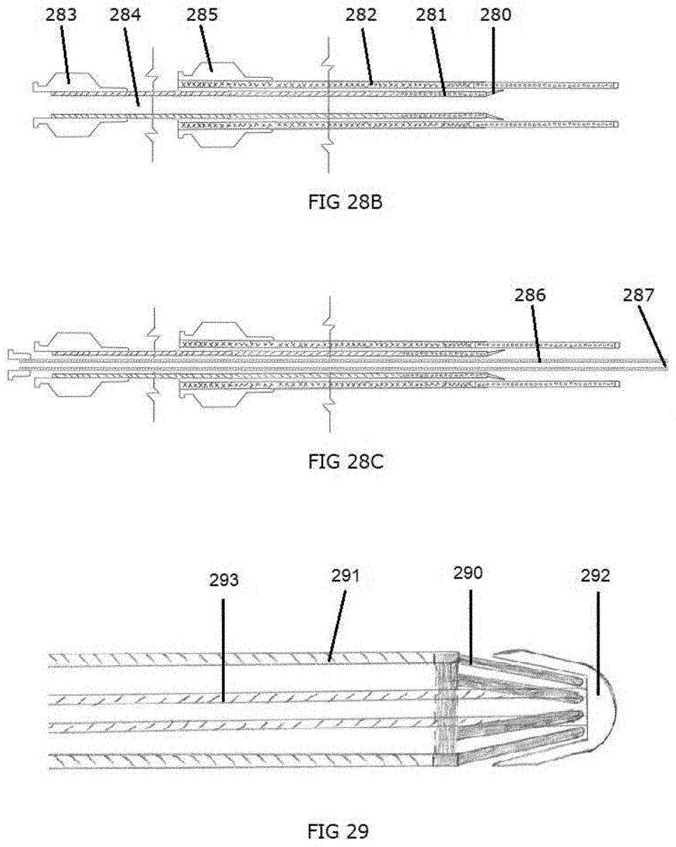

FIG. 28A shows a preferred example in which the device comprises a self-expanding scaffold 280 attached to an inner elongated tubular body 281 which lies inside an outer elongated tubular body 282, with sufficient clearance between the inner and outer elongated tubular bodies to allow one to move distal and/or proximal with respect to the other. As manufactured and during delivery through the vasculature to the clot, the scaffold is kept sheathed in the collapsed state by the outer elongated tubular body. In this example the two tubular bodies are advanced together to the site of treatment, then the outer body is moved proximal with respect to the inner body, or the inner body is moved distal with respect to the outer body, thereby uncovering the self-expanding scaffold and allowing it to expand to the larger configuration for treatment. Alternatively, the physician may opt to advance the outer tubular body separately from the inner tubular body if such may provide a deliverability or other benefit, and then advance the inner tubular body with self-expanding scaffold within the outer tubular body as a secondary step.

FIG. 28B shows the full length of the device of FIG. 28A, in which the inner elongated tubular body 281 has a proximal hub 283 and an aspiration lumen 284. The outer elongated tubular body 282 also has a proximal hub 285 to facilitate flushing the annular area between the two elongated tubular bodies with saline in order to prevent introduction of air into the patient during the procedure.

FIG. 28C shows the device of FIGS. 28A and 28B after it has been loaded over a microcatheter 286 and is ready for insertion into the body and delivery to the site of treatment. The tip of the microcatheter 287 will be loaded over a guidewire, and the guidewire and microcatheter will provide support for the outer devices during deliverability.

In a preferred example, the outer tubular body has sufficient axial rigidity to allow it to be pulled back with respect to the inner tubular body to allow the self-expanding scaffold to expand, as well as be again advanced to close the self-expanding scaffold after aspiration. In another example the outer tubular body is intended only to be used in tension, which allows the outer tubular body to be pulled back and to release the self-expanding scaffold to expand, but not in compression in which the sheath requires sufficient compressive strength and buckling resistance to allow it to be advanced to re-collapse the self-expanding scaffold upon completion of aspiration. This example may be preferable when minimum profile and/or maximum aspiration lumen size is more desirable than the ability to return the self-expanding scaffold to the crimped state after aspiration is complete. The portion of the outer tubular body over the catheter intermediate segment and/or proximal segment may be drilled, notched, slotted, or otherwise cut to increase flexibility without significantly compromising tensile strength and stiffness.

In an alternate example, the constraining sheath only covers the scaffold, and possibly part of the catheter shafts, and is manipulated used a wire or catheter running through the aspiration lumen of the device and which is attached to the sheath. The wire or catheter may exit the distal end of the aspiration lumen through the scaffold distal tip, or through a port made for the purpose in the side of the device outer member.

FIG. 29 shows a preferred example in which the self-expanding scaffold 290 is attached to the distal end of the outer elongated tubular body 291 and is kept in the constrained state by a distal cap 292 attached to a removable inner elongated tubular body 293. In this example the two elongated tubular bodies are advanced together to the site of treatment, then the outer elongated tubular body is moved proximal with respect to the inner elongated tubular body, or the inner elongated tubular body is moved distal with respect to the outer elongated tubular body, thereby uncovering the self-expanding scaffold and allowing it to expand to the larger configuration. The inner elongated tubular body with distal cap is then withdrawn through the lumen of the outer elongated tubular body and removed from the device and patient's body, thereby providing an open aspiration lumen.

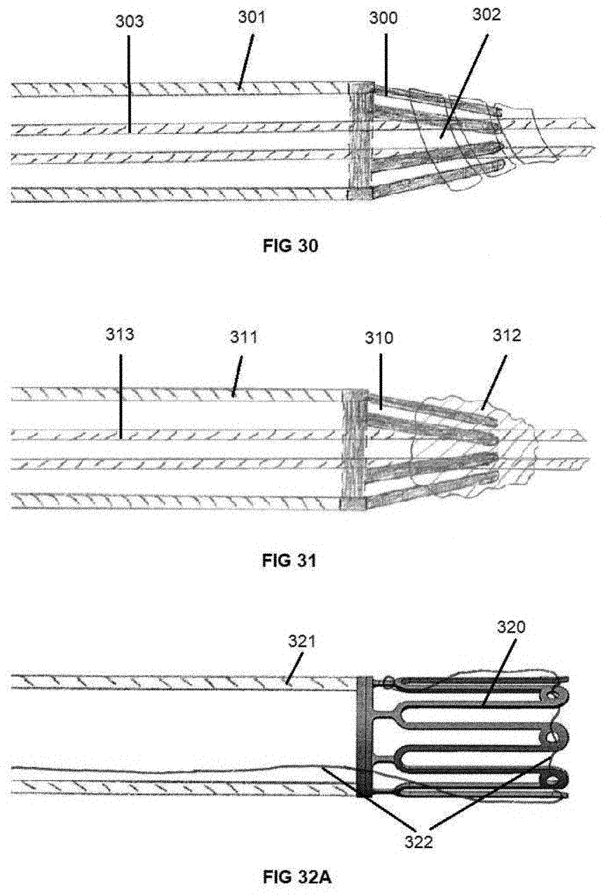

FIG. 30 shows a preferred example in which the self-expanding scaffold 300 is attached to the distal end of the outer elongated tubular body 301 and is kept in the constrained state by a wire, filament, or ribbon 302 wrapped around the at least distal end of the self-expanding scaffold and attached to a removable inner elongated tubular body 303. The wire, filament, or ribbon wraps over itself over the self-expanding scaffold thereby securing the end of the wire, filament, or ribbon not attached to the removable inner elongated tubular body, yet distal tension on the wire, filament, or ribbon causes it to unwrap easily and come free of the self-expanding scaffold. In one example, the wire is made from stainless steel, nitinol, a cobalt chrome alloy, titanium, or other metal of sufficient tensile strength and biocompatibility. In another example, the filament is made from nylon, PTFE, FEP, ePTFE, a suture material, or other polymer of sufficient tensile strength and biocompatibility. In another example, the wire or filament is of a substantially flattened cross-section such that the material resembles a ribbon more than a rod. In this example featuring a constraining wrapped wire, filament, or ribbon, the two elongated tubular bodies are advanced together to the site of treatment, then the outer elongated tubular body is moved proximal with respect to the inner elongated tubular body, or the inner elongated tubular body is moved distal with respect to the outer elongated tubular body, thereby unwrapping the wire, filament or ribbon from the self-expanding scaffold and allowing it to expand to the larger configuration. The inner elongated tubular body with the wire, filament, or ribbon is then withdrawn through the lumen of the outer elongated tubular body and removed from the device and patient's body, thereby providing an open aspiration lumen. A relative advantage of this example versus the example featuring a cap is that as wrapped the wire, filament, or ribbon adds minimal stiffness to the system, and also once unwrapped can be easily withdrawn through the self-expanding scaffold and catheter shafts.

FIG. 31 shows an example in which the self-expanding scaffold 310 is attached to the distal end of the outer elongated tubular body 311 and is kept in the constrained state by a frangible material 312 which seals the at least distal end of the self-expanding scaffold to the removable inner member 313. The frangible material may be a water-soluble solid like a sodium chloride or potassium chloride salt crystal, a biodegradable polymer such as PLLA, or an adhesive gel. It may also be a solid scaffold made from polymer or metal which is securely attached to the removable inner member and which has loops or tabs covering the struts of the self-expanding scaffold to constrain it which can be broken away to release it. The two elongated tubular bodies are advanced together to the site of treatment, then the outer elongated tubular body is moved proximal with respect to the inner elongated tubular body, or the inner elongated tubular body is moved distal with respect to the outer elongated tubular body, thereby causing the self-expanding scaffold to break free of the frangible material and allowing it to expand to the larger configuration. The inner elongated tubular body and any remaining frangible material is then withdrawn through the lumen of the outer elongated tubular body and removed from the device and patient's body, thereby allowing an unoccluded aspiration lumen.

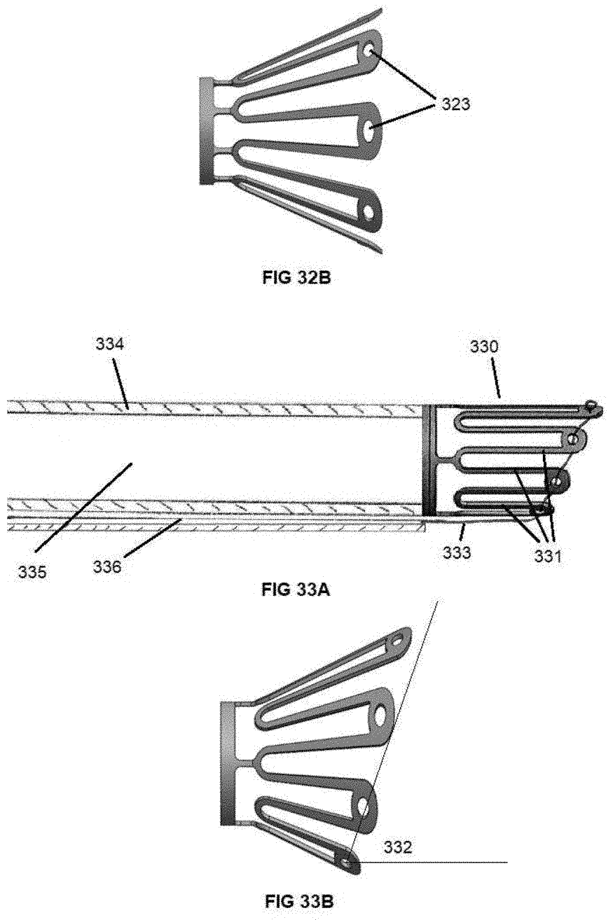

FIGS. 32A and 32B show another preferred example of the present invention in which the self-expanding scaffold 320 is attached to the distal end of an elongated tubular body 321 and is kept in the constrained state by a drawstring filament 322. FIG. 32B shows a detail of the self-expanding scaffold featuring circular holes 323 at the distal end of the scaffold through which the filament is threaded. Pulling tension on the filament draws the arms of the scaffold together to reduce it to the collapsed state, while releasing the tension allows the self-expanding scaffold to reopen. In another example of the present invention featuring a filament, the self-expanding scaffold is initially constrained by another method of constraint described herein and the filament is used primarily to allow re-collapse of the scaffold after release and expansion. This may allow for a tighter initially collapsed profile, and also for easier expansion because scaffold deployment is not hindered by friction of the filament sliding through the features.

Instead of holes the self-expanding scaffold may contain features such as slots, loops, rings or hooks instead of circular holes through which the filament is threaded, or the filament may be wound directly around the struts, crowns, or other struts in the self-expanding scaffold. In an alternative example, a second filament may wrap around the perimeter of the self-expanding scaffold and protrude through features in the scaffold like those described above or between natural gaps in the scaffold pattern, and the primary filament only laces through and pulls on the perimeter filament. An advantage to this approach is that filament does not need to be threaded directly through multiple struts of the scaffold, and/or it interfaces only with the perimeter filament, resulting in less friction in the assembly and smoother/easier operation.

In one example the filament runs the length of the catheter body to a slider or other mechanism on the handle which allows the physician to put it in tension or release said tension, thereby expanding or collapsing the scaffold. In another example the filament attaches to a wire, tube, or other component with torsional rigidity which runs the length of the catheter body, and this torsion component is rotated to wind or unwind the filament around it thereby pulling tension on it or releasing such tension. An advantage of using such a torque element arrangement is it omits any stretch in the filament being tensioned along the length of the shaft, and also eliminates any tendency of the filament tension causing the shaft to deflect.

The filament may be made from polymeric materials such as nylon, PEEK, FEP, PTFE, ePTFE, or UHMWPE filaments or ribbons, metals such as stainless steel, NiTi, cobalt chrome alloys, or titanium wires or ribbons, or any material providing similarly sufficient tensile strength and biocompatibility. The filament may be made from two or more components, for example with stiffer and more axially rigid components running along the proximal portions of the elongated tubular bodies, and more flexible and/or lower friction materials used in the more distal portions of the device. The filament may run inside the aspiration lumen of the device, in a separate channel substantially within the wall of the elongated tubular body, and/or immediately outside of the elongated tubular body in an attached channel.

If the design uses a torque element to wind or unwind the filament, the construction of such torque element would be as has been previously described for an inner torque member used for a coil distal segment design, except that in this case the torque element may run fully or partially outside the aspiration lumen, either free floating or in its own channel in either case.

FIGS. 33A and 33B show a variant of the above example in which the self-expanding scaffold 330 features struts 331 of different lengths, thereby reducing the angle 332 at which the filament 333 engages the first contact positions in the self-expanding scaffold and reducing friction of operation. In another example, two or more filaments are used to reduce the amount of contact points of each filament and friction of operation. FIG. 33A also depicts the use of a multi-lumen catheter shaft 334 with one dedicated aspiration lumen 335 and one dedicated filament lumen 336.

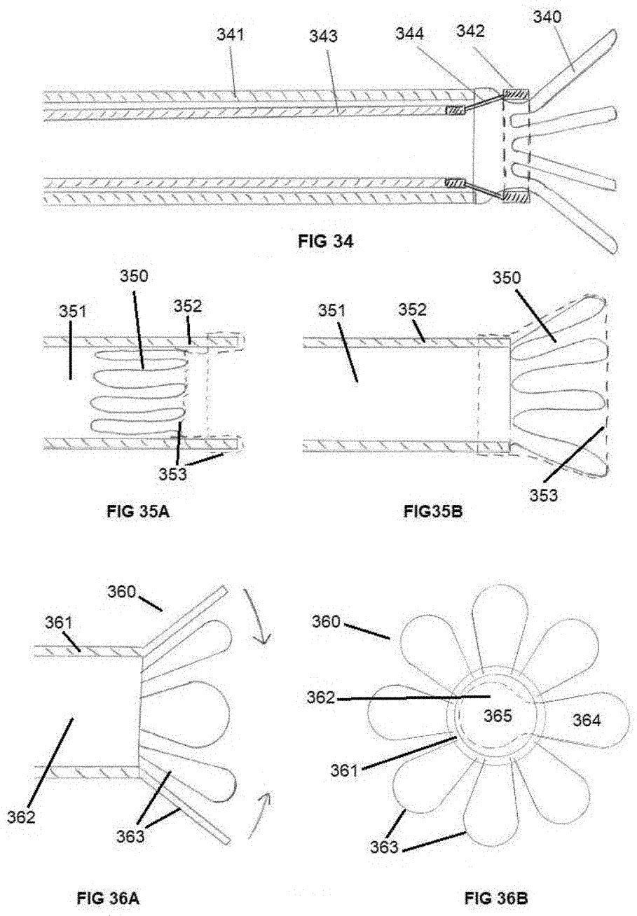

FIG. 34 shows another example of the present invention in which the self-expanding scaffold 340 is attached to the distal end of an elongated tubular body 341 and is kept in the constrained state by a ring 342 on the outside of the self-expanding scaffold, and the two are designed such that the ring can slide partially or completely along the self-expanding scaffold, such that when the ring is in a more distal position, the self-expanding scaffold is kept in the collapsed state, and when the ring is in a more proximal position the self-expanding scaffold is able to expand. The ring may be made of metallic, polymeric, or other material. Its position on the self-expanding scaffold is controlled from the proximal end of the device by wires, rods, or a tubular inner member 343 which extends continuously to the proximal end of the device. The ring may be corrected directly to the control wires, rod, or tubular inner member, or be part of a structure which includes for example links 344 connecting the constraint ring 342 to the inner member. The method of proximal control, whether wire(s), rod(s), and/or an elongated tubular member, may be positioned inside of or outside of the elongated tubular member to which the self-expanding strut is attached. In the preferred example, the constraining ring is laser cut from a nickel-titanium alloy and incorporates struts connecting it to a second ring bonded to an elongated inner member riding inside the outer elongated tubular member to which the self-expanding strut is attached. One key advantage to this design is that upon completion of aspiration, the constraining ring can be advanced to re-collapse the self-expanding strut for minimum vessel trauma during withdrawal from the patient.

FIGS. 35A and 35B show an example of the present invention in which the self-expanding scaffold 350 is capable of being compressed and folded into the fixed-diameter aspiration lumen 351 of the distal end of the catheter shaft 352, where it is retained by friction against the lumen or other components. The expandable segment is deployed by pushing it free of the lumen using a plunger wire or tube inside the aspiration lumen for this purpose, or the inner member can be used. In the example shown, the self-expanding scaffold comprises a sinusoidal ring scaffold which is only attached to the rest of the device at its distal end by the overlying vacuum resistant membrane 353, such that the sinusoidal ring scaffold can be crimped into a smaller cylindrical shape and inserted inside the catheter shaft 352, essentially turning the sleeve inside out. This example has the advantage that the self-expanding sinusoidal ring scaffold will continue to press outwards, keeping it firmly in position inside the catheter shaft while also maintaining a substantially clear lumen for passage of guidewire, microcatheters, and the like.

FIGS. 36A and 36B shows another example of the concept in which the scaffold is constrained by the aspiration lumen. In this example the self-expanding scaffold 360 is attached to the outer catheter shaft 361 which surrounds the aspiration lumen 362. The scaffold comprises loop struts 363 of outer perimeter slightly less that of the aspiration lumen 362, which are pressed into a circular shape and then folded across and slightly into the aspiration lumen like the petals of a flower. In the expanded state each loop is in the position 364, and when folded each loop is in the position 365. After being folded inside the aspiration lumen the loops will seek to return to a less circular state, thereby pressing against the inside of the aspiration lumen and remaining in the collapsed state until pressed free by a plunger wire, tube, or inner member in the aspiration lumen.

In another example of the present invention, the self-expanding scaffold is attached to the distal end of the outer elongated tubular body and is kept in the constrained state by features on the self-expanding scaffold such as struts, tines, hooks, linear or curved struts, flares, or other physical additions or alterations to the scaffold, which are themselves constrained from the inside of the self-expanding scaffold, thereby holding the entire self-expanding scaffold in the constrained state. In a preferred example the constraint-enabling features consist of linear struts attached to the distal end of the self-expanding scaffold which are then captured within an inner elongated tubular body. In this example the two elongated tubular bodies are advanced together to the site of treatment, then the outer elongated tubular body is moved distal with respect to the inner elongated tubular body, or the inner elongated tubular body is moved proximal with respect to the outer elongated tubular body, thereby releasing the linear struts and allowing the self-expanding scaffold to expand to the larger configuration. The inner elongated tubular body is then withdrawn through the lumen of the outer elongated tubular body and removed from the device and patient's body, thereby allowing an non-occluded aspiration lumen. In another example, the linear struts are of different lengths to aid in assembly of the device.

In another example of the present invention, the self-expanding scaffold is attached to the distal end of the outer elongated tubular body and is kept in the constrained state by capture features on the self-expanding scaffold such as holes, loops, or curves in the linear struts or sinusoidal ring which interface with a complementary geometry on the elongated inner member, thereby holding the entire self-expanding scaffold in the constrained state. In a preferred example the capture features consist of loops within the design of the self-expanding scaffold, and the complementary geometry is a crown-shaped structure bonded to or cut into the inner elongated tubular body. When the self-expanding scaffold is in the collapsed state, the peaks of the crown-shaped structure hook the loops within the self-expanding scaffold, thereby holding the system in the collapsed state. In this example the two elongated tubular bodies are advanced together to the site of treatment, then the outer elongated tubular body is moved distal with respect to the inner elongated tubular body, or the inner elongated tubular body is moved proximal with respect to the outer elongated tubular body, thereby disconnecting the crown-shaped geometry at the distal end of the elongated tubular inner member from the self-expanding scaffold such that it can expand to the larger configuration. The inner elongated tubular body is then withdrawn through the lumen of the outer elongated tubular body and removed from the device and patient's body, providing an non-occluded aspiration lumen. In another example, the self-expanding scaffold is held in the constrained state by one or more wires or hooked or curved rods attached to the inner member which are hooked into or looped through the capture features in the self-expanding scaffold. Alternatively, the elongated tubular inner member can be omitted and the capturing crown-shaped structure, wires, hooked or curved rods, or other means of capture extend directly to the proximal end of the device such that it can be manipulated by the user to release the constraint on the self-expanding scaffold and allow it to deploy.

The elongated tubular member(s) which may be used to constrain and deploy self-expanding distal scaffolds are manufactured from a cylindrical polymeric tube. The tube can be manufactured from nylon, Pebax, polyurethane, silicone, polyethylene, PET, PTFE, FEP, PEEK, polyimide, or other materials. Single wall thickness of the tubes would be between 0.001″ and 0.020″, preferably between 0.002″ and 0.010″, and most preferably between 0.003″ and 0.008″. Material hardness of the polymeric tube components would be between 50A and 80D. The elongated tubular member(s) can be constructed from a single polymer extrusion, or be assembled from multiple pieces of varying wall thicknesses and stiffnesses bonded together. The multiple pieces could be bonded together using adhesives, laser, RF, ultrasonic, or hot air heat bonds, be melted in an oven to merge with each other, or using other methods widely known in the industry. Any elongated tubular member(s) may be reinforced by coils and/or braids of metals or polymers to improve mechanical properties, in particular axial stiffness to provide for efficient push force transmission to the device tip in order to release a constrained self-expanding scaffold. Such reinforcing materials may include but are not limited to various alloys of stainless steel, cobalt chrome, nickel-titanium, platinum and platinum-iridium, PEEK, polyimide, Kevlar, and UHMWPE. Any coil may be a spring guide in which the adjacent loops of the coil are in direct contact with each other in order to provide maximum axial stiffness, shaft push, collapse resistance, and radiopacity. In an example of the present invention in which the self-expanding scaffold is laser cut from a tube, an additional portion of said tube not used for the self-expanding scaffold can be cut into a non-expanding coil, ring, spine, braid, and/or other geometry to aid in attaching the self-expanding scaffold to the adjacent catheter shaft, and/or to reinforce or provide the foundation for construction of such shaft. In particular a design with an axial spine provides for improved axial stiffness and push and pull force transmission along the length of the device.

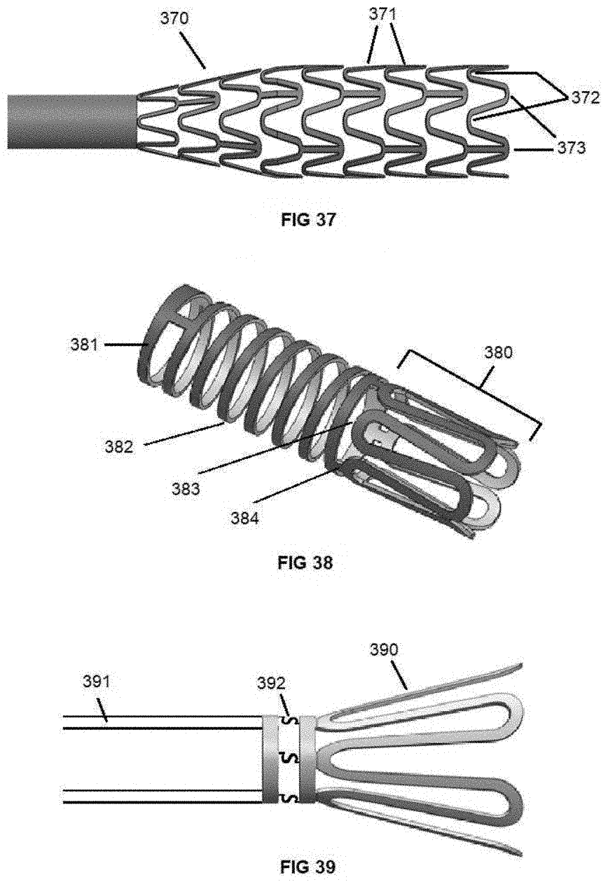

In another example of the present invention, the distal expandable segment only seeks to expand when exposed to moisture and/or heat. Exposure to such conditions causes the struts within the expandable scaffold to swell in width and/or length which due to the design of the scaffold thereby causes the entire scaffold to open. A slotted tube or sinusoidal ring type scaffold would be most suitable for this sort of design. FIG. 37 shows an example of a scaffold 370 composed of sinusoidal rings 371 made from a polymer which swells when exposed to moisture. When introduced into the body in the crimped state, the moisture in the patient's blood is pulled into the polymer, increasing stress on the inside of the folded crowns 372 more than it increases the stress on the outside of the folded crowns 373, thereby causing the crowns to unfold and the scaffold to expand. In this sort of design, the expandable distal segment may be constrained by any of the features, methods, or techniques describe herein for constraining a self-expanding scaffold, or the expandable distal segment may remain unconstrained and the device designed to expand at the appropriate rate in vivo.

Polymers examples suitable for use as a self-expanding scaffold which swell when exposed to moisture include graft polymers, block polymers, polymers with special functional groups or end groups such as acidic or hydrophilic type, or blend of two or more polymers. Polymeric material examples comprise one or more of Poly(lactide-co-caprolactone), Poly(L-lactide-co-ε-caprolactone), Poly(L/D-lactide-co-ε-caprolactone), Poly(D-lactide-co-ε-caprolactone), poly(glycolic acid), poly(lactide-co-glycolide, polydioxanone, poly(trimethyl carbonate), polyglycolide, poly(L-lactic acid-co-trimethylene carbonate), poly(L/D-lactic acid-co-trimethylene carbonate), poly(L/DL-lactic acid-co-trimethylene carbonate), poly(caprolactone-co-trimethylene carbonate), poly(glycolic acid-co-trimethylene carbonate), poly(glycolic acid-co-trimethylene carbonate-co-dioxanone), or blends, copolymers, or combination thereof. The polymeric material in this invention can be blends of two or more homopolymers such as polylactide, poly(L-lactide), poly(D-lactide), poly(L/D lactide) blended with poly(caprolactone), polyglycolide, polydioxanone, poly(trimethyl carbonate), or the like.

Polymers suitable for use as a self-expanding scaffold which change shape when heated to body temperature include poly(methacrylates), polyacrylate, polyurethanes, and blends of polyurethane and polyvinylchloride, t-butylacrylate-co-poly(ethyleneglycol) dimethacrylate (tBA-co-PEGDMA) polymers, combination thereof, or the like. These polymers exhibit shape memory properties and undergo a phase transformation at body temperature and seek to return to a pre-established state.

In another example of the present invention in which the distal expandable segment expands when exposed to moisture and/or heat, only part of the scaffold is composed of materials or struts sensitive to such stimuli, but which act on other non-sensitive parts within the scaffold to induce the entire scaffold to open.

In another example of the present invention, the distal expandable segment only expands when charged with an electric current. Upon application of the current the elements within the expandable structure seek to either shorten or lengthen which due to the design of the structure thereby causes the entire structure to open.

### Methods of Distal-Segment Attachment

The means by which a distal expandable segment is attached to the elongated tubular body of the intermediate segment can significantly impact the performance of the device with respect to profile, flexibility, deliverability, and aspiration, especially with a self-expanding scaffold design which tends to be stiffer than a coil design. In the simplest configuration, the distal expanding segment terminates proximally in a ring of approximately the same diameter as the adjacent shaft, and is intended to be butt-joined to the shaft or lap-joined inside or outside the shaft (see FIG. 32A for an example). The ring may be have a notch or slit allowing it to be stretched to crimp over the shaft or compressed to squeeze inside of it. While simple to manufacture and robust in tension or compression, this attachment approach may result in a locally stiff junction. A more flexible junction is desirable because it allows the distal portion of the device including the expanding structure to turn easily to follow the guidewire and track through vascular tortuosity. This aids ease of delivery to the site of treatment. Furthermore, a flexible junction is desirable because when the distal segment is expanded, it will flex or swivel at the junction and self-align with the vessel. This aids vessel sealing and clot aspiration, especially in tortuosity.

FIG. 38 shows an example of a flexible junction design in which the distal expanding segment 380 is detached from the base attached to the catheter shaft 381 by a coil or pigtail structure 382. The coil or pigtail structure can flex easily improving the ability of the distal structure to conform to the vessel in both the crimped and expanded states, while an extension of the vacuum resistant membrane allows the system to maintain vacuum integrity. If axial movement in tension or compression is undesirable, and/or twisting needs to be resisted as part of an expanding coil design, the loops of the coil can be joined by links to restrict this without significantly impacting flexibility. If all such links are straight then it forms a spine-and-loops structure, or alternately one or more “M”, “S”, “U”, “W”, or other such curved links can be employed. Alternately or additionally, a polymer layer can be bonded over or into the pigtail to reinforce it against axial movement and/or provide for vacuum resistance. One or more of the proximal ends of the struts or crowns 383 of a self-expanding scaffold may be free floating and not connected to the pigtail structure except through adjacent struts or sinusoidal curves, which are either directly connected themselves or indirectly connected through links 384.

FIG. 39 shows another example of the present invention in which the distal expanding structure 390 is connected to the adjacent catheter shaft 391 using one or more “S”, “M”, “U”, “W”, or other such flexible links 392.

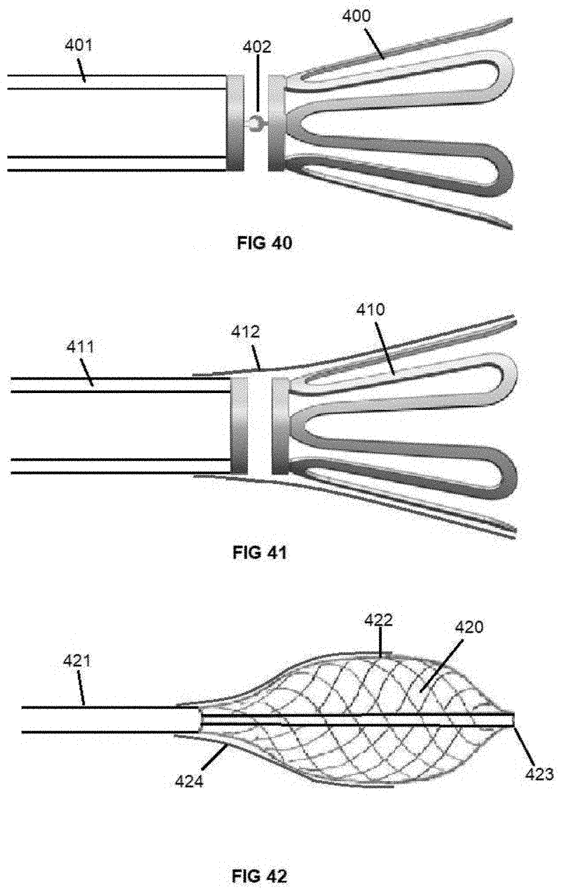

FIG. 40 shows another example of the present invention in which the distal expanding structure 400 is connected to the adjacent catheter shaft 401 using one or more ball-and-socket type joints 402. Such joints may be substantially 2 dimensional or 3 dimensional in nature.

FIG. 41 shows another example of the present invention in which the distal expanding structure 410 is entirely disconnected from the adjacent catheter shaft 411 except by the vacuum resistant membrane 412. The distal expanding structure may be a single uniform structure, or comprise multiple independent elements with free distal and/or proximal ends coupled only by the membrane.

### Alternate Designs and Mechanisms for the Distal Expandable Segment

In addition to the various coil and self-expanding scaffold designs previously described, there are several alternate means of creating a reversibly driven distal expandable segment utilizing a design which has a crimped/collapsing/opening or folding/unfolding structure which expands and collapses when acted upon by a mechanical force such as a pushrod, pull wire, torque shaft, or hydraulic force.

FIG. 42 shows an example of the present invention in which the distal expandable segment comprises a braided structure 420 which flares outwards from the catheter shaft 421 to the intended maximum expansion diameter 422 and then tapers down to connect to an inner member at its distal end 423. The inner member is torqued and/or pushed and pulled to open and close the distal expandable segment. The vacuum resistant membrane 424 covers the braided structure up to approximately its point of maximum diameter, while distal to that the structure is an open mesh through which the clot can be aspirated. In the preferred example, the braid uses thin wires and/or a smaller number of wires so as to provide the most open mesh possible and not obstruct clot aspiration, and/or is designed such that the braid wires splay during expansion of the distal segment thereby leaving distal areas with more concentrated wires and areas that are substantially open and more amenable to unobstructed clot aspiration.

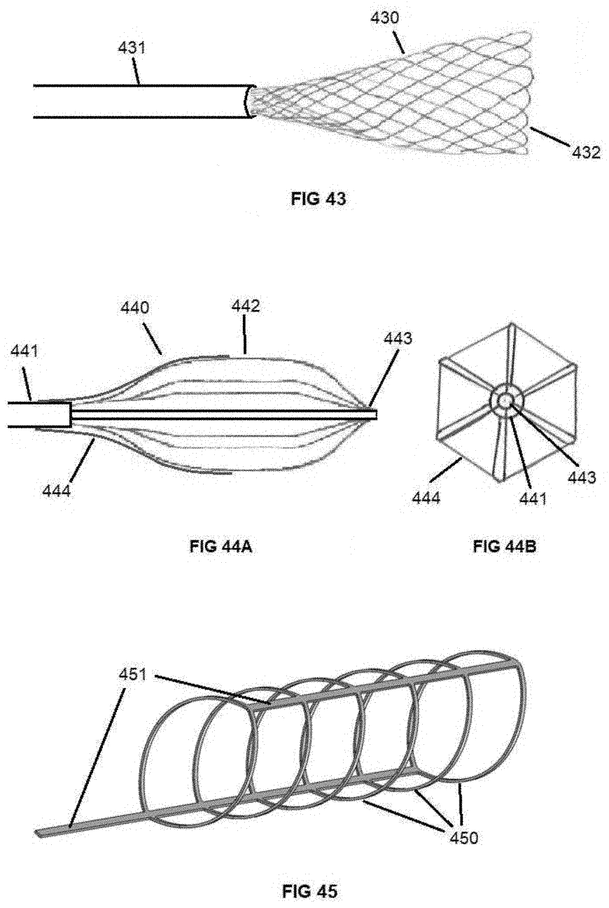

FIG. 43 shows an example of the present invention in which the distal expandable segment comprises a braided structure 430 attached to and flaring outward from the outer catheter shaft 431, which at its distal end 432 connects to a second inner braided structure attached to and flaring outward from an inner catheter shaft. In a manner of operation similar to that of the two-coil system shown in FIGS. 12 and 13, the inner member and inner braid is rotated against the outer catheter and braid causing the two braids to push against each other and expand.

FIGS. 44A and 44B show an example of the present invention in which the distal expandable segment comprises a longitudinal ribbed structure 440 which flares outwards from the catheter shaft 441 to the intended maximum expansion diameter 442 and then tapers down to connect to an inner member at its distal end 443. When the inner member is pulled the ribs are put in compression which causes them to bow outwards thereby expanding their profile, and when the inner member is pushed the rib are put in tension which causes them to stretch flatter thereby contracting their profile. In a preferred variant of the present example, one or more V links or other means are used to attach the ribs to each other in order to maintain their circumferential alignment. The vacuum resistant membrane 444 covers the ribbed structure up to approximately its point of maximum diameter, while distal to that the structure the ribs are open through which the clot can be aspirated.

FIG. 45 shows an example of a distal expandable segment comprising multiple rings 450 connected on opposite sides to spines 451. One spine is attached to the catheter outer member of the intermediate segment, such that pushing or pulling the other spine causes the rings to fold open, thereby expanding the structure. The rings do not need to be circular and may be able to be further squeezed by a sheath or other constraint for minimize profile in the collapsed state. Optimally this sort of design would be laser cut from a NiTi tube in order to create a single robust but functional structure.

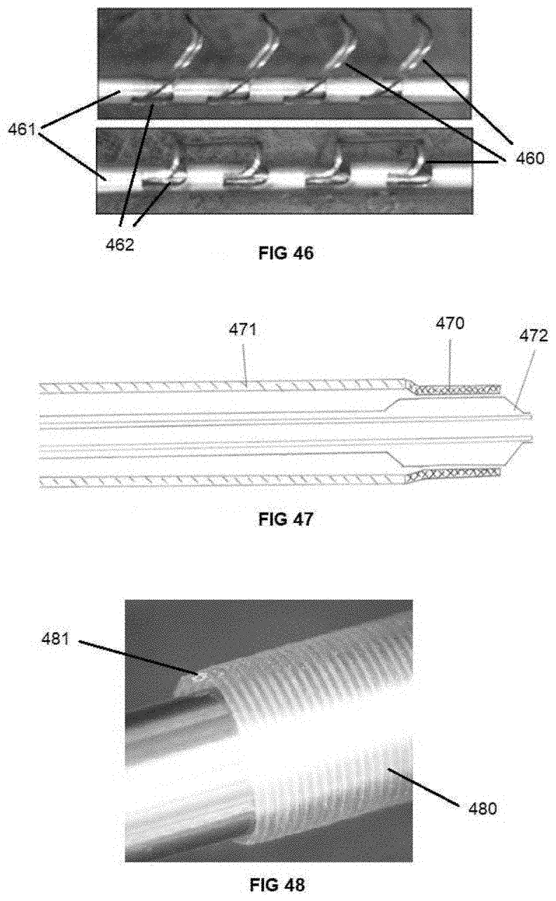

FIG. 46 illustrates a variant of the above example a structure consisting of rings 460 and a single spine has the spine covered by a tubular structure 461 with cuts 462, such that as the spine is pulled proximal into the tubular structure, the rings of the expandable distal segment are forced into a collapsed position by cuts in the tubular structure, and likewise when the spine is advanced the rings become unconstrained and expand.

FIG. 47 shows another example of the present invention in which the distal expandable segment comprises a slotted tube, sinusoidal ring, spine with ribs, or other plastically deformable scaffold 470 mounted to the end of an outer elongated tubular body 471, inside of which is a balloon catheter 472 which is inflated to expand the scaffold. After the scaffold has been expanded the balloon catheter is deflated and is then withdrawn through the lumen of the outer elongated tubular body and removed from the device and patient's body, thereby allowing an non-occluded aspiration lumen.

FIG. 48 shows another example of the present invention in which the distal expandable segment is constructed from a coiled polymer tubing 480 wherein the coil loops are bonded together. Pressurization of the lumen 481 within the polymer tubing from which the coil is constructed causes the material to elastically and/or plastically stretch and/or unfold any folds in the material, thereby causing the distal expandable element to expand or to unfold from a crimped configuration. Removal of such pressure causes the material to relax back to an at least partially collapsed state.

### Vacuum Resistant Membrane

In order to ensure integrity of the vacuum lumen over the distal expandable segment, a vacuum resistant membrane is attached to the scaffold. The membrane may lay on top of the scaffold, be bonded to the inner surface, or coated over the scaffold such that it forms a film between the ribbons and struts of the structure. In a preferred example the membrane is attached to the intermediate segment proximal to the scaffold and may also be attached to elements of the scaffold at one or more points or be free to move independently of them. In an alternate example, the membrane is attached to at least the distal portion of the scaffold. As the scaffold expands, the membrane stretches or unfolds with it, approximately matching the diameter of the scaffold. In designs involving a coil distal segment in which the coil is unwound to expand, the vacuum resistant membrane may cling to the coil and twist as the unit expands, potentially compromising expansion of the coil and functionality of the device. A key intent of the present invention is to disclose a number of techniques by which such membrane twisting can be mitigated or avoided.

For example, the membrane can be attached firmly only at the distal end of the coil such that it spins with the coil while the latter expands, and/or be anchored at the proximal end in a manner that allows the membrane to spin with respect to the shaft as the coil expands, yet not move proximal or distal. Typically such an arrangement involves two circumferential rings or ridges around the distal end of the catheter shaft, and a compatible ring or rib on the inside of the proximal end of the membrane which fits between the two. Alternately, a separate and more structurally robust element with such a ring or rib may be used to which the proximal end of the vacuum resistant membrane is then attached.

In another example, the vacuum resistant membrane comprises several independent pieces of material in a series of overlapping skirts, which are each attached to the coil and can rotate independently of each other, yet are pulled together under aspiration to provide a substantially vacuum-tight structure. In an extension of this concept, the vacuum resistant membrane may comprise a polymer ribbon bonded to the entire length of the coil ribbon, in which the polymer ribbon is sufficiently wider than the coil ribbon to overlap the adjacent coil loops in the expanded state, thereby providing a substantially vacuum-tight structure under aspiration.

There are many means of creating the vacuum resistant membrane. The membrane may be fully elastic, and fit snugly onto the scaffold in the collapsed state. As the scaffold expands the membrane stretches to accommodate the increased diameter, then when the scaffold is re-collapsed the elastic membrane relaxes back to a small diameter.

Membranes may also be semi-elastic or non-elastic, and in their natural unstressed state be of a diameter larger than that of the fully collapsed scaffold, either similar to the vessel size or at a convenient intermediate dimension. The membrane is then twisted, wrapped, folded, furled, or otherwise reduced in profile to match the profile of the scaffold in the collapsed state to aid device deliverability. A heat set may be used to help keep membranes of this sort at a reduced profile, and/or a very thin elastic tube or bands may be placed over the folded membrane. Non-elastic membranes of this type simply unfold as the scaffold are expanded, then refold naturally as the scaffold are collapsed or remain loose and unobstructive around the collapsed scaffold. Typically the scaffold will be collapsed only after the clot has been extracted, in which case aspiration will be active and the vacuum will help refold the membrane.

Elastic membranes may be made from a variety of soft polymers in the silicone, polyurethane, and polyamide families. Examples included C-flex (silicone), fluorosilicone, Tecothane (polyurethane), and Pebax (polyamide). Some name brand polymers suitable for this application which generally fall into one or more of the above polymer families include Chronoflex, Chronoprene, and Polyblend. Membranes in the hardness range of Shore 50A through 40 Durometer work best. At the upper end of this scale a portion of the membrane stretch is plastic, not elastic, but enough of it is elastic to fulfill the recovery needs.

Non-elastic membranes may be made from any of the materials used for the elastic membranes, just manufactured at a larger diameter, or from firmer materials in the 50-80 Durometer hardness range. Examples included various polyurethanes, Pebax 55D, 63D, 70D, and 72D, Nylon 12, PTFE, FEP, and HDPE. Thin metallic foils or foil-polymer laminates may also be used for a vacuum resistant membrane, providing a low friction and potentially radiopaque membrane. ePTFE (expanded polytetrafluoroethylene) is soft and flexible and makes an excellent vacuum resistant membrane, but is slightly porous which can compromise vacuum force application. An ePTFE membrane can be coated or covered with a thin layer of another material to eliminate the porosity. Typically this secondary material would be of the same materials and mechanical properties as those used for the elastic membranes described above. Other slightly porous meshes may find similar utility as a vacuum resistant membrane, with or without an additional porosity-eliminating layer.

In another example of the present invention, the vacuum resistant membrane may be made from a polymeric material which tends to absorb moisture and/or relax when warmed. Particularly useful for unfolding membrane designs, use of these materials may help the membrane to expand easily with the distal expandable segment. Such moisture and heat sensitive materials may also be coated over ePTFE or other membrane materials to promote the expansion of the latter, either as a continuous coated layer or in stripes or segments. Polymers suitable for use as a vacuum resistant membrane which swell when exposed to moisture include graft polymers, block polymers, polymers with special functional groups or end groups such as acidic or hydrophilic type, or blend of two or more of Poly(lactide-co-caprolactone), Poly(L-lactide-co-ε-caprolactone), Poly(L/D-lactide-co-ε-caprolactone), Poly(D-lactide-co-ε-caprolactone), poly(glycolic acid), poly(lactide-co-glycolide, polydioxanone, poly(trimethyl carbonate), polyglycolide, poly(L-lactic acid-co-trimethylene carbonate), poly(L/D-lactic acid-co-trimethylene carbonate), poly(L/DL-lactic acid-co-trimethylene carbonate), poly(caprolactone-co-trimethylene carbonate), poly(glycolic acid-co-trimethylene carbonate), poly(glycolic acid-co-trimethylene carbonate-co-dioxanone), or blends, copolymers, or combination thereof. The polymeric material in this invention can be blends of two or more homopolymers such as polylactide, poly(L-lactide), poly(D-lactide), poly(L/D lactide) blended with poly(caprolactone), polyglycolide, polydioxanone, poly(trimethyl carbonate), or the like. Polymers suitable for use as a vacuum resistant membrane which change shape when heated to body temperature include poly(methacrylates), polyacrylate, polyurethanes, and blends of polyurethane and polyvinylchloride, t-butylacrylate-co-poly(ethyleneglycol) dimethacrylate (tBA-co-PEGDMA) polymers, combination thereof, or the like. These polymers exhibit shape memory properties and undergo a phase transformation at body temperature and seek to return to a pre-established state.

The membranes may be extruded, dip coated on a mandrel, sprayed over a mandrel, electrospun, or manufactured using other means common in the industry. The membranes may be used “as is”, or further necked, stretched, or blow molded to achieve desired dimensions and properties. Wall thicknesses are ideally low to maintain a low device profile, ranging from 0.0005″ to 0.005″. The membranes may be configured in a cylindrical, tapered, reverse tapered, convex profile, concave profile, or other shape as preferred in order to expand smoothly and without twisting and perform as desired.

The membranes may be attached to the catheter shaft and the scaffold struts by any of the means in common use in the industry, including adhesives, heat shrink tubing entrapment, heat bonding, mechanically crimping under a swaged metal band, tying or riveting, etc.

The outside of the membrane may be coated with a lubricious coating to aid deliverability into the anatomy. In some cases the membrane may be inclined to twist as the coil or other rotating scaffolds in the distal segment are expanded or collapsed in profile. If the twisting is not desirable, the outside of the scaffolds and/or inside of the membrane may be lubricated to aid free movement of the scaffolds inside the membrane. Preferred lubricants include a hydrophilic coating of chemistry known in the industry, silicone oil, and PTFE spray coatings. The membrane can also be designed to incorporate wires or a braid to resist twisting.

Another example to mitigate or eliminate membrane twisting over the coil, to provide for a more circular distal end to the aspiration lumen, and to otherwise influence distal segment expansion dynamics is to place an expandable/collapsible structure between the coil scaffold and the membrane inside of which the coil scaffold can freely spin, such as a NiTi wire braid or PTFE slotted tube. More than one such structure may provide improved performance compared to a single structure. In a preferred example the expandable/collapsible structure, also referred to as the liner, is designed to resist twisting while at the same time requiring minimum force to expand. Suitable materials for this application include PTFE, FEP, HPDE, and other low friction polymers. Self-expanding materials such as nickel-titanium alloys and the various polymers which swell when exposed to moisture and/or change shape with heat (previously described) are also suitable for use as a liner, since their self-expansion force can be tuned to substantially counteract any compressive force exerted by an elastic vacuum resistant membrane, or to promote opening of a folding vacuum resistant membrane design. Such liners are typically laser cut from a tube into a slotted tube pattern, preferably with a spiral aspect to aid flexibility and while maintaining a continuous torque-resisting pattern. The liners can also be made from a polymer mesh or filter material with similar expandable properties. Liners may range in thickness from 0.0005″ to 0.008″, more preferably 0.001″ to 0.005″, and most preferably about 0.003″. The outside and/or inside of the liner may be coated with hydrophilic coating, silicone oil, PTFE spray, or other lubricant to aid in allowing the components to slide freely past each other during distal segment expansion and contraction. Alternately, one or more surfaces of the liner may be increased in roughness using sandpaper, microblasting, or other means in order to promoted adherence of one component to another where advantageous, for example helping the membrane to stick to the liner such that the combined structure is more resistant to twisting than the sum of the two individual parts.

In another example of the liner concept, the liner(s) are shorter than the membrane and positioned selectively. For example, a 2 mm to 3 mm long liner at the distal end of the membrane may aid membrane robustness during track and promoted a circular and collapse-resistant aspiration lumen. In another example, a liner in the middle of the distal expandable segment is used to selectively reinforce the membrane and promote or retard expansion in that area.

In an alternate example, one or more free-rolling wires are positioned between the coil and the vacuum resistant membrane and are used to prevent the membrane from clinging to the coil and twisting, in a manner akin to that of a needle bearing. Such wires will typically be in the range of 0.001″ to 0.005″ in diameter and may be made from stainless steel, cobalt chrome, nickel-titanium, polyimide rod, or any other sufficiently robust material.

In a further example of the present invention, the vacuum resistant membrane is attached to a sheath on the outside of the outermost elongated tubular member of the device, and the sheath extends from the proximal end of the vacuum resistant membrane to the proximal end of the catheter where it is integrated into the handle. This outer sheath is used to provide tension and/or counter-torque force to the vacuum resistant membrane during expansion of the distal segment to prevent membrane bunching or twisting. The portion of the sheath over the catheter intermediate segment and/or proximal segment may be drilled, notched, slotted, or otherwise cut to increase flexibility without significantly compromising tensile and/or rotational strength and stiffness.

It may also be advantageous for the vacuum resistant membrane to cover only part of the scaffold, such that scaffold extends distal to the distal end of the membrane.

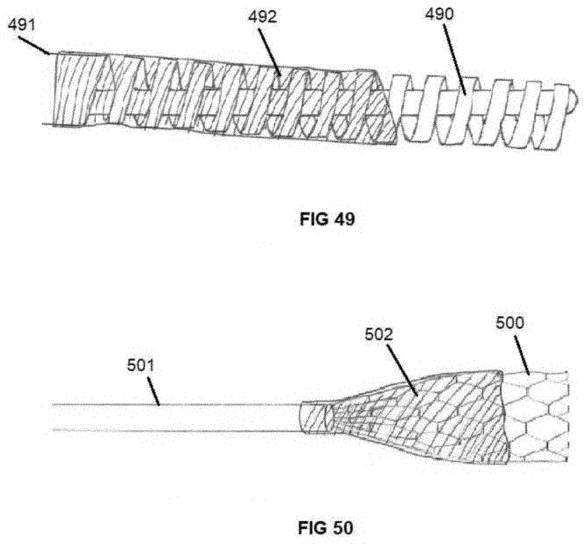

FIG. 49 shows an example of a distal expanding segment comprising a coil 490 attached to a catheter shaft 491 in which the vacuum resistant membrane 492 extends from the end of the catheter shaft to a point substantially proximal to the distal end of the coil.

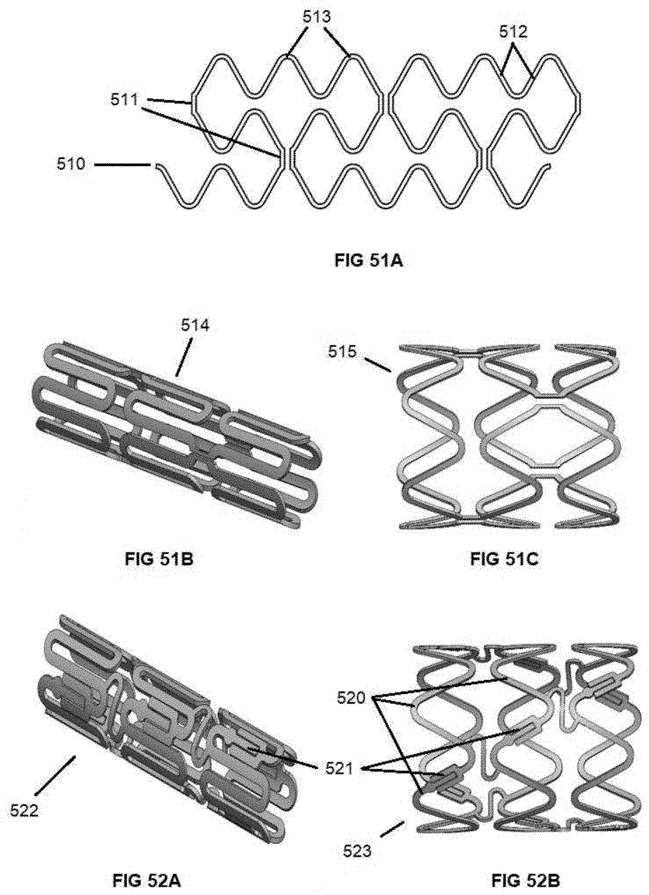

FIG. 50 shows an example of a distal expanding segment comprising a self-expanding scaffold 500 attached to a catheter shaft 501 in which the vacuum resistant membrane 502 extends from the end of the catheter shaft to a point substantially proximal to the distal end of the self-expanding scaffold.

One potential advantage of a configuration in which the distal portion of the scaffold is not covered by the membrane is that the uncovered portion of the scaffold in its collapsed state can be used to penetrate the clot, such that when the scaffold is expanded it disrupts the clot aiding aspiration and removal from the body. The expanding scaffold may break up the clot as the ribbons or struts are forced through the clot, or it may stretch the clot into a ring such that when the device is withdrawn the clot is invaginated for better aspiration or otherwise well anchored to the scaffold assist the vacuum force in pulling out the clot intact. In one example of the design, the scaffold comprises features designed to assist in mechanically disrupting the clot during expansion, such as sharp edges, metallic protrusions, fins, hook elements, or slots which serve to improve cutting or gripping of the clot.

### Scaffolds Comprising Single, Continuous Element

In another example of the present invention the distal expandable segment comprises a self-expanding scaffold of a generally sinusoidal or serpentine ring design, and the structure of the scaffold is provided by a single continuous undulating element or strut. FIG. 51A shows the pattern of the single undulating element 510 in a flattened state, as if the scaffold was bisected longitudinally and unrolled. The element contains longitudinally straight sections 511, angled sections 512, and curved sections or crowns 513. FIG. 51B shows the scaffold in the collapsed state 514 and FIG. 51C in the expanded state 515.

The primary advantage of this design is that the scaffold has superior flexibility in bending, tension, compression, and torsion compared to conventional sinusoidal ring scaffold designs with multiple continuous sinusoidal rings and/or multiple connection points within the pattern. The superior flexibility allows for easier delivery in tortuous anatomy, better conformance to the vessel in the expanded state, improved vessel sealing and less blood leakage during aspiration, and reduced vessel trauma. At the same time the scaffold of the present example maintains substantially equivalent radial strength and ability to support the vessel and resist vacuum compression as a conventional scaffold of similar material and dimensions.

FIGS. 52A and 52B show another example of the present invention in which the scaffold comprises multiple continuous undulating elements 520, which are not in continuity with each other but are maintained in position by tab-and-slot joints 521. FIG. 52A shows the scaffold in the collapsed state 522 and FIG. 52B in the expanded state 523. Alternately the joints may be of a ball-and-socket, hook and hole, male and female, nesting “S” curves, or other design which restrict movement of the multiple elements in at least one direction but allow movement in other directions, thereby granting the scaffold increased flexibility compared to a scaffold with metallic material continuity at the junctions. One or more joints may be bonded with a polymeric or elastomeric material configured to hold one or more of the multiple continuous undulating elements together during expansion and to thereafter form at least one discontinuity in the circumferential ring and the axial link after expansion of the scaffold in a physiologic environment. In the preferred example any such bonding material is a biodegradable polymer and/or adhesive.

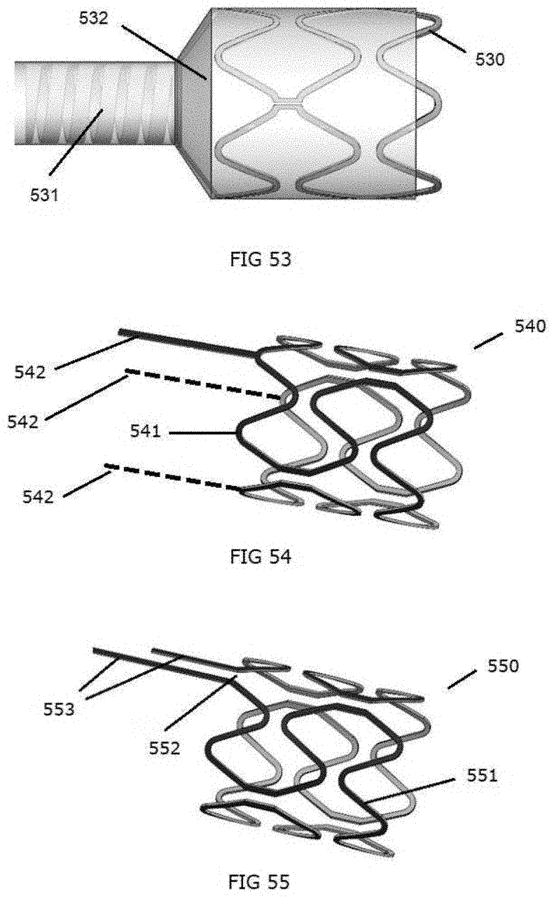

FIG. 53 shows an example of a distal expanding segment scaffold 530 featuring a single continuous undulating element as attached to the intermediate segment 531 of the aspiration catheter of the present invention and covered with the vacuum resistant membrane 532. The scaffold is shown in the expanded state after constraint removal. Compared to an aspiration catheter feature a self-expanding scaffold design with a conventional sinusoidal ring structure, the distal segment of the aspiration catheter of FIG. 53 would be more flexible during delivery and more conformable to the vessel wall after expansion, improving sealing with the vessel and minimizing leakage around the edge of the scaffold during aspiration.

In another example of the invention in which the scaffold comprises one or more continuous undulating elements, the scaffold features tensile elements which allow it to be unraveled (axially and/or radially collapsed) in order to facilitate collapse and/or removal from the body. In order to facilitate removal, the scaffold may comprise an element which follows a single path to form a cylindrical or conical envelope. The single path may be a closed loop, or the single paths may be open. The single path may form a single continuous string, or bifurcate at one or more points to form a closed loop. The scaffold may feature one or more tensile elements or pull struts which when placed in tension apply a localized stress on the scaffold to induce its unraveling. A scaffold with a single continuous undulating element may be unraveled into a single strip or loop of material, while a scaffold with multiple continuous undulating elements may have one or more such elements able to be unraveled. Removal of a single element of a multiple-element scaffold may be sufficient to allow the scaffold to recollapse enough for easy pullback from the anatomy in a low profile state.

FIG. 54 shows a scaffold 540 featuring a single continuous undulating element 541 with one or more pull struts 542 attached to the continuous undulating element to facilitate unraveling and removal of the scaffold. It can be seen by tracing the pathway of the curve that the structure is one large loop of material connected to the pull strut, and that there are no points at which the structure connects to itself anywhere along the loop. The strength of the structure and its ability to maintain a cylindrical scaffold is provided by the circumferential curvature of the scaffold and of the individual strut crowns. The latter can be radially compressed to allow the scaffold to collapse and expand without causing the scaffold to unravel. However when pulled using the pull strut(s), the forces are concentrated on a subset of such crowns and in an axial direction against which by design the structure has much less ability to resist, allowing the structure to progressively unravel. The pull strut or struts may be continuous and run the entire length of the catheter, or attach to wires, filaments, tubes, or other members which serve the same tensile function. The pull strut(s) may run through the aspiration lumen and collapse the device through the same, or run outside the aspiration lumen of the catheter on the outside of the aspiration catheter.

FIG. 55 shows a further variant of a scaffold 550 in which the single continuous undulating element 551 has been interrupted at a point 552 and in which pull struts 553 have been attached to each end of the element. In another example more than one pull struts are attached to the same single closed loop continuous element.

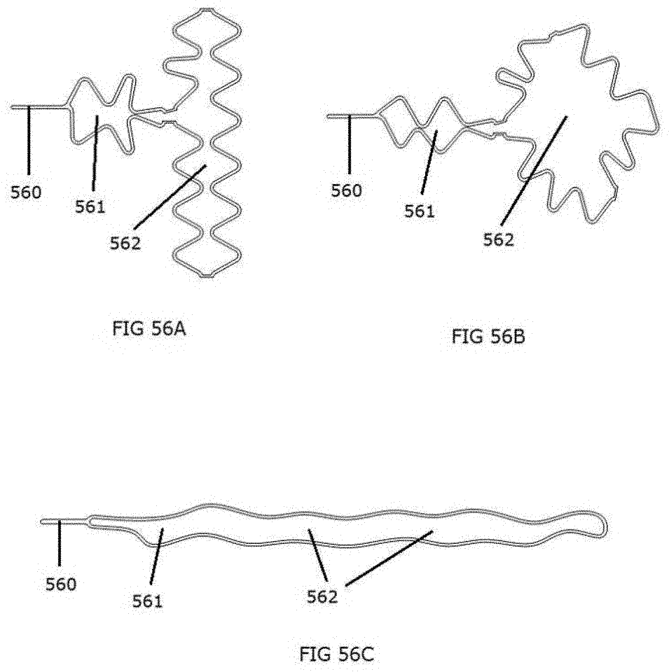

FIGS. 56A-56C show a two-dimensional perspective of the scaffold of FIG. 54 through several stages of being unraveled and collapsed. As the pull strut 560 is pulled in tension, first the most proximal structure 561 of the scaffold will collapse (FIG. 56A), followed by more distal structures 562 (FIG. 56B), until eventually the entire continuous undulating element has been drawn out into single loop (FIG. 56C).

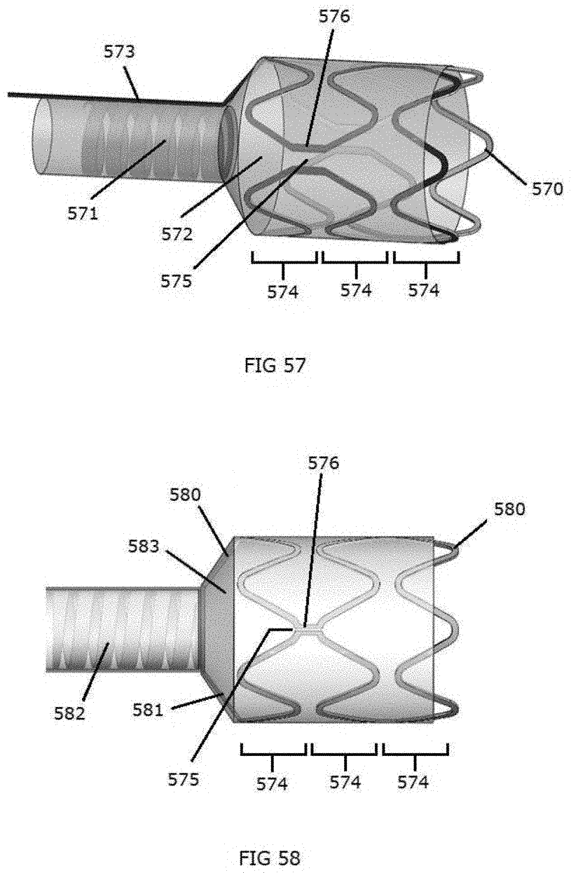

FIG. 57 shows an example of an unraveling scaffold of the present invention in which the scaffold 570 is positioned at the end of the catheter shaft 571 and covered by the vacuum resistant membrane 572, and wherein the pull strut 573 runs outside the aspiration catheter shaft. In one example, the scaffold may comprise circumferential rings 574 having circumferential separation regions 575 on axial links 576 that join the rings. The circumferential rings 574 are collapse joined by the axial links, and the scaffold is configured to circumferentially separate along separation interfaces defined by the separation regions and to form one continuous structure after all axial links have separated along said circumferential separation regions. Each circumferential ring will have at least one separation region provided by an axial link, sometimes having two or more. Similar scaffold structures are shown in FIGS. 54 and 55.

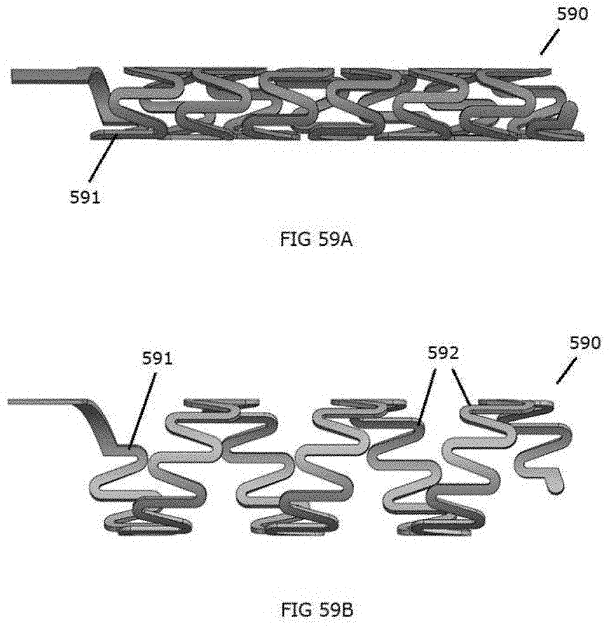

FIG. 58 shows an alternate version the unraveling scaffold of the prior example in which two pull struts 581 of the scaffold 580 are connected directly to the end of the aspiration catheter, the intermediate segment 582 under the vacuum resistant membrane 583. In these variants the scaffold is not independently collapsible but will controllably collapse when a vacuum is applied at specific vacuum thresholds or when the aspiration catheter is withdrawn.

The unraveling scaffold can be placed at the distal end of an aspiration catheter or along a distal segment of the aspiration catheter where the segment ranges from 1 cm to 25 cm. The scaffold can be delivered in the expanded configuration (extraction configuration), or can be delivered in a delivery configuration and expanded to the extraction configuration in the body.

FIGS. 59A and 59B show another example of an unraveling scaffold or distal expandable scaffold 590 in which the continuous undulating element 591 is formed into a single spiraling sinusoidal ring. FIG. 59A shows the scaffold in the collapsed state, and FIG. 59B shows the scaffold in the expanded state. This embodiment has the advantage of unravelling into a single continuous element rather than a loop, and the unraveling is very predictively progressive from proximal to distal. Expansion of the scaffold may be due to an internally applied force, for example by balloon dilation, or the scaffold may be constructed from a material that will self-expand when released from constraint. In both cases the scaffold may expand through opening of the individual sinusoids 592 in the pattern and/or through unwrapping of the structure as it is deployed or released from constraint.

In the preferred examples of the present invention featuring a scaffold with one or more continuous undulating elements, as depicted in FIGS. 51-59, the scaffold may be laser cut from a NiTi hypotube and after cleaning and polish is heat set to the desired configuration in the expanded state. After assembly onto the catheter shafts, the scaffold is then pressed into the collapsed state and constrained with a sheath, cap, or other means as previously described for self-expanding scaffolds. In an alternate example, the scaffold can be made from a material which self-expands when exposed to moisture, heat, and/or electricity such that a separate constraint is unnecessary. The strut widths and thicknesses, expansion diameters, straight and tapered profiles, catheter construction, and other features of the self-expanding scaffold are otherwise the same as has been previously described for self-expanding scaffold designs comprising sinusoidal rings.

In another example of the present invention featuring a scaffold with one or more continuous undulating elements, as depicted in FIGS. 51-59 the scaffold is made from stainless steel, a cobalt chrome alloy, titanium, or other non-superelastic material and is expanded using a balloon as depicted in FIG. 47. In another example the scaffold is made from a malleable material which upon contact with the clot will flare, preferably irreversibly flare, to engage the clot and provide a larger distal mouth for clot aspiration. In another example the scaffold is made from an elastic or superelastic material such as nickel-titanium which can be unraveled to collapse as described above, but upon release of tension on the pull strut(s) will seek to reform into the original scaffold configuration.

### Method of Manufacture and Assembly—Example 1 for Twin Coil Distal Expandable Segment

In the exemplary dual coil example, nickel-titanium hypotubes are laser cut to create the coils used in the distal expandable element. The coils are then chemically and/or mechanically de-slagged and then electro-polished. The electropolishing process smooths the surface of the coils and rounds the edges, causes the cross-section geometry of the ribbon to become more circular. The more circular cross-section has lower contact area between the outer and inner coils which reduces friction between the two and aids collapse and expansion.

The coils are then placed over a stainless-steel rod or hypotube and heat treated in a fluidized temperature bath filled with aluminum oxide sand to set the desired neutral state. They are then removed from the bath and quenched in water. The heat treatment process allows the coils to accommodate greater diametric expansion due to the change in geometry.

The various catheter shafts are cut to length and heat bonded to each other using conventional means such as laser bonding or a hot air nozzle. If the materials are chemically incompatible then adhesives may be used. The catheter outer member constructed as follows. First a PTFE liner is stretched over a steel mandrel. Next the proximal portion of the lined mandrel (eventually forming the proximal shaft segment) is braided with a stainless-steel braid. Then the distal portion of the lined mandrel is wound with a coil (eventually forming the intermediate shaft segment). Polymer sections of appropriate length and wall thickness are slid over the braided and coiled portions of the assembly, then the entire assembly covered with heat shrink tubing. The assembly is placed in an oven at 160 C for approximately 10 minutes to cause the polymer outer jacket to melt and flow around the braid and coil, thereby forming a robust cohesive structure after the heat shrink tubing is removed. The catheter inner member is formed in the same manner as described for the outer member above.

The outer coil is then bonded to the catheter outer tubular member using adhesive, a heat melt, overlying heat shrink, or other methods. Typically, the proximal end of the outer coil will be designed with a slot or other gap allowing the hypotube stub to be crimped down to the desired diameter before bonding, and may have axially aligned legs to aid bonding. The coil may be bonded inside, outside, or in a butt joint with the adjacent shaft. Alternately, the component can be laser cut from a single piece in which one portion of the coil becomes the expandable distal segment and another portion of the coil is polymer jacketed and bonded to form the intermediate segment as described above, thereby saving the need for a separate distal segment to intermediate segment bond.

The inner coil is likewise bonded to a catheter inner tubular member which can rotate inside the outer tubular member. The inner coil assembly is threaded through the outer coil assembly until the distal end of the outer and inner coils align, then the coils are attached together using wires, tabs, or welds.

The proximal ends of the catheter outer and inner tubular members are trimmed to length and bonded to their receptive parts in the handle mechanism. The handle mechanism is then used to rotate the catheter inner tubular member concentrically within the outer tubular member such that the coil is collapsed to the desired size. At this point the vacuum-resistant membrane is slid over the coils and bonded to the distal end of the catheter shafts to form the complete expandable distal segment. If a non-elastic membrane is used, it may be heat set into the folded shape either before or after attachment to the device.

The portion of the device which will be in contact with the blood vessels will be coated with a hydrophilic coating or other lubricious coating to aid device delivery in vivo. A lubricious coating or material may also be applied to the inside surface of the scaffold and/or aspiration lumen of the catheter shafts in order to facilitate smooth movement of the device over guidewires and microcatheters, and to promote rapid clot aspiration. The completed device is then packaged and sterilized.

Construction of a single coil example is generally similar, except that there is only one coil and the catheter tubular inner member will extend to the tip of the single coil. Various alternative means of assembling the device of the present invention are envisioned. For example, the coils may be wrapped separately and secured in the fully collapsed state using special fixturing, the order of assembly may vary.

### Method of Manufacture and Assembly—Example 2 for Self-Expanding Scaffold

The self-expanding structure is laser cut from a tube made from a super-elastic nickel-titanium alloy, which is afterwards heat set into the desired expanded shape. In the preferred method, the expansion process is performed in multiple heat set steps using various mandrels with increasing diameters at each step.

The heat set scaffold is then electropolished to provide a smooth surface finish. The catheter shafts are constructed in the same manner as described for a coil design above. A short section of molded polymer sleeve is bonded to the distal end of the inner member. The scaffold is then bonded to the catheter shaft in the same manner as described for a coil design above. The vacuum resistant membrane is attached to the scaffold in the same manner as described for a coil design above. The inner member is inserted through the outer member and scaffold. A crimp fixture is used to press the scaffold and membrane to the collapsed state, whereupon the inner member is drawn back so that the collapsed scaffold and membrane is inserted into the polymer sleeve on the distal end of the inner member, thereby forming a constraining cap. The proximal ends of the catheter outer and inner tubular members are trimmed to length and bonded to their receptive parts in the handle mechanism. The portion of the device which will be in contact with the blood vessels will be coated with a hydrophilic coating or other lubricious coating to aid device delivery in vivo. A lubricious coating or material may also be applied to the inside surface of the scaffold and/or aspiration lumen of the catheter shafts in order to facilitate smooth movement of the device over guidewires and microcatheters, and to promote rapid clot aspiration. The completed device is then packaged and sterilized.

### Scaffold Structure Configured to Controllably Collapse Under Vacuum

In another example of the present invention, the scaffold or self-expanding scaffold delivered in the extraction configuration or expanded to the extraction configuration in the body is intended to be advanced at least partially into the clot and designed to at least partially collapse upon application of vacuum above an intended threshold, for example any of a range from 150 mmHg to 680 mmHg (approximately 0.2 to 1.0 atm negative pressure). Contact with the clot temporarily seals the distal end of the device thereby allowing the vacuum level to build until the scaffold beings to collapse. The partial collapse causes pieces of the clot to be compressed and disrupted by the scaffold struts thereby aiding aspiration. Upon release of vacuum—either by pulling the scaffold away from the clot or by manual cessation of the applied vacuum by the operator—the scaffold will again re-expand towards its unconstricted diameter (expansion diameter) and such may be used repetitively in the same manner to progressively disrupt and aspirate the clot. In a preferred example the scaffold collapse is limited to a diameter between 20% and 90% of its fully expanded diameter, in order to provide a balance between achieving a low profile for ease of device movement within the artery and maintaining a lumen of sufficient size for aspiration and removal of the clot. In a preferred example the scaffold collapse is limited to between 25% and 75% of its fully expanded diameter.

There are several means of limiting the amount by which the scaffold will collapse under vacuum. In general a scaffold configured to controllably collapse will be of the same construction for scaffolds previously specified in this application but may use softer grades of material and/or a thinner wall thickness and/or strut widths to reduce the strength of the scaffold sufficiently to allow it to controllably collapse at least partially under vacuum. Alternatively, the scaffold may be manufactured from a polymeric, elastomeric, and/or shape memory material. The elastic membrane may be attached to the scaffold as previously described, or the scaffold may be embedded into the elastic membrane. The amount of collapse may be limited by the spring stiffness of the self-expanding material and geometry, by the width or curvature of the struts in the scaffold pattern, or the scaffold may feature collapse-limiting elements designed to contact each other at a certain diameter and prevent further scaffold closure. Furthermore, the scaffold may feature angled and/or sharpened tips designed radially limit the collapse of the scaffold and/or to cut the clot during collapse of the scaffold thereby further disrupting the clot and facilitating aspiration. The angled tips may also be used to grip the clot thereby aiding mechanical removal of hard clots not amenable to aspiration.

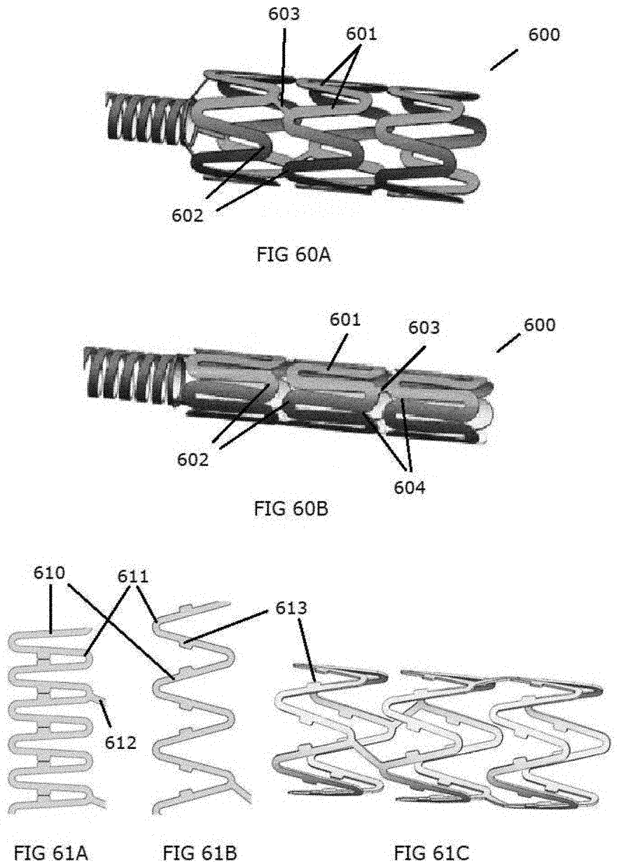

FIGS. 60A and 60B show an example of a self-expanding scaffold 600 configured to controllably collapse under vacuum (the overlying vacuum membrane has been omitted for ease of viewing the scaffold pattern). FIG. 60A shows the scaffold in the expanded state, and FIG. 60B shows the scaffold in the collapsed state. In the figures shown the scaffold comprises a sinusoidal ring pattern wherein substantially straight struts 601 and curved crowns 602 form one or more circumferential sinusoidal rings, which may be connected to any adjacent rings by links 603. In another example the sinusoidal ring scaffold design features a continuous undulating element as previously described. In other variants the scaffold may comprise a slotted tube pattern, series of linked closed diamond shaped cells, or other geometry. Regardless of geometry, the amount of collapse in this example is limited primarily by the mechanical strength of the structure, such as the spring stiffness of the self-expanding material and geometry, and/or by the width or curvature of the struts in the scaffold pattern. For example, the scaffold may be constructed from a shape memory material which when unconstrained expands to the geometry depicted in FIG. 60A, but under vacuum the scaffold reduces in profile to the geometry depicted in FIG. 60B, at which point either stress in the crowns 602 prevent further closure and collapse of the scaffold, or the struts come into contact with each other (such as at points 604) physically preventing further closure. In a preferred example, the unraveling scaffold can be used to controllably collapse the scaffold under certain vacuum thresholds as described in this application.

FIGS. 61A-61C show another example of a self-expanding scaffold configured to controllably collapse under vacuum (the overlying vacuum membrane has been omitted for ease of viewing the scaffold pattern). Like the scaffold of the previous example, the exemplary scaffold is shown as a sinusoidal ring design and features struts 610, crowns 611, and links 612. This example further features collapse-limiting elements 613 which prevent collapse of the scaffold to the profile as shown in FIG. 60B. FIG. 61A shows a flat view of the scaffold pattern in the fully collapsed state, where the collapse-limiting elements are in contact with each other and preventing further closure of the scaffold. FIG. 61B shows a flat view of the scaffold pattern with collapse-limiting elements in the expanded state. FIG. 61C shows a three-dimensional view of the scaffold with collapse-limiting elements in the expanded state. The collapse-limiting elements may be substantially rectangular as shown, square, hemispherical, or of another geometry. The collapse-limiting elements may be present on both adjacent struts or only on one. They may be substantially in the middle of the strut as shown or biased towards one end. Each pair of struts may have one or more collapse-limiting elements, or the elements may only be present on some of struts around the perimeter. In another example the struts themselves feature areas of increased width or are wavy in nature such that they contact adjacent struts at a larger diameter thereby limiting the amount of collapse versus what would occur if the areas of increased width or waves were absent.

In a preferred example any scaffolds configured to controllably collapse may be of a generally cylindrical profile intended to engage an inner wall of a blood vessel, and may feature a tapered transition region from the larger diameter expanded configuration to the relatively smaller diameter aspiration catheter shaft.

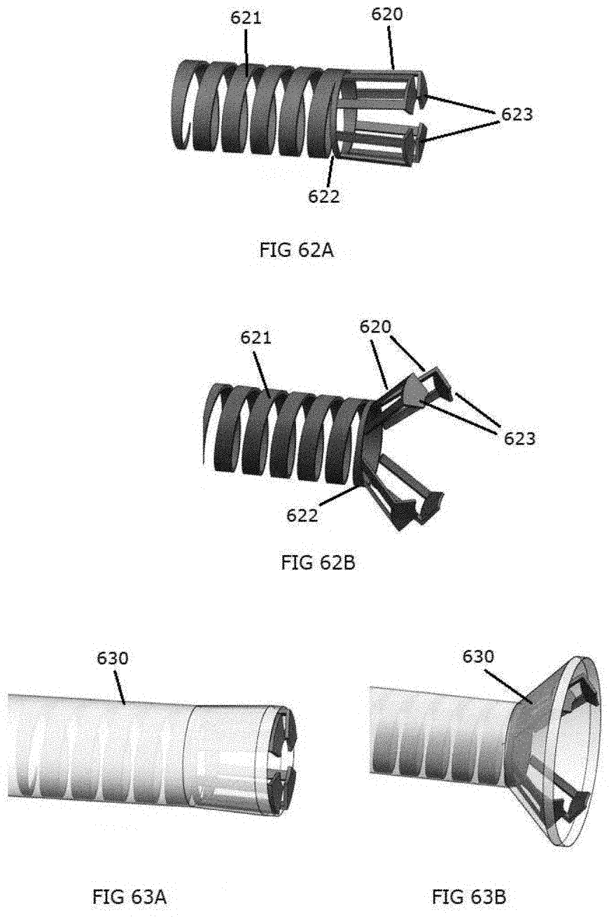

FIGS. 62A-62B show another example of a self-expanding scaffold configured to controllably collapse under vacuum, in which the scaffold comprises a cone-shaped configuration having pivoting struts with distal ends which form a jaw opening. The elastic membrane has been omitted from the figures for clarity of viewing. FIG. 62A shows the scaffold in the substantially collapsed configuration, and FIG. 62B shows the scaffold in the substantially expanded configuration. The scaffold comprises two or more jaws elements 620 which connect to the catheter shaft reinforcement 621 at the neck of the device 622. The jaw elements feature bent tips 623 which are designed to contact each other and limit scaffold collapse. In a preferred example the jaws interface along their sides such that a lumen for aspiration is maintained between the jaws in the fully closed configuration. Furthermore the jaws are designed to cut or bite into a clot during vacuum collapse of the scaffold to aid in disrupting the clot and facilitating the aspiration of the clot through the aspiration lumen. The tips and/or edges of the jaw tips may be serrated, sharpened, angled, or otherwise contoured to aid in cutting the clot. The angled tips may also be used to grip the clot thereby aiding mechanical removal of hard clots not amenable to aspiration.

FIGS. 63A and 63B show the scaffold of FIGS. 62A and 62B covered by the vacuum resistant membrane 630, as would be required to maintain vacuum integrity of the device and enable the vacuum collapse ability of the device to function.

The scaffold structure may be constructed of sinusoidal rings wrapped radially, where each ring is connected by one or more links. The scaffold material having self-expanding properties is heat set to a diameter greater than the targeted lesion diameter (TLD). The heat set diameter typically is 1.0 mm greater than the TLD, though it can be heat set between 0.5-3.0 mm above the TLD. The scaffold is coupled to an aspiration catheter and covered with an elastic membrane to hold a vacuum and is advanced in the crimped configuration towards a clot blocking a blood vessel. The scaffold is then expanded to the extraction configuration and further advanced to engage said clot. Vacuum is applied at the proximal end of the aspiration catheter to initiate extraction of the clot. The covered scaffold disrupts at least part of the clot, compressing it and aspirating it through the aspiration lumen. When the vacuum is applied the scaffold is configured to contract or collapse as in FIGS. 60A and 60B wherein the scaffold is collapsed to a smaller configuration from the expanded (extraction) configuration. The scaffold may collapse to a substantially flat configuration, or to a smaller cylindrical or conical configuration with an inner diameter ranging from 0.25 to 0.75 times the inner diameter of the expanded or extraction configuration, wherein the vacuum before collapse of said scaffold ranges from 0.2 atmospheres to 1 atmosphere. The scaffold as it is collapsing compresses the disrupted clot and advances it into the aspiration lumen of the aspiration catheter. In another example as shown in FIGS. 61A to 61C, the scaffold features tab elements such that when a vacuum is applied the struts and tabs collide radially, thereby limiting the diameter reduction of the scaffold and maintaining a minimum diameter configuration to enhance the aspiration of the clot into the aspiration lumen.

### Multiple Expandable Scaffolds

In another embodiment of the present invention, the device incorporates two or more expandable scaffolds which when both or more are deployed form two or more vacuum regions. One scaffold is at the distal end of the device and may comprise an expandable coil, self-expanding structure, malleable structure, or other type of expandable scaffold as previously discussed. Proximal to the distal expandable scaffold are one or more additional expandable scaffolds. The additional scaffold(s) could be as little as a few millimeters apart from adjacent scaffolds—whatever minimum is required for each to expand without interfering with each other—or they could be as much as approximately 15 cm proximal to the distal scaffold. While many of the variants of expandable scaffolds previously described could be used for an additional scaffold mounted to the catheter outer member, a self-expanding structure is preferred due to simplicity of constraint and deployment but one can use other type of scaffolds as well. This type of configuration is useful in being able to retrieve clots and minimize distal embolization when extracting clots.

In a preferred example, the device comprises two or three expandable scaffolds made from self-expanding conical structures, with the second (and third, if applicable) scaffolds located between 5 mm and 5 cm proximal to the next most distal scaffold.

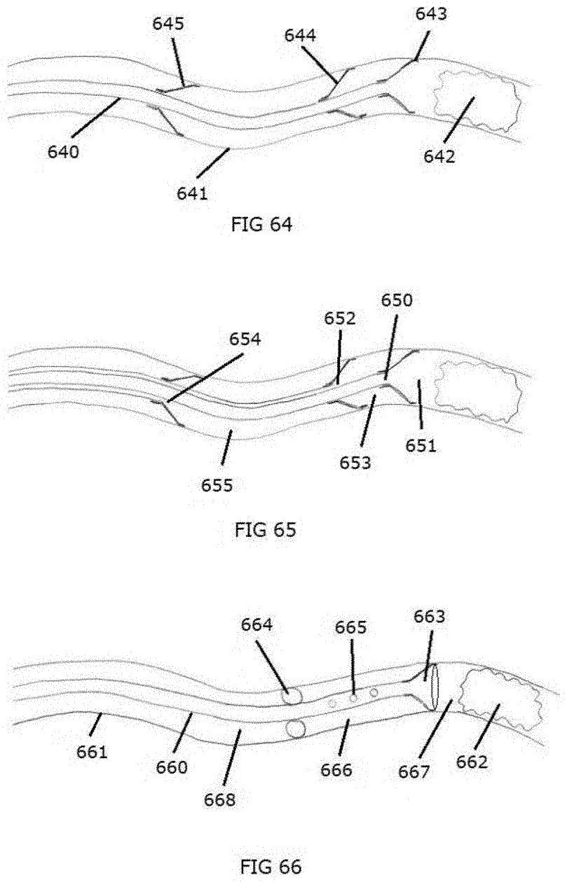

FIG. 64 shows an example of an aspiration catheter 640 featuring multiple expandable scaffolds deployed in an artery 641 close to a clot 642. In this example the scaffolds are all self-expanding substantially conical structures. The distalmost scaffold 643 is a conformable scaffold which is folded inside the aspiration catheter lumen for constraint during delivery as described (see FIGS. 28A-C), while the middle scaffold 644 and proximal scaffold 645 have a superelastic nitinol cores (see FIG. 19 or 20 for example) covered by an elastic membrane and are reversibly constrained by an outer sheath.

There are several potential advantages to a device with multiple expandable scaffolds. A primary advantage is that the additional scaffolds provide additional areas of contact with the vessel to further ensure a good seal between the device and the vessel, since any blood leakage around the expanded scaffold during aspiration reduces the vacuum level distal to the device and effective vacuum force applied to the clot. A further advantage is the additional contact points help maintain the position of the device in the vessel, since systolic/diastolic vessel movement, clot impacting the tip of the device during aspiration, and/or operator carelessness during handling could cause the device to slip proximal. Since the guidewire and any supporting delivery catheter or inner member would have been removed prior to aspiration, any proximal device slippage during aspiration could induce a significant procedure delay if the aspiration has to be stopped and the accessories reintroduced to allow the device to be repositioned in an optimal area for aspiration (for example, distal to all major side branches and tortuosities).

In an alternate example, one or more of the more proximal scaffolds comprises an inflatable balloon mounted to the outside of the catheter shafts, which can be inflated to achieve a seal with the vessel and achieve the same advantages of the design with the other sorts of scaffolds previously described herein.

A further advantage to a device with two or more expandable scaffolds is that they can be used to form one or more additional vacuum regions proximal to the most distal expandable scaffold. Vacuum in the area between the scaffolds during aspiration can be achieved by adding one or more holes or ports in the catheter shaft between the aspiration lumen and the scaffolds, or through additional and independent vacuum lumen(s) running along the catheter to the proximal end of the device. In a preferred embodiment the vacuum is applied to the additional vacuum regions at the same time as the primary aspiration is performed, but if the additional vacuum regions have their own vacuum lumens then valves or additional vacuum pumps could be used to apply vacuum to them independently. A secondary vacuum region can have the same amount of vacuum applied as that applied to the primary vacuum region distal to the device, or though port or lumen size control be made to have a lesser amount of vacuum even if a common vacuum source is used.

FIG. 65 shows the device of FIG. 64 further featuring unique vacuum lumens for each vacuum region. The primary vacuum and aspiration lumen 650 provide for a region of vacuum 651 distal to the catheter and adjacent to the clot. A second vacuum lumen 652 provides for a region of vacuum 653 between the distal and middle scaffolds. A third vacuum lumen 654 provides for a region of vacuum 655 between the middle and proximal scaffolds. The multiple vacuum lumens could be achieved through the use of multiple adjacent lumens in the catheter shaft, or by having a tube within a tube within a tube sort of coaxial design.

One advantage of having additional vacuum regions proximal to the distal-most expandable scaffold is that it reduces the vacuum force being applied to the distal scaffold, sleeve, and shaft, allowing these components to be made of lighter construction for superior deliverability in distal or tortuous anatomy without risking collapse or leakage during vacuum. A further advantage is that any loose clot or clot fragments in the vessel proximal to the primary clot can be captured during aspiration, during such procedures where the distal tip of the device is positioned close to the primary clot. An additional advantage is that if a clot substantially clogs the distal tip of the device, the secondary vacuum regions may serve to maintain some level of vacuum in the vessel and prevent loose clot debris from migrating distal and causing secondary ischemic events.

FIG. 66 shows another example of an aspiration catheter 660 featuring multiple scaffolds deployed in an artery 661 close to a clot 662. In this example the distal scaffold 663 has a self-expanding nickel titanium core (see FIG. 19 for an example) covered with an elastic membrane, while the more proximal scaffold 664 comprises an inflatable balloon mounted to the outside of the catheter shafts. The balloon can be inflated to achieve a seal with the vessel and achieve the same advantages of a design with the other sorts of scaffolds previously described herein. In a preferred example the balloon is spherical or ring-shaped and is made from an elastic silicone, which connects to a small tube within the aspiration lumen or second axial lumen within the catheter which allows pressurized fluid or vacuum to be applied to the balloon in isolation to allow it to inflate and deflate. The aspiration catheter further features vacuum ports 665 between the two scaffolds. The size of the ports is such that during aspiration the vacuum level in the region between the two scaffolds (666) is between that of the fully applied vacuum in the region distal to the distal most scaffold (667) and the near-ambient blood pressure in the region proximal to the most proximal scaffold (668), thereby reducing the vacuum gradient each scaffold must resist.

### Malleable Structures for Expandable Distal Segment

In another example of the present invention, the expandable distal segment comprises a malleable scaffold or other structure which naturally remains or is configured to remain in the collapsed state or in a partially expanded state until engagement with the clot, or engagement with an object opposite to the malleable scaffold, at which point the scaffold or other structure malleably flares, unfolds, and/or unfurls to further expand and conform to the shape of the clot. The structure may be designed to expand only up to the vessel diameter, or to press between the clot and the vessel wall and partially or completely engulf the clot before the vacuum pressure is applied. One advantage of an aspiration catheter utilizing a malleable structure is that it maximizes the contact area between the catheter and clot to both increase aspiration force and to prevent clot debris from slipping past the catheter and potential causing secondary embolisms elsewhere (such as distal embolism). If the malleable structure is designed to press its way between the clot and the vessel wall it may also help separate the clot from the vessel to aid extraction. Another advantage is that it is typically passive in operation and does not require a separate expansion operation by an operator (active expansion) prior to aspiration, or collapse operation after aspiration of the clot.

In another example of the present invention, the malleable structure is composed of one or more pieces of ductile material which deform to flare and/or unfold or unfurl to change from a substantially cylindrical collapsed state to a conical or bell-shaped expanded state. In one example, the malleable structure has axially aligned folds or pleats which allow it to achieve the transformation in shape. The malleable structure is introduced into the body in the low profile configuration or collapsed state in which the folds or pleats are substantially closed, while in the expanded state the pleats or folds still maintain a partially pleated or folded shape in order to provide the rigidity necessary to resist collapse during vacuum application. At the tip of the expanded structure where it is in contact with the clot and the vessel, the contact forces will force the folds or pleats of the structure into a fully open and substantially flat shape which will seal best with the vessel. In another example, the folds are spiral in nature to minimize impingement of the pleats into the aspiration lumen and also to better maintain a circular leading edge to the device.

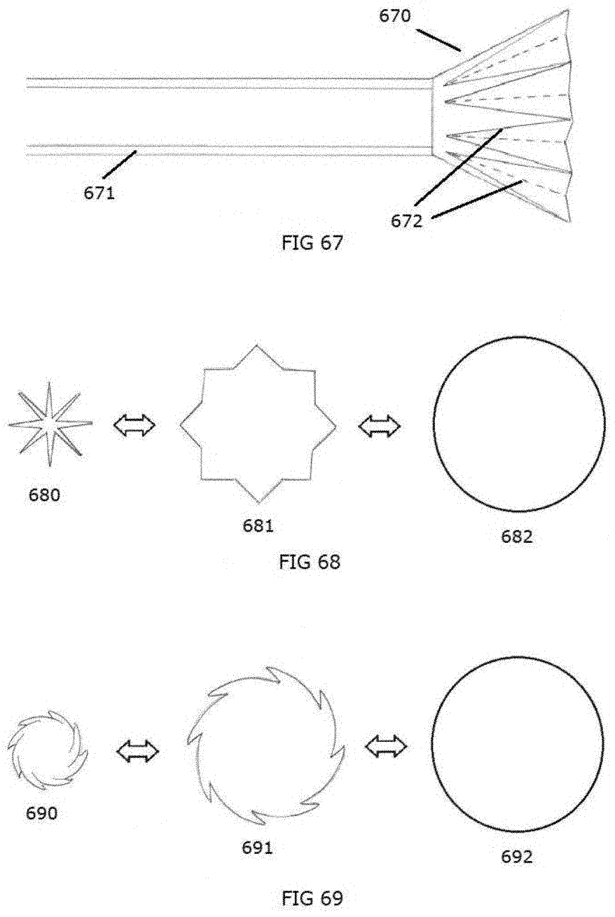

FIG. 67 shows an example of a malleable conformable scaffold 670 of a conical nature attached to a catheter shaft 671. In this example, the malleable conforming scaffold has axially-aligned folds or pleats 672 which allow it to achieve the transformation in shape. While in the collapsed state the folds or pleats are substantially closed, and when the scaffold is pressed against the clot, the clot partially enters the mouth of the scaffold, pushing outwards on the scaffold causing the pleats to open or unfurl until the scaffold reaches the expanded state. In one example the pleats unfold entirely resulting in a substantially circular scaffold mouth. In another example, the scaffold still maintains a partially pleated or folded shape in the expanded state so that the pleats help provide the rigidity necessary to resist collapse during vacuum application. In an alternative aspect, the scaffold of 670 may alternatively be formed from a shape memory material such as a polymeric shape memory material where the scaffold is introduced typically constrained in the delivery or crimped configuration and is released or configured to expand by unfolding of the pleats to the expanded configuration (extraction configuration), and then after aspiration of the clot the scaffold is refolded or partially refolded as it is retracted into the sheath or withdrawn from the body. The scaffold is crimped or collapsed to the delivery configuration by partially or fully folding the pleats.

FIG. 68 shows the cross-section of the malleable conformable scaffold of the preceding example as it progresses from the fully furled configuration 680 to a partially unfurled configuration 681 to a fully open configuration 682.

In another example, the folds are spiral in nature to minimize impingement of the pleats into the aspiration lumen and also to better maintain a circular leading edge to the device. FIG. 69 shows an alternate cross-section profile of a malleable conformable scaffold utilizing circumferential folds, as it progresses from the fully furled configuration 690 to a partially unfurled configuration 691 to a fully open configuration 692. Such a spiral fold may also allow for a lower device profile in the collapsed state.

In one example, the malleable structure is composed of a ductile but non-self-expanding material formed into one of the many geometries described for a self-expanding structure above, such as an array of linear elements, loops, sinusoidal rings, or combination thereof. As manufactured and introduced into the body, the structure is substantially cylindrical in profile or may even have a slight taper towards the distal tip of the device, but it will expand into a roughly conical or bell shape upon contact with the clot. The force of contact with the clot causes the arms of the structure to bend outwards from each other, thereby increasing the tip diameter of the device. The leading edges of the structure may be rounded or capped with a spherical shape to reduce vessel trauma, and/or flared outward giving the distal end of the device a generally trumpet-shaped leading edge designed to assist the malleable structure in conforming to the face of the clot and expanding.

The malleable structure so described could be manufactured from a variety of metals including stainless steel, titanium, a cobalt chrome alloy, Elgiloy, a magnesium-zinc alloy, or other metals able to accommodate the required deformation during expansion, while maintaining sufficient strength in the struts to prevent collapse during vacuum application. The malleable structure may also be made from polymers including but not limited to biodegradable polymers with glass transition temperature (Tg) above body temperature such as poly(lactide-co-glycolide), poly(lactide-co-caprolactone), polylactide, or the like, non-resorbable polymers such as polystyrene, polyethylene terephthalate, polyamide, polyvinyl acetate, polyvinyl alcohol, or the like. Alternatively, a shape memory or self-expandable material can be used in a non-self-expanding configuration, for example shape memory material which has not been heat set into shape or a nickel-titanium alloy with an austenite finish (Af) temperature above body temperature. Other viable shape memory alloys include copper-aluminum-nickel, iron-manganese-silicon, copper-zinc-aluminum, and the like. The metallic or polymeric frame of the malleable structure of the types described above would be covered with a distal sleeve to maintain vacuum lumen integrity during aspiration. Any pleats or folds in the malleable structure would typically be formed by the manufacturer by pressing it with a heated multiple jaw folding fixture of the sort commonly used in the industry to fold angioplasty balloons. The malleable structure can be a separate component attached to the aspiration catheter shaft using adhesives or heat bonding, or it may be integrated into the shaft during manufacture. In a preferred example the tubing used to form the malleable structure is also laser cut into a coil or other shaft reinforcement structure used for the distal catheter shaft. The malleable structure may be designed to collapse when exposed to a negative pressure of between 0.05 atm and 0.9 atm, preferably 0.7-0.9 atm. This collapse may occur over the clot, or delayed until the clot has been drawn proximal to the tip of the aspiration catheter.

In another example, the scaffold material can elastically stretch or plastically deform such that the increase in diameter at the scaffold mouth is at least partially due to material elongation, which may be further supplemented by an unfolding manner of operation. The material used may be laser cut with microscopic holes or slots to facilitate this. In a preferred example the size of such holes or slots are small enough to not significantly compromise the ability of the material to hold vacuum during aspiration (i.e. the size of the laser cut features with the structure in the expanded state is approximately that of a red blood cell or smaller, for example 10 microns, such that the features are rapidly clogged and blocked by blood cells during aspiration in order to maintain the vacuum lumen).

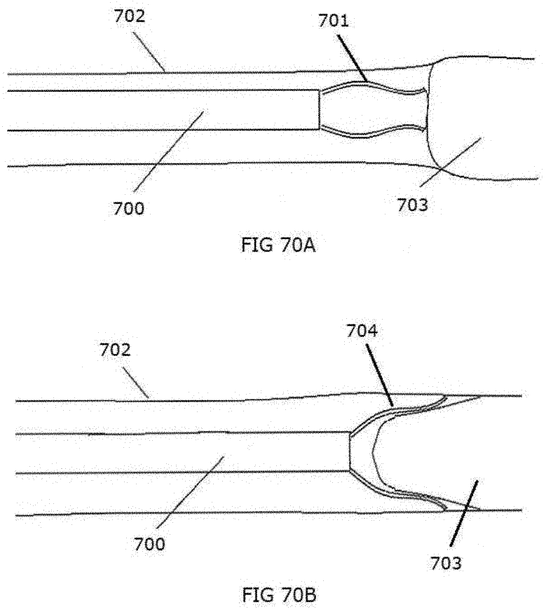

FIGS. 70A and 70B show an example of a malleable conforming scaffold in operation, in which the catheter 700 with malleable conforming scaffold in the collapsed state 701 is advanced into the vessel 702 with a clot 703. As the tip of the device is gently pushed against the clot, the malleable conforming scaffold flares into the expanded state 704 to engage the clot and seal against the clot and vessel. A vacuum force or negative pressure can then be applied at the proximal end of the aspiration catheter in order to draw the clot into the catheter to remove it from the artery. The malleable structure may be further designed to partially collapse in response to the vacuum application in order to prevent the clot from being dislodged during aspiration.

The deformation of the malleable structure during expansion may be predominately plastic (permanent) in nature, or partially or predominately elastic (recoverable) in nature. While a substantially plastically-malleable structure would likely require less force to expand to accommodate the clot, an advantage to a substantially elastically-malleable structure would be that it would at least partially recollapse after aspiration of the clot. However due to the malleable nature of the structure, the way in which the structure tapers outwards towards the distal end, and typical vessel geometry in which the more proximal vessels are larger, there would be no issues with withdrawing the device after aspiration with the malleable structure still in the substantially expanded state.

A hybrid design combining aspects of both a malleable tip and a self-expanding structure may provide enhanced performance to either design separately. In this embodiment the expandable distal segment initially self-expands to begin opening from the fully collapsed delivery state, and then relies on its malleable properties at least in part thereafter to complete expansion and engulf the clot. In this design the partial self-expansion serves to aid the initial opening when the tip of the device is substantially perpendicular to the clot and further reduces the amount of material ductility needed to complete expansion to the extraction configuration.

A hybrid self-expanding and malleable distal expanding segment can be formed from one of the many geometries described for a self-expanding structure above, such as an array of linear elements, loops, sinusoidal rings, or combination thereof. Materials suitable for this application include any of the self-expanding metals or polymers described elsewhere herein, or a normally non-self-expanding material which is neutral in the partially expanded state and then elastically deformed to the fully collapsed profile. In either case the hybrid design will typically utilize a means of constraint to prevent the initial self-expansion such as a sleeve, cap, friable bonding material, drawstring, retention ring, or other technique(s) such as described previously.

### Clot Disruptors

Some clots have significant fibrin content and are quite firm, especially in patients presenting many hours after first becoming symptomatic. These high fibrin clots are typically too cohesive to break up when exposed to a high vacuum force at an aspiration catheter tip in preferably the first pass. Such clots may sometimes be able to be captured by the tip of an aspiration catheter and pulled from the body intact as the aspiration catheter is withdrawn, but there is a significant risk that the distal end of the clot is not as firm and sheds embolic fragments as the clot is withdrawn, or that the clot detaches from the tip of the aspiration catheter and drifts downstream to re-obstruct the arterial vasculature (distal embolization). A preferred procedure is one in which the clot is removed entirely through the aspiration lumen, as typically occurs with less aged and more friable clots, in preferably the first pass. One means of achieving this is to physically disrupt the clot into smaller pieces capable of being aspirated from the patient. While many physicians are hesitant to due this due to the risk of embolic debris, the devices of the present invention substantially seal the vessel proximal to the clot which blocks any such embolic debris from escaping elsewhere into the neuro anatomy as a result of antegrade flow. In combination with such a device, clot disruption is a safe procedure option and may serve to reduce procedure times and/or allow removal of clots which would otherwise be unable to be aspirated in one or more successive attempts. A variety of options for clot disruption are described herein.

In another example of the present invention the device consists of an aspiration catheter featuring one or more blades, fine wires, or other cutting elements intended to disrupt the clot and aid its passage through the aspiration lumen and out of the body. The aspiration catheter with clot disrupting elements may be a conventional tubular design and may optionally feature an expandable segment and/or other novel features described elsewhere herein. The blades, fine wires, or other cutting elements are passive in nature and are contained within the body of the catheter shaft and/or any expandable segments (if present) in the distal end of the aspiration catheter, in order to prevent potential injury to the patient. After the catheter is advanced adjacent to the clot the radial expandable distal segment (if present) is expanded, such as by withdrawing a constraining sheath or otherwise actuating a structure on the catheter as per methods previously described herein. During aspiration the clot is pulled into the lumen and against the cutting elements, thereby being cut into smaller pieces for easier removal from the body. The cutting elements may also be used to disrupt the clot by physically pushing the device of the present invention into the clot; this is additionally useful in conjunction with the malleable designs as described above.

The cutting elements may extend straight across the lumen perpendicular to the axis, or may be angled. They may be straight or have a rounded or angled concave or convex profile. The cutting elements may only partially protrude into the lumen. In a preferred example, the clot disruptor consists of a ring-shaped circular cutter of diameter approximately equal to the inner diameter of the aspiration lumen in the catheter shafts, and is mounted within the distal expandable segment. The cutting elements may be mounted in the center of the aspiration lumen of either the fixed or expandable portions of the device, or may be off-center. In particular an off-center cutting element can be used in conjunction with an inner member designed to snag and mechanically pull back a portion of the clot as the inner member is removed, thereby shaving off part of the clot prior to initiation of aspiration.

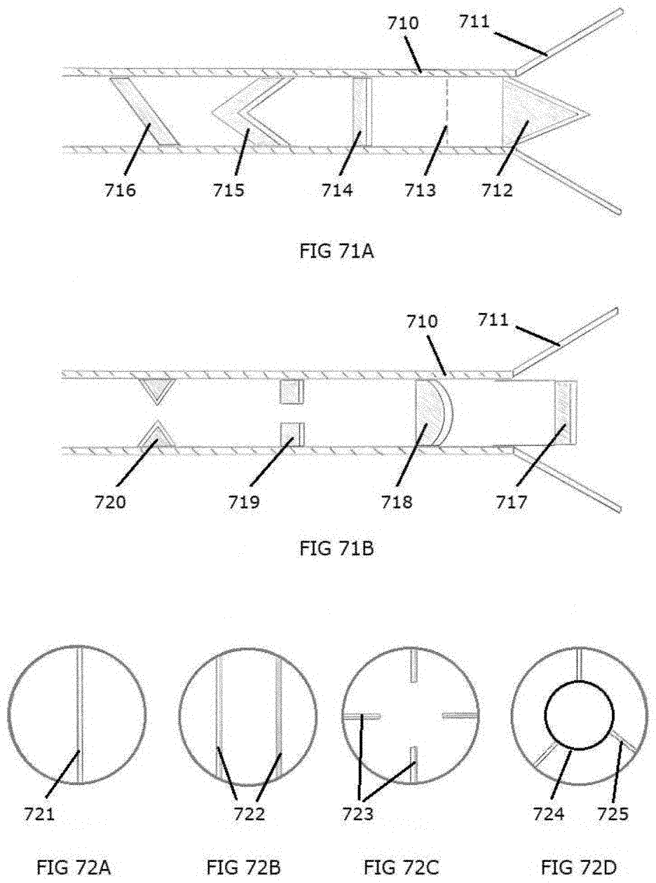

FIGS. 71A and 71B show examples of various passive cutting elements attached to an aspiration catheter 710 with a distal conical conformable scaffold 711. Note however that cutting elements may feasibly be attached to catheters utilizing any of the expandable scaffolds designs described herein. An aspiration catheter may feature just one element, or multiple elements of the same or different types. FIG. 71A shows options including an arrowhead blade 712, tensioned wire 713, straight blade 714, V-notch blade 715, and angled blade 716. FIG. 71B shows options including a ring-shaped blade within the distal expanding segment 717, a D-shaped blade with convex surface 718, partially impinging square blades 719, and partially impinging triangular blades 720.

FIGS. 72A-72D show end-on views of the aspiration lumen of devices containing various clot disrupting elements. FIG. 72A illustrates a device containing a single wire or blade cutter 721. FIG. 72B illustrates a device containing dual wire or blade cutters 722. FIG. 72C illustrates a device containing four partially impinging blades 723. FIG. 72D illustrates a device containing a ring-shaped blade 724 supported by struts 725.

If located in the expandable segment, the cutting elements may either be fix and smaller than the inner diameter of the expandable segment in the collapsed state, or may be folded or otherwise closed when the expandable segment is in the closed state and stretched open into a cutting position when the expandable segment is expanded.

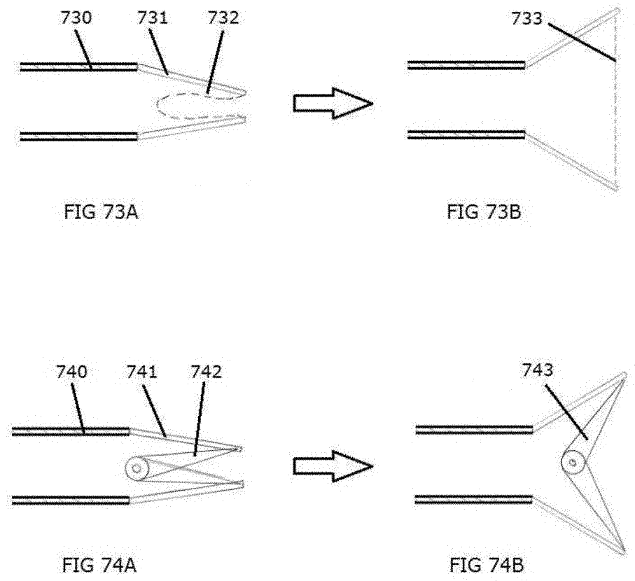

FIGS. 73A-73B show an example of a device containing a collapsible clot disrupting element. The aspiration catheter 730 features a self-expanding scaffold 731 at the distal end, for example comprising an array of nitinol struts covered by an elastic membrane. Attached at or near the tip of the struts is a fine wire or filament 732 that folds when the distal expanding segment is in the delivery state (FIG. 73A), and is under tension 733 and capable of cutting when the distal expanding segment is in the expanded state (FIG. 73B).

FIGS. 74A-74B show another example of a device containing a collapsible clot disrupting element. The aspiration catheter 740 features a self-expanding scaffold 741 attached to which is a two-piece V-shaped blade 742 that scissors closed when the scaffold is in the delivery state (FIG. 74A), and is pulled open 743 when the distal expanding segment is in the expanded state (FIG. 74B).

The cutting elements may be made from a filament or wire of 0.003″ diameter or less stretched across the lumen and then tied or bonded into the body of the catheter shaft. Alternatively, small blades can be laser cut from a flat metallic foil, preferably of 0.005″ thickness or less and more preferably of 0.002″ thickness of less, which is then micro-sanded or polished to create a razor-fine leading edge. Such blades would typically have tabs or hooks on the sides allowing them to securely interface with the catheter shaft and remain in position during device advance, aspiration, and withdrawal. In another means of manufacture, a hypotube which is laser cut into a coil or other geometry to provide a kink-resistant and crush-resistant core to a catheter shaft has barbs, tabs, or strips cut adjacent to the coils, which are manually pushed into the lumen before or after the cut hypotube is polymer coated or jacketed to form the catheter shafts.

Materials suitable for use for the cutting elements include metallic wires, tubes, and foils of stainless steel, cobalt chrome alloys, titanium and its alloys, nickel-titanium, platinum-iridium alloys, and others. Polymeric materials suitable for use include PEEK and polyimide. The cutting elements may be also be made from cast, machined, or sintered ceramics including aluminum oxide, silicon carbide, titanium nitride, boron carbide, diamond, and others. The cutting elements may benefit from being coated with a FEP, PTFE, or hydrophilic coating to improve lubricity against the clot and ease of cutting.

An alternate technique for disrupting a clot is to use a reversibly expandable coil in the distal expandable segment (as previously described) to compress a clot which has been aspirated into the distal expandable segment but which was not able to be fully aspirated through the lumen and removed from the body. The open coil is then torqued to close it over the clot, thereby compressing any clot material remaining within the expandable segment. After one or more compression and expansion cycles, the clot is release to travel into the aspiration lumen and be extracted from the patient. Alternately, a reversibly collapsible coil may be incorporated into the catheter shaft specifically for this purpose.

In a similar example, a coil is located within the aspiration lumen of the catheter shaft, with the distal end of the coil attached to the shaft body and the proximal end to a torque element extending within the aspiration lumen to the proximal end of the device where it can be manipulated by the operator. The torque element may be a large diameter tubular member such that the aspiration lumen continues through this member, or a smaller wire, mandrel, or tube such that the primary aspiration lumen is otherwise unobstructed and clot being aspirated travels adjacent to the torque element. If during the aspiration procedure clot appears to have become caught within the catheter shaft, the torque member can be rotated to tighten the coil onto the clot and compress it to a smaller diameter more easily able to be aspirated through the catheter shaft, in the same manner as described for a single coil or dual coil distal expandable segment described above. If desired, the coil within the aspiration lumen can be detachably connected at the distal end to the catheter body, such that it can be extracted from the device both to increase the area of the aspiration lumen and as a one-time means of physically pulling clot from the device and body separate from aspiration effect.

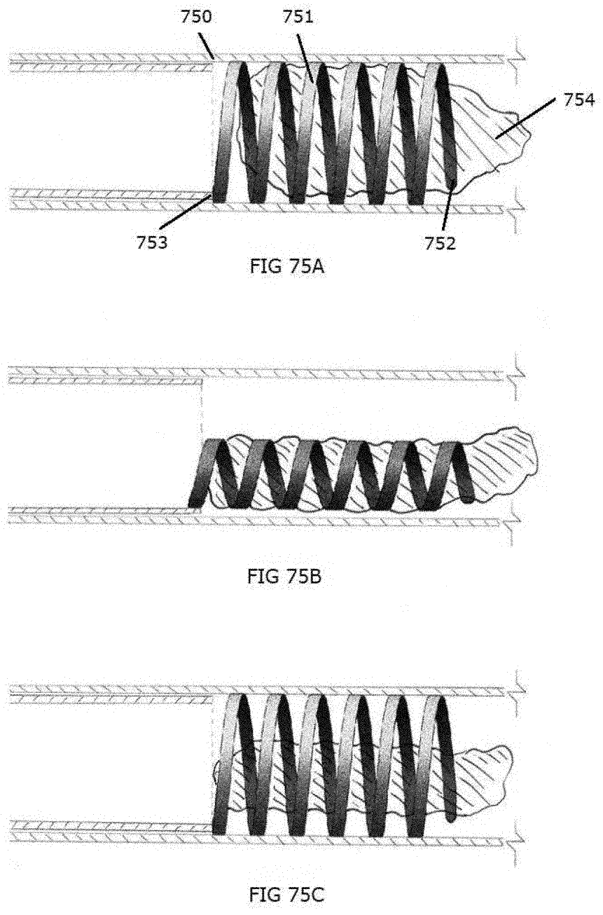

FIGS. 75A-75C show an example of a device containing a clot compression coil within the aspiration lumen of the catheter. The device consists of the catheter shaft 750 and the constricting coil 751. The distal end of the coil is attached to the shaft body at a point 752 (in background) and the proximal end of the coil is attached to a torque element 753 which extends to the proximal end of the device where it can be manipulated by the operator. The torque element may be a large diameter tubular member (as shown in the example) such that the aspiration lumen continues through this member, or the torque member may be a smaller wire, mandrel, or tube such that the primary aspiration lumen is otherwise unobstructed and clot being aspirated travels adjacent to the torque element. If during the aspiration procedure the clot 754 becomes caught within the catheter shaft (as indicated in FIG. 75A), the torque member can be rotated to tighten the coil onto the clot and compress it to a smaller diameter (FIG. 75B). The torque is then reversed to open the coil, leaving the compressed clot more easily able to be aspirated through the catheter shaft (FIG. 75C). If desired, the coil within the aspiration lumen can be detachably connected at the distal end to the catheter body, such that it can be extracted from the device both to increase the area of the aspiration lumen and as a one-time means of physically pulling clot from the device and body.

### Removable Inner Catheter for Improved Delivery

One challenge often faced by physicians during an aspiration procedure is difficulty tracking and/or pushing the aspiration catheter through the tortuous neurovascular anatomy to the site of the clot. Devices which are too stiff or can't track can fail to make tight turns and dig into the vessel wall, while excessively flexible catheters have tips or distal ends that tend to wander, buckle, or prolapse rather than advance forward down the artery as intended, or lack the ability to transmit sufficient push from the proximal catheter end to the distal end of the catheter. In order to mitigate these issues aspiration catheters are typically tracked over a guidewire (coaxial advance), and often with a microcatheter over the guidewire and inside the aspiration catheter (triaxial advance). The addition of the microcatheter helps device delivery by breaking the significant increase in stiffness and profile from the guidewire to the aspiration catheter into two smaller steps, at the cost of an overall increase in stiffness due to the additional device. In difficult cases physicians may want to add a second microcatheter to the system resulting in a quadriaxial or even pentaxial setup. This use of multiple internal support devices adds significant cost and time to the procedure. Also, even with these assistive devices in place, aspiration catheters still have an abrupt transition in profile at their distal end such that the exposed leading edge of the catheter can catch on side vessel takeoffs from the main vessel, and in tortuosity the catheter tip can scrape the vessel wall or dig into it. This contact can retard or prevent delivery of the device to the clot and can also scrape off endothelial cells, irritate or injure the vessel, and result in increased risk of post-procedure thrombosis or other complications.

The present invention describes one or more designs to enhance track and/or push and/or provide a transition at the distal end of the aspiration catheter to facilitate smoother advancement of the distal end of the aspiration catheter through the blood vessel or to the site of treatment. The inner catheter contains a guidewire lumen allowing the inner member and overlying device to be slid smoothly over the guidewire and to follow the guidewire up to or near to the clot or treatment site. The presence of the inner catheter described in this invention smooths the transitions in profile and stiffness occurring at the distal end of the device, minimizing the chance the device will catch on the vessel wall during advance, resulting in less vessel trauma and improving the ability of the guidewire alone to steer the device through the vessel anatomy and improving ease of deliverability, and/or enhances trackability, and/or enhances pushability of the distal end of the aspiration catheter. The removable inner catheter described in this invention may further incorporate a means to assist in the constraint and/or release a self-expanding distal structure at the distal end of the aspiration catheter. Prior to aspiration the inner catheter is typically removed from the aspiration catheter (outer device) so as not to occlude the aspiration lumen and reduce aspiration effectiveness. The removable inner catheter may be provided pre-assembled within the aspiration catheter (preloaded) by the manufacturer prior to sterilization, or the removable inner catheter may be packaged separately and is inserted into the aspiration catheter by the physician at the start of the procedure.

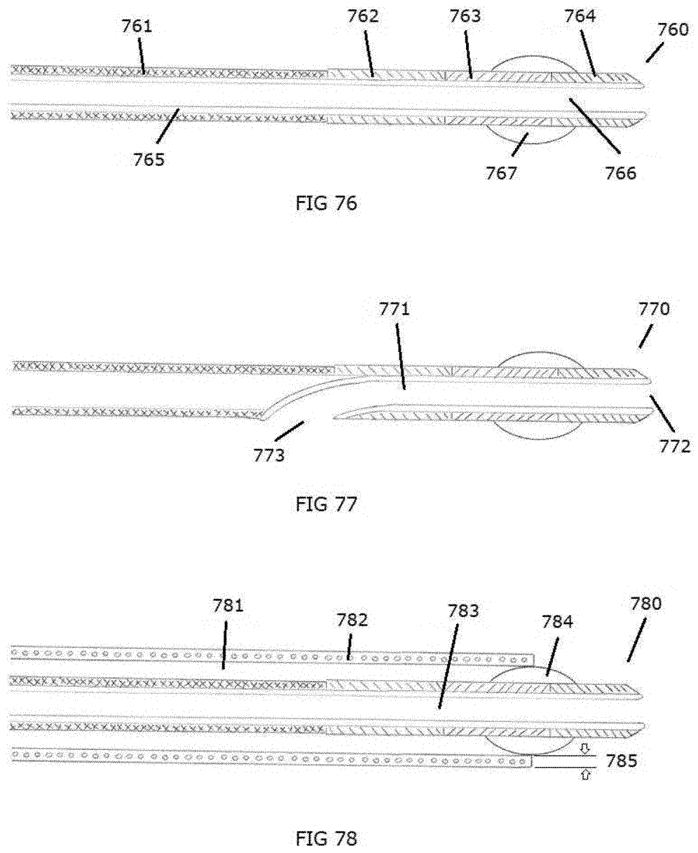

FIG. 76 shows an example of a removable inner catheter 760 of the present invention showing a design comprising a proximal shaft 761 and a distal shaft composed of one or more segments (sections) 762, 763, 764 of distally decreasing profile and/or stiffness and/or of different material. Typically, the catheter will also incorporate a lubricious inner liner 765. On the proximal end of the device (not shown) is a conventional catheter hub with a luer fitting for attachment of accessories, for example a syringe enabling the removable inner catheter to be flushed with fluid. The catheter shown is of an “over the wire” configuration, in which the guidewire lumen 766 extends for the full length of the inner catheter. Furthermore, the device may feature a distal interface section 767 towards the distal end of the inner catheter, discussed further below in more detail. The inner and/or outer diameters of the inner catheter may be the same along the length of the device or may vary. Typically the inner diameter is substantially constant while the outer diameter is larger in profile proximally to provide increase push and kink resistance in the areas where less flexibility is required, than a distal segment of the inner catheter.

FIG. 77 shows a “rapid exchange” variant of the removable inner catheter of FIG. 76, in which the guidewire lumen 771 extends only through the distal portion of the inner catheter 770, from the distal tip of the device 772 to an exit port 773. After exiting the inner catheter at the guidewire exit port, the guidewire continues proximally adjacent to the inner catheter inside the aspiration lumen of the outer device. An advantage of a rapid exchange design versus a fully coaxial over-the-wire design is that it can be withdrawn from the aspiration system and removed from the guidewire without requiring an excessively long guidewire to be used. The rapid exchange design allows for easy removal of the inner catheter without needing to remove the guidewire from the patient and provides for the use of a shorter guidewire which is easier for the physician to manipulate during the procedure. In one example, the inner catheter rides over the guidewire for a length of 3 to 50 cm, more preferably for a length ranging from 5 to 30 cm.

In both over-the-wire and rapid exchange variants the guidewire lumen allows the inner catheter and overlying device (such as an aspiration catheter) to be slid smoothly over the guidewire and to follow the guidewire up to or near to the clot. The guidewire lumen in the inner catheter is appropriately sized to fit the guidewire, with the inner diameter of the guidewire lumen of the inner catheter typically 0.001″-0.005″ larger than the outer diameter of the guidewire it is intended to receive. The clearance is typically smallest at the tip of the inner catheter, typically ranging from 0.001″ to 0.002″, allowing for a smooth transition in profile from the guidewire up onto the inner catheter.

The distal segment of the removable inner catheter is constructed from one or more polymer material along the distal segment length, with the segments increasing in stiffness proximally to provide better support for and aid delivery of the outer device. The increases in stiffness can be achieved through use of firmer polymers and/or sections of larger diameter and/or increased wall thickness. Typically the inside of the inner catheter is constructed from two or more layers in which the innermost layer uses PTFE, FEP, HDPE or other low-friction material in order to provide for smooth movement over a guidewire. The outer layers may be constructed from any of the other polymers disclosed elsewhere herein but preferably is constructed from polyamides and/or polyurethanes for 35D-80D durometer hardness. One or more layers may contain holes, slots, or other flexibility-increasing features. One or more distal segments of the removable inner catheter may be reinforced with a coil or braided structure to prevent ovalization, collapse, or kinking while maintaining sufficient flexibility. The inner catheter construction including any reinforcement may be designed to provide for increased axial stiffness for efficient transmission of push and/or pull force from one end of the device to the other, as would be important for applications requiring such force transmission to release a constrained self-expanding structure. In another example, the distal portion of the removable inner catheter comprises a tight spring guide or coil made from polymer, metal, alloy, superelastic nitinol, or shape memory alloy. The tight coil is flexible due to the absence of a polymer jacket but high compressive stiffness for transmission of push force and sufficient pull force for withdrawal of the transition structure. In another example, the tip of the spring guide or coil is attached to a soft tip made from polymer or has an atraumatic tip.

The proximal portion of the removable inner catheter typically comprises a metallic hypotube providing increased axial stiffness for best push and advance of the combined inner catheter and outer device. The hypotube may be laser cut with slots, spirals, or holes to increase flexibility where desired, for example at its distal end where it transitions into the distal portion of the removable inner catheter. Alternatively, a polymer shaft may be used for the proximal portion of the removable inner catheter. Alternatively, a braided shaft may be used for the proximal portion of the removable inner catheter. Such a polymer shaft may be constructed from Nylon, PEEK, polyimide, or other polymer of similar strength, and it may further be reinforced with coils or braids. The proximal portion, stiffer and firmer relative to the distal portion and intended only for use in the less tortuous aortic and lower carotid anatomy, allows for more effective push transmission even with rapid exchange designs where only the distal portion of the inner catheter runs over the guidewire.

The proximal end of the removable inner catheter may contain a hub and luer fitting which attaches to the proximal guidewire exit or aspiration port of the outer device. This keeps the two components locked together and the removable inner catheter in position during device delivery. Alternatively, the proximal end of the inner catheter may be a simple hypotube and is cinched in place by a Tuohy fitting is attached to the proximal guidewire exit or aspiration port of the outer device.

A key advantage of the device of the present invention is that it has a smooth and gradual transition in profile from the guidewire up to the maximum outer diameter of the outer device such as the aspiration catheter. While the inner catheter will be sized to the guidewire as tightly as possible as discussed above, a further key feature of the removable inner catheter design of the current invention is that it contains a distal interface section, also referred to as a transition structure or stiffening member, in which the outer diameter of the inner catheter tapers up to a larger profile and then interfaces with the outer device (such as an aspiration catheter distal end). The transition structure may comprise any one or more of a metallic scaffold, a polymeric membrane, a polymeric scaffold, a combination of metallic scaffold and polymeric membrane, a sleeve, an expandable member, a shape memory alloy scaffold, or other structure that that may be configured to (1) cover or partially cover or (2) fill or partially fill the open distal end and/or a distal segment of the lumen of the aspiration catheter.

The presence of the transition structure may serve to support the aspiration catheter during advancement into or through the blood vessels, and improve the track and/or push of the aspiration catheter through the arteries. Neurovascular aspiration catheters typically range in inner diameter from about 1 mm to about 3 mm, with the most commonly used ranging from 1.5 mm to 2.35 mm. In contrast, the outer diameter of an inner catheter may range from 0.25 mm to 2 mm, with the most commonly used ranging from 0.5 mm to 1.54 mm. (While currently available devices are about 0.5 mm or larger, alternate smaller inner catheters intended to be used without a guidewire or integrated with a guidewire are contemplated herein.) The invention is also applicable to larger devices intended to use elsewhere in the body, for example for removal of pulmonary thromboses, in which case the aspiration catheter may have an inner diameter as high as 30 mm with an outer diameter perhaps 0.05 mm-1 mm higher, and the associated inner catheter will be reasonably larger as well. Therefore in most combinations an annular gap exists between the outside of the inner catheter and the inside of the aspiration catheter which the distal interface section serves to fill. In the case of neurovascular aspiration systems the annular gap will typically range from 0.025 mm to 2 mm, preferably 0.05 mm to 0.1 mm, and more preferably 0.1 mm to 1.25 mm. The interface section, also referred to herein as a transition structure, may be designed to fill the annular space, or may further abut against the front of an outer catheter and/or cover it. While the end of the inner member may be flush with the end of the aspiration catheter, more often the inner catheter is longer than the aspiration catheter and extends distal of the aspiration catheter in order to provide the smooth transition in profile and stiffness. In a preferred example the inner catheter extends distal to the aspiration catheter in a range from 1 mm to 10 cm, more preferably 2 mm to 5 cm, and most preferably from 3 mm to 3 cm. In another example, the inner catheter is slightly shorter than the aspiration catheter such that the tip of the inner catheter does not extend distal to the tip of the aspiration catheter. This may allow for maximum flexibility since stiffness of the inner catheter is not adding to that of the total system at the critical transition at the tip of the aspiration catheter.

In a preferred example, the profile of the inner catheter at the position of the outer device (such as the aspiration catheter) distal tip matches the inner diameter of the outer device at that point so there is no gap between the inner member and the distal end of the outer device. The distal interface section or transition structure may taper linearly forming a conical shape, non-linearly forming a convex or concave transition profile, be a series of small steps, or any combination of those. It may be spherical, hemispherical, ovoid, conical, bullet shaped, cylindrical, or other. It may be designed to contact the outer device only on the inside of the aspiration lumen, or feature a reverse step or abrupt taper such that the distal interface section at least partially protects the leading distal edge of the outer device, or alternatively It may be designed to not contact (or have a smaller profile) than the inner lumen diameter of the aspiration catheter). The distal interface section or transition structure may be a solid structure, a hollow structure, or comprise a sleeve or other structure which at its distal end connects to or couples to the removable inner member and at its proximal end meets or overlaps or couples to the distal tip or distal end of the outer device. The transition structure may be detachably coupled to a distal segment, end or tip of the inner catheter and may be configured to be slidably retracted through the lumen of the aspiration catheter lumen after detaching. In some instances, the transition structure when detached may be smaller than the open distal end of the aspiration catheter. In other instances, the transition structure when detached may be larger than the open distal end of the aspiration catheter and may be configured to be compressible to slidably be retracted through the aspiration catheter lumen. In some instances, a proximal end of the transition structure may be configured to be decoupled from a distal tip of the distal segment of the inner catheter. In other instances, a distal end of the transition structure may be configured to be decoupled from a distal tip of the distal segment of the inner catheter. In still other instances, the inner catheter may be configured to retract the transition structure proximally into the aspiration lumen causing the transition structure to invert as it is retracted.

FIG. 78 shows an example of the removable inner catheter 780 of FIG. 76 positioned within the aspiration lumen 781 of the aspiration catheter 782. Both components are arranged coaxially around a guidewire lumen 783 which may run the full length of the inner catheter (see FIG. 76) or only a portion of the length (see FIG. 77). In one example the coaxial guidewire lumen extends for a length ranging between 1 cm and 40 cm within the distal segment of the inner catheter and/or aspiration catheter, more preferably between 1 cm and 25 cm, and proximal to this segment the inner catheter comprises a braided shaft, coil reinforced shaft, mandrel, rod, hypotube coupled to the coaxial distal segment which allows the inner catheter to be advanced and withdrawn. The distal interface section 784 comprises a solid bullet or slug of material of outer diameter substantially matching the inner diameter of the aspiration catheter, thereby minimizing the step-up at the distal end of the aspiration catheter to the wall thickness of the catheter itself (785), alternatively, the solid bullet or slug outer diameter maybe smaller than the distal tip or distal end diameter of the aspiration catheter. The bullet may be made from polymer, metal, or ceramic, and may be extruded, cast, injection molded, or otherwise created as known to those skilled in the art. Preferred materials include any suitable for use for the catheter shafts as has been previously described. Softer polymers and elastomers are also suitable for this application. In one example the bullet is made from a tubular section of Pebax 35D polymer cut from an extrusion which is then heat melted over the inner member resulting in the final tapered shape. In another example the bullet is built up from an applied adhesive gel. In another example the bullet is made from a high density metal such as gold or platinum in order to simultaneously provide radiopacity to the system. In yet another example the bullet can be 3D printed from porous polymer or foam whereby upon extrusion from a head nozzle or heating the filaments in a hot box, the solid material expands and becomes low to high density foam linearly dependent on temperature. The bullet can be shaped by 3D printing or heating in a mold. Said porous or foam bullet can have outer dimensions same as the outer diameter of the aspiration catheter and can compress when pulled into a smaller diameter aspiration lumen.

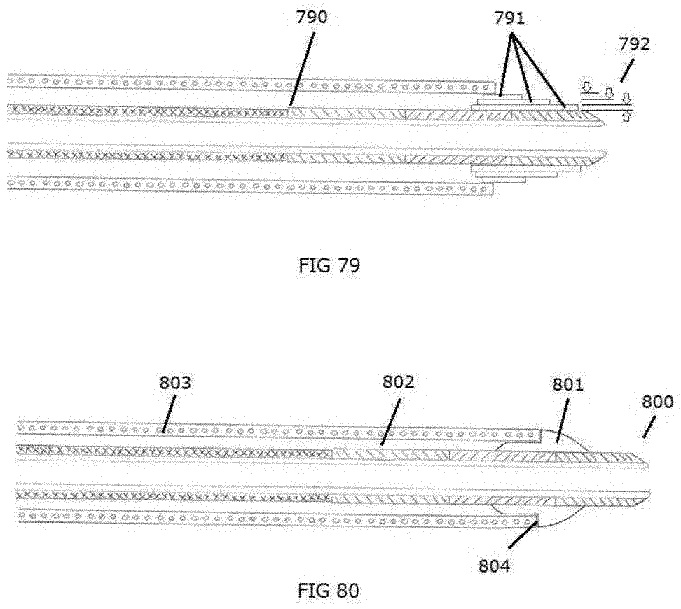

FIG. 79 shows an example of a removable inner catheter 790 featuring a stepped cylindrical distal interface section, as would be easily manufacturable using layered tubular extrusions 791 which are heat bonded to the removable inner catheter. The use of numerous small steps 792 provides a generally smooth transition in profile and stiffness, and the bonding process may impart a chamfer to the end of each step further improving transitions.

FIG. 80 shows an example of a removable inner catheter 800 with a distal interface section 801 contacting the inside of the aspiration lumen 802 of an aspiration catheter 803. The interface section features a reverse step 804 detachably coupled to the distal end of the aspiration catheter allowing the inner catheter to abut and protect the distal leading edge of the outer catheter. The reverse step may be present in the middle of the transition structure as shown, or be biased towards or entirely at the proximal or distal end of the transition structure. In this example the interface section is made from a soft and flexible polymer such as a silicone in the 25A-40A hardness range which is soft enough to distort under tension and allow the structure to be pulled inside the aspiration catheter and removed from the body. In one variant, a proximal portion of the transition structure is coupled to the distal segment of the inner catheter but the distal portion is not connected, thereby allowing the transition structure to elongate under tension to reduce its profile and facilitate withdrawal through the aspiration lumen.

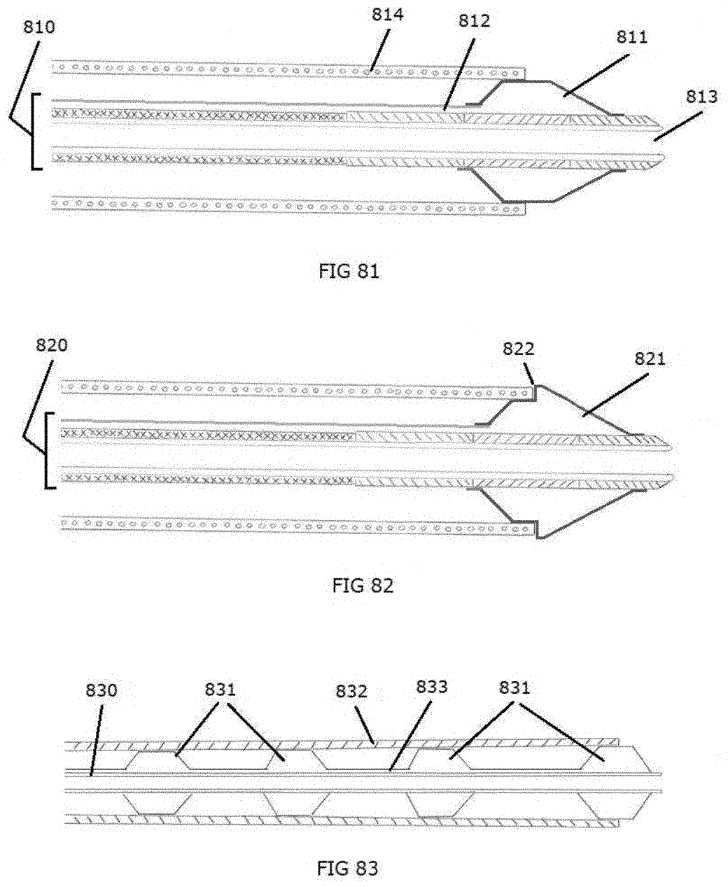

FIG. 81 shows an example of a removable inner catheter featuring an inflatable balloon to provide the interface between the removable inner catheter and the aspiration catheter. The removable inner catheter 810 is constructed in a manner similar to that described above, except that the distal interface section consists of an inflatable polymer balloon 811. The balloon may be substantially plastic in nature and expand primarily due to an unwrapping/unfolding action similar to an angioplasty balloon made from HDPE, PET, nylon, Pebax, or polyurethane, or it may be elastic like an occlusion balloon made from silicone or other soft stretchable material. The removable inner catheter will furthermore feature a separate inflation lumen 812 providing fluid communication between the inside of the balloon and a separate inflation/deflation port on the proximal hub of the catheter. The inflation lumen may be a separate roughly circular, crescent shaped, or D-shaped lumen running parallel to the guidewire lumen 813 (as shown) or may be formed by covering the guidewire lumen with a second tubular member such that the space between the two tubes forms the inflation lumen. Construction of a removable inner catheter with distal balloon may therefore resemble that of an angioplasty or occlusion balloon catheter, except for the differences in dimensions and material stiffnesses as appropriate to neurovascular use. The balloon is inflated to make firm contact with the inside of the aspiration catheter 814 thereby anchoring the two together for improved deliverability as described above. The balloon may be as short as a few millimeters long—just enough to engage the aspiration catheter—or much longer, feasibly up to 10 cm long. A longer balloon grants the physician the ability to deflate the balloon and reposition the inner catheter with respect to the aspiration catheter during the procedure, as may be desirable to help the aspiration catheter negotiate a particularly tight bend in the artery. The balloon distal end may be rounded, conically tapered, or of another shape which assists in providing a smooth transition in profile and stiffness between the guidewire, distal end of the removable inner member, and the end of the aspiration catheter. After the aspiration catheter has been delivered to the site of treatment, the balloon is deflated and the inner catheter is withdrawn prior to clot aspiration.

FIG. 82 shows an example of a removable inner catheter 820 featuring an interfacing balloon 821 with a reverse step 822 in order to fully shield the distal end of the aspiration catheter from contact with the vessel. Depending on the balloon material and manufacturing process used, the step may be molded into the balloon in advance (for example a nylon balloon blow molded in a stepped mold) or it may be formed during interface (for example, a compliant balloon pressurized against the end of the catheter will have the non-constrained distal part flare around the distal end of the catheter).

FIG. 83 shows an example of a removable inner catheter 830 featuring multiple friction anchors such as balloons 831 which are inflated to contact the inner diameter of the aspiration catheter 832 or are configured when inflated to reduce the annular gap between the outer surface of the inner catheter and the inner surface of the aspiration catheter. In one example the aspiration catheter has an inner diameter ranging from 1.5 mm to 3.0 mm and the inner catheter has an outer diameter ranging from 0.5 to 2.75 mm, such that inflation of the balloons reduces or eliminates the annular gap between the devices. While the distal balloon provides the smooth profile and stiffness transitions discussed above, one or more additional balloons located more proximal serve as friction anchors between the removable inner catheter and the aspiration catheter. When pressurized, the plurality of balloons cause the aspiration catheter and inner catheter to be reversibly locked together, thereby stiffening the composite system for enhanced pushability. The balloons may share a common inflation/deflation lumen 833 or may be independently controllable. If they are independently controllable then the inner catheter may be selectively locked against the outer catheter in different areas to locally add stiffness and pushability as required for best device deliverability. In one example the additional balloons or friction anchors are distributed over a range from 0.5 cm to 15 cm proximal to the distal end of the aspiration catheter, and are spaced between 5 mm and 25 mm apart. The balloons may be of the same material and/or diameter, or of different materials and/or diameters in order to provide differing levels of engagement with and adherence to the aspiration catheter. For example a more distal balloon may be of a more lubricious material and/or smaller in diameter such that for the same applied pressure it exerts less force on the aspiration catheter than a more proximal balloon of stickier material and/or larger diameter, such that in the distal region the devices are less firmly locked together. This may allow relatively greater stiffness and push proximal and relatively greater flexibility and track distal.

In another example, the friction anchors 831 of FIG. 83 may alternatively be a plurality of frictional anchors formed from one or more of a solid, hollow, solid flared area, bumps, or other structures, said structures configured to reduce or eliminate an annular gap between the aspiration catheter aspiration lumen and the inner catheter outer surface and/or enhance the track and/or enhance the push of the aspiration catheter, along a distal segment of the aspiration catheter. Features described in the present inventions including frictional anchors, multiple balloons, balloons, and other aspects apply herein. The structures maybe circumferentially covering an outer surface (as shown in FIG. 83 or discrete structures on one or more regions of a distal segment of an outer surface along one axis, or patterned along said distal length. Each of these structures can have a length, preferably ranging from 1 mm to 1 cm, preferably ranging from 1 mm to 5 mm, more preferably ranging from 1 mm to 3 mm. The frictional structure may have same or different profile or thickness, length, and/or height. The frictional anchors may cover one side of the outer surface of the inner catheter along the distal segment of the inner catheter outer surface, or the frictional anchors may have a helical pattern or other patterns such as 60, 90, 120, 180 degrees offsets, along a distal segment of the outer surface of the inner catheter, or the frictional anchors may circumferentially cover an outer surface of the inner catheter such as a donut shape or other shapes, or other. In a preferred example, the frictional anchors protrude radially from the outer surface of the inner catheter along a distal segment of an inner catheter outer surface. In a preferred example, the frictional anchors provide one or more of: reduces or eliminates the annular gap between an aspiration lumen inner diameter and the outer surface of the inner catheter, to enhance trackability, to enhance pushability, to enhance transition between an inner catheter having a small configuration and an aspiration catheter having a larger inner diameter configuration, and to provide an enhanced tip transition, of the aspiration catheter as it is advanced through the blood vessel to provide a treatment, such as clot removal. The one or more anchors have additional advantages when the annular space between the outer surface of the inner catheter and the inner surface of the aspiration catheter is large, typically ranging from 0.1 mm to 2 mm, more typically ranging from 0.2 mm to 2 mm, wherein the multiple anchors reduce or eliminate the annular gap by making the annular gap range from zero to 0.5, more typically ranging from zero to 0.3.

In another example some or all of the friction anchors on the inner catheter are not intended to contact the inner diameter of the aspiration catheter but rather adjust the spacing of the annular gap between the devices. This may be done in order to maintain coaxiality of the components for improved trackability and/or pushability or provide partial temporary adherence between the devices for the same benefit. Such friction anchors may comprise inflatable balloons as previously described, or may be separately attached or integrated bumps, slugs, spheres, or other material additions designed to locally increase the profile of the inner catheter. Any inflatable balloons and solid friction anchors may be designed to not engage the inner diameter of the aspiration catheter, or to only partially engage it (for example a rib or bump on the outside of the friction anchor), or to full engage it.

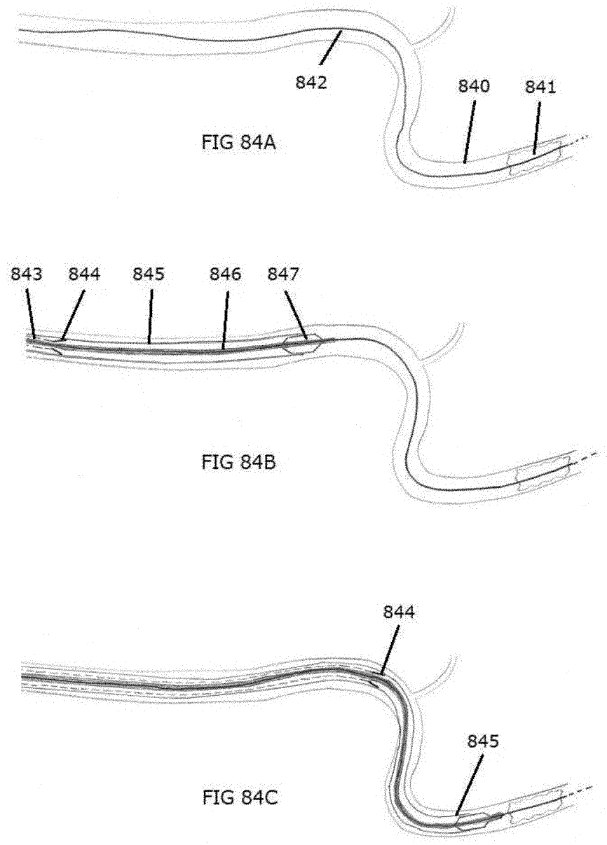

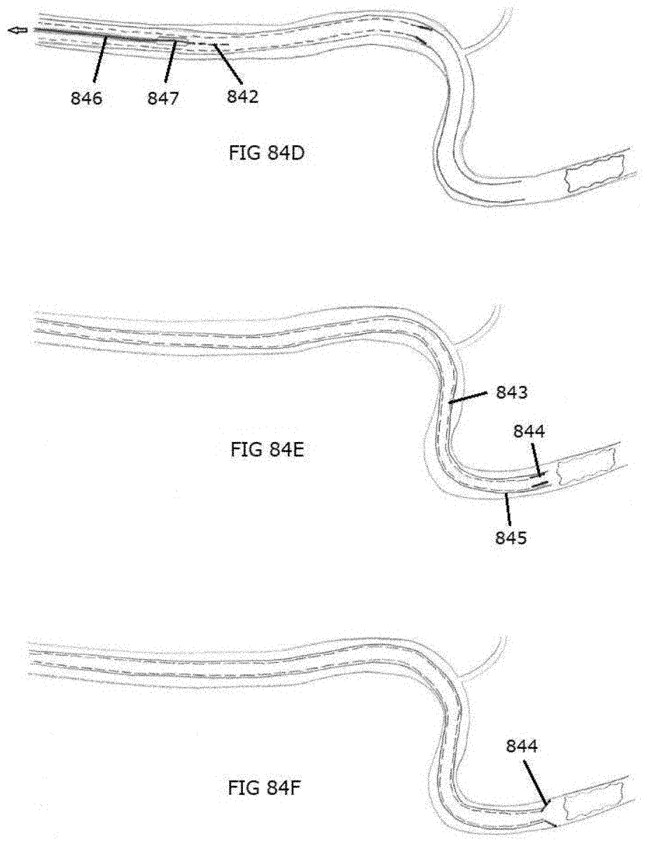

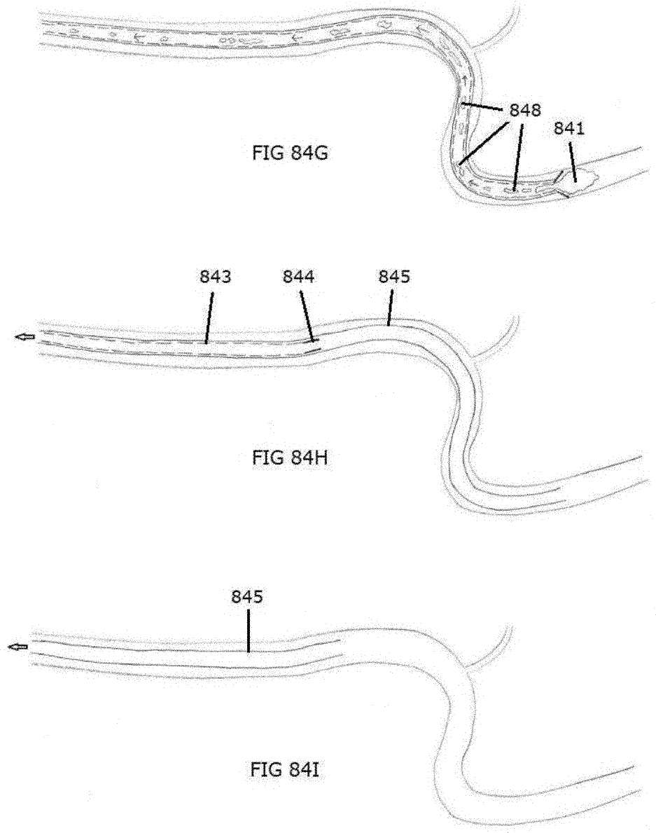

In another example, the removable inner catheter with balloon seals against the tip of an outer elongated tubular body constraining a self-expanding scaffold (refer to FIGS. 28A-C for example). This allows the inner catheter and outer constraint sheath to be advanced towards the clot independent of the inner elongated tubular body and scaffold, thereby providing for improved deliverability of the entire system. A procedure using a device of this sort is depicted in FIGS. 84A-84I.

FIG. 84A shows a neurovascular vessel 840 containing a vessel lumen for blood which is occluded by a clot 841, and in which a guidewire 842 has been tracked through the vessel and clot in preparation for device delivery.

FIG. 84B shows the device comprising an inner elongated tubular body 843 with self-expanding scaffold 844, a constraining outer elongated tubular body 845, and a removable inner catheter 846 with inflated balloon 847, after insertion into the neurovascular anatomy. (Note that the inner elongated tubular body is a continuous non-porous structure, but has been illustrated with dashed lines for ease of differentiation from adjacent items in the Figures.)

FIG. 84C shows the device of FIG. 84B after completion of the first stage of delivery, in which the distal end of the outer elongated tubular body 845 is near the clot while the inner elongated tubular body and self-expanding scaffold 844 remains proximal to significant tortuosity.

FIG. 84D shows the device of FIG. 84B after completion of the second stage of delivery, in which the removable inner catheter 846 has had the balloon 847 deflated and is being removed from the body along with the guidewire 842.

FIG. 84E shows the device of FIG. 84B after completion of the third stage of delivery, in which the inner elongated tubular body 843 with self-expanding scaffold 844 has been advanced within the constraining outer elongated tubular body 845 up to the clot.

FIG. 84F shows the device of FIG. 84B after deployment of the self-expanding scaffold 844.

FIG. 84G shows the device of FIG. 84B in the process of aspirating the clot 841, which is broken apart by the vacuum into smaller clot fragments 848 which can be fully removed from the lumen.

FIG. 84H shows the device of FIG. 84B after the inner elongated tubular body 843 with self-expanding scaffold 844 has been withdrawn into the outer elongated tubular body 845 and is being removed from the patient.

FIG. 84I shows the last step of the procedure in which the outer elongated tubular body 845 is withdrawn from the body.

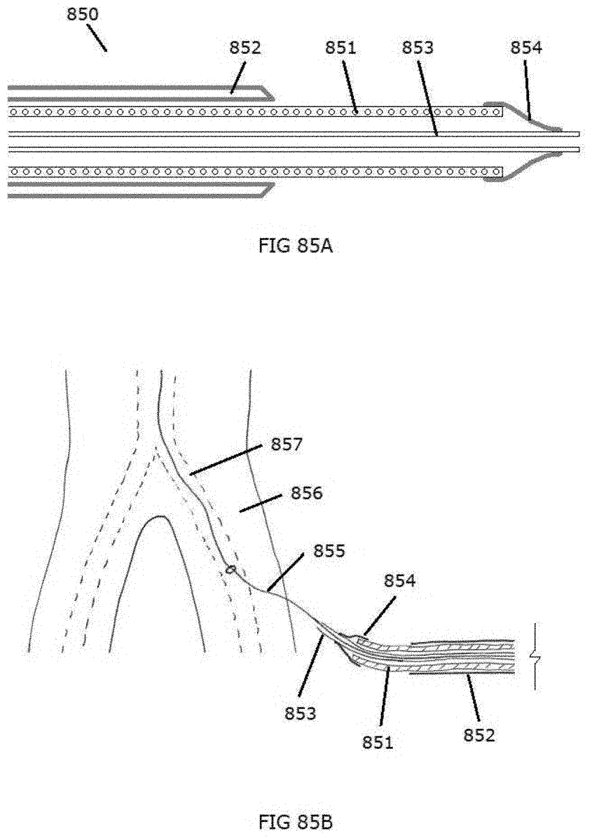

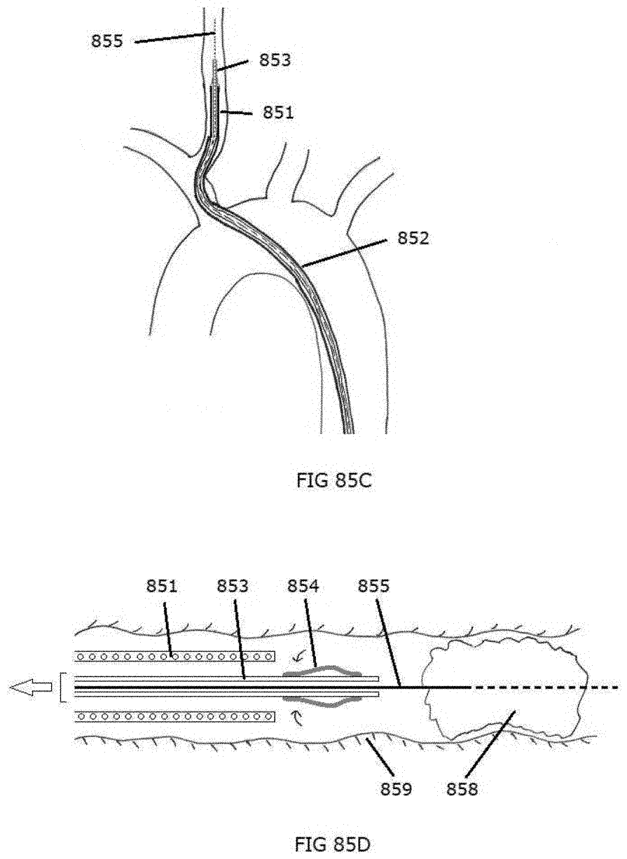

FIG. 85A shows an example of an aspiration catheter assembly 850 consisting of an aspiration catheter 851, an outer guiding sheath 852, and a removable inner catheter 853. The components are aligned coaxially over at least a portion of their lengths. The presence of the outer sheath may serve to support the aspiration catheter during advancement into or through the blood vessels, and improve the track and/or push of the system through the arteries. The inner catheter further features a distal interface section, also referred to as a transition structure, comprising a collapsing sleeve 854 which has been stretched to overlap the distal leading edge of the aspiration catheter. In some examples the collapsing sleeve may be of a substantially cylindrical profile, hemispherical, conical, or bullet-shaped. It may be substantially blunt or tapered. In a preferred example the sleeve is of a tapered conical shape. The sleeve may cover just a small portion of the outer surface of the distal end of the aspiration catheter or a longer distance. In one example the sleeve covers distal segment of the aspiration catheter ranging from 1 mm to 10 cm in length, preferably 5 mm to 10 cm, and more preferably 1 cm to 10 cm. It may overlap the aspiration catheter as shown, be designed to fill the annular gap and/or abut up against the distal end of the outer device. The proximal end of the transition structure may be smaller, the same, or larger than the open distal end of the aspiration catheter. In any variant the sleeve provides a smooth profile transition from the outer diameter of the inner member up to the outer diameter of the aspiration catheter with no forward facing steps, lips, or other catch points. Once the system is in position, the inner member is advanced slightly with respect to the aspiration catheter, and the tensile forces on the sleeve cause it to decouple and detach from the end of the aspiration catheter, whereupon it elastically collapses back onto the inner member at which point both can be removed from the anatomy prior to initiation of aspiration.

The sleeve may be made from one or more of a variety of standard and shape memory polymers in the silicone, polyurethane, and polyamide families. Examples included C-flex (silicone), fluorosilicone, Tecothane (polyurethane), and Pebax (polyamide). Some name brand polymers suitable for this application which generally fall into one or more of the above polymer families include Chronoflex, Chronoprene, and Polyblend. Sleeves in the hardness range of Shore 50A through 40 Durometer work best. At the lower end of this scale the sheath material is predominantly elastic while at the upper end of this scale a portion of the sleeve stretch is plastic, not elastic, but enough of it is elastic to fulfill the recovery needs. Typically, the sleeve will be extruded and/or molded. In another example the collapsible component comprises a metallic scaffold, a polymeric scaffold, or a combination thereof which may then be covered with an elastic membrane. In a preferred example the scaffold is made from a shape memory material such as nickel-titanium heat set in a fully crimped state which is then expanded to cover the distal tip of the outer device, and which then recloses onto the removable inner catheter after it is pushed off the outer device. The scaffold may be of a sinusoidal ring design, a flat ribbon or other coil design, or other. The collapsing sleeve may be attached to the inner catheter at its distal end or somewhat proximal of the end. It may be attached or coupled at a relatively finite point, or coupled to it along a significant portion or all of the segment extending distal to the aspiration catheter, for example up to 10 cm. In the example shown the proximal end of the sleeve is not attached to the inner catheter but is stretched to overlap the outer devices. Standard methods of attachment or coupling include heat fusing, adhesive bonding, soldering, crimping, and suturing. The sleeve may also be molded into the inner catheter or otherwise integrated into it during manufacture, using the same material as the outer layer of the inner catheter or from a different (and typically softer) material. The sleeve may be coextruded during extrusion of the inner catheter. It may be stretched over and necked upon the underlaying catheter.

In a preferred example the collapsing sheath is further used as a transition structure to smooth the profile and stiffness transitions at the distal tip of the system to allow the devices to be inserted into the patient together without a separate dilator catheter being needed. In effect the inner catheter with transition structure is the vessel dilator. This saves both time and cost. In practice a 0.035″ or other large diameter guidewire is used to gain initial entry to the anatomy, and then the aspiration catheter and inner catheter are loaded over the wire and pushed directly into the patient. The taper on the transition structure reduces the force required to insert the devices into the vessel, providing for atraumatic insertion into the anatomy and advance through it. The 0.035″ wire may be exchanged for a smaller neurovascular delivery wire before or after device insertion as desired. Typically a further outer sheath will be pre-positioned over the aspiration catheter in order to help provide a hemostatic seal at the puncture site and to reduce trauma at the vessel entry point from the sliding aspiration catheter. The outer sheath may be a short sheath intended primarily for the above purposes, or be a longer guiding sheath which aids in delivery of the device through and possibly past the aorta. The outer sheath may be the same length as the aspiration catheter or may be shorter depending on how deep into the anatomy this support component is intended to advance. In one example the outer sheath ranges in length from 1 to 110 cm, preferably 10 to 100 cm, and more preferably from 10 to 90 cm. In one example the outer sheath ranges in length from 10 to 50 cm, preferably 10 to 40 cm, and more preferably from 10 to 30 cm. The outer sheath may fit reasonably snugly against the outer surface of the aspiration catheter for maximum profile gain or may be slightly looser for ease of relative device movement. In one example the annular gap between the aspiration catheter and outer sheath ranges between 0.025 mm and 0.25 mm, more preferably between 0.025 mm and 0.1 mm. FIGS. 85B-D demonstrate use of the device in this manner.

FIG. 85B shows the system as prepared for insertion into the patient. The guidewire 855 has been inserted into the patient's leg 856 and femoral artery 857. Tri-axially loaded over the guidewire are the removable inner catheter 853 with collapsing transition sheath 854, aspiration catheter 851, and guiding sheath 852. In this example the removable inner catheter features a collapsing transition sheath as the interface section, but it could as feasibly be a solid plug, inflatable balloon, or other design described previously. At the point where the system has been introduced into the patient, the outer sheath serves as an access sheath and substantially seals the artery against blood loss at the site of vascular penetration.

FIG. 85C shows the system in the midst of delivery, in which the guiding sheath 852 has been advanced sufficiently far to support the aspiration catheter across the aorta, and the aspiration catheter 851 and removable inner catheter 853 have been advanced further towards the neurovascular anatomy.

FIG. 85D shows the system at the conclusion of delivery, in which the removable inner catheter 853 and aspiration catheter 851 have reached the clot 858 in neurovascular blood vessel 859, and the inner catheter has been advanced slightly to allow the transition sheath 854 to disengage and collapse back against the inner catheter. The inner catheter and guidewire 855 can now be withdrawn from the patient (left pointing arrow), the aspiration catheter position adjusted as desired, and clot aspiration initiated.

Proximal to the interface section the inner catheter may remain substantially the size of the lumen it occupies such that friction between the inner catheter and aspiration catheter serves to enhance the pushability of the combined system and/or allow it to serve as a blood vessel dilator, or the inner catheter may decrease in profile in order to provide for better flexibility and ease of removal from the outer device after delivery. Alternatively different areas of the inner catheter have different outer diameters. In another example the inner catheter features one or more balloons serving as friction locks (see FIGS. 81-83 XX) to increase the friction between the inner catheter and aspiration catheter in order to provide the stiffness required for the combined system to be directly inserted into the vessel and serve as the blood vessel dilator.

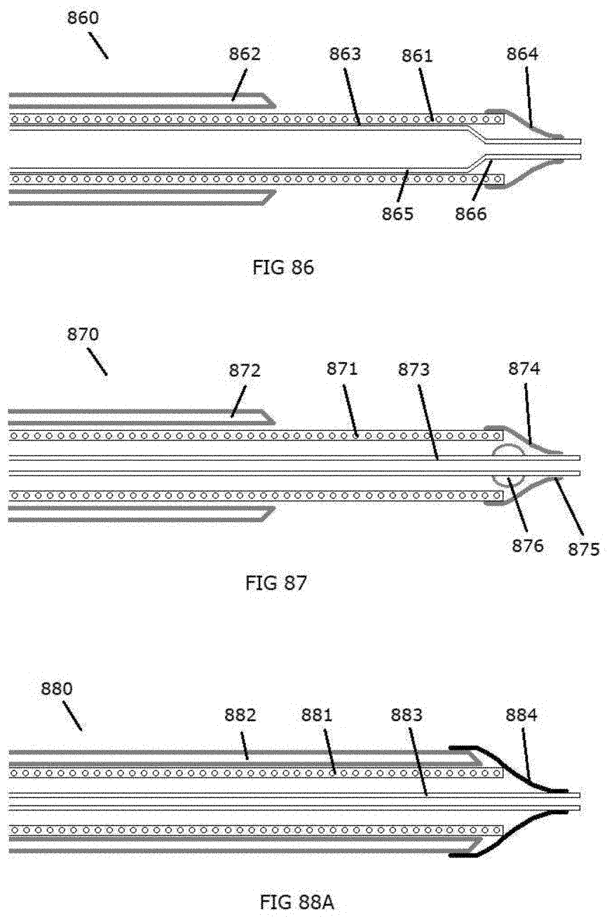

FIG. 86 shows an example of an aspiration catheter assembly 860 consisting of an aspiration catheter 861 supported by an outer guiding sheath 862 and a removable inner catheter 863 with a collapsing sleeve transition structure 864 which overlaps the distal leading edge of the aspiration catheter. In this example the inner member features a large proximal region 865 designed to regularly contact the inside of the aspiration catheter to increase stiffness and push, and a smaller distal region 866 sized to accept the collapsing sleeve after it is pushed off the aspiration catheter. I another example the smaller distal section extends proximally for a distance to increase the flexibility of the distal end of the combined systems, while the larger diameter proximal end increases their push. In one example the low profile distal section is between 1 cm and 25 cm in length, more preferably between 2 cm and 15 cm.

In another variant of the present invention, the collapsible sleeve or transition structure is not connected to the inner catheter but can slide along it such that the inner and outer catheters can move freely in the axial direction with respect to each other to aid in system delivery to the site of treatment, and the inner catheter features a flared section or other bumper designed to engage the collapsible sleeve or structure and allow it to be pulled off the outer catheter.

FIG. 87 shows an example of an aspiration catheter assembly 870 consisting of an aspiration catheter 871 supported by an outer guiding sheath 872 and a removable inner catheter 873 with a collapsing sleeve transition structure 874 which overlaps the distal leading edge of the aspiration catheter. The collapsing sleeve is not physically attached to the inner member at point 875, such that the inner catheter and aspiration catheter can be moved independently of each other in order to allow the physician more flexibility when negotiating difficult tortuosity. The collapsing sleeve is semi-rigid at its distal end, and there is sufficient clearance at the position 875 between the sleeve and the removable inner member to allow the inner member to slide back and forth with respect to the sleeve. The removable inner catheter further features a short flared section 876 which can push on the collapsing sleeve to pull it off the end of the outer catheter. The sleeve will then collapse over the flared section and can be removed from the anatomy along with the inner catheter prior to initiation of aspiration. The flared section may be a polymer bullet or slug as previously discussed, a metallic ring, band or cage, or of other construction. It may be made of radiopaque material to aid the physician in visualizing the position and state of the device.

In a preferred example the aspiration catheter of the present invention is provided by the manufacturer as part of an integrated system comprising an aspiration catheter and removable inner catheter, or an aspiration catheter and an outer sheath, or an aspiration catheter with a removable inner catheter and an outer sheath. All components in the integrated system are designed for enhanced synergy, resulting in a neurovascular treatment system of overall superior performance compared to a collection of conventional off-the-shelf devices selected independently by the physician. The components may be packaged and sterilized pre-assembled in a coaxial configuration, or intended to be integrated by the physician at the time of the procedure. Some advantages of the integrated system compared to a collection of conventional devices are (1) the integrated system can be easily inserted into a blood vessel directly over a guidewire with no extra introducer sheath or vessel dilator needed, (2) the integrated system can track over the wire through the aorta, over the aortic arch, and into the carotid arteries without need for a separate guiding sheath or guiding catheter to provide support and direction, (3) the integrated system can flexibly navigate through tortuous neurovascular anatomy without hanging up on the ophthalmic artery or other vascular side branches, (4) use of the integrated system may reduce procedure time and time is brain is neurological stroke cases. By eliminating the need for a separate introducer sheath, guiding sheath, guiding catheter, and/or vessel dilator the integrated system reduces procedure time which has been shown to improve patient outcomes. The enhanced performance of the integrated system enhances one or more of: dimensional compatibility and being able to maximize the aspiration lumen size, stiffness compatibility and being able to have an assembly having optimal stiffness to enter into and navigate through the blood vessel, and the incorporation of a transition structure to facilitate ease of entry and/or navigation through the blood vessel.

First, the inner and outer diameters of the components are designed for optimum dimensional compatibility with each other. The aspiration catheter itself, with or without a distal expandable scaffold as discussed previously, is the key component of the system since it is the largest component that must be tracked all the way through tortuous neurovascular anatomy and up to the clot. The inner diameter, outer diameter, and wall thickness of the aspiration catheter will be determined as part of the general device design based on the vessel size and location to be treated. The outer sheath, if present, can then be sized around the aspiration catheter. Typically, the inner diameter of the outer sheath is larger than the outer diameter of the aspiration catheter, with clearance allowing the two components can slide freely with respect to each other. In one example the clearance between the aspiration catheter and outer sheath ranges from 0.003″ and 0.008″, more preferably 0.004″-0.005″. The removable inner catheter is also sized to the aspiration catheter. In one example the outer diameter of the inner catheter may be close to the inner diameter of the aspiration catheter, as shown in FIG. 86 to provide enhanced pushability and track. Alternatively the inner catheter may be smaller and designed to interface optimally with the guidewire, and the space between the inner and aspiration catheters at the tip of the devices filled by the transition structure described further below. Blood loss from leakage in the annular space between any two components can be arrested with conventional hemostatic valves attached to the catheter hubs, or hemostasis can be achieved by sizing the inner diameter of an outer device very close to the outer diameter of an inner device to minimize the annular space. The outer device, whether a guiding sheath sealing against an aspiration catheter or an aspiration catheter sealing against an inner catheter, may feature elastomeric rings or other seals to improve the seal.

Second, the relative stiffnesses of the components are such that the combined system has a smooth stiffness transition from the guidewire all the way to the proximal shaft of the devices. In a conventional system there are significant step changes in stiffness from distal to proximal: guidewire only, then wire+microcatheter, then wire+microcatheter+aspiration catheter. While each individual component may gradually increase in stiffness proximally, there are still step increases at the start of each new component, and these increases will tend to initially resist turning into a curve in the vasculature, instead plowing into the vessel wall and increasing friction and vessel trauma. In comparison, the integrated system features components designed and used together and will provide more consistent performance. A step up in stiffness at the tip of one component can be offset by a reduction in the stiffness of the adjacent inner component, and/or the tip of the outer component can be softer than would typically needed as a standalone version, since it is designed to work with the other components and may require less rigidity to push along the vessels.

Third, the integrated system incorporates a transition structure providing a smooth leading edge and profile transition from the guidewire up to the maximum diameter of the overall system. In a preferred embodiment the transition structure comprises a collapsing sheath as previously shown in FIGS. 85-87. In one example, the transition structure extends from the aspiration catheter to the guiding sheath. In another example, the transition structure consists of two collapsing sheaths, one from the inner catheter to the aspiration catheter and a second from the aspiration catheter to the guiding sheath. In another and most preferred example a single collapsing sheath transition structure extends from the inner catheter over the aspiration catheter and to the outer sheath, providing a single smooth transition structure across the three components. In all variants in a preferred example the transition structure serves to provide a smooth and preferably continuous leading edge to the integrated system allowing the components to be inserted over the guidewire and into the vessel together without a separate dilator. The taper on the transition structure reduces the force required to insert the devices into the vessel, providing for atraumatic insertion into the anatomy and preventing the system from getting hung up on side branches that the guidewire may be bridging. The transition structure may be loosely overlapping the aspiration catheter and/or outer sheath, or any components may be more firmly but detachably coupled until the time of their release. It may slightly cover the outer device(s) by a few millimeters in some examples, or extend along a significant portion of the distal segment, for example up to 10 cm.

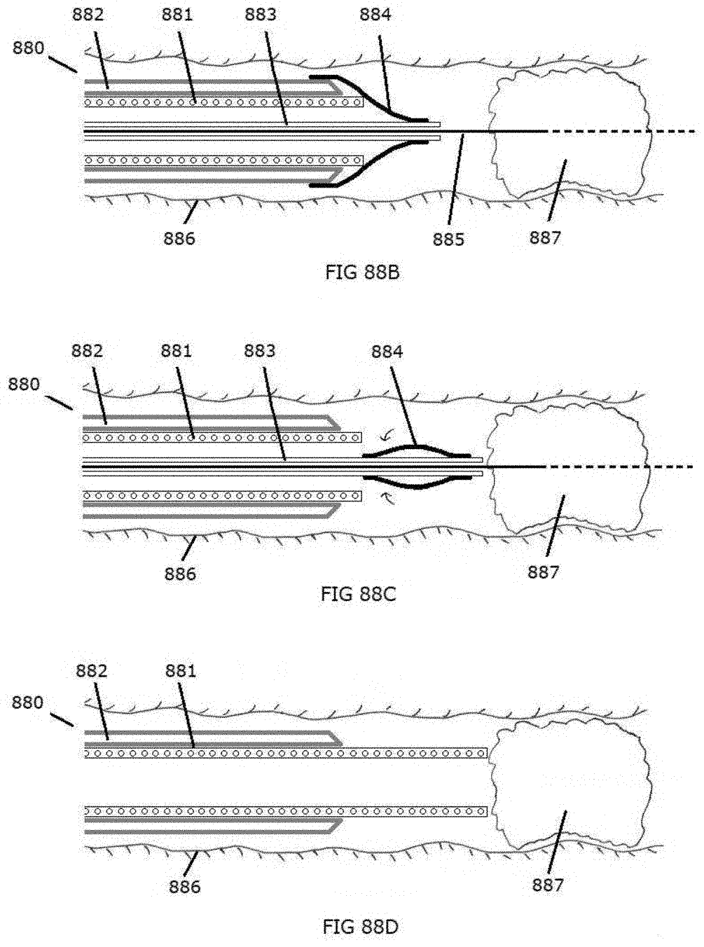

FIG. 88A shows an example of an aspiration catheter assembly 880 consisting of an aspiration catheter 881 supported by an outer guiding sheath 882 and a removable inner catheter 883. The inner catheter features a collapsing sleeve transition structure 884 which overlaps the distal leading edges of both the aspiration catheter and the guiding sheath such that the combined system has sufficient stiffness and a smooth distal profile transition to be introduced into the patient directly over the guidewire, with no separate dilator needed. In its natural state the proximal end of the transition structure has an inner diameter smaller than that of the distal end of the aspiration catheter, such that when the sleeve is decoupled from aspiration catheter it will collapse towards the inner catheter.

FIG. 88B shows the same assembly after delivery over a guidewire 885 into a neurovascular vessel 886 with a clot 887.

FIG. 88C shows the system after the inner member has been advanced a sufficient amount to pull the transition structure off the aspiration catheter and guiding sheath, and allowed it to collapse against the inner catheter.

FIG. 88D shows the system after the inner catheter and guidewire have been removed and the aspiration catheter advanced into contact with the clot, at which point aspiration would be initiated.

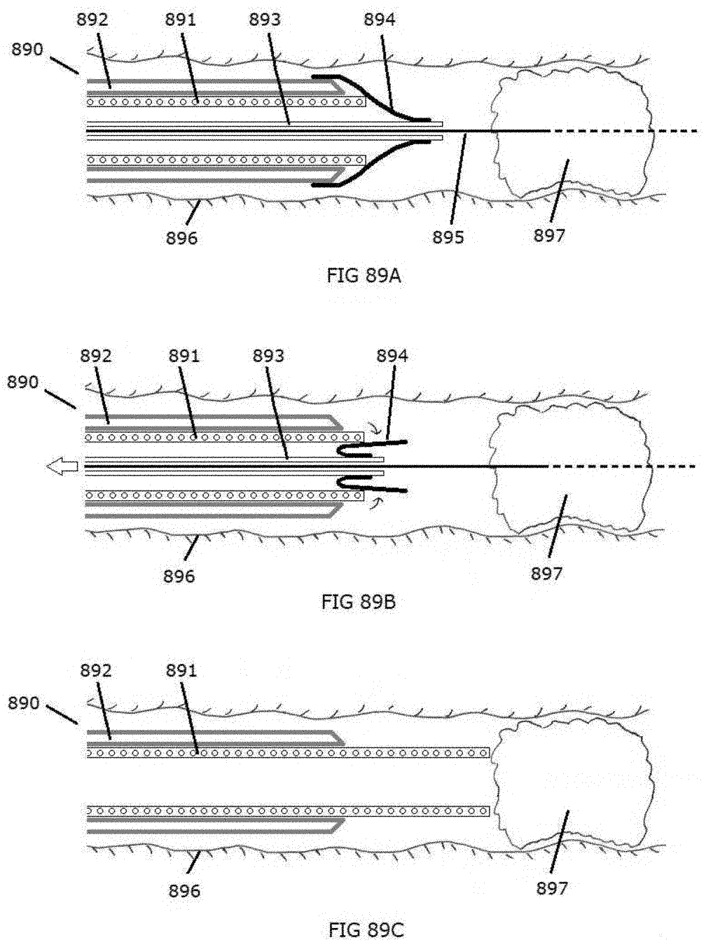

FIGS. 89A-C shows an example of an aspiration catheter assembly 890 consisting of an aspiration catheter 891 supported by an outer guiding sheath 892 and a removable inner catheter 893. Like the prior examples the inner catheter features a sleeve transition structure 894 which overlaps the distal ends of both the aspiration catheter and the guiding sheath, but in this case the inner catheter is pulled back directly causing the sleeve to invert to release it from the aspiration catheter and guiding sheath. FIG. 89A shows the assembly after delivery over a guidewire 895 into a neurovascular vessel 896 with a clot 897. FIG. 89B shows the system with the inner catheter 893 being withdrawn from the anatomy (left arrow) causing the transition structure 894 to invert such that it can be drawn into and through the aspiration catheter 891. Depending on the relative diameter and overall length of the sleeve, as the sleeve turns inside out its proximal end may enter the aspiration lumen after its distal end has done so. FIG. 89C shows the system after the inner catheter and guidewire have been removed and the aspiration catheter advanced into contact with the clot, at which point aspiration would be initiated.

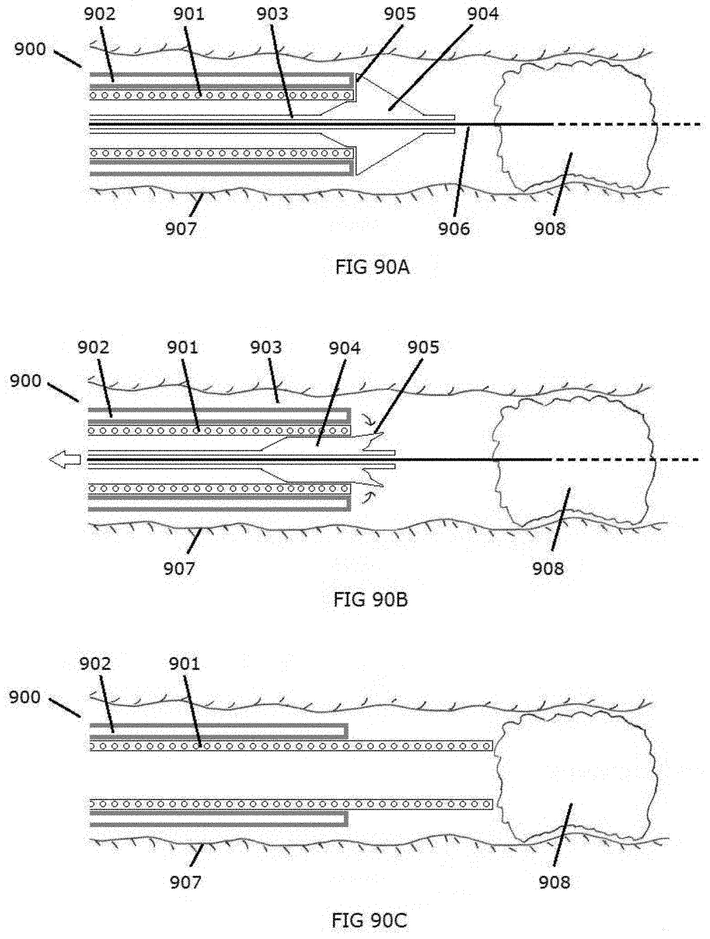

FIGS. 90A-C shows an example of an aspiration catheter assembly 900 consisting of an aspiration catheter 901 supported by an outer guiding sheath 902 and a removable inner catheter 903. In this example the transition structure is molded into the inner catheter rather than originally being a separately attached piece. The example shown shows a conically tapered transition structure, but hemispherical, bullet shaped, stepped profiles, other profiles, and combinations thereof are also included. The integrated transition structure 904 features a proximal-direct lip 905 which overlaps the distal ends of both the aspiration catheter and the guiding sheath. The transition structure may be molded from a variety of soft and flexible polymeric compounds, preferably one from the same selection previously described for use as collapsing sleeves. Whether attached or decoupled from the aspiration catheter the transition structure may have a larger profile than the distal end of the aspiration catheter, but the material used is soft enough that the inner catheter may be pulled directly back into the aspiration catheter, which causes the lip of the transition structure to invert thereby releasing it from the aspiration catheter and guiding sheath. In one variant the material used is sufficient compressible that it collapsed upon pullback into the aspiration catheter rather than inverting or deflecting. FIG. 90A shows the assembly after delivery over a guidewire 906 into a neurovascular vessel 907 with a clot 908. FIG. 90B shows the system with the inner catheter 903 being withdrawn (left arrow) causing the lip of the transition structure to invert such that it can be drawing into and through the aspiration catheter 901. FIG. 90C shows the system after the inner catheter and guidewire have been removed and the aspiration catheter advanced into contact with the clot, at which point aspiration would be initiated.

The transition structure may also be a collapsing sheath which is attached to the aspiration catheter and extends proximally over the outer guiding sheath. This transition structure would similarly require the aspiration catheter to be pushed distal to pull off the collapsing sheath and allow it to fall back against the aspiration catheter for removal, or depending on the design of the sheath it may be pulled proximally and simply invert to detach from the outer guiding sheath.

In another example the transition structure is coupled to the outer catheter and/or aspiration catheter and extends distal to cover and protect the ends of underlying devices. The coupling may comprise a relatively finite point, or extend along a significant portion of the outer surface of the devices, for example up to 10 cm. In this case the transition structure will have some ability to self-expand such that when it is released it will expand away from the inner device(s) allowing them to be removed from the system. In a preferred example a distally-extending transition structure comprises a shape memory polymer, a nickel-titanium alloy covered with a polymer sleeve, or both. The transition structure may be constrained in position during delivery by adhesive, sutures, or it may be folded or tucked inside the distal end of an underlying component. Axial tensile and/or compressive forces applied between the components will serve to dislodge or detach a distally-extending transition structure causing the distal tip of the structure to relax larger thereby allowing any inner devices to be removed. In a preferred example the transition structure is preformed into a generally conical shape to provide the smoothest and most atraumatic profile transition.

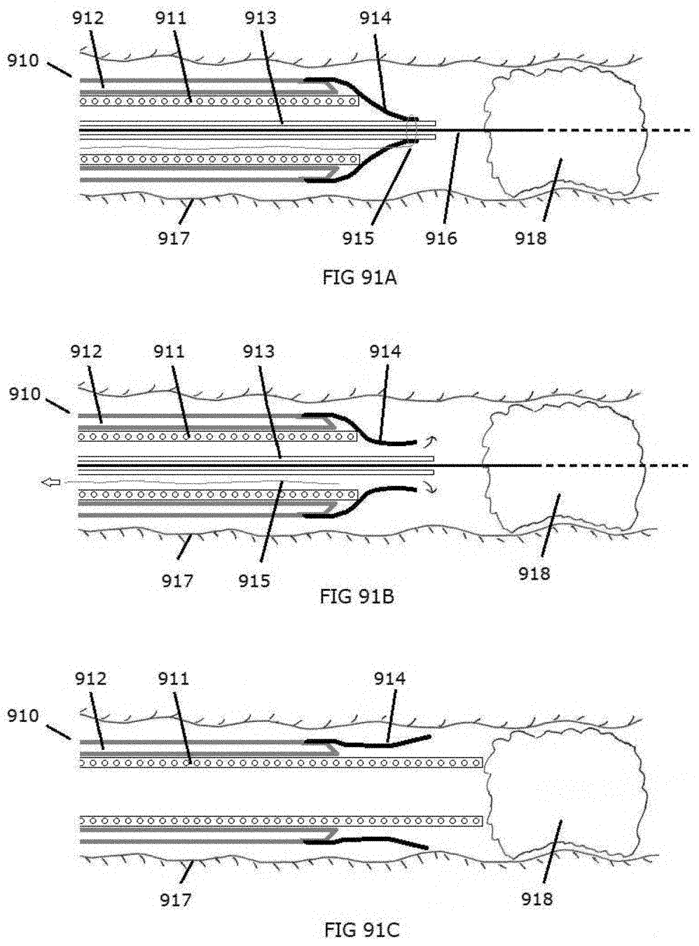

FIGS. 91A-C shows an example of an aspiration catheter assembly 910 consisting of an aspiration catheter 911 supported by an outer guiding sheath 912 and a removable inner catheter 913. Integrated with or separately attached to the outer sheath is a distally-extending transition structure 914 which is tethered to the inner catheter by a loop of filament 915 with a slip knot, which is pulled to cause the transition structure to expand thereby allowing the inner member to be removed and the aspiration catheter position to be adjusted prior to aspiration. In a preferred embodiment the filament is braided ultra-high molecular weight polyethylene fiber such as Spectra™ or Dyneema™. It may also be a loop of suture, metallic wire, or any other small diameter linear element of reasonable flexibility and tensile strength. FIG. 91A shows the assembly after delivery over a guidewire 916 into a neurovascular vessel 917 with a clot 918. FIG. 91B shows the system after the filament 915 has been pulled (left arrow) causing the transition structure 914 release away from the inner catheter 913. FIG. 91C shows the system after the inner catheter and guidewire have been removed and the aspiration catheter pushed forward through the transition structure and into contact with the clot, at which point aspiration would be initiated. In another example the suture or other filament used to constrain the distal end of the transition structure to the inner catheter does not need to use a slip knot and be tensioned to release the transition structure but more conventionally ties the components together with knots or loops of sufficient to keep components together during delivery to the clot, but the coupling is sufficiently compliant that the transition structure can be released by advancing or retracting either the aspiration catheter and/or inner catheter.

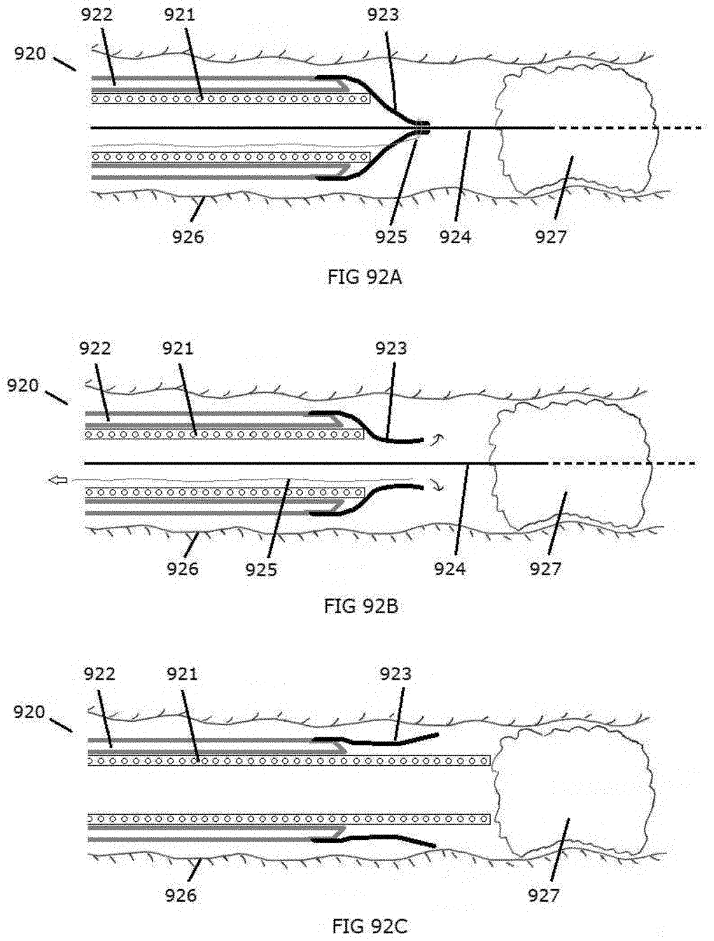

FIGS. 92A-C show an example of an aspiration catheter assembly 920 consisting of an aspiration catheter 921 supported by an outer guiding sheath 922 with integral distally-extending transition structure 923. In this example an inner catheter is omitted or optional, and the transition structure is configured to extend down to the guidewire 924 to allow the aspiration catheter and outer sheath to track smoothly along the wire. The end of the transition structure will typically be folded into a star or spiral shape and is constrained in the closed position by a loop of filament 925 with a slip knot, which is pulled to cause the transition structure to expand. In the constrained state the distal tip of the transition structure is close to the guidewire but not binding against it thereby allowing the system to be advanced smoothly over the guidewire. FIG. 92A shows the assembly after delivery over the guidewire into a neurovascular vessel 926 with a clot 927. FIG. 92B shows the system after the filament 925 has been pulled (left arrow) causing the transition structure 923 release away from the guidewire 924. FIG. 92C shows the system after the guidewire has been removed and the aspiration catheter pushed forward through the transition structure and into contact with the clot, at which point aspiration would be initiated. In a variant of the present example the distal tip of the transition structure has sufficient clearance over the guidewire that an inner catheter may be advanced over the guidewire and pass through the transition structure if such becomes necessary during the procedure.

In another example the integrated system may further incorporate an intermediate catheter intended to support the system during introduction into the anatomy and advancement through the femoral artery, and potentially up the aorta and over the aortic arch. The intermediate catheter is intended to partially or substantially fill the annular space between the inner catheter (or guidewire, if inner catheter is absent) and the aspiration lumen, thereby preventing flexing or buckling of the aspiration catheter and/or outer sheath (if present) during introduction into the artery. The distal tip of the intermediate catheter is contoured to match with the transition structure, for example the intermediate catheter may have a conical shape molded into its tip which slides partially under a conical collapsing sleeve transition structure and reinforces it during introduction into the artery. The intermediate catheter supports the aspiration catheter and inner catheter during advancement into the anatomy until the firmer proximal ends of the aspiration catheter and inner catheter are far enough inside the vascular that they themselves can be used to push the catheters farther in. At this point the intermediate catheter can be retracted from the tip of the other devices or withdrawn entirely, and the remaining system tracked to the clot as previously described.

In one example the intermediate catheter is intended to be retracted a distance of at least 10 cm, preferably at least 20 cm, and more preferably at least 40 cm, sufficient to allow the distal ends of the inner and aspiration catheters to freely navigate the neurovascular anatomy while maintaining support of the proximal end. Since the inner catheter will typically have a proximal hub allowing the lumen to be flushed and to ease guidewire insertion or exchange, the intermediate catheter will not be able to be fully removed and the inner catheter is sufficiently longer than the aspiration catheter to allow the required retraction of the intermediate catheter. In another example, the inner and intermediate catheters are withdrawn such that the intermediate catheter can be disengaged from the system, and then the inner catheter is re-inserted and the procedure continues as previously described. However in a preferred example the intermediate catheter can be peeled away from the inner catheter and removed from the system entirely without having to reposition or remove inner catheter. In this example the intermediate catheter may be made from a polymer with an axially aligned microstructure, which can be peeled apart without difficulty (I.E. FEP). In another example the intermediate catheter is a rapid exchange type design (see FIG. 77) and only the distal portion of the intermediate catheter is coaxially aligned over the inner catheter and/or guidewire. In a preferred example the distal portion is between 10 cm and 25 cm long, and proximal to the exit junction the intermediate catheter comprises a braided shaft, coil reinforced shaft, mandrel, rod, or hypotube of metal or stiff polymer which runs adjacent to the inner catheter within the proximal remainder of the aspiration catheter. In another example the proximal portion of the intermediate catheter coaxial and peelable but the distal portion is coaxial but not peelable, as may be a superior balance of provided support and ease of use.

In another example the intermediate catheter is reversibly retractable, and its position may be adjusted through the procedure as required to provide best overall deliverability of the system. For example, the tip of the intermediate catheter may be maintained somewhat proximal to a challenging bend in the vascular tortuosity, such that the push force applied by the physician is well transmitted to that location but the additional stiffness of the intermediate catheter does not push the aspiration catheter into the vessel wall. The system is advanced a few centimeters so the aspiration catheter goes around the bend and until the tip of the intermediate catheter approaches the bend, then the intermediate catheter is pulled back a few centimeters while the aspiration catheter is held constant, then the combined system is advance again. Performed repeatedly in this manner the intermediate catheter helps the inner and/or aspiration catheters inch forward around a tight curve in the anatomy.

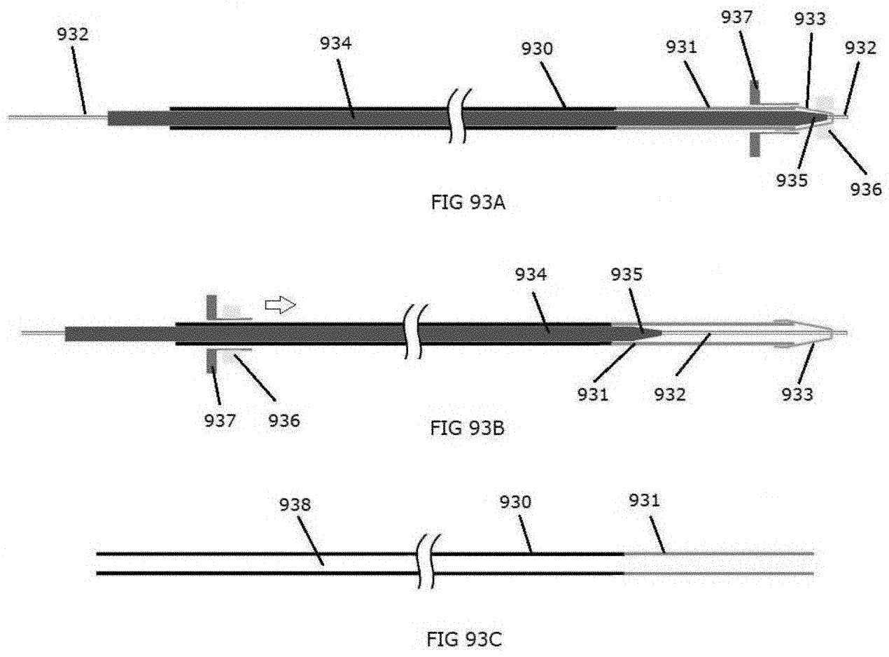

FIGS. 93A-C illustrate the operation of a system incorporating an intermediate catheter. FIG. 93A shows the system at time of introduction into the patient comprising an aspiration catheter with a proximal shaft 930 and a distal shaft 931, inside of which is an inner catheter 932 with conical collapsing sleeve transition structure 933. The intermediate catheter 934 lays over the inner catheter and within the aspiration lumen of the aspiration catheter. The intermediate catheter tip 935 has been molded into a conical shape to conform to and support the transition structure 933. Together the distal end of the system is rigid enough to be easily inserted into the artery 936 without a separate dilator catheter being necessary. Optionally the system may also be used with a short thin wall introducer sheath 937 to aid in reducing vessel trauma during device advancement and to reduce blood loss at the puncture site.

FIG. 93B shows the system after the inner catheter, intermediate catheter, and aspiration catheter have been inserted further into the patient (arrow), and after the intermediate catheter 934 has been pulled back such that its tip 935 has been substantially retracted from the aspiration catheter distal shaft 931.

FIG. 93C shows the system after the intermediate catheter, inner catheter and transition structure have been removed leaving the aspiration catheter 930, 931 with an unoccluded aspiration lumen 938 ready to aspirate the clot.

The intermediate catheter may be made from any of the catheter and sleeve materials previously discussed herein. In a preferred example the intermediate catheter distal tip is made from a moderately soft polymer such as Pebax 35D which is firm enough to push cleanly into the vessel without buckling but can provide some flexibility for following the guidewire up the aorta. More proximal the intermediate catheter may be made the same polymer or other polymers, such as increasingly firm grades of Pebax such as 40D, 55D, then 63D. In the preferred examples the proximal portion of the intermediate catheter may be made from the same polymers, firmer compounds such as Pebax 72D or Nylon 12, peelable polymers such as FEP or PTFE, or a hypotube of stainless steel or nickel titanium. The outer and/or inner surfaces of the intermediate catheter may be coated with a silicone oil or hydrophilic coating in order to ease its retraction from and/or repositioning within the aspiration catheter. In another example the intermediate catheter comprises an elongated rod with a tapered tip and containing an inner lumen intended to receive an inner catheter and/or guidewire. The elongated rod may be relatively stiffer than the other components in the system and is primarily intended only for aiding introduction of the system into the patient's vasculature.

The presence of the intermediate catheter and added support it provides may also allow for the outer guiding sheath to be optionally omitted from the system, or for the guide sheath to be replaced with a short thin wall sheath intended primarily to aid hemostasis at the vascular access site. Elimination of the outer guiding sheath provides a key profile advantage, allowing the inner diameter of the aspiration catheter to be increased and/or the overall profile of the system (and blood vessel puncture hole size) to be reduced. Similarly, the presence of the intermediate catheter may allow for the inner catheter to be reduced in size or eliminated altogether, since the intermediate catheter serves to provide the same track and push assistance to the aspiration catheter.

In yet another example an integrated system incorporates an outer sheath or guiding sheath which further features a distal scaffold or other mechanism of sealing against the vessel to provide the advantages of vessel sealing previously discussed. Alternatively, the outer sheath may be intended to advance sufficiently distal to the point where its outer diameter matches the inner diameter of the vessel.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

What is claimed is:

1. An aspiration catheter for removing clot from a blood vessel, said aspiration catheter comprising:
a catheter body having a proximal end, a distal end, and an aspiration lumen therebetween;
a scaffold extending distally from the distal end of the catheter body and having a central clot-receiving passage contiguous with the aspiration lumen of the catheter body; and
a membrane comprising an elastic sleeve covering the scaffold to establish a clot aspiration path from a distal end of the scaffold to a proximal end of the lumen in the catheter body so that applying a vacuum to a proximal end of the aspiration lumen can draw clot into the central clot-receiving passage;
wherein at least a distal portion of the scaffold is radially expandable from a delivery configuration to an extraction configuration and wherein the distal portion of the scaffold is configured to controllably collapse from the extraction configuration to a partially collapsed configuration in response to a vacuum applied within the central clot-receiving passage, wherein said collapsed configuration is sufficient to allow the aspiration of the clot into the aspiration lumen.

2. The aspiration catheter as in claim 1, wherein the scaffold is embedded in the membrane.

3. The aspiration catheter as in claim 1, wherein membrane is attached to the scaffold.

4. The aspiration catheter as in claim 1, wherein the width of the central clot-receiving passage when the distal portion is in its partially collapsed configuration is in a range from 0.25 to 0.75 of a width of the central clot-receiving passage when the distal portion is in the radially expanded configuration.

5. The aspiration catheter as in claim 1, wherein the distal portion of the scaffold is configured to collapse to one of a substantially flat configuration, a smaller cylindrical, or smaller conical configuration.

6. The aspiration catheter as in claim 1, wherein the scaffold is configured to partially collapse when a vacuum in a range from 0.2 atm to 1 atm is applied to the central clot-receiving passage.

7. The aspiration catheter as in claim 1, wherein the scaffold self-expands to the extraction configuration when a pressure in the central clot-receiving passage is above 0.2 atm.

8. The aspiration catheter as in claim 1, wherein the radially expandable distal portion of the scaffold is configured to be reversibly reconfigured between a radially contracted configuration, a radially expanded configuration, and a partially collapsed configuration

9. The aspiration catheter as in claim 1, wherein the radially expanded extraction configuration comprises a substantially cylindrical distal region configured to engage an inner wall of the blood vessel and a tapered transition region between the cylindrical distal region and the distal end of the catheter body, wherein the cylindrical distal region has an open distal end configured to direct clot into the central clot-receiving passage when the vacuum is applied to a proximal end of the aspiration lumen.

10. The aspiration catheter as in claim 1, wherein the radially expanded extraction configuration comprises a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen.

11. The aspiration catheter of claim 1, wherein the scaffold comprises struts joined by crown,

12. The aspiration catheter of claim 11, further comprising stops on adjacent struts to limit the collapse of the scaffold under pressure.

13. The aspiration catheter of claim 13, wherein the stops comprise circumferentially aligned tabs.

14. The aspiration catheter of claim 1, wherein the scaffold comprises a polymeric material.

15. The aspiration catheter of claim 1, wherein the scaffold comprises a plastically deformable material.

16. The aspiration catheter of claim 1, wherein the scaffold comprises an elastomeric material.

17. A method for extracting clot from a blood vessel, said method comprising:
positioning a radially expandable distal portion of an aspiration catheter in a blood vessel proximal to the clot;
radially expanding the radially expandable distal portion of the aspiration catheter in the blood vessel to form an enlarged central clot-receiving passage through the radially expandable distal portion contiguous with an aspiration lumen in the aspiration catheter; and
applying a first level of vacuum to a proximal portion of the aspiration lumen to draw clot from the blood vessel into the radially expandable distal portion of the aspiration catheter;
increasing the vacuum level after the clot has been drawn into the radially expandable distal portion of the aspiration catheter, wherein the increased level of vacuum causes the radially expandable distal portion to partially collapse to disrupt the clot.

18. The method of claim 17, wherein the radially expandable distal portion of the aspiration catheter comprises a scaffold covered with a vacuum-resistant membrane and wherein struts of the scaffold act to break and/or shear the clot as the radially expandable distal portion is partially collapsed by increasing the vacuum level.

19. The method of claim 17, wherein the radially expandable distal portion of the aspiration catheter is partially collapsed to an average width in a range from 0.25 to 0.75 of an initial width of the radially expandable distal portion of the aspiration catheter.

20. The method of claim 17, wherein the first level of vacuum is in a range from 0 to 0.5 atmospheres

21. The method of claim 17, wherein the increased vacuum level is in a range from 0.2 atm to 1 atm.

22. The method of claim 21, wherein the vacuum level is cycled up and down to enhance clot disruption after the clot has been drawn into the radially expandable distal portion of the aspiration catheter.

23. A method for extracting clot from a blood vessel, said method comprising:
positioning a distal portion of an aspiration catheter in a blood vessel proximal to the clot;
said distal portion of the aspiration catheter comprise a central clot-receiving passage through the distal portion and is contiguous with an aspiration lumen in the aspiration catheter; and
applying a first level of vacuum to a proximal portion of the aspiration lumen to draw clot from the blood vessel into the distal portion of the aspiration catheter;
increasing the vacuum level after the clot has been drawn into the distal portion of the aspiration catheter, wherein the increased level of vacuum causes the distal portion to partially collapse to disrupt and/or extract the clot.

24. The method of claim 23, wherein the distal portion of the aspiration catheter comprises a scaffold covered with a vacuum-resistant membrane and wherein struts of the scaffold act to break and/or shear the clot as the distal portion is partially collapsed by increasing the vacuum level.

25. The method of claim 23, wherein the distal portion of the aspiration catheter is partially collapsed to an average width in a range from 0.25 to 0.75 of an initial width of the distal portion of the aspiration catheter.

26. The method of claim 23, wherein the first level of vacuum is in a range from 0 to 0.5 atmospheres.

27. The method of claim 26, wherein the increased vacuum level is in a range from 0.2 atm to 1 atm.
